(19) United States
(12) Patent Application Publication
(10) Pub. No.: US 2021/0147870 A1
(43) Pub. Date: May 20, 2021

(54) COMPOSITIONS AND METHODS FOR THE TREATMENT OF STARGARDT DISEASE

(71) Applicants: NightstaRx Limited, London (GB); Oxford University Innovation Limited, Oxford Oxfordshire (GB)

(72) Inventors: Gregory S. Robinson, Waltham, MA (US); Michelle E. McClements, Oxford (GB); Robert Maclaren, Oxford (GB)

(21) Appl. No.: 17/045,118

(22) PCT Filed: Apr. 5, 2019
(86) PCT No.: PCT/US19/26061

§ 371 Date: Oct. 2, 2020

Related U.S. Application Data
(60) Provisional application No. 62/653,085, filed on Apr. 5, 2018.
(60) Provisional application No. 62/765,181, filed on Aug. 16, 2018.
(60) Provisional application No. 62/734,479, filed on Sep. 21, 2018.
(60) Provisional application No. 62/774,004, filed on Nov. 30, 2018.

(51) Int. Cl. C12N 15/86; A61K 48/00; C07K 14/47; A61P 27/02

(52) U.S. Cl. CPC C12N 15/86; A61K 48/005; C07K 14/47; C12N 2830/48; C12N 2830/42; C12N 2750/14143; C12N 2830/001; A61P 27/02

(57) ABSTRACT

The present disclosure provides an adeno-associated viral (AAV) vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence of contiguous nucleotides corresponding to nucleotides 105 to 3597 of SEQ ID NO: 1; wherein the second nucleic acid sequence comprises a sequence of contiguous nucleotides corresponding to nucleotides 3806 to 6926 of SEQ ID NO: 1; wherein the first nucleic acid sequence and the second nucleic acid sequence each comprise a region of sequence overlap with the other; and wherein the region of sequence overlap comprises at least about 20 contiguous nucleotides of a nucleic acid sequence corresponding to nucleotides 3598 to 3805 of SEQ ID NO: 1. Also provided are uses of AAV vector systems in the prevention or treatment of disease.

### RELATED APPLICATIONS

This application claims the benefit of provisional application U.S. Ser. No. 62/653,085, filed Apr. 5, 2018, U.S. Ser. No. 62/765,181, filed Aug. 16, 2018, U.S. Ser. No. 62/734,479, filed Sep. 21, 2018, and U.S. Ser. No. 62/774,004, filed Nov. 30, 2018, the contents of each of which are herein incorporated by reference in their entirety.

### INCORPORATION OF SEQUENCE LISTING

The contents of the text file named “NIGH-010/001WO_SeqList.txt,” which was created on Apr. 3, 2019 and is 279 KB in size, are hereby incorporated by reference in their entirety.

### FIELD OF THE DISCLOSURE

The present disclosure relates to adeno-associated viral (AAV) vector systems and AAV vectors for expressing human ABCA4 protein in a target cell. The AAV vector systems and AAV vectors of the disclosure may be used in preventing or treating diseases associated with degradation of retinal cells such as Stargardt disease.

### BACKGROUND

Stargardt disease is an inherited disease of the retina that can lead to blindness through the destruction of light-sensing photoreceptor cells in the eye. The disease commonly presents in childhood leading to blindness in young people.

The most common form of Stargardt disease is a recessive disorder linked to mutations in the gene encoding the protein ATP Binding Cassette, sub-family A, member 4 (ABCA4). In Stargardt disease, mutations in the ABCA4 gene lead to a lack of functional ABCA4 protein in retinal cells. This in turn leads to the formation and accumulation of bisretinoid by-products, producing toxic granules of lipofuscin in Retinal Pigment Epithelial (RPE) cells. This causes degradation and eventual destruction of the RPE cells, which leads to loss of photoreceptor cells causing progressive loss of vision and eventual blindness.

There has been a long-felt and unmet need for an effective treatment for Stargardt disease that addresses the underlying cause of the disease.

### SUMMARY OF THE DISCLOSURE

The disclosure provides an AAV vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence of contiguous nucleotides corresponding to nucleotides 105 to 3597 of SEQ ID NO: 1; wherein the second nucleic acid sequence comprises a sequence of contiguous nucleotides corresponding to nucleotides 3806 to 6926 of SEQ ID NO: 1; wherein the first nucleic acid sequence and the second nucleic acid sequence each comprise a region of sequence overlap with the other; and wherein the region of sequence overlap comprises at least about 20 contiguous nucleotides of a nucleic acid sequence corresponding to nucleotides 3598 to 3805 of SEQ ID NO: 1.

The region of sequence overlap may be between 20 and 550 nucleotides in length; preferably between 50 and 250 nucleotides in length; more preferably between 175 and 225 nucleotides in length; and most preferably between 195 and 215 nucleotides in length.

The region of sequence overlap may also comprise at least about 50 contiguous nucleotides of a nucleic acid sequence corresponding to nucleotides 3598 to 3805 of SEQ ID NO: 1; preferably at least about 75 contiguous nucleotides; more preferably at least about 100 contiguous nucleotides; even more preferably at least about 150 contiguous nucleotides; and most preferably at least about 200 contiguous nucleotides.

In some embodiments, the first nucleic acid sequence comprises a sequence of contiguous nucleotides consisting of nucleotides 105 to 3597 of SEQ ID NO: 1. In some embodiments, the second nucleic acid sequence comprises a sequence of contiguous nucleotides consisting of nucleotides 3806 to 6926 of SEQ ID NO: 1.

In some embodiments, the first nucleic acid sequence comprises a sequence of contiguous nucleotides consisting of nucleotides 105 to 3597 of SEQ ID NO: 2. In some embodiments, the second nucleic acid sequence comprises a sequence of contiguous nucleotides consisting of nucleotides 3806 to 6926 of SEQ ID NO: 2.

In some embodiments, the region of sequence overlap comprises at least about 20 contiguous nucleotides of a nucleic acid sequence consisting of nucleotides 3598 to 3805 of SEQ ID NO: 1. In some embodiments, the region of sequence overlap comprises at least about 20 contiguous nucleotides of a nucleic acid sequence consisting of nucleotides 3598 to 3805 of SEQ ID NO: 2.

In some embodiments, the region of sequence overlap comprises at least about 50 contiguous nucleotides of a nucleic acid sequence consisting of nucleotides 3598 to 3805 of SEQ ID NO: 1; preferably at least about 75 contiguous nucleotides; more preferably at least about 100 contiguous nucleotides; even more preferably at least about 150 contiguous nucleotides; and most preferably at least about 200 contiguous nucleotides. In some embodiments, the region of sequence overlap comprises at least about 50 contiguous nucleotides of a nucleic acid sequence consisting of nucleotides 3598 to 3805 of SEQ ID NO: 2; preferably at least about 75 contiguous nucleotides; more preferably at least about 100 contiguous nucleotides; even more preferably at least about 150 contiguous nucleotides; and most preferably at least about 200 contiguous nucleotides.

In some embodiments, the first nucleic acid sequence comprises a sequence of contiguous nucleotides corresponding to nucleotides 105 to 3805 of SEQ ID NO: 1; and the second nucleic acid sequence comprises a sequence of contiguous nucleotides corresponding to nucleotides 3598 to 6926 of SEQ ID NO: 1.

In some embodiments, the first nucleic acid sequence comprises a sequence of contiguous nucleotides consisting of nucleotides 105 to 3805 of SEQ ID NO: 1; and the second nucleic acid sequence comprises a sequence of contiguous nucleotides consisting of nucleotides 3598 to 6926 of SEQ ID NO: 1.

In some embodiments, the first nucleic acid sequence comprises a sequence of contiguous nucleotides consisting of nucleotides 105 to 3805 of SEQ ID NO: 2; and the second nucleic acid sequence comprises a sequence of contiguous nucleotides consisting of nucleotides 3598 to 6926 of SEQ ID NO: 2.

The first AAV vector may comprise a GRK1 promoter operably linked to the 5′ end portion of an ABCA4 coding sequence (CDS).

The first nucleic acid sequence may comprise an untranslated region (UTR) located upstream of the 5′ end portion of an ABCA4 coding sequence (CDS).

The second nucleic acid sequence may comprise a post-transcriptional response element (PRE); preferably a Woodchuck hepatitis virus post-transcriptional response element (WPRE).

The second nucleic acid sequence may comprise a bovine Growth Hormone (bGH) poly-adenylation sequence.

The disclosure provides a method for expressing a human ABCA4 protein in a target cell, the method comprising the steps of: transducing the target cell with the first AAV vector and the second AAV vector as defined above, such that a functional ABCA4 protein is expressed in the target cell.

The disclosure provides an AAV vector comprising a nucleic acid sequence comprising a 5′ end portion of an ABCA4 CDS, wherein the 5′ end portion of an ABCA4 CDS consists of a sequence of contiguous nucleotides corresponding to nucleotides 105 to 3805 of SEQ ID NO: 1. In some embodiments, this AAV vector comprises the nucleic acid sequence of SEQ ID NO: 9. In some embodiments, the 5′ end portion of an ABCA4 CDS consists of nucleotides 105 to 3805 of SEQ ID NO: 1. In some embodiments, the 5′ end portion of an ABCA4 CDS consists of nucleotides 105 to 3805 of SEQ ID NO: 2.

The disclosure provides an AAV vector comprising a nucleic acid sequence comprising a 3′ end portion of an ABCA4 CDS, wherein the 3′ end portion of an ABCA4 CDS consists of a sequence of contiguous nucleotides corresponding to nucleotides 3598 to 6926 of SEQ ID NO: 1. In one embodiment, this AAV vector comprises the nucleic acid sequence of SEQ ID NO: 10. In one embodiment, the 3′ end portion of an ABCA4 CDS consists of nucleotides 3598 to 6926 of SEQ ID NO: 1. In one embodiment, the 3′ end portion of an ABCA4 CDS consists of nucleotides 3598 to 6926 of SEQ ID NO:2.

The disclosure provides a nucleic acid comprising the first nucleic acid sequence as defined above.

The disclosure provides a nucleic acid comprising the second nucleic acid sequence as defined above.

The disclosure provides a nucleic acid comprising the nucleic acid sequence of SEQ ID NO: 9, and a nucleic acid comprising the nucleic acid sequence of SEQ ID NO: 10.

The disclosure provides a kit comprising the AAV vector system as described above, or the upstream AAV vector and the downstream AAV vector as described above.

The disclosure provides a kit comprising a nucleic acid comprising the first nucleic acid sequence and a nucleic acid comprising the second nucleic acid sequence, as described above, or a nucleic acid comprising the nucleic acid sequence of SEQ ID NO: 9 and a nucleic acid comprising the nucleic acid sequence of SEQ ID NO: 10, as described above.

The disclosure provides a pharmaceutical composition comprising the AAV vector system as described above and a pharmaceutically acceptable excipient.

The disclosure provides an AAV vector system as described above, a kit as described above, or a pharmaceutical composition as described above, for use in preventing or treating disease characterized by degradation of retinal cells; preferably for use in preventing or treating Stargardt disease.

The disclosure provides a method for preventing or treating a disease characterized by degradation of retinal cells, such as Stargardt disease, comprising administering to a subject in need thereof an effective amount of an AAV vector system as described above, a kit as described above, or a pharmaceutical composition as described above.

The disclosure provides an AAV vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 105 to 3597 of SEQ ID NO: 1; wherein the second nucleic acid sequence comprises a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 3806 to 6926 of SEQ ID NO: 1; wherein the first nucleic acid sequence and the second nucleic acid sequence each comprise a region of sequence overlap with the other; and wherein the region of sequence overlap comprises at least about 20 contiguous nucleotides of a nucleic acid sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 3598 to 3805 of SEQ ID NO: 1.

The disclosure provides an AAV vector system for expressing a human ABCA4 protein in a target cell, the AAV vector system comprising a first AAV vector comprising a first nucleic acid sequence and a second AAV vector comprising a second nucleic acid sequence, wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the 5′ end portion of an ABCA4 CDS consists of a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 105 to 3805 of SEQ ID NO: 1, and wherein the 3′ end portion of an ABCA4 CDS consists of a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 3598 to 6926 of SEQ ID NO: 1.

The disclosure provides an AAV vector comprising a nucleic acid sequence comprising a 5′ end portion of an ABCA4 CDS, wherein the 5′ end portion of an ABCA4 CDS consists of a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 105 to 3805 of SEQ ID NO: 1.

The disclosure provides an AAV vector comprising a nucleic acid sequence comprising a 3′ end portion of an ABCA4 CDS, wherein the 3′ end portion of an ABCA4 CDS consists of a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 3598 to 6926 of SEQ ID NO: 1.

The disclosure provides an AAV vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence having at least 90% (e.g. at least 90%, 95%, 96%, 97%, 98%, 99%, 99.1%, 99.2%, 99.3%, 99.4%, 99.5%, 99.6%, 99.7%, 99.8%, 99.9% or 100%) sequence identity to nucleotides 105 to 3597 of SEQ ID NO: 2; wherein the second nucleic acid sequence comprises a sequence having at least 90% (e.g. at least 90%, 95%, 96%, 97%, 98%, 99%, 99.1%, 99.2%, 99.3%, 99.4%, 99.5%, 99.6%, 99.7%, 99.8%, 99.9% or 100%) sequence identity to nucleotides 3806 to 6926 of SEQ ID NO: 2; wherein the first nucleic acid sequence and the second nucleic acid sequence each comprise a region of sequence overlap with the other; and wherein the region of sequence overlap comprises at least about 20 contiguous nucleotides of a nucleic acid sequence having at least 90% (e.g. at least 90%, 95%, 96%, 97%, 98%, 99%, 99.1%, 99.2%, 99.3%, 99.4%, 99.5%, 99.6%, 99.7%, 99.8%, 99.9% or 100%) sequence identity to nucleotides 3598 to 3805 of SEQ ID NO: 2.

The disclosure provides an AAV vector system for expressing a human ABCA4 protein in a target cell, the AAV vector system comprising a first AAV vector comprising a first nucleic acid sequence and a second AAV vector comprising a second nucleic acid sequence, wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the 5′ end portion of an ABCA4 CDS consists of a sequence having at least 90% (e.g. at least 90%, 95%, 96%, 97%, 98%, 99%, 99.1%, 99.2%, 99.3%, 99.4%, 99.5%, 99.6%, 99.7%, 99.8%, 99.9% or 100%) sequence identity to nucleotides 105 to 3805 of SEQ ID NO: 2, and wherein the 3′ end portion of an ABCA4 CDS consists of a sequence having at least 90% (e.g. at least 90%, 95%, 96%, 97%, 98%, 99%, 99.1%, 99.2%, 99.3%, 99.4%, 99.5%, 99.6%, 99.7%, 99.8%, 99.9% or 100%) sequence identity to nucleotides 3598 to 6926 of SEQ ID NO: 2.

The disclosure provides an AAV vector comprising a nucleic acid sequence comprising a 5′ end portion of an ABCA4 CDS, wherein the 5′ end portion of an ABCA4 CDS consists of a sequence having at least 90% (e.g. at least 90%, 95%, 96%, 97%, 98%, 99%, 99.1%, 99.2%, 99.3%, 99.4%, 99.5%, 99.6%, 99.7%, 99.8%, 99.9% or 100%) sequence identity to nucleotides 105 to 3805 of SEQ ID NO: 2.

The disclosure provides an AAV vector comprising a nucleic acid sequence comprising a 3′ end portion of an ABCA4 CDS, wherein the 3′ end portion of an ABCA4 CDS consists of a sequence having at least 90% (e.g. at least 90%, 95%, 96%, 97%, 98%, 99%, 99.1%, 99.2%, 99.3%, 99.4%, 99.5%, 99.6%, 99.7%, 99.8%, 99.9% or 100%) sequence identity to nucleotides 3598 to 6926 of SEQ ID NO: 2.

The disclosure provides a nucleic acid comprising a nucleic acid sequence having at least 90% (e.g. at least 90%, 95%, 96%, 97%, 98%, 99%, 99.1%, 99.2%, 99.3%, 99.4%, 99.5%, 99.6%, 99.7%, 99.8%, 99.9% or 100%) sequence identity to SEQ ID NO: 9, and a nucleic acid comprising a nucleic acid sequence having at least 90% (e.g. at least 90%, 95%, 96%, 97%, 98%, 99%, 99.1%, 99.2%, 99.3%, 99.4%, 99.5%, 99.6%, 99.7%, 99.8%, 99.9% or 100%) sequence identity to SEQ ID NO: 10.

The disclosure provides an adeno-associated viral (AAV) vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence of contiguous nucleotides corresponding to nucleotides 105 to 3597 of SEQ ID NO: 1; wherein the second nucleic acid sequence comprises a sequence of contiguous nucleotides corresponding to nucleotides 3806 to 6926 of SEQ ID NO: 1; wherein the first nucleic acid sequence and the second nucleic acid sequence each comprise a region of sequence overlap with the other; and wherein the region of sequence overlap comprises at least about 20 contiguous nucleotides of a nucleic acid sequence corresponding to nucleotides 3598 to 3805 of SEQ ID NO: 1. In some embodiments of the AAV vector system, the region of sequence overlap is between 20 and 550 nucleotides in length; preferably between 50 and 250 nucleotides in length; preferably between 175 and 225 nucleotides in length; or preferably between 195 and 215 nucleotides in length. In some embodiments of the AAV vector system, the region of sequence overlap comprises at least about 50 contiguous nucleotides of a nucleic acid sequence corresponding to nucleotides 3598 to 3805 of SEQ ID NO: 1; preferably at least about 75 contiguous nucleotides; preferably at least about 100 contiguous nucleotides; preferably at least about 150 contiguous nucleotides; preferably at least about 200 contiguous nucleotides; or preferably all 208 contiguous nucleotides.

In some embodiments of AAV vector systems of the disclosure, the first nucleic acid sequence comprises a sequence of contiguous nucleotides corresponding to nucleotides 105 to 3805 of SEQ ID NO: 1; and wherein the second nucleic acid sequence comprises a sequence of contiguous nucleotides corresponding to nucleotides 3598 to 6926 of SEQ ID NO: 1.

In some embodiments of AAV vector systems of the disclosure, the first nucleic acid sequence comprises a CBA promoter operably linked to the 5′ end portion of an ABCA4 coding sequence (CDS). In some embodiments, the first nucleic acid sequence further comprises a sequence encoding an intron. In some embodiments, the first nucleic acid sequence further comprises a sequence encoding an exon. In some embodiments, the first nucleic acid sequence further comprises a sequence encoding an Intron and an Exon (IntEx).

In some embodiments of AAV vector systems of the disclosure, the first nucleic acid sequence comprises an untranslated region (UTR) located upstream of the 5′ end portion of an ABCA4 coding sequence (CDS). In some embodiments, the UTR comprises a sequence encoding an enhancer. In some embodiments, the sequence encoding the enhancer comprises a sequence isolated or derived from a cytomegalovirus (CMV) (a CMV enhancer). In some embodiments, the sequence encoding the enhancer does not comprise a sequence isolated or derived from a cytomegalovirus (CMV) (a CMV enhancer).

In some embodiments, the first nucleic acid sequence further comprises a sequence encoding an Intron and an Exon (IntEx) and a UTR, wherein the UTR does not comprise a sequence isolated or derived from a cytomegalovirus (CMV) (a CMV enhancer).

In some embodiments of AAV vector systems of the disclosure, the second nucleic acid sequence comprises a post-transcriptional response element (PRE); preferably a Woodchuck hepatitis virus post-transcriptional response element (WPRE).

In some embodiments of AAV vector systems of the disclosure, the second nucleic acid sequence comprises a bovine Growth Hormone (bGH) poly-adenylation sequence.

In some embodiments of AAV vector systems of the disclosure, the first nucleic acid sequence or the second nucleic acid sequence further comprises a sequence encoding a 5′ inverted terminal repeat (ITR) and a sequence encoding a 3′ ITR. In some embodiments, the sequence encoding a 5′ ITR comprises a wild type sequence isolated or derived of a serotype 2 AAV (AAV2). In some embodiments, the sequence encoding the 5′ ITR comprises the sequence of SEQ ID NO: 27 or a deletion variant thereof. In some embodiments, the sequence encoding a 3′ ITR comprises a wild type sequence isolated or derived of an AAV2. In some embodiments, the sequence encoding the 3′ ITR comprises the sequence of SEQ ID NO: 30 or a deletion variant thereof. In some embodiments, the deletion variant comprises or consists of 10, 20, 30, 40, 50, 70, 80, 90, 100, 110, 120, 130, 140, 144 nucleotides or any number in between of nucleotides. In some embodiments, the deletion variant comprises one or more deletions. In some embodiments, the deletion variant comprises at least two deletions. In some embodiments, the at least two deletions are not contiguous. In some embodiments, the one or more deletions comprises a truncation of the ITR at either the 5′ or the 3′ end. In some embodiments, the deletion variant comprises a deletion of any one of the nucleotides of SEQ ID NO: 27. In some embodiments, the deletion variant comprises a deletion of any 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, or any number of nucleotides in between of SEQ ID NO: 27. In some embodiments, the deletion variant comprises a deletion of any one of the nucleotides of SEQ ID NO: 30. In some embodiments, the deletion variant comprises a deletion of any 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, or any number of nucleotides in between of SEQ ID NO: 30.

In some embodiments of AAV vector systems of the disclosure, the first nucleic acid sequence or the second nucleic acid sequence further comprises a sequence encoding a 5′ inverted terminal repeat (ITR) and a sequence encoding a 3′ ITR. In some embodiments, the sequence encoding a 5′ ITR comprises a wild type sequence isolated or derived of a serotype 2 AAV (AAV2). In some embodiments, the sequence encoding the 5′ ITR comprises the sequence of

(SEQ ID NO: 36)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCT.

In some embodiments, the sequence encoding a 3′ ITR comprises a wild type sequence isolated or derived of an AAV2. In some embodiments, the sequence encoding the 3′ ITR comprises the sequence of

(SEQ ID NO: 37)
AGGAACCCCTAGTGATGGAGTTGGCCACTCCCTCTCTGCGCGCTCGCTCG
CTCACTGAGGCCGGGCGACCAAAGGTCGCCCGACGCCCGGGCGGCCTCAG
TGAGCGAGCGAGCGCGCAGAG.

In some embodiments of AAV vector systems of the disclosure, the first nucleic acid sequence or the second nucleic acid sequence further comprises a sequence encoding a 5′ inverted terminal repeat (ITR) and a sequence encoding a 3′ ITR. In some embodiments, the sequence encoding a 5′ ITR comprises a wild type sequence isolated or derived of a serotype 2 AAV (AAV2). In some embodiments, the sequence encoding the 5′ ITR comprises the sequence of

(SEQ ID NO: 34)
CTGCGCGCTCGCTCGCTCACTGAGGCCGCCCGGGCGTCGGGCGACCTTTG
GTCGCCCGGCCTCAGTGAGCGAGCGAGCGCGCAGAGAGGGAGTGGCCAAC
TCCATCACTAGGGGTTCCT.

In some embodiments, the sequence encoding a 3′ ITR comprises a wild type sequence isolated or derived of an AAV2. In some embodiments, the sequence encoding the 3′ ITR comprises the sequence of

(SEQ ID NO: 35)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG.

The disclosure provides a cell comprising an AAV vector of the disclosure.

The disclosure provides a cell comprising a nucleotide encoding an AAV vector of the disclosure.

The disclosure provides a cell comprising a composition of the disclosure.

In some embodiments of the cells of the disclosure, the cell is a retinal cell. In some embodiments, the cell is a neuronal cell. In some embodiments, the cell is a photoreceptor cell. In some embodiments, the cell is a hexagonal cell of the retinal pigment epithelium (RPE).

The disclosure provides a method for expressing a human ABCA4 protein in a target cell, the method comprising the steps of: transducing the target cell with a first AAV vector and a second AAV vector of the disclosure, such that a functional ABCA4 protein is expressed in the target cell.

The disclosure provides a nucleic acid sequence comprising a 5′ end portion of an ABCA4 CDS, wherein the 5′ end portion of an ABCA4 CDS consists of a sequence of contiguous nucleotides corresponding to nucleotides 105 to 3805 of SEQ ID NO: 1.

The disclosure provides an AAV vector comprising a nucleic acid sequence comprising a 3′ end portion of an ABCA4 CDS, wherein the 3′ end portion of an ABCA4 CDS consists of a sequence of contiguous nucleotides corresponding to nucleotides 3598 to 6926 of SEQ ID NO: 1.

The disclosure provides a pharmaceutical composition comprising an AAV vector or an AAV vector system of the disclosure and a pharmaceutically acceptable excipient.

The disclosure provides a nucleic acid, a vector, an AAV vector, a composition, an AAV vector system, or a pharmaceutical composition of the disclosure for use in gene therapy.

The disclosure provides a nucleic acid, a vector, an AAV vector, a composition, an AAV vector system, or a pharmaceutical composition of the disclosure for use in preventing or treating disease characterized by degradation of retinal cells.

The disclosure provides a nucleic acid, a vector, an AAV vector, a composition, an AAV vector system, or a pharmaceutical composition of the disclosure for use in preventing or treating Stargardt disease.

The disclosure provides a method for preventing or treating a disease characterized by degradation of retinal cells comprising administering to a subject in need thereof an effective amount of a nucleic acid, a vector, an AAV vector, a composition, an AAV vector system, or a pharmaceutical composition of the disclosure.

The disclosure provides a method for preventing or treating Stargardt Disease comprising administering to a subject in need thereof an effective amount of a nucleic acid, a vector, an AAV vector, a composition, an AAV vector system, or a pharmaceutical composition of the disclosure.

### DESCRIPTION OF FIGURES

The patent or application file contains at least one drawing executed in color. Copies of this patent or patent application publication with color drawing(s) will be provided by the Office upon request and payment of the necessary fee.

FIG. 1 is a schematic drawing showing upstream and downstream transgene structures that combine to form a complete ABCA4 transgene.

FIG. 2A-C is a series of diagrams of transgene outcomes following transduction with an ABCA4 overlapping dual vector system. (A) Upstream and downstream transgene single-stranded DNA forms. These can anneal by single-strand annealing (SSA) via their regions of homology on complementary transgenes (B), following which the complete recombined large transgene can be generated (C). Abbreviations: CDS=coding sequence; DSB=double-stranded break; HR=homologous recombination; ITR=inverted terminal repeat; pA=polyA signal; SSA=single-strand annealing; WPRE=Woodchuck hepatitis virus post-transcriptional regulatory element.

FIG. 3 is a plot showing ABCA4 protein detection in Abca4−/− retinae 6 weeks post-injection with dual vector variant C with (5′C) and without (C) the extra UTR sequence. Units represent fold increase relative to uninjected KO samples. Error bars represent SEM. One-way ANOVA, Tukey post-hoc, p=**0.009.

FIG. 4 is a schematic diagram showing overlapping upstream and downstream dual vectors and a gel showing amplification of ABCA4 targeting a region spanning the overlap zones of dual vector variants from injected Abca4−/− eyes (n=4). A forward primer binding ABCA4 CDS in the upstream transgene and a reverse primer binding ABCA4 CDS in the downstream transgene were used to amplify transcripts from recombined transgenes. Amplicons were sequenced to confirm the correct ABCA4 CDS was contained across the overlap regions of the transcripts. B/C=eyes injected with dual vector variants B or C (see Table 2); 5′B=eyes injected with dual vector variant B in which the upstream transgene contains a 5′UTR; Bx=eyes injected with dual vector variant B in which the downstream transgene is without a WPRE; CDS=coding sequence; GFP=eyes injected with GRK1.GFP.pA AAV2/8 Y733F injected eyes; KO=uninjected Abca4^{-/-} eyes; Up=eyes injected with upstream B only; Up+Do=pooled cDNA from upstream vector only injected eyes and downstream vector only injected eyes; +=ABCA4 plasmid control.

FIG. 5 is a series of diagrams showing the overlapping upstream and downstream dual vectors and a gel of PCR products confirming 5′ UTR splicing from dual vector 5′C injected Abca4^{-/-} pooled retinae (n=4). A forward primer binding just downstream of the GRK1 transcriptional start site (TSS) and a reverse primer binding within the upstream ABCA4 CDS were used to assess transcript forms from dual vector C injected eyes and dual vector 5′C injected eyes (variants depicted above). ABCA4 transcripts from dual vector C injected eyes generated a single amplicon representing the original reference sequence. Transcripts from dual vector 5′C injected eyes generated three defined products which were sequenced and confirmed to be unspliced, partially spliced and fully spliced variants.

FIG. 6 is a graph showing the detection of full length ABCA4 protein from HEK293T cells transduced with dual vector variant B with and without a WPRE. Samples treated with AAV2/8 Y733F dual vector variant B (B) generated more ABCA4 than those treated with dual vector variant B without the WPRE (Bx) (unpaired two-tailed parametric t test, n=3, *p=0.01, F(2, 2)=17.06). Error bars represent SEM.

FIG. 7A-B are a pair of plots showing protein production from the dual vector upstream and downstream transgenes that make up overlap variants A, B, C, D, E, F and X. (A) ABCA4 protein detection following transduction with the different overlap zone vector variants (A) in vitro and (B) in vivo. Units represent fold increase relative to untreated samples (−=untreated HEK293T cells; KO=uninjected Abca4^{-/-} retinae). Error bars represent SEM. One-way ANOVA, Tukey post-hoc analyses revealed that in vitro, dual vector variants B and C generated more ABCA4 protein than all other samples but there was no significant difference between B and C. In vivo, dual vector variant C generated more ABCA4 protein than all other variants (except B).

FIG. 8A-D is a pair of gels, a table and a plot looking at ABCA4 expression. (A) Truncated ABCA4 protein variants detectable in HEK293T cells treated with unrecombined downstream vectors; (B) truncated and full length ABCA4 protein detected in Abca4^{-/-} retinae samples injected with dual vector 5′B or 5′C; (C) Table presents percentage full length ABCA4 present in the total ABCA4 protein population detected by western blot of injected retinae (D) difference in fold change of ABCA4 expression between overlap C dual vector variant injected retinae and overlap B dual vector variant injected retinae at transcript and protein level. Error bars represent SEM.

FIG. 9A-F is a diagram showing dual vector upstream and downstream variants A, B, C, D, E, F, G and X and a series of 4 gels and 5 plots showing the levels of ABCA4 expression from the different dual vector overlap variants. FIG. 9 shows an assessment of optimal combinations of upstream and downstream AAV2/8 Y733F ABCA4 dual vectors. Levels of full length and truncated ABCA4 (tABCA4) were influenced by the overlapping region of the dual vector system. Detection of full length ABCA4 protein was normalized to glyceraldehyde 3-phosphate dehydrogenase (GAPDH) per sample and presented as levels above untreated negative control samples. (A, E) Dual vector transductions of HEK293T cells successfully generated full length ABCA4. AAV2/8 Y733F dual vector transductions of HEK293T cells identified a significant influence of the overlapping region on the levels of ABCA4 generated in vitro (one-way ANOVA, n=6, p<0.0001, F(6,35)=12.81). Variant A generated significantly more ABCA4 than variants F and X whilst variants B and C generated significantly more ABCA4 than variants D, E, F and X (one-way ANOVA, Tukey's multiple comparisons test, A *p≤0.03, B ***p≤0.0002, C *p≤0.04). Variant A generated significantly more ABCA4 than variant X whilst variants B and C generated significantly more ABCA4 than variant X (one-way ANOVA, Tukey's multiple comparisons test, A **p=0.004, B ***p<0.0001, C ***p≤0.0004). Error bars represent SEM. (B) Abca4^{-/-} mice received sub-retinal injections of AAV2/8 Y733F dual vector variants with ABCA4 expression assessed 6 weeks post-injection. The overlap region influenced the levels of ABCA4 detected (one-way ANOVA, n=3-16, p=0.001, F(6,36)=4.453). Increasing the dose of the well performing variant (5′C) from 1×10⁹ to 10¹⁰ (5′C+) genome copies per eye improved levels of ABCA4 (one-way ANOVA, 5′C vs 5′C+**p=0.006). Grouped analysis of eyes injected with variants without and (B, C, D) with the intron (5′B, 5′C, 5′D) confirmed the influence of the overlap region and identified a influence of the intron on ABCA4 expression levels (two-way ANOVA, overlap p=0.01, intron p=0.04, interaction ns). C. HEK293T cells transfected with downstream transgene constructs revealed that generation of truncated ABCA4 (tABCA4) was influenced by the transgene variant (Kruskal-Wallis, n=6, p=0.0003) with only variants A and B producing detectable levels of tABCA4. D. The percentage of full length and tABCA4 forms in treated cells from (a). Error bars represent SEM. ABCA4=ATP-binding cassette transporter protein family member 4; Do=downstream transgene variant; GAPDH=glyceraldehyde 3-phosphate dehydrogenase; tABCA4=truncated ABCA4; UTC=untransduced HEK293T cells; 5′=dual vector variant containing an intron in the upstream transgene; 5′C+=dual vector variant 5′C at 10 times the dose of all other samples (1×10¹⁰ genome copies per eye). (F) Dual vector overlap variant InC was injected into Abca4−/− mouse eyes with consistent detection of full length ABCA4 achieved 6 weeks post-injection (n=1 eye per lane). +=HEK293T cells transfected with pCAG.ABCA4; ABCA4=ATP-binding cassette transporter protein family member 4; Do=Abca4−/− eyes injected with downstream vector only; GAPDH=glyceraldehyde 3-phosphate dehydrogenase; In=dual vector variants containing an intron in the upstream vector; KO=uninjected Abca4−/− eye; Up=Abca4−/− eyes injected with upstream vector only; UTC=untransduced HEK293T cells; WT=SVEV 129 wild-type eye.

FIG. 10A-B area gel and a pair of plots showing truncated ABCA4 detection from unrecombined downstream vectors. Detection of truncated ABCA4 protein is normalized to GAPDH per sample and presented as levels above untreated negative controls. (A) HEK293T cells were transfected with plasmids carrying different downstream transgenes for assessment of truncated ABCA4 (tABCA4) production. The levels of tABCA4 detected were influenced by the downstream transgene variant (one-way ANOVA, n=3, p<0.0001, F(7,16)=97.04). Downstream variant A produced more tABCA4 than variants Bx, C, D, E, F and X while variant B generated more tABCA4 than all other downstream variants (one-way ANOVA, Tukey's multiple comparisons test, *p<0.0l/****p<0.0001). (B) Shows the percentage of full length ABCA4 and tABCA4 forms identified from HEK293T cells transduced with AAV2/8 Y733F dual vector variants A-D. Error bars represent SEM.

FIG. 11A-B area diagram showing dual vector overlap variants and a gel and two plots comparing ABCA4 detection following sub-retinal injection in Abca4^{-/-} eyes of four dual vector variants with and without a 5′UTR in the upstream transgene. Nucleotides of the ABCA4 coding sequence (SEQ ID NO: 11) are included in each transgene are shown. Detection of full length ABCA4 protein was normalized to GAPDH per sample and presented as levels above untreated negative control samples. (A) Abca4^{-/-} eyes injected with AAV2/8 Y733F variants were assessed 6 weeks post-injection and ABCA4 levels were assessed when a 5′UTR was added to the upstream transgene (two-way ANOVA, n=3 (Bx/5′D), 5 (B/C), 6 (5′Bx/D), 7 (5′B/5′C), 5′UTR influence p=0.03, overlap influence p=0.005, interaction not significant, ns). (B) Full length ABCA4 versus truncated ABCA4 was detected from Abca4^{-/-} eyes that received a sub-retinal injection of 2E+10 total genome copies of the optimized dual vector variant 5′C, compared to eyes that received 2E+9 total genome copies (unpaired non-parametric Mann Whitney test, n=9 & 17, *p=0.01).

FIG. 12A-B are a series of diagrams. (A) Shows the overlap C sequence with out-of-frame AUG codons prior to an in-frame AUG codon; (B) shows predicted secondary structures of overlap zones C and B.

FIG. 13A-B area diagram, a plot (13A) and a gel (13B) showing ABCA4 transcripts isolated from upstream vector only treated samples. The diagram shows a segment of nucleotide sequence from the upstream transgene variant B. The sequence from the SwaI site was consistent in all upstream transgene variants and the features of a possible cryptic poly A signal are highlighted. (A) Shows the detection of ABCA4 transcripts from HEK293T cells treated with WT or codon optimized AAV2/2 upstream vector (n=1). (B) Shows ABCA4 transcripts isolated from upstream vector only injected Abca4^{-/-} eyes (n=4) that were assessed for transcript length by RT PCR. A forward primer binding at the beginning of the ABCA4 CDS and a reverse primer binding beyond the SwaI site were used. CO=HEK293T cells treated with upstream AAV2/2 vector containing codon-optimized ABCA4 CDS; KO=Abca4−/− eyes; Ov=overlap PCR; Up=upstream PCR; WT=HEK293T cells treated with upstream AAV2/2 vector containing wild-type ABCA4 CDS.

FIG. 14A-B are a series of plots showing the detection of ABCA4 mRNA transcriptions from single vector injected Abca4^{-/-} eyes. (A) ABCA4 transcript detection from eyes injected only with upstream vector variant B or 5′B (including the 5′ UTR). Including the 5′UTR sequence in upstream vector B reduced the levels of ABCA4 transcripts detected (unpaired two-tailed t test, n=3-4, ***p=0.0003). (B) Removal of the WPRE from downstream vector B (Bx) reduced the levels of ABCA4 transcripts detected from single vector injected eyes (Kruskal-Wallis, Dunn's multiple comparisons test, n=3, *p=0.03). B/C=eyes injected with upstream or downstream vector variant B or C (see Table 2); 5′B=eyes injected with upstream vector B with additional 5′UTR sequence; Bx=eyes injected with downstream vector B without WPRE. Error bars represent SEM.

FIG. 15A-B are each four gels and a plot showing the absence of truncated ABCA4 protein from upstream vector only treated samples. (A) HEK293T samples were transfected with plasmids carrying a FLAG-tagged upstream transgene and assessed for ABCA4 transcript presence using primers directed to an upstream region of the ABCA4 CDS (graph and panel above of RT-PCR). ABCA4 transcripts were abundant (n=3) yet no truncated protein was detected by Western Blot. pD=HEK293T cells transfected with the downstream transgene plasmid; pF=HEK293T cells transfected with a FLAG-tagged upstream transgene plasmid; UTC=transfected HEK29T cells; +=positive control. (B) Abca4^{-/-} eyes were injected with a FLAG-tagged upstream vector AAV2/8 Y733F and assessed for ABCA4 mRNA transcript presence using primers directed to an upstream region of the ABCA4 CDS (graph and panel above of RT-PCR). ABCA4 transcripts were abundant in upstream vector only injected eyes (n=2) yet no truncated protein was detected by western blot. Error bars represent SEM. D=downstream vector only injected eyes; F=FLAG-tagged upstream vector only injected eyes; KO=uninjected Abca4^{-/-} eyes; −=empty lane; +=positive control.

FIG. 16 is a series of images showing staining of ABCA4 (green) in the outer segments of photoreceptor cells in an Abca4^{-/-} retina harvested 6 weeks post-injection. HCN1 (red) staining marks the inner segments. Staining example of native Abca4 localization in a WT retina is also included plus evidence of absence of staining in an uninjected Abca4^{-/-} retina.

FIG. 17 is a series of images showing Abca4/ABCA4 (green) and Hcn1 (red) staining in wild-type (WT) and Abca4^{-/-} eyes.

FIG. 18 is a series of images of Abca4/ABCA4 (green) and rhodopsin (red) staining in photoreceptor cell outer segments in wild-type (WT) and Abca4^{-/-} eyes.

FIG. 19 is a series of images of abca4/ABCA4 (green) and rhodopsin (red) apical RPE staining in wild-type (WT) and Abca4^{-/-} eyes. The boxed image at the bottom right depicts GFP (green) and rhodopsin (red) apical RPE staining in wild-type (WT) and Abca4^{-/-} eyes.

FIG. 20A-J is a series of 28 images of sections of mouse eyes stained for the outer segment protein ABCA4 using a polyclonal antibody directed against the C-terminal of ABCA4 (green) and for the inner segment marker protein Hcn1 (red) (A-F), or for rho (red) (G-H) or ABCA4 alone (I-J). Nuclei were stained with Hoescht (blue). ABCA4 marks photoreceptor the outer segments whereas Hcn1 labels the inner segments. All eyes were injected at 4-5 weeks of age and harvested 6 weeks post-injection. (A) ABCA4 staining in photoreceptor outer segments of WT SVEV 129; (B) absence of ABCA4 staining in uninjected Abca4^{-/-} eyes; (C) absence of Abca4/ABCA4 in upstream vector injected Abca4^{-/-} eyes; (D) absence of ABCA4 staining in downstream vector injected Abca4^{-/-} eyes; (E-F) ABCA4 staining in photoreceptor outer segments of dual vector injected Abca4^{-/-} eyes (two different eyes); (G) Rho and Abca4 co-localization in photoreceptor outer segments of WT SVEV 129; (H) Rho and ABCA4 co-localization in photoreceptor outer segments of dual vector injected Abca4^{-/-} eyes; (I) absence of ABCA4 staining in downstream vector injected Abca4^{-/-} eyes at 6 months post-injection; (J) ABCA4 staining in photoreceptor out segments of dual vector injected Abca4^{-/-} 6 months post-injection. Abca4/ABCA4=ATP-binding cassette transporter protein family member 4; Dual=dual vector injected Abca4^{-/-} eyes; Downstream=downstream vector injected Abca4^{-/-}; Hcn1=hyperpolarization activated cyclic nucleotide gated potassium channel 1; IS=inner segments; KO=Abca4^{-/-}; ONL=outer nuclear layer; OS=outer segments; RPE=retinal pigment epithelium; Upstream=upstream vector injected Abca4^{-/-}; WT=SVEV 129 wild-type. Upstream KO (single AAV vector at 5′ end), Downstream KO (single AAV vector from 3′ end) show no ABCA4 production as expected, but Dual KO (combined AAV vectors) leads to robust ABCA4 protein expression (F and H).

FIG. 21A-F is a series of 8 images showing ABCA4 staining (green) and rhodopsin staining (red) in injected Abca4−/− eyes. Nuclei were stained with Hoescht. (A) Absence of ABCA4 staining in Abca4−/− eyes injected with downstream vector only 6 months post-injection. (B) ABCA4 staining in photoreceptor outer segments of dual vector injected Abca4−/− eyes 6 months post-injection. (C) RPE GFP expression in AAV2/2 CAG.GFP.WPRE.pA injected Abca4−/−. (D) Co-localization of Rho and Abca4 in the apical region of RPE cells in WT SVEV 129. (E) Co-localization of Rho and ABCA4 in the apical region of RPE cells in dual vector injected Abca4−/−. (F) Rho staining in the apical region of RPE cells in uninjected Abca4−/−. Abca4/ABCA4=ATP-binding cassette transporter protein family member 4; Dual=dual vector injected Abca4−/−; KO=Abca4−/−; Rho=rhodopsin; RPE=retinal pigment epithelium; WT=SVEV 129 wild-type. White arrows indicate co-localization of Abca4/ABCA4 and Rho in the apical region of RPE cells.

FIG. 22 is a diagram depicting exemplary overlapping vectors.

FIG. 23 is a diagram depicting the normal retinoid cycle is shown on the left-hand side of the diagram. The generation of bisretinoids and A2E that occurs to an enhanced degree in Abca4 deficient mice and humans is shown on the right. The molecules highlighted in boxes on the right-hand side of the diagram were assessed in Abca4^{-/-} mice. (Example 6.)

FIG. 24 is a plot of the levels of bisretinoids and A2E isoforms in paired eyes for 13 Abca4^{-/-} mice that received either sham or treatment injection. A decrease in bisretinoid and A2E levels was observed between sham and treatment eyes (p=0.017, F=5.849). Furthermore, for all bisretinoid and A2E measurements, the lowest levels were seen in the dual vector treated eyes. (Example 6.)

FIG. 25A-D is a series of plots (A, C and D) and a table (B) showing Bisretinoid/A2E levels in Abca4^{-/-} eyes injected with the optimized dual vector (treatment) compared to paired sham (A) injected eyes. Example chromatogram traces are shown for sham (A) and treatment (C) injected eyes, with labeled peaks indicated in table (B). Bisretinoid/A2E levels from eyes that received the dual vector treatment compared to those that received the sham injection are shown in (D). A reduction in bisretinoid/A2E levels was observed in eyes that received the dual vector treatment compared to those that received the sham injection (two-way ANOVA with matching values, n=13, treatment effect p=0.03, F(1,60)=4.516). A difference between A2PE-H2 levels in paired eyes was also observed (two-way ANOVA, Sidak's multiple comparisons test, n=13, p=0.01). Error bars represent SEM. atRALdi-PE=all-trans-retinal dimer-phosphatidylethanolamine; A2PE-H2=di-hydro-A2PE; A2PE=N-retinylidene-N-retinylphosphatidylethanolamine; A2E=conjugated N-retinylidene-N-reintylphosphatidylethanolamine; iso-A2E=double bond isomer of A2E; WT=SVEV 129 controls.

FIG. 26 is a plot showing the reduction in bisretinoid and A2E levels in Abca4^{-/-} eyes injected with dual vector (treatment) compared to eyes injected with upstream vector dose control (sham). Two groups of mice (n=11) received either a treatment or sham injection in one eye while the paired eye remained uninjected. Levels of bisretinoids and A2E were assessed in each eye 3 months post-injection and presented as the fold change in levels per mouse between eyes. A difference in the fold change of bisretinoid and A2E levels in the treatment group compared to the sham group was identified (two-way ANOVA, n=11, p=0.05, F(1,100)=3.695). Error bars represent SEM.

FIG. 27A-B is a series of images (a) and a plot (b) showing Lipofuscin-related 488 nm and melanin-related 790 nm autofluorescence mean grey values from Abca4^{-/-} mice 6 months post-injection that each received 2E+10 total genome copies of dual vector (treatment, left images) in one eye and a PBS injection (sham, right images) in the contralateral eye. (b) The difference in mean grey value compared to the mean grey value average of four sham injected wild-type SVEV 129 eyes. Dual vector treatment reduced lipofuscin and melanin-related autofluorescence in Abca4^{-/-} mice 6 months post-injection (one-way ANOVA, n=12, treatment influence p=0.01, F(1,2)=6.762). Error bars represent SEM. 488 nm=lipofuscin-related autofluorescence; 790 nm=melanin-related autofluorescence; Sham=PBS injected Abca4-; Treatment=dual vector injected Abca4^{-/-} mice; WT=wild-type SVEV 129 mice.

FIG. 28A-C is a series of gels and plots showing ABCA4 protein detection following treatment with wild-type (WT) or codon-optimized (CO) ABCA4 coding sequence. Detection of full length ABCA4 protein was normalized to GAPDH per sample and presented as levels above untreated negative control samples. (A) A. Plasmids identical but for containing WT or CO ABCA4 coding sequence were used to transfect HEK293T cells with a difference in subsequent ABCA4 protein levels determined (two-tailed unpaired t-test, n=4, ***p=0.0002, F(3,3)=2.973). (B) AAV2/2 overlapping dual vectors identical but for containing WT or CO ABCA4 coding sequence were used to transfect HEK293T cells, no difference in subsequent ABCA4 protein levels was observed (two-tailed unpaired t-test, n=3, F(2,2)=18.74). (C) Abca4^{-/-} mice received sub-retinal injection of AAV2/8 overlapping dual vectors containing either WT or CO ABCA4 coding sequence. The coding sequence used in the dual vector system had an influence on the levels of ABCA4 detected (two-way ANOVA, n=8-9, coding sequence influence p=0.04, time point influence ns, interaction p=0.01). At 6 weeks post-injection, WT injected eyes had more ABCA4 than CO injected eyes (two-way ANOVA, Sidak's multiple comparisons test, **p=0.005) and ABCA4 detection in WT injected eyes was greater at 6 weeks than at 2 weeks post-injection (two-way ANOVA, Sidak's multiple comparisons test, *p=0.05). Error bars represent SEM. ABCA4=ATP-binding cassette transporter protein family member 4; CO=samples treated with transgenes containing codon-optimised ABCA4 coding sequence; COd=eyes injected with CO downstream vector; COu=eyes injected with CO upstream vector; GAPDH=glyceraldehyde 3-phosphate dehydrogenase; KO=uninjected Abca4^{-/-} retina lysate; tABCA4=truncated ABCA4; UTC=untransduced HKE293T cell lysate; WT=samples treated with transgenes containing wild-type ABCA4 coding sequence; WTd=eyes injected with WT downstream vector; WTu=eyes injected with WT upstream vector; +=ABCA4 transfected HEK293T cell lysate.

FIG. 29 is a plot showing a comparison of ABCA4 expression levels in Abca4−/− eyes injected with AAV2/8 or AAV2/8 Y773F dual vectors carrying identical transgenes. mRNA transcripts were isolated from Abca4−/− injected retinae 6 weeks post-injection and qPCR analysis was performed on cDNA. More ABCA4 transcripts were detected from AAV2/8 Y733F dual vector injected eyes (Mann-Whitney two-tailed test, n=4, ***p=0.0002). Error bars represent SEM.

FIG. 30A-B is a pair of diagrams of the development of the ABCA4 dual vector system. A. Different aspects of vector design were considered and assessed, including the genetic elements and structure of the transgene and the vector capsid and dose. B. Dual vector variants carrying different overlap lengths were compared to determine the optimal region for recombination between two transgenes. AAV=adeno-associated virus; ABCA4=ATP-binding cassette transporter protein family member 4; Do=downstream transgene variant; GRK1=human rhodopsin kinase promoter; In=intron; ITR=inverted terminal repeat; pA=polyA signal; Up=upstream transgene variant; WPRE=Woodchuck hepatitis virus post-transcriptional regulatory element.

FIG. 31A-D area flowchart of an experiment (A, top row), two example chromatograph traces and a table of peaks (A, middle row) and a plot (A, at bottom), a series of 12 images of eyes (B, top) and a plot (B, bottom), (C) an additional 4 images of sham and treated eyes, (D) and an additional plot showing dual vector therapeutic effects observed as a reduction in bisretinoid accumulation and fundus autofluorescence in treated Abca4^{-/-} eyes that were injected. The data show evidence of dual vector therapeutic effects in the Abca4^{-/-} mouse model. A. Dual vector therapeutic effect observed as a reduction in bisretinoid accumulation in treated Abca4^{-/-} eyes at 3 months post-injection. Example chromatogram traces are shown for upstream vector (sham) and dual vector (treatment) injected eyes. Bisretinoid levels in WT eyes are presented in the graph for reference only and were not included in the analysis. A significant reduction in bisretinoid levels was observed in eyes that received the dual vector treatment compared to those that received the sham injection (two-way ANOVA with matching values, n=13, treatment effect p=0.03, F(1,60)=4.516). A significant difference between A2PE-H2 levels in paired eyes was also observed (two-way ANOVA, Sidak's multiple comparisons test, n=13, *p=0.01). A-D. Increased autofluorescence is an early feature of Stargardt disease. 790 nm autofluorescence increased over time in Abca4^{-/-} eyes but there was a significant difference in the increase observed in eyes that received dual vector (treatment) compared to paired PBS (sham) injected eyes (paired t-test, n=12, *p=0.04). Dual vector therapeutic effect observed as a reduction in 790 nm autofluorescence 6 months post-injection. Abca4^{-/-} eyes were imaged by scanning laser ophthalmology (SLO) 3 and 6 months post-injection and changes in 790 nm autofluorescence were significantly reduced in eyes that received dual vector (treatment) compared to paired PBS (sham) injected eyes (paired t-test, n=12, *p=0.04). Dual vector treated mice show a reduction in retinal autofluorescence compared with saline injected controls. Mice were treated in early adult life (<3 months) and retinal autofluorescenec imaging (Heidelberg Spectralis) was perfomed at 3 and 6 months. (E) The highlighted area just below the optic nerve is shown at high power for comparison. atRALdi-PE=all-trans-retinal dimer-phosphatidylethanolamine; A2PE-H2=di-hydro-A2PE; A2PE=N-retinylidene-N-retinylphosphatidylethanolamine; A2E=conjugated N-retinylidene-N-reintylphosphatidylethanolamine; iso-A2E=double bond isomer of A2E; SLO=scanning laser ophthalmoscopy; WT=SVEV 129 age-matched controls.

FIG. 32A-B are each a diagram and a gel showing RT-PCR of ABCA4 from injected Abca4^{-/-} eyes (n=4, pooled) confirmed transcripts from recombined transgenes had the correct coding sequence at the overlap region with successful removal of the intron. (A) A forward primer binding ABCA4 CDS provided by the upstream transgene and a reverse primer binding ABCA4 CDS in the downstream transgenes were used to amplify transcripts from recombined transgenes. Amplicons were sequenced to confirm the correct ABCA4 CDS was contained across the overlap regions of the transcripts. (B) A forward primer binding downstream of the predicted GRK1 transcriptional start site (TSS) and a reverse primer binding within the upstream ABCA4 CDS were used to assess transcript forms from dual vector C injected eyes and dual vector 5′C injected eyes. ABCA4 transcripts from dual vector C injected eyes generated a single amplicon representing the original reference sequence. Transcripts from dual vector 5′C injected eyes generated three defined products that were sequenced and confirmed to be: unspliced; partially spliced and fully spliced variants. ABCA4=ATP-binding cassette transporter protein family member 4; B/C=eyes injected with dual vector variants B or C (see Table 2); 5′B=eyes injected with dual vector variant B in which the upstream transgene contains an intron; CDS=coding sequence; GFP=eyes injected with GRK1.GFP.pA AAV2/8 Y733F injected eyes; ITR=inverted terminal repeat; KO=uninjected Abca4^{-/-} eyes; pA=polyA signal; WPRE=Woodchuck hepatitis virus post-transcriptional regulatory element; Up=eyes injected with Up1 only; Up+Do=pooled cDNA from upstream vector only injected eyes and downstream vector only injected eyes; +=ABCA4 plasmid control.

FIG. 33 is a diagram of promoters and additional sequences that can be used to drive expression of the ABCA4 upstream sequence. RK=GRK1 promoter, IntEx=intron and exon sequence, CMV=cytomegalovirus early enhancer; CBA=chicken beta actin promoter; SA/SD =splice acceptor and splice donor.

FIG. 34 is a diagram of AAV vectors used to express the ABCA4 upstream sequence or GFP. ITR=Inverted Terminal Repeat, WPRE=Woodchuck hepatitis virus post-transcriptional regulatory element, GFP=green fluorescent protein, IntEx=intron and exon sequence, CBA=chicken beta actin promoter, CMV=cytomegalovirus enhancer, RK=rhodopsin kinase promoter (GRK1 promoter), RBG=Rabbit beta globin, SA/SD=splice acceptor and splice donor sequence.

FIG. 35 is a sequence of a CMVCBA.In.GFP.pA vector (SEQ ID NO: 17).

FIG. 36 is a sequence of a CMVCBA.GFP.pA vector (SEQ ID NO: 18).

FIG. 37 is a sequence of a CBA.IntEx.GFP.pA vector (SEQ ID NO: 19).

FIG. 38 is a sequence of a CAG.GFP.pA vector (SEQ ID NO: 20).

FIG. 39 is a sequence of an AAV.5′CMVCBA.In.ABCA4.WPRE.kan vector (SEQ ID NO: 21).

FIG. 40 is a sequence of an AAV.5′CMVCBA.ABCA4.WPRE.kan vector (SEQ ID NO: 22).

FIG. 41 is a sequence of an AAV.5′CBA.IntEx.ABCA4.WPRE.kan vector (SEQ ID NO: 23).

FIG. 42 is a series of schematic diagrams depicting exemplary ABCA4 expression constructs of the disclosure.

FIG. 43 is a sequence of the ITR to ITR portion of pAAV.RK.5′ABCA4.kan (SEQ ID NO: 26), comprising a sequence encoding a 5′ ITR (SEQ ID NO: 27), a sequence encoding an RK promoter (SEQ ID NO: 28), a sequence encoding a Rabbit Beta-Globin (RBG) Intron/Exon (Int/Ex) (SEQ ID NO: 39), a sequence encoding a 5′ portion of the coding sequence of an ABCA4 gene (SEQ ID NO: 29), and a sequence encoding a 3′ ITR (SEQ ID NO: 30).

FIG. 44 is a sequence of the ITR to ITR portion of pAAV.3′ABCA4.WPRE.kan (SEQ ID NO: 30), comprising a sequence encoding a 5′ ITR (SEQ ID NO: 27), a sequence encoding a 3′ portion of the coding sequence of an ABCA4 gene (SEQ ID NO: 31), a sequence encoding WPRE (SEQ ID NO: 32), a sequence encoding bGH polyA and a sequence encoding a 3′ ITR (SEQ ID NO: 33).

FIG. 45 is an image of the fundus of an eye of a patient with Mid-Stage Stargardt disease.

FIG. 46 is a picture adapted from Sears et al. TVST 6(5): 6 (2017), showing localization of ABCA4 and the retinoid cycle (sometimes called visual cycle) in photoreceptor cells. ABCA4 localizes to the outer segment disc membranes of rod and cone photoreceptors. OS, light sensitive outer segment; CC, connecting cilium; IS, inner segment.

FIG. 47A-C are a series of pictures showing the conversion of a transgene encoded by a double stranded DNA (dsDNA) to single stranded sense and antisense DNAs (ssDNA), and encapsidation of the ssDNAs in AAV viral particles.

FIG. 48A-D are a series of pictures showing the uptake of the AAV viral particles containing the sense and antisense ssDNAs by the nucleus (A), release of the sense and antisense strands from the viral particles (B), synthesis of the complementary strand to regenerate dsDNA (C) and transcription of the transgene (D).

FIG. 49A-H are a series of pictures that depict encapsidation, transduction, and reformation of a large transgene in an AAV dual vector system through single strand annealing and second strand synthesis. The large transgene is initially encoded as dsDNA (A-B). Subsequently, ssDNAs of overlapping 5′ and 3′ fragments of the large transgene are encapsidated by AAV viral particles (C). Viral particles comprising complementary strands of the 5′ and 3′ fragments of the large transgene are generated, and these ssDNAs comprise a region of complementary, overlapping sequence (shown in red). In this example, the antisense ssDNA of the 5′ fragment and the sense strand of the 3′ are depicted. AAV particles comprising the ssDNAs are transduced (D), and the ssDNAs are released from the viral particles into the nucleus (E). The 5′ and 3′ fragments hybridize at the complementary, overlapping sequence in the nuclear environment (F), a dsDNA of the entire large transgene is generated through second strand synthesis (G), and this dsDNA is subsequently transcribed and the transgene expressed (H).

FIG. 50 is an outline of an ABCA4 overlapping dual vector system of the disclosure. The elements of an adeno-associated virus (AAV) transgene were split across two independent transgenes, “upstream” and “downstream”. The upstream transgene contained the promoter and upstream fragment of ABCA4 coding sequence whilst the downstream transgene carried the downstream fragment of ABCA4 coding sequence plus a WPRE and a bovine growth hormone (bGH) pA signal. In the optimized overlapping dual vector system depicted, both transgenes carried a 207 bp region of overlap formed from ABCA4 coding sequence bases 3,494-3,701. Once inside the same host cell nucleus, the two transgenes align and recombine via the region of overlap. ABCA4=ATP-binding cassette transporter protein family member 4; GRK1=human rhodopsin kinase promoter; In=intron; ITR=inverted terminal repeat; pA=polyA signal; WPRE=Woodchuck hepatitis virus post-transcriptional regulatory element.

FIG. 51 is a table showing transgene details for the dual vector combinations tested. The final row contains the details for the optimized overlapping dual vector system. ABCA4=ATP-binding cassette transporter protein family member 4; bp=base pairs; CDS=coding DNA sequence; GRK1=human rhodopsin kinase promoter; pA=polyA signal; WPRE=Woodchuck hepatitis virus post-transcriptional regulatory element.

FIG. 52 is an annotated sequence of exemplary plasmid pAAV.stbIR.3′ABCA4.WPRE.kan (SEQ ID NO: 40), comprising a sequence encoding a 5′ ITR (AAV2 derived ITR, nucleotides 16-130, SEQ ID NO: 41), a sequence encoding a 3′ABCA4 (nucleotides 176-3509, SEQ ID NO: 42), a sequence encoding a WPRE (nucleotides 3516-4108, SEQ ID NO: 43), a sequence encoding a BGH PolyA (nucleotides 4115-4278, SEQ ID NO: 44), and a sequence encoding a 3′ IR (AAV derived ITR, nucleotides 4422-4542, SEQ ID NO: 45).

FIG. 53 is an annotated sequence of exemplary plasmid pAAV.stbITR.CBA.InEx.5′ABCA4.kan (SEQ ID NO: 46), comprising a sequence encoding a 5′ IR (AAV2 derived ITR, nucleotides 16-130, SEQ ID NO: 47), a sequence encoding a CBA promoter (nucleotides 190-467, SEQ ID NO: 48), a sequence encoding an intron (nucleotides 468-590, SEQ ID NO: 49), a sequence encoding an exon (nucleotides 591-630, SEQ ID NO: 50), a sequence encoding a 5′ABCA4 (nucleotides 650-4351, SEQ ID NO: 51), and a sequence encoding a 3′ IR (AAV2 derived ITR, nucleotides 4389-4509, SEQ ID NO: 52).

FIG. 54 is an annotated sequence of exemplary plasmid pAAV.stbITR.CBA.RBG.5′ABCA4.kan (SEQ ID NO: 53), comprising a sequence encoding a 5′ IR (AAV2 derived ITR, nucleotides 16-130, SEQ ID NO: 54), a sequence encoding a CBA promoter (nucleotides 190-467, SEQ ID NO: 55), a sequence encoding a RGB intron (nucleotides 704-876, SEQ ID NO: 56), a sequence encoding a 5′ABCA4 (nucleotides 919-4620, SEQ ID NO: 57), and a sequence encoding a 3′ IR (nucleotides 4658-4778, SEQ ID NO: 58).

FIG. 55 is an annotated sequence of exemplary plasmid pAAV.stbITR.CMV.CBA.5′ABCA4.kan (SEQ ID NO: 59), comprising a sequence encoding a 5′ IR (AAV2 derived ITR, nucleotides 16-130, SEQ ID NO: 60), a sequence encoding a CMV enhancer (nucleotides 322-556, SEQ ID NO: 61), a sequence encoding a CBA promotor (nucleotides 571-849, SEQ ID NO: 62), a sequence encoding a 5′ABCA4 (nucleotides 856-4557, SEQ ID NO: 63), and a sequence encoding a 3′ IR (nucleotides 4595-4715, SEQ ID NO: 64).

FIG. 56 is an annotated sequence of exemplary plasmid pAAV.stbITR.RK.5′ABCA4.kan (SEQ ID NO: 65), comprising a sequence encoding a 5′ IR (AAV2 derived ITR, nucleotides 16-130, SEQ ID NO: 66), a sequence encoding a RK promoter (nucleotides 186-384, SEQ ID NO: 67), a sequence encoding a 5′ABCA4 (nucleotides 576-4267, SEQ ID NO: 68), and a sequence encoding a 3′ IR (nucleotides 4275-4425, SEQ ID NO: 69).

FIG. 57 is a schematic diagram depicting an exemplary AOSLO system for direct visualization of retinal cells of a subject, including photoreceptor cells of an inner segment, an outer segment or a combination thereof.

### LIST OF SEQUENCES

SEQ ID NO: 1 Human ABCA4 nucleic acid sequence. SEQ ID NO: 1, corresponding to NCBI Reference Sequence NM_000350.2.
SEQ ID NO: 2 Human ABCA4 nucleic acid sequence variant. SEQ ID NO: 2 is identical to SEQ ID NO: 1 with the exception of the following mutations: nucleotide 1640 G>T, nucleotide 5279 G>A, nucleotide 6173 T>C.
SEQ ID NO: 3 Example upstream vector sequence, comprising 5′ ITR, promoter, CDS, 3′ ITR.
SEQ ID NO: 4 Example downstream vector sequence, comprising 5′ ITR, CDS, post-transcriptional response element, poly-adenylation sequence, 3′ ITR.
SEQ ID NO: 5 GRK1 promoter sequence.
SEQ ID NO: 6 UTR sequence.
SEQ ID NO: 7 Woodchuck Hepatitis Virus post-transcriptional response element (WPRE).
SEQ ID NO: 8 Bovine Growth Hormone poly-adenylation sequence.
SEQ ID NO: 9 Example partial upstream vector sequence, comprising promoter, CDS.
SEQ ID NO: 10 Example partial downstream vector sequence, comprising CDS, post transcriptional response element, poly-adenylation sequence.
SEQ ID NO: 11 Human ABCA4 cDNA sequence. This sequence corresponds to nucleotides 105-6926 of NM_000350.2 (SEQ ID NO: 1).
SEQ ID NO: 12 AAV8 capsid protein sequence. This sequence corresponds to the AAV8 capsid protein sequence of GenBank record AF513852.1.
SEQ ID NO: 13 Intron.
SEQ ID NO: 14 Exon.
SEQ ID NO: 15 CMV enhancer.
SEQ ID NO: 16 CBA promoter.
SEQ ID NO: 17 CMVCBA.In.GFP.poly(A) vector sequence.
SEQ ID NO: 18 CMVCBA.GFP.poly(A) vector sequence.
SEQ ID NO: 19 CBA.IntEx.GFP.poly(A) vector sequence.
SEQ ID NO: 20 CAG.GFP.poly(A) vector sequence.
SEQ ID NO: 21 AAV.5′CMVCBA.In.ABCA4.WPRE.kan vector sequence.
SEQ ID NO: 22 AAV.5′CMVCBA.ABCA4.WPRE.kan vector sequence.
SEQ ID NO: 23 AAV.5′CBA.IntEx.ABCA4.WPRE.kan vector sequence.
SEQ ID NO: 24 CBA promoter.
SEQ ID NO: 25 Bovine Growth Hormone poly-adenylation sequence.
SEQ ID NO: 26 The ITR to ITR portion of pAAV.RK.5′ABCA4.kan, comprising a sequence encoding a 5′ ITR (SEQ ID NO: 27), a sequence encoding an RK promoter (SEQ ID NO: 28), a sequence encoding a Rabbit Beta-Globin (RBG) Intron/Exon (Int/Ex) (SEQ ID NO: 39), a sequence encoding a 5′ portion of the coding sequence of an ABCA4 gene (SEQ ID NO: 29), and a sequence encoding a 3′ ITR (SEQ ID NO: 30).
SEQ ID NO: 27 a sequence encoding an exemplary 5′ ITR
SEQ ID NO: 28 a sequence encoding an RK promoter
SEQ ID NO: 29 a sequence encoding a 5′ portion of the coding sequence of an ABCA4 gene
SEQ ID NO: 30 The ITR to ITR portion of pAAV.3′ABCA4.WPRE.kan, comprising a sequence encoding a 5′ ITR (SEQ ID NO: 27), a sequence encoding a 3′ portion of the coding sequence of an ABCA4 gene (SEQ ID NO: 31), a sequence encoding a WPRE (SEQ ID NO: 32), a sequence encoding bGH polyA (SEQ Id NO: 38) and a sequence encoding a 3′ ITR (SEQ ID NO: 33).
SEQ ID NO: 31 a sequence encoding a 3′ portion of the coding sequence of an ABCA4 gene
SEQ ID NO: 32 a sequence encoding a WPRE
SEQ ID NO: 33 a sequence encoding an exemplary 3′ ITR
SEQ ID NO: 34 a sequence encoding an exemplary 5′ ITR
SEQ ID NO: 35 a sequence encoding an exemplary 3′ ITR
SEQ ID NO: 36 a sequence encoding an exemplary 5′ ITR
SEQ ID NO: 37 a sequence encoding an exemplary 3′ ITR
SEQ ID NO: 38 a sequence encoding a bGH polyA
SEQ ID NO: 39 a sequence encoding a Rabbit Beta-Globin (RBG) Intron/Exon (Int/Ex)
SEQ ID NO: 40 pAAV.stbIR.3′ABCA4.WPRE.kan comprising a sequence encoding a 5′ ITR (AAV2 derived ITR, nucleotides 16-130, SEQ ID NO: 41), a sequence encoding a 3′ABCA4 (nucleotides 176-3509, SEQ ID NO: 42), a sequence encoding a WPRE (nucleotides 3516-4108, SEQ ID NO: 43), a sequence encoding a BGH PolyA (nucleotides 4115-4278, SEQ ID NO: 44), and a sequence encoding a 3′ IR (AAV derived ITR, nucleotides 4422-4542, SEQ ID NO: 45).
SEQ ID NO: 41 a sequence encoding a 5′ ITR (AAV2 derived ITR, nucleotides 16-130 of SEQ ID NO: 40)
SEQ ID NO: 42 a sequence encoding a 3′ABCA4 (nucleotides 176-3509 of SEQ ID NO: 40)
SEQ ID NO: 43 a sequence encoding a WPRE (nucleotides 3516-4108 of SEQ ID NO: 40)
SEQ ID NO: 44 a sequence encoding a BGH PolyA (nucleotides 4115-4278 of SEQ ID NO: 40)
SEQ ID NO: 45 a sequence encoding a 3′ IR (AAV derived ITR, nucleotides 4422-4542 of SEQ ID NO: 40)
SEQ ID NO: 46 pAAV.stbITR.CBA.InEx.5′ABCA4.kan comprising a sequence encoding a 5′ IR (AAV2 derived ITR, nucleotides 16-130, SEQ ID NO: 47), a sequence encoding a CBA promoter (nucleotides 190-467, SEQ ID NO: 48), a sequence encoding an intron (nucleotides 468-590, SEQ ID NO: 49), a sequence encoding an exon (nucleotides 591-630, SEQ ID NO: 50), a sequence encoding a 5′ABCA4 (nucleotides 650-4351, SEQ ID NO: 51), and a sequence encoding a 3′ IR (AAV2 derived ITR, nucleotides 4389-4509, SEQ ID NO: 52).
SEQ ID NO: 47 a sequence encoding a 5′ IR (AAV2 derived ITR, nucleotides 16-130 of SEQ ID NO: 46)
SEQ ID NO: 48 a sequence encoding a CBA promoter (nucleotides 190-467 of SEQ ID NO: 46)
SEQ ID NO: 49 a sequence encoding an intron (nucleotides 468-590 of SEQ ID NO: 46)
SEQ ID NO: 50 a sequence encoding an exon (nucleotides 591-630 of SEQ ID NO: 46)
SEQ ID NO: 51 a sequence encoding a 5′ABCA4 (nucleotides 650-4351 of SEQ ID NO: 46)
SEQ ID NO: 52 a sequence encoding a 3′ IR (AAV2 derived ITR, nucleotides 4389-4509 of SEQ ID NO: 46)
SEQ ID NO: 53 pAAV.stbITR.CBA.RBG.5′ABCA4.kan comprising a sequence encoding a 5′ IR (AAV2 derived ITR, nucleotides 16-130, SEQ ID NO: 54), a sequence encoding a CBA promoter (nucleotides 190-467, SEQ ID NO: 55), a sequence encoding a RGB intron (nucleotides 704-876, SEQ ID NO: 56), a sequence encoding a 5′ABCA4 (nucleotides 919-4620, SEQ ID NO: 57), and a sequence encoding a 3′ IR (nucleotides 4658-4778, SEQ ID NO: 58)
SEQ ID NO: 54 a sequence encoding a 5′ IR (AAV2 derived ITR, nucleotides 16-130 of SEQ ID NO: 53)
SEQ ID NO: 55 a sequence encoding a CBA promoter (nucleotides 190-467 of SEQ ID NO: 53)
SEQ ID NO: 56 a sequence encoding a RGB intron (nucleotides 704-876 of SEQ ID NO: 53)
SEQ ID NO: 57 a sequence encoding a 5′ABCA4 (nucleotides 919-4620 of SEQ ID NO: 53)
SEQ ID NO: 58 a sequence encoding a 3′ IR (nucleotides 4658-4778 of SEQ ID NO: 53)

SEQ ID NO: 59 pAAV.stbITR.CMV.CBA.5′ABCA4.kan comprising a sequence encoding a 5′ IR (AAV2 derived ITR, nucleotides 16-130, SEQ ID NO: 60), a sequence encoding a CMV enhancer (nucleotides 322-556, SEQ ID NO: 61), a sequence encoding a CBA promotor (nucleotides 571-849, SEQ ID NO: 62), a sequence encoding a 5′ABCA4 (nucleotides 856-4557, SEQ ID NO: 63), and a sequence encoding a 3′ IR (nucleotides 4595-4715, SEQ ID NO: 64).
SEQ ID NO: 60 a sequence encoding a 5′ IR (AAV2 derived ITR, nucleotides 16-130 of SEQ ID NO: 59)
SEQ ID NO: 61 a sequence encoding a CMV enhancer (nucleotides 322-556 of SEQ ID NO: 59)
SEQ ID NO: 62 a sequence encoding a CBA promotor (nucleotides 571-849 of SEQ ID NO: 59)
SEQ ID NO: 63 a sequence encoding a 5′ABCA4 (nucleotides 856-4557 of SEQ ID NO: 59)
SEQ ID NO: 64 a sequence encoding a 3′ IR (nucleotides 4595-4715 of SEQ ID NO: 59)
SEQ ID NO: 65 pAAV.stbITR.RK.5′ABCA4.kan comprising a sequence encoding a 5′ IR (AAV2 derived ITR, nucleotides 16-130, SEQ ID NO: 66), a sequence encoding a RK promoter (nucleotides 186-384, SEQ ID NO: 67), a sequence encoding a 5′ABCA4 (nucleotides 576-4267, SEQ ID NO: 68), and a sequence encoding a 3′ IR (nucleotides 4275-4425, SEQ ID NO: 69).
SEQ ID NO: 66 a sequence encoding a 5′ IR (AAV2 derived ITR, nucleotides 16-130 of SEQ ID NO: 65)
SEQ ID NO: 67 a sequence encoding a RK promoter (nucleotides 186-384 of SEQ ID NO: 65)
SEQ ID NO: 68 a sequence encoding a 5′ABCA4 (nucleotides 576-4267 of SEQ ID NO: 65)
SEQ ID NO: 69 a sequence encoding a 3′ IR (nucleotides 4275-4425 of SEQ ID NO: 65)
SEQ ID NO: 70 amino acid sequence encoding ABCA4 protein (as shown as GenBank Accession No. NP_000341.2)

### DETAILED DESCRIPTION

Stargardt disease is an inherited disease of the retina that can lead to blindness through the destruction of light-sensing photoreceptor cells in the eye. The disease commonly presents in childhood leading to blindness in young people. The development of Stargardt disease during childhood and adolescence can lead to severe vision loss in patients in their early twenties. Stargardt disease is the most common form of inherited juvenile macular dystrophy. Stargardt disease is an orphan disease that affects approximately 1 in 10,000 people. There are 65,000 Stargardt patients in the United States, France, Germany, Italy, Spain and United Kingdom.

The most common form of Stargardt disease is a recessive disorder linked to mutations in the gene encoding the protein ATP Binding Cassette, sub-family A, member 4 (ABCA4). ABCA4 is a large, transmembrane protein that plays a role in the recycling of light-sensitive pigments in retinal cells. The ABCA4 transmembrane protein plays a key role in clearing away toxic byproducts from the visual cycle. In Stargardt disease, mutations in the ABCA4 gene lead to a lack of functional ABCA4 protein in retinal cells. This in turn leads to the formation and accumulation of bisretinoid by-products, producing toxic granules of lipofuscin in Retinal Pigment Epithelial (RPE) cells. This causes degradation and eventual destruction of the RPE cells, which leads to loss of photoreceptor cells causing progressive loss of vision and eventual blindness. The absence of functional ABCA4 leads to degeneration of photoreceptors. Progressive photoreceptor degeneration leads to blindness.

Prior to the development of the compositions and methods of the disclosure, there were no treatment options. Prior to the development of the compositions and methods of the disclosure, there was a long-felt but unmet need for an effective treatment for Stargardt disease that addresses the underlying cause of the disease.

Gene therapy is a promising treatment for Stargardt disease. The aim of gene therapy is to correct the deficiency underlying the disease by using a vector to introduce a functional ABCA4 gene into the affected photoreceptor cells, thus restoring ABCA4 function.

The disclosure provides vectors derived from adeno-associated virus (AAV) for retinal gene therapy. AAV is a small virus that presents very low immunogenicity and is not associated with any known human disease. The lack of an associated inflammatory response means that AAV does not cause retinal damage when injected into the eye.

The size of the AAV capsid may imposes a limit on the amount of DNA that can be packaged within it. The AAV genome is approximately 4.7 kilobases (kb) in size, and, for some AAV vectors and serotypes, the corresponding upper size limit for DNA packaging in AAV may be approximately 5 kb. Thus, a limiting factor includes the size restriction of the encodable AAV transgene at under 5 kb. Stargardt disease is the most prevalent form of recessively inherited blindness and is caused by mutations in ABCA4.

The coding sequence of the ABCA4 gene is approximately 6.8 kb in size (with further genetic elements for gene expression). Thus, the size of coding sequence of the ABCA4 gene with further genetic elements appear to be larger than the standard AAV vector upper size limit.

A number of approaches to overcome this upper size limit and express large genes such as ABCA4 from AAV vectors are described herein. These approaches include “oversize” vector approaches and “dual” vector approaches.

### “Oversize” Vectors

A number of attempts have been made to force genes considerably larger than the native 4.7 kb genome into AAV vectors, with some success in transducing target cells. By way of example, Allocca et al. (J. Clin. Invest. vol. 118, No. 5, May 2008) prepared oversize AAV vectors packaging the murine ABCA4 and human MYO7A genes and demonstrated protein expression following transduction of mouse retinal cells. However, while it was proposed by Allocca et al. that certain AAV capsids could accommodate up to 8.9 kb, subsequent studies have found that the “oversize” approach does not in fact overcome the packaging upper size limit, but rather leads to truncation of the transgene in a random manner, providing a heterogeneous population of AAV vectors each comprising a fragment of the transgene (Dong et al., Molecular Therapy, vol. 18, No. 1, January 2010). It is believed that a proportion of oversize vectors in a given population package large enough fragments of the oversized transgene such that regions of overlap between the fragments exist, allowing re-assembly into a full length gene following transduction of a target cell. However, this method is unpredictable and inefficient, with the lack of packaging control and subsequent failure of recombination providing a significant barrier to consistent, detectable success.

### “Dual” Vectors

A more successful approach is to prepare dual vector systems, in which a transgene larger than the approximately 5 kb limit is split approximately in half into two separate vectors of defined sequence: an “upstream” vector containing the 5′ portion of the transgene, and a “downstream” vector containing the 3′ portion of the transgene. Transduction of a target cell by both upstream and downstream vectors allows a full-length transgene to be re-assembled from the two fragments using a variety of intracellular mechanisms.

In a so-called “trans-splicing” dual vector approach, a splice-donor signal is placed at the 3′ end of the upstream transgene fragment and a splice-acceptor signal placed at the 5′ end of the downstream transgene fragment. Upon transduction of a target cell by the dual vectors, inverted terminal repeat (ITR) sequences present in the AAV genome mediate head-to-tail concatermerization of the transgene fragments and trans-splicing of the transcripts results in the production of a full-length mRNA sequence, allowing full-length protein expression.

An alternative dual vector system uses an “overlapping” approach. In an overlapping dual vector system, part of the coding sequence at the 3′ end of the upstream coding sequence portion overlaps with a homologous sequence at the 5′ of the downstream coding sequence portion. AAVs package linear single stranded DNAs (ssDNAs). In an overlapping dual vector approach, a double-stranded transgene is split into a 5′ portion (sense, upstream)) and a 3′ portion (antisense, downstream), which overlap at the 3′ end of the 5′ portion and the 5′ end of the 3′ portion. The 5′ portion and 3′ portion are each encoded by an AAV vector (upstream and downstream vectors), which are each encapsidated in AAV viral particles as ssDNAs. Upon transduction of a cell by the upstream AAV particle and the downstream AAV particle, the infected cell or a nucleus thereof comprises an upstream AAV vector and a downstream AAV vector. When the same infected cell comprises a complementary upstream AAV vector and downstream AAV vector, each having a single-stranded sequence to which the other can hybridize, the complementary ssDNAs encoding the 5′ portion and the 3′ portion can generate the full length transgene.

Without wishing to be bound by any particular theory, a full length transgene (e.g. ABCA4) may be generated from an overlapping dual vector system by second strand synthesis, followed by homologous recombination. Upon transduction of cell by an upstream AAV particle and a downstream particle, a corresponding ssDNA upstream AAV vector and a downstream AAV vector is released into the cell or a nucleus thereof, and a dsDNA comprising the 5′ (upstream) portion of the transgene and the 3′ (downstream) portion of the transgene are generated from each of the ssDNAs by second strand synthesis. The dsDNA then undergoes homologous recombination at the region of overlap between the upstream and downstream portions of coding sequence, which allows for the recreation of a full-length transgene, from which a corresponding mRNA can be transcribed and full-length protein expressed. For example, WO 2014/170480 describes a dual AAV vector system encoding a human ABCA4 protein (the contents of which are incorporated herein in their entirety).

In some embodiments of the compositions and methods of the disclosure, a first AAV vector comprises a 5′ portion of an ABCA4 coding sequence. In some embodiments, a second AAV vector comprises a 3′ portion of an ABCA4 coding sequence. In some embodiments, the 5′ end portion and the 3′ end portion overlap by at least about 20 nucleotides. In some embodiments, the first AAV vector and the second AAV vector each comprise a single stranded DNA (ssDNA). In some embodiments, the first AAV vector comprises a sequence of the ABCA4 coding sequences and/or a sequence complementary to the ABCA4 coding sequence. In some embodiments, the second AAV vector comprises a sequence of the ABCA4 coding sequences and/or a sequence complementary to the ABCA4 coding sequence. In some embodiments, the first AAV vector comprises a sequence of the 5′ ABCA4 coding sequences and a sequence complementary to a portion of the 3′ ABCA4 coding sequence. In some embodiments, the second AAV vector comprises a sequence of the 3′ ABCA4 coding sequence and a sequence complementary to a portion of the 5′ ABCA4 coding sequence. In some embodiments, the first AAV vector and the second AAV vector undergo second strand synthesis to generate a first dsDNA AAV vector and a second dsDNA AAV vector. In some embodiments, the first dsDNA AAV vector and the second dsDNA AAV vector generate a full length ABCA4 transgene through homologous recombination.

Without wishing to be bound by any particular theory, a full length transgene may also be generated from an overlapping dual vector system through single-strand annealing and second strand synthesis. Upon transduction of a cell by an upstream AAV vector and a downstream AAV vector, wherein each of the upstream AAV vector and the downstream AAV vector comprises a ssDNA, and wherein the upstream AAV vector comprises a sequence encoding a 5′ portion of the transgene and the downstream AAV vector comprises a sequence encoding a 3′ portion of the transgene, the complementary upstream and downstream vectors are released into the cell or a nucleus thereof. In some embodiments, the upstream AAV vector comprises a sequence encoding a 5′ portion of the transgene and a sequence complementary to a 3′ portion of the transgene. In some embodiments, the upstream AAV vector comprises a sense sequence encoding a 5′ portion of the transgene and a sequence complementary to a 3′ portion of the transgene. In some embodiments, the upstream AAV vector comprises an antisense sequence encoding a 5′ portion of the transgene and a sequence complementary to a 3′ portion of the transgene. In some embodiments, the downstream AAV vector comprises a sequence encoding a 3′ portion of the transgene and a sequence complementary to a 5′ portion of the transgene. In some embodiments, the downstream AAV vector comprises an antisense sequence encoding a 3′ portion of the transgene and a sequence complementary to a 5′ portion of the transgene. In some embodiments, the downstream AAV vector comprises a sense sequence encoding a 3′ portion of the transgene and a sequence complementary to a 5′ portion of the transgene. In some embodiments, the upstream and downstream vectors hybridize at the region of complementarity (overlap). Following hybridization, a full length transgene is generated by second strand synthesis.

In some embodiments of the compositions and methods of the disclosure, a first AAV vector comprises a 5′ portion of an ABCA4 coding sequence, a second AAV vector comprises a 3′ portion of an ABCA4 coding sequence, and the 5′ portion and the 3′ portion overlap by at least 20 contiguous nucleotides. In some embodiments, the first AAV vector and the second AAV vector each comprise a single stranded DNA (ssDNA). In some embodiments, the first AAV vector comprises a sequence of the ABCA4 coding sequence and the second AAV vector comprises a sequence complementary to the ABCA4 coding sequence. In some embodiments, the second AAV vector comprises a sequence of the ABCA4 coding sequence and the first AAV vector comprises a sequence complementary to the ABCA4 coding sequence. In some embodiments, the first AAV vector and the second AAV vector anneal at a complementary overlapping region to generate a full length dsDNA ABCA4 transgene by subsequent second strand synthesis. In some embodiments, the full length dsDNA ABCA4 transgene is generated in vitro or in vivo (in a cell or in a subject).

The disclosure addresses the above prior art problems by providing adeno-associated viral (AAV) vector systems as described in the claims.

Dual vector approaches increase the capacity of AAV gene therapy, but may also substantially reduce levels of target protein which may be insufficient to achieve a therapeutic effect. In some embodiments of dual vector systems, the efficacy of recombination of dual vectors depends on the length of DNA overlap between the plus and minus strands (sense and antisense strands).The size of the ABCA4 coding sequence allows for the exploration of various lengths of overlap between the plus and minus strands to identify zones for optimal dual vector strategies for the treatment of disorders caused by mutations in large genes. These strategies can lead to production of enough target protein to provide therapeutic effect. In the Stargardt mouse model, therapeutic effect can be readily assessed as the target protein, ABCA4, is required in abundance in the photoreceptor cells of the retina and its absence induces the accumulation of bisretinoid compounds, which in turn leads to an increase in 790 nm autofluorescence. The therapeutic potential of the overlapping dual vector system can be validated in vivo by observing a reduction in this bisretinoid accumulation and subsequent 790 nm autofluorescence levels following treatment.

Advantageously, the AAV vector system of the disclosure provides surprisingly high levels of expression of full-length ABCA4 protein in transduced cells, with limited production of unwanted truncated fragments of ABCA4. With an optimized recombination, the full length ABCA4 protein is expressed in the photoreceptor outer segments in Abca4−/− mice and at levels sufficient to reduce bisretinoid formation and correct the autofluorescent phenotype on retinal imaging. These observations support a dual vector approach for AAV gene therapy to treat Stargardt disease.

Stargardt disease resulting from mutations in the ABCA4 gene is the most common inherited macular dystrophy, affecting 1 in 8,000-10,000 people and resulting from mutations in the ABCA4 gene. ABCA4 mutations which responsible for Stargardt disease and other cone and cone-rod dystrophies. Stargardt disease resulting from mutations in the ABCA4 gene is the most common cause of blindness in children in the developed world. The disease often presents in childhood and becomes progressively worse over the course of a patient's lifetime therefore therapeutic intervention at any point could prevent or slow further sight loss. This disease is progressive, and often becomes symptomatic in childhood but after the period of visual development, which provides ample opportunity for therapeutic intervention to prevent or slow further sight loss.

ABCA4 clears toxic metabolites from the photoreceptor outer segments discs. The absence of functional ABCA4 leads to photoreceptor degeneration. Photoreceptor outer segment discs comprise the light sensing protein rhodopsin and the transmembrane protein ABCA4. ABCA4 controls the export of certain toxic visual cycle byproducts. Visual pigments comprise an opsin and a chromophore, for example a retinoid such as 11-cis-retinal. In the visual cycle, sometimes termed the retinoid cycle, retinoids are bleached and recycled between the photoreceptors and the retinal pigment epithelium (RPE). Upon activation of rhodopsin during phototransduction, 11-cis-retinal is isomerized to all-trans-retinal, which dissociates from the opsin. All-trans-retinal is transported to the RPE, and either stored or converted back to 11-cis-retinal and transported back to photoreceptors to complete the visual cycle.

Mutations in ABCA4 prevent the transport of retinoids from photoreceptor cell disc outer membranes to the retinal pigment epithelium (RPE), which leads to a build-up of undesired retinoid derivatives in the photoreceptor outer segments. Due to constant generation of photoreceptor outer segments, as older discs become more terminal they are consumed by the RPE. In photoreceptor cells carrying mutant, non-functional ABCA4, bisretinoids retained in the disc membranes build up in the RPE cells with further biochemical processes taking place that lead to formation of the toxicity compound A2E, a key element of lipofuscin. ABCA4 mutations are associated with the build-up of toxins in the photoreceptors and the RPE. Exemplary toxins include, but are not limited to, all-trans-retinal, bisretinoids and lipofuscin.

Lack of functional ABCA4 prevents the transport of free retinaldehyde from the luminal to the cytoplasmic side of the photoreceptor cell disc outer membranes, resulting in increased formation, or amplifying, the formation of retinoid dimers (bisretinoids). Upon daily phagocytosis of the distal outer segments of photoreceptor cells by the retinal pigment epithelium (RPE), the retinoid derivatives are processed further, leading to accumulation of bisretinoids. The retinoid derivatives are processed but are insoluble and accumulate. The outcome of this accumulation leads to dysfunction and eventual death of the RPE cells with subsequent secondary loss of the overlying photoreceptors through degeneration and subsequent death. The inventors have characterized the fundus changes in the pigmented Abca4^{-/-} mouse model and documented the positive effects of deuterised vitamin A on fundus fluorescence and bisretinoid accumulation. In this disclosure, the inventors show that delivery of ABCA4 to the photoreceptors of the Abca4^{-/-} mouse model using an overlapping AAV dual vector system reduces the buildup of toxic bisretinoids, such an effect in a patient with Stargardt disease could prevent death of the RPE cells and the degeneration and death of the photoreceptor cells they support.

In some embodiments of the compositions and methods of the disclosure, the AAV dual vector system of the disclosure generates as a full length ABCA4 transgene in one or more cells of an eye of subject. In some embodiments, the subject has Stargardt disease. In some embodiments, the one or more cells comprise photoreceptor cells. In some embodiments, the one or more cells comprise RPE cells. In some embodiments, the one or more cells comprise RPE cells, photoreceptor cells, or a combination thereof.

In some embodiments of the compositions and methods of the disclosure, expression of the ABCA4 transgene in the one or more cells of the eye of the subject slows the degeneration of photoreceptor cells. In some embodiments, the one or more cells comprise RPE cells, photoreceptor cells, or a combination thereof. In some embodiments, expression of the ABCA4 transgene in the one or more cells of the eye of the subject prevents the death of photoreceptor cells. In some embodiments, expression of the ABCA4 transgene in the one or more cells of the eye of the subject prevents the degeneration of photoreceptor cells. In some embodiments, expression of the ABCA4 transgene in the one or more cells of the eye of the subject restores the photoreceptor cells to healthy or viable photoreceptor cells.

In some embodiments, expression of the ABCA4 transgene in the one or more cells of the eye of the subject prevents the death of RPE cells. In some embodiments, expression of the ABCA4 transgene in the one or more cells of the eye of the subject prevents the death of RPE cells and the degeneration of photoreceptor cells.

Viral vectors are derived from wild type viruses which are modified using recombinant nucleic acid technologies to incorporate a non-native nucleic acid sequence (or transgene) into the viral genome. The ability of viruses to target and infect specific cells is used to deliver the transgene into a target cell, leading to the expression of the gene and the production of the encoded gene product.

The disclosure relates to vectors derived from adeno-associated virus (AAV).

A potentially limiting factor of adeno-associated viral (AAV) vectors is the size restriction of the encodable DNA transgene at under 5 kb.

Stargardt disease is the most prevalent form of recessively inherited blindness and is caused by mutations in ABCA4, which has a coding sequence length of 6.8 kb. Dual vector approaches increase the capacity of AAV gene therapy but questions have been raised regarding whether the levels of target protein generated would be sufficient to achieve a therapeutic effect. Additionally, dual vectors commonly produce unwanted truncated proteins. Here, we describe a systematic approach for optimizing an overlapping AAV dual vector system for delivery of the 6.8 kb coding sequence of human ABCA4, mutations in which cause Stargardt disease, the most common form of recessively inherited blindness in young people. The data of the disclosure demonstrate an optimized overlapping dual vector strategy to deliver full length ABCA4 to the photoreceptor outer segments of Abca4^{-/-} mice at levels that enabled a therapeutic effect whilst reducing to undetectable levels truncated protein forms.

In vitro and in vivo assessments generated full-length ABCA4 protein following transduction with overlapping dual vectors and improvements were achieved by identifying an optimal region of overlap and including a 5′ untranslated region in the upstream transgene and a Woodchuck hepatitis virus post-transcriptional regulatory element in the downstream transgene. Truncated ABCA4 was reduced through specific sequence selections in the downstream transgene. The optimized overlapping dual vector system generated functional ABCA4 protein in the photoreceptor outer segments of Abca4^{-/-} mice, leading to a therapeutic effect. This was quantified by a reduction in bisretinoid and A2E levels in treated eyes 3 months post-injection and in lipofuscin and melanin-related autofluorescence at 6 months post-injection. These observations support a dual vector approach in future clinical trials using AAV gene therapy to treat Stargardt disease.

The disclosure provides an adeno-associated viral (AAV) vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence of contiguous nucleotides corresponding to nucleotides 105 to 3597 of SEQ ID NO: 1; wherein the second nucleic acid sequence comprises a sequence of contiguous nucleotides corresponding to nucleotides 3806 to 6926 of SEQ ID NO: 1; wherein the first nucleic acid sequence and the second nucleic acid sequence each comprise a region of sequence overlap with the other; and wherein the region of sequence overlap comprises at least about 20 contiguous nucleotides of a nucleic acid sequence corresponding to nucleotides 3598 to 3805 of SEQ ID NO: 1.

AAV vectors in general are well known in the art and a skilled person is familiar with general techniques suitable for their preparation from his common general knowledge in the field. The skilled person's knowledge includes techniques suitable for incorporating a nucleic acid sequence of interest into the genome of an AAV vector.

The term “AAV vector system” is used to embrace the fact that the first and second AAV vectors are intended to work together in a complementary fashion.

The first and second AAV vectors of the AAV vector system of the disclosure together encode an entire ABCA4 transgene. Thus, expression of the encoded ABCA4 transgene in a target cell requires transduction of the target cell with both first (upstream) and second (downstream) vectors.

The AAV vectors of the AAV vector system of the disclosure can be in the form of AAV particles (also referred to as virions). An AAV particle comprises a protein coat (the capsid) surrounding a core of nucleic acid, which is the AAV genome. The present disclosure also encompasses nucleic acid sequences encoding AAV vector genomes of the AAV vector system described herein.

SEQ ID NO: 1 is the human ABCA4 nucleic acid sequence corresponding to NCBI Reference Sequence NM_000350.2. SEQ ID NO: 1 is identical to NCBI Reference Sequence NM_000350.2. The ABCA4 coding sequence spans nucleotides 105 to 6926 of SEQ ID NO: 1.

The first AAV vector comprises a first nucleic acid sequence comprising a 5′ end portion of an ABCA4 CDS. A 5′ end portion of an ABCA4 CDS is a portion of the ABCA4 CDS that includes its 5′ end. Because it is only a portion of a CDS, the 5′ end portion of an ABCA4 CDS is not a full-length (i.e. is not an entire) ABCA4 CDS. Thus, the first nucleic acid sequence (and thus the first AAV vector) does not comprise a full-length ABCA4 CDS.

The second AAV vector comprises a second nucleic acid sequence comprising a 3′ end portion of an ABCA4 CDS. A 3′ end portion of an ABCA4 CDS is a portion of the ABCA4 CDS that includes its 3′ end. Because it is only a portion of a CDS, the 3′ end portion of an ABCA4 CDS is not a full-length (i.e. is not an entire) ABCA4 CDS. Thus, the second nucleic acid sequence (and thus the second AAV vector) does not comprise a full-length ABCA4 CDS.

The 5′ end portion and 3′ end portion together encompass the entire ABCA4 CDS (with a region of sequence overlap, as discussed below). Thus, a full-length ABCA4 CDS is contained in the AAV vector system of the disclosure, split across the first and second AAV vectors, and can be reassembled in a target cell following transduction of the target cell with the first and second AAV vectors.

The first nucleic acid sequence as described above comprises a sequence of contiguous nucleotides corresponding to nucleotides 105 to 3597 of SEQ ID NO: 1. The ABCA4 CDS begins at nucleotide 105 of SEQ ID NO: 1.

The second nucleic acid sequence as described above comprises a sequence of contiguous nucleotides corresponding to nucleotides 3806 to 6926 of SEQ ID NO: 1.

In order to encompass the entire ABCA4 CDS, the first and second nucleic acid sequences each further comprise at least a portion of the ABCA4 CDS corresponding to nucleotides 3598 to 3805 of SEQ ID NO: 1, such that when the first and second nucleic acid sequences are aligned the entirety of ABCA4 CDS corresponding to nucleotides 3598 to 3805 of SEQ ID NO: 1 is encompassed. Thus, when aligned, the first and second nucleic acid sequences together encompass the entire ABCA4 CDS.

Furthermore, the first and second nucleic acid sequences comprise a region of sequence overlap allowing reconstruction of the entire ABCA4 CDS as part of a full-length transgene inside a target cell transduced with the first and second AAV vectors of the disclosure.

When the first and second nucleic acid sequences are aligned with each other, a region at the 3′ end of the first nucleic acid sequence overlaps with a corresponding region at the 5′ end of the second nucleic acid sequence. Thus, both the first and second nucleic acid sequences comprise a portion of the ABCA4 CDS that forms the region of sequence overlap.

In some embodiments, the region of overlap between the first and second nucleic acid sequences comprises at least about 20 contiguous nucleotides of the portion of the ABCA4 CDS corresponding to nucleotides 3598 to 3805 of SEQ ID NO: 1.

In some embodiments, the region of overlap may extend upstream and/or downstream of said 20 contiguous nucleotides. Thus, the region of overlap may be more than 20 nucleotides in length.

The region of overlap may comprise nucleotides upstream of the position corresponding to nucleotide 3598 of SEQ ID NO: 1. Alternatively, or in addition, the region of overlap may comprise nucleotides downstream of the position corresponding to nucleotide 3805 of SEQ ID NO: 1.

Alternatively, the region of nucleic acid sequence overlap may be contained within the portion of the ABCA4 CDS corresponding to nucleotides 3598 to 3805 of SEQ ID NO: 1.

Thus, in one embodiment, the region of nucleic acid sequence overlap is between 20 and 550 nucleotides in length; preferably between 50 and 250 nucleotides in length; preferably between 175 and 225 nucleotides in length; preferably between 195 and 215 nucleotides in length.

In one embodiment, the region of nucleic acid sequence overlap comprises at least about 50 contiguous nucleotides of a nucleic acid sequence corresponding to nucleotides 3598 to 3805 of SEQ ID NO: 1; preferably at least about 75 contiguous nucleotides; preferably at least about 100 contiguous nucleotides; preferably at least about 150 contiguous nucleotides; preferably at least about 200 contiguous nucleotides; preferably all 208 contiguous nucleotides.

In certain preferred embodiments, the region of nucleic acid sequence overlap commences at the nucleotide corresponding to nucleotide 3598 of SEQ ID NO: 1. The term “commences” means that the region of nucleic acid sequence overlap runs in the direction 5′ to 3′ starting from the nucleotide corresponding to nucleotide 3598 of SEQ ID NO: 1. Thus, in a preferred embodiment, the most 5′ nucleotide of the region of nucleic acid sequence overlap corresponds to nucleotide 3598 of SEQ ID NO: 1.

In certain preferred embodiments, the region of nucleic acid sequence overlap between the first nucleic acid sequence and the second nucleic acid sequence vector corresponds to nucleotides 3598 to 3805 of SEQ ID NO: 1.

A construction of dual AAV vectors comprising a region of nucleic acid sequence overlap as described above can reduce the level of translation of unwanted truncated ABCA4 peptides.

The problem of translation of truncated ABCA4 peptides may arise in dual AAV vector systems when translation is initiated from mRNA transcripts derived from the downstream vector only. In this regard, AAV ITRs such as the AAV2 5′ ITR may have promoter activity; this together with the presence in a downstream vector of WPRE and bGH poly-adenylation sequences (as discussed below) may lead to the generation of stable mRNA transcripts from unrecombined downstream vectors. The wild-type ABCA4 CDS carries multiple in-frame AUG codons in its downstream portion that cannot be substituted for other codons without altering the amino acid sequence. This creates the possibility of translation occurring from the stable transcripts, leading to the presence of truncated ABCA4 peptides.

In certain preferred embodiments of the disclosure wherein the region of nucleic acid sequence overlap commences at the nucleotide corresponding to nucleotide 3598 of SEQ ID NO: 1, the starting sequence of the overlap zone includes an out-of-frame AUG (start) codon in good context (regarding the potential Kozak consensus sequence) prior to an in-frame AUG codon in weaker context in order to encourage the translational machinery to initiate translation of unrecombined downstream-only transcripts from an out-of-frame site. In certain particularly preferred embodiments of the disclosure, there are in total four out-of-frame AUG codons in various contexts prior to the in-frame AUG. All of these translate to a STOP codon within 10 amino acids, thus preventing the translation of unwanted truncated ABCA4 peptides.

In certain preferred embodiments, the first nucleic acid sequence comprises a sequence of contiguous nucleotides corresponding to nucleotides 105 to 3805 of SEQ ID NO: 1, and the second nucleic acid sequence comprises a sequence of contiguous nucleotides corresponding to nucleotides 3598 to 6926 of SEQ ID NO: 1, so encompassing the region of nucleic acid sequence overlap as described above.

Thus, in certain preferred embodiments, the 5′ end portion of an ABCA4 CDS consists of a sequence of contiguous nucleotides corresponding to nucleotides 105 to 3805 of SEQ ID NO: 1, and the 3′ end portion of an ABCA4 CDS consists of a sequence of contiguous nucleotides corresponding to nucleotides 3598 to 6926 of SEQ ID NO: 1.

In certain preferred embodiments, the 5′ end portion of an ABCA4 CDS consists of nucleotides 105 to 3805 of SEQ ID NO: 1, and the 3′ end portion of an ABCA4 CDS consists of nucleotides 3598 to 6926 of SEQ ID NO: 1.

Thus, in certain preferred embodiments, the disclosure provides an AAV vector system for expressing a human ABCA4 protein in a target cell, the AAV vector system comprising a first AAV vector comprising a first nucleic acid sequence and a second AAV vector comprising a second nucleic acid sequence, wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the 5′ end portion of an ABCA4 CDS consists of a sequence of contiguous nucleotides corresponding to nucleotides 105 to 3805 of SEQ ID NO: 1, and wherein the 3′ end portion of an ABCA4 CDS consists of a sequence of contiguous nucleotides corresponding to nucleotides 3598 to 6926 of SEQ ID NO: 1.

In certain preferred embodiments, the disclosure provides an AAV vector system for expressing a human ABCA4 protein in a target cell, the AAV vector system comprising a first AAV vector comprising a first nucleic acid sequence and a second AAV vector comprising a second nucleic acid sequence, wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the 5′ end portion of an ABCA4 CDS consists of nucleotides 105 to 3805 of SEQ ID NO: 1, and wherein the 3′ end portion of an ABCA4 CDS consists of nucleotides 3598 to 6926 of SEQ ID NO: 1.

In accordance with the term “consists of”, in embodiments wherein the 5′ end portion of an ABCA4 CDS and the 3′ end portion of an ABCA4 CDS consist of specific sequences of contiguous nucleotides as described above, then the first nucleic acid sequence and the second nucleic acid sequence each do not comprise any additional ABCA4 CDS.

In certain embodiments, each of the first AAV vector and the second AAV vector comprises 5′ and 3′ Inverted Terminal Repeats (ITRs).

In certain embodiments, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. AAV ITRs are believed to aid concatemer formation in the nucleus of an AAV-infected cell, for example following the conversion of single-stranded vector DNA into double-stranded DNA by the action of host cell DNA polymerases. The formation of such episomal concatemers may serve to protect the vector construct during the life of the host cell, thereby allowing for prolonged expression of the transgene in vivo.

Thus, in some embodiments, the ITRs are AAV ITRs (i.e. ITR sequences derived from ITR sequences found in an AAV genome).

The first and second AAV vectors of the AAV vector system of the disclosure together comprise all of the components necessary for a fully functional ABCA4 transgene to be re-assembled in a target cell following transduction by both vectors. A skilled person is aware of additional genetic elements commonly used to ensure transgene expression in a viral vector-transduced cell. These may be referred to as expression control sequences. Thus, the AAV vectors of the AAV viral vector system of the disclosure may comprise expression control sequences (e.g. comprising a promoter sequence) operably linked to the nucleotide sequences encoding the ABCA4 transgene.

5′ expression control sequences components can be located in the first (“upstream”) AAV vector of the viral vector system, while 3′ expression control sequences can be located in the second (“downstream”) AAV vector of the viral vector system.

Thus, in some embodiments, the first AAV vector may comprise a promoter operably linked to the 5′ end portion of an ABCA4 CDS. The promoter may be required by its nature to be located 5′ to the ABCA4 CDS, hence its location in the first AAV vector.

Any suitable promoter may be used, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter). The promoter may show inducible expression in response to presence of another factor, for example a factor present in a host cell. In those embodiments where the vector is administered for therapy, the promoter should be functional in the target cell background.

In some embodiments, the promoter shows retinal-cell specific expression in order to allow for the transgene to only be expressed in retinal cell populations. Thus, expression from the promoter may be retinal-cell specific, for example confined only to cells of the neurosensory retina and retinal pigment epithelium.

An exemplary promoter suitable for use in the present disclosure is the chicken beta-actin (CBA) promoter, optionally in combination with a cytomegalovirus (CMV) enhancer element. Another exemplary promoter for use in the disclosure is a hybrid CBA/CAG promoter, for example the promoter used in the rAVE expression cassette (GeneDetect.com).

Examples of promoters based on human sequences that induce retina-specific gene expression include rhodopsin kinase for rods and cones, PR2.1 for cones only, and RPE65 for the retinal pigment epithelium.

Gene expression may be achieved using a GRK1 promoter. Thus, in certain embodiments, the promoter is a human rhodopsin kinase (GRK1) promoter.

In some embodiments, the GRK1 promoter sequence of the disclosure comprises or consists of 199 nucleotides in length and comprises or consists of nucleotides−112 to +87 of the GRK1 gene. In certain preferred embodiments, the promoter comprises or consists of the nucleic acid sequence of SEQ ID NO: 5 or a variant thereof having at least 90% (e.g. at least 90%, 95%, 96%,97%, 98%,99%, 99.1%,99.2%,99.3% 99.4%, 99.5% 99.6%,99.7% 99.8% or 99.9%) sequence identity to SEQ ID NO: 5.

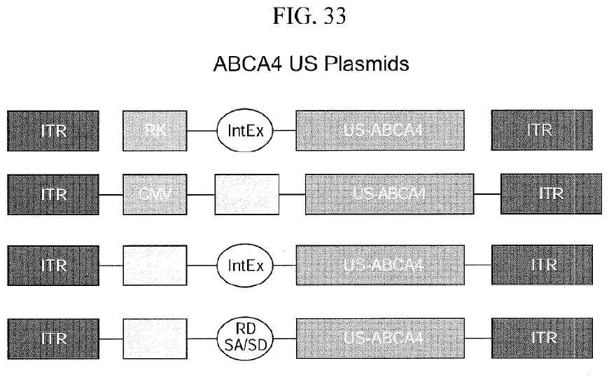

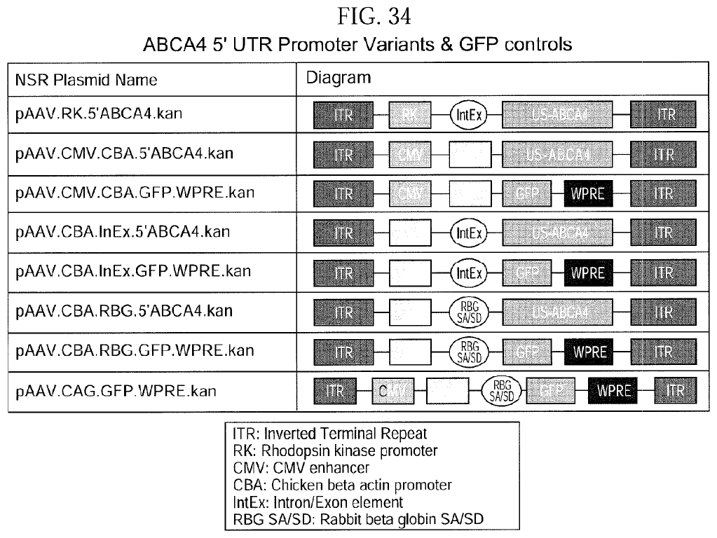

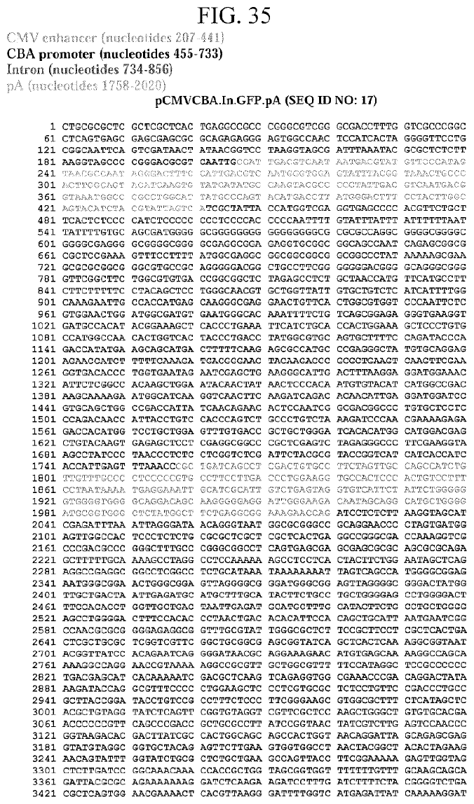

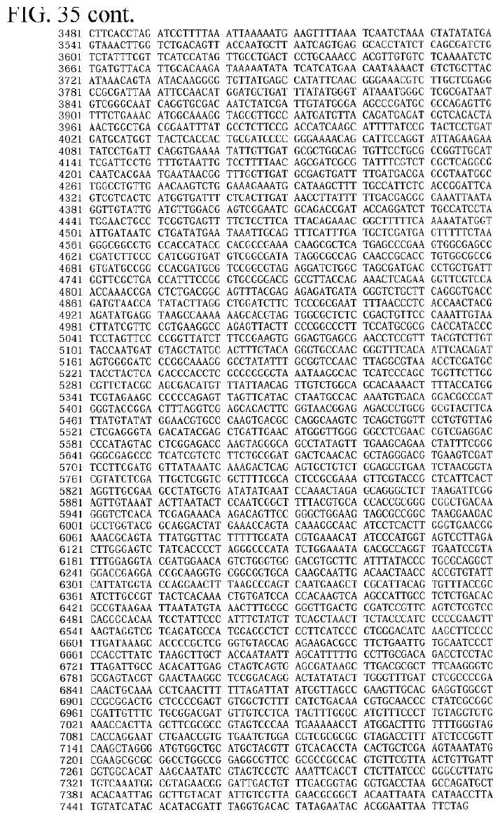

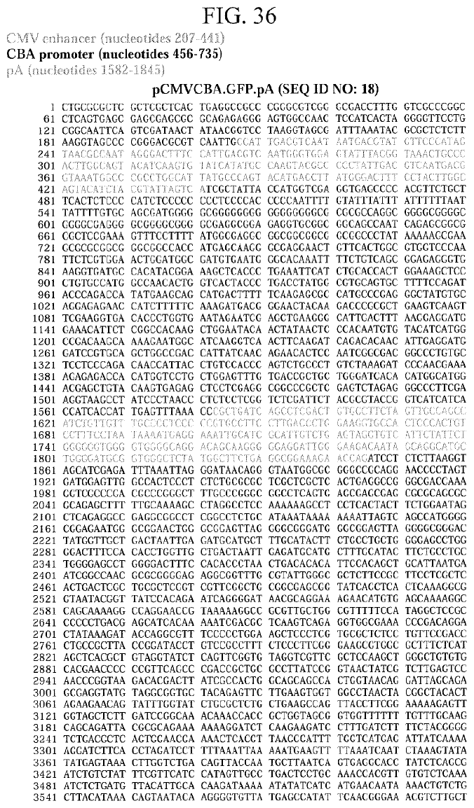

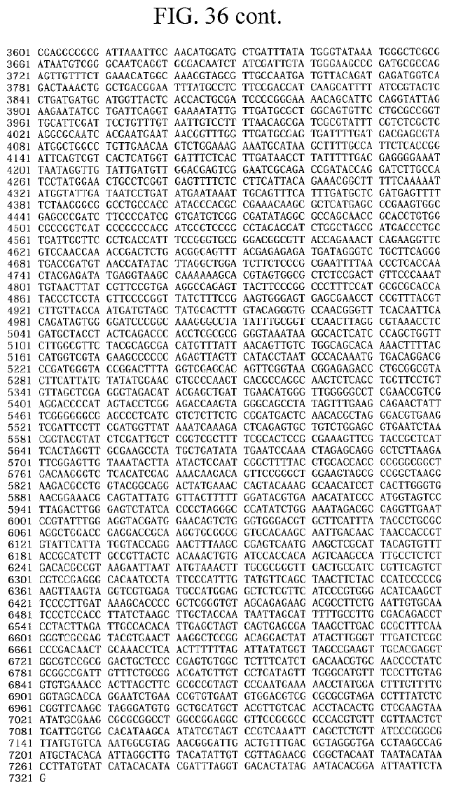

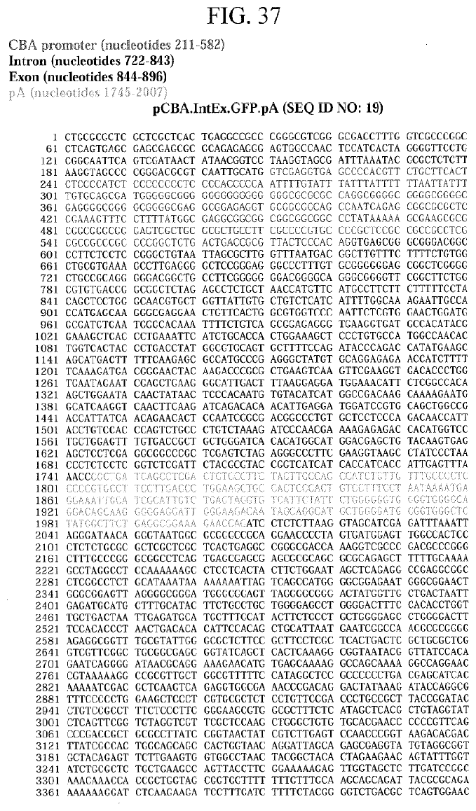

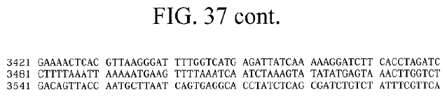

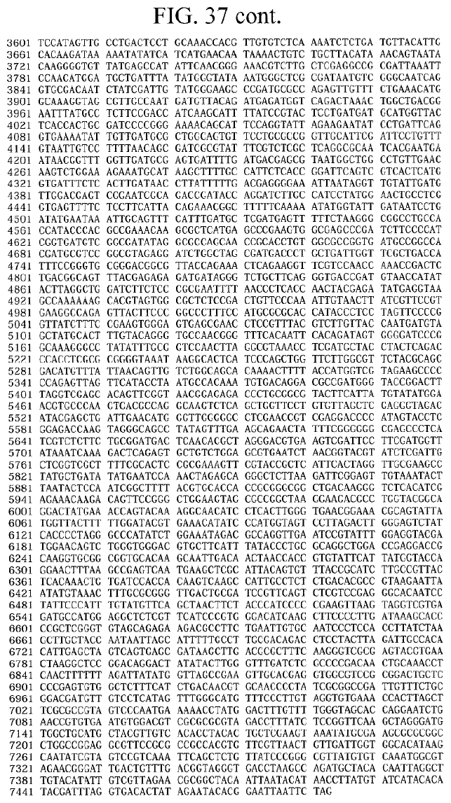

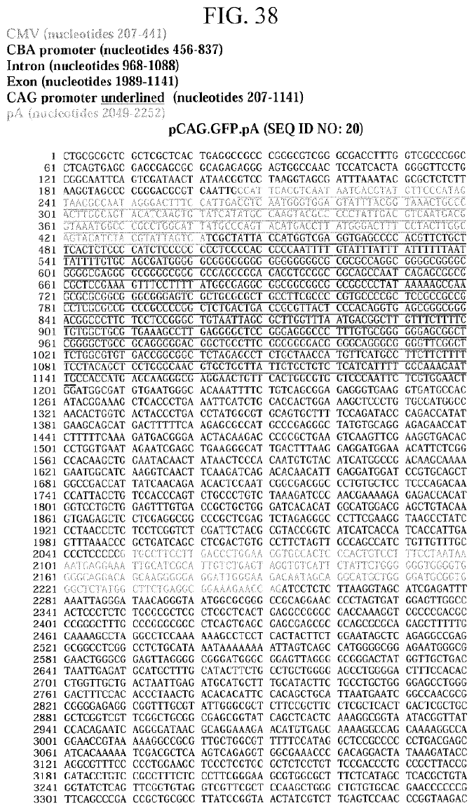

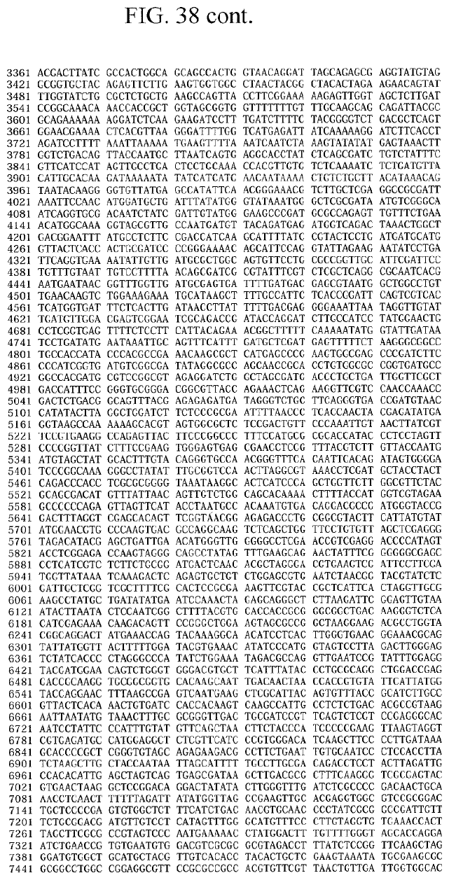

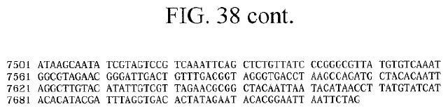

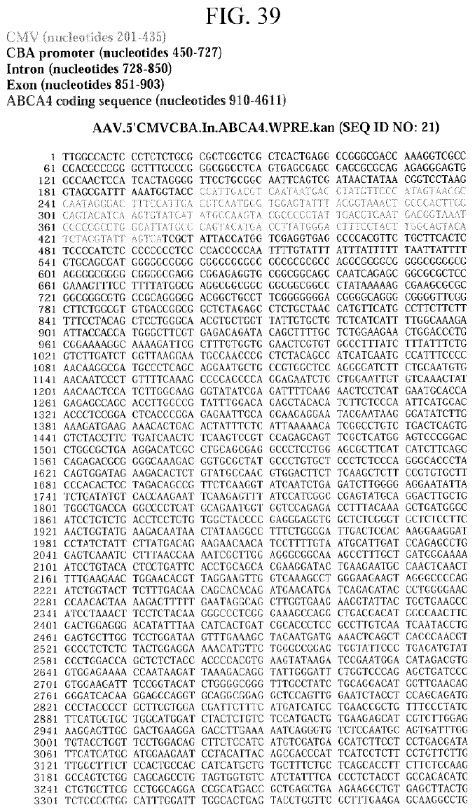

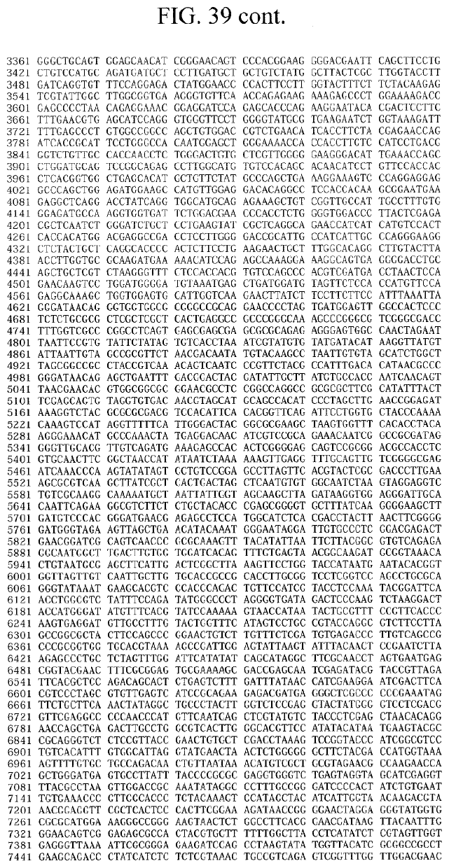

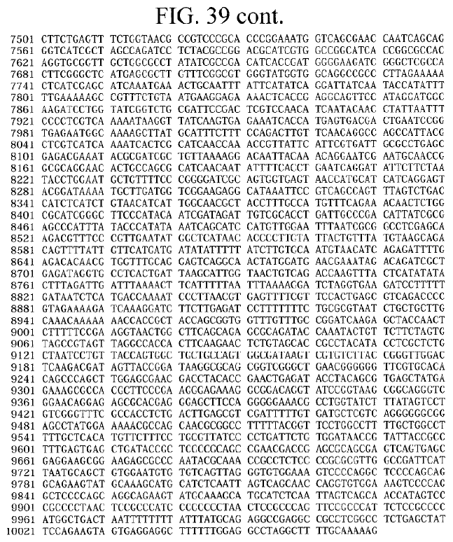

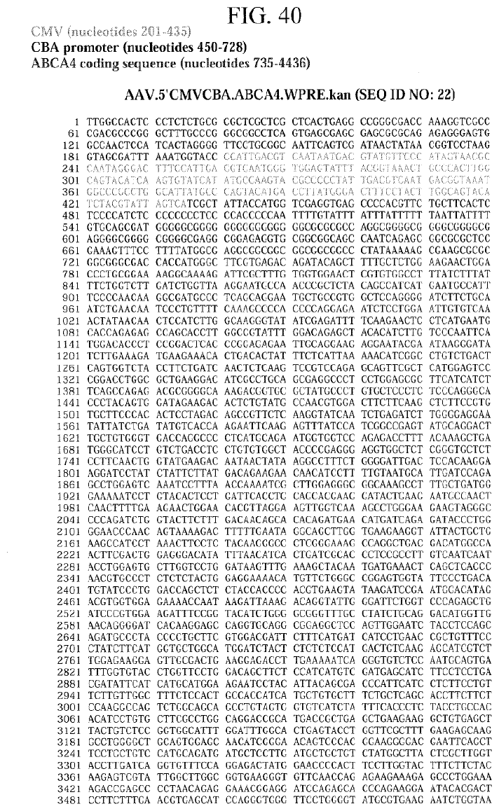

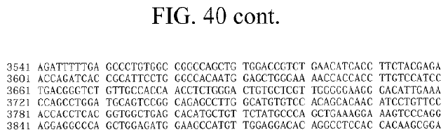

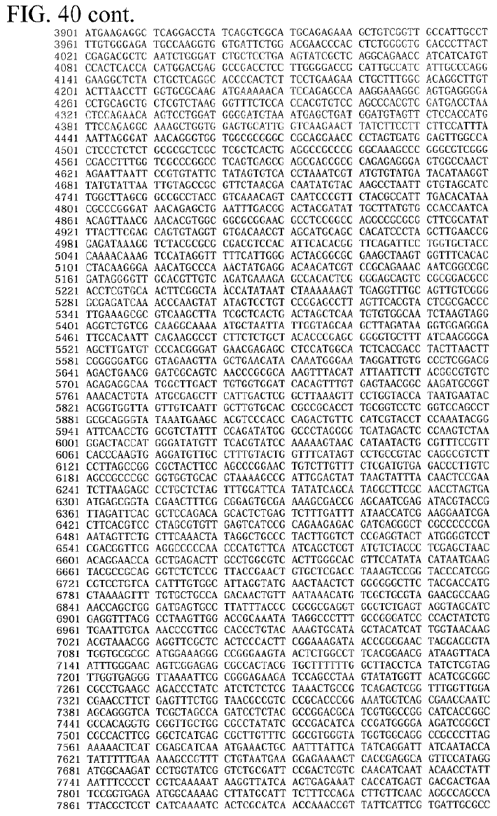

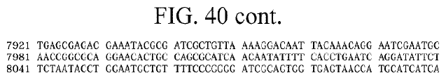

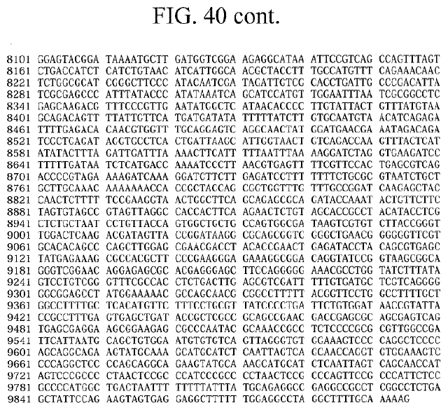

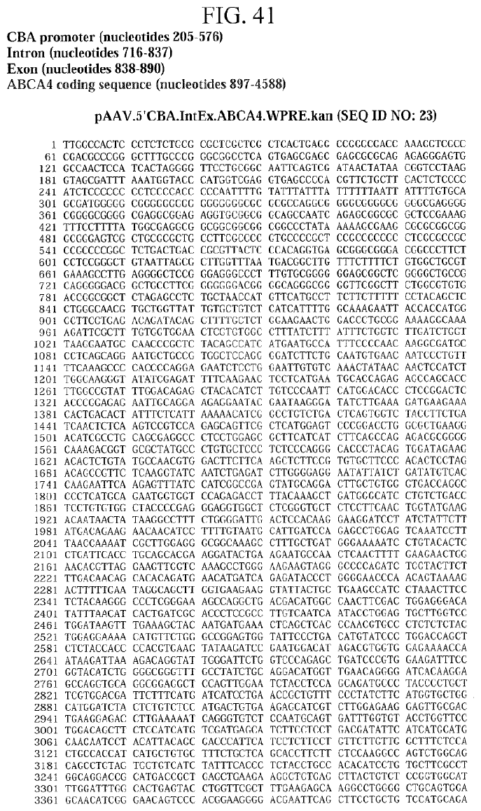

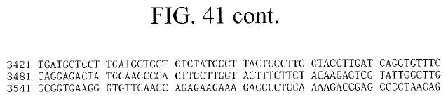

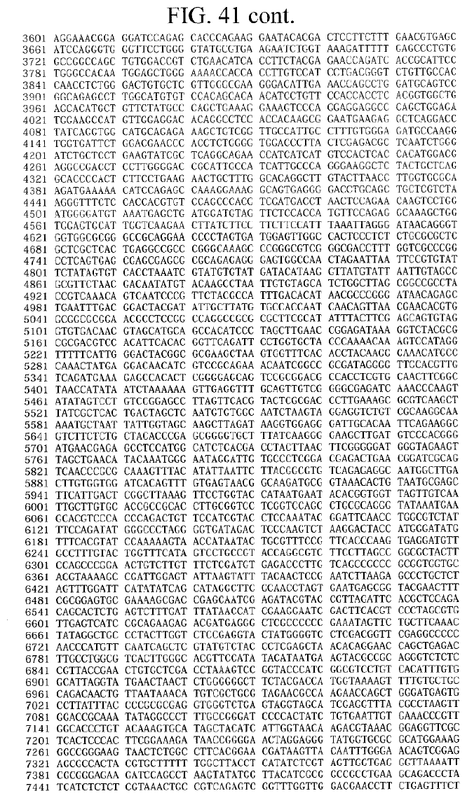

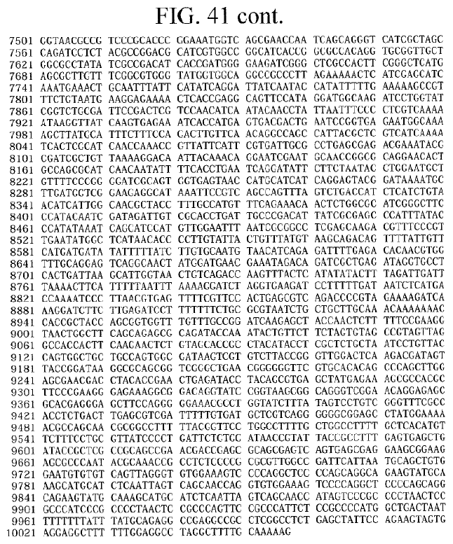

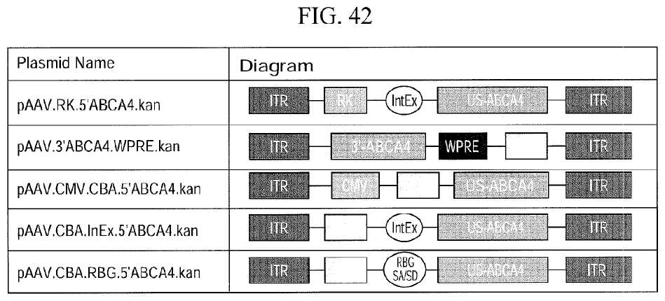

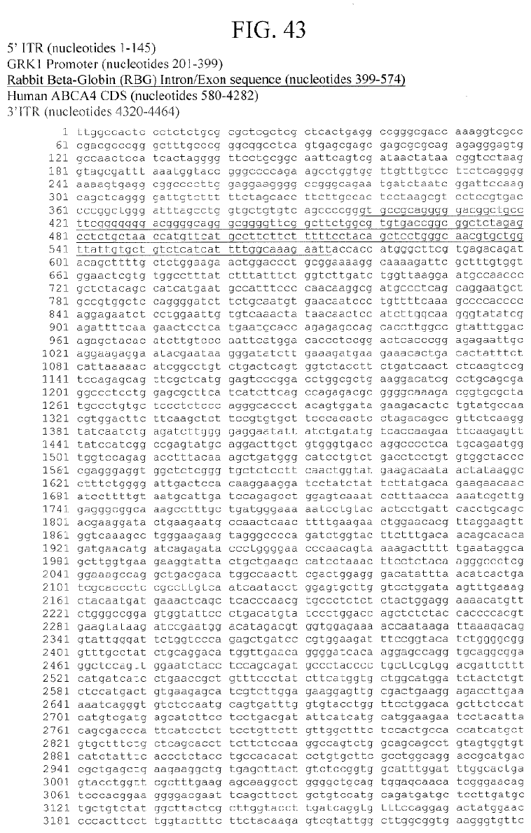

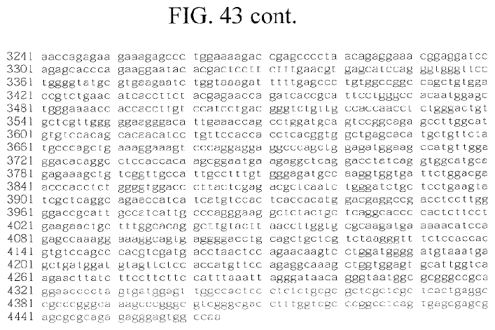

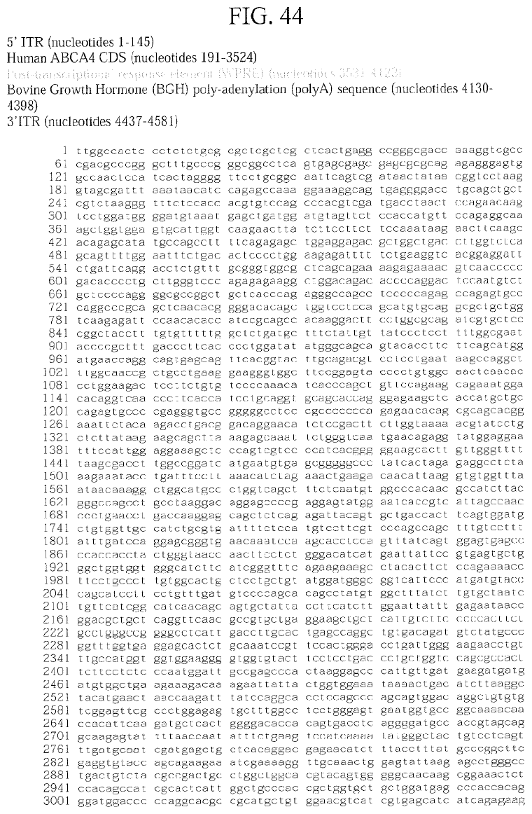

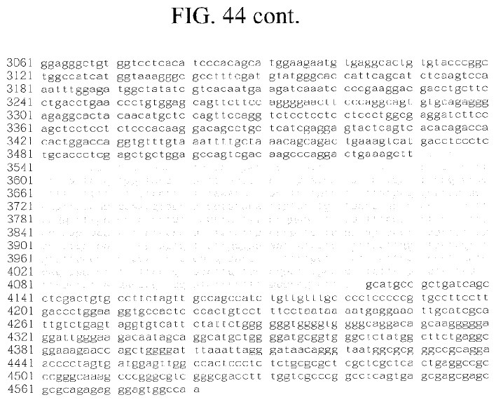

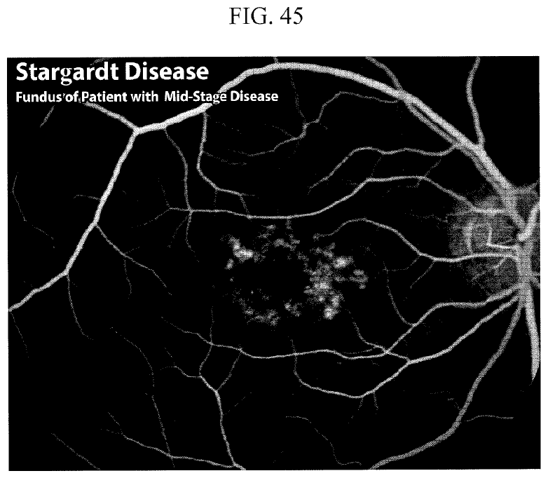

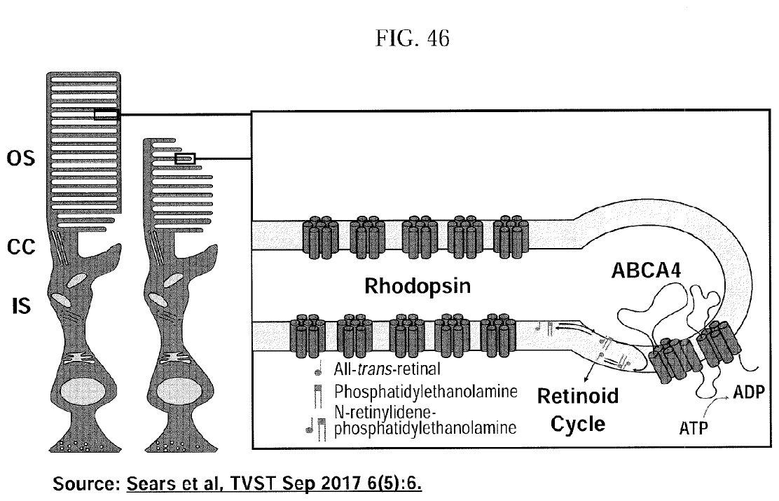

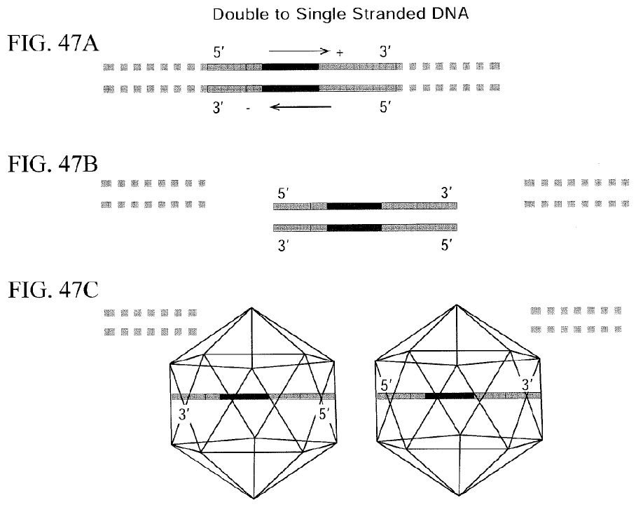

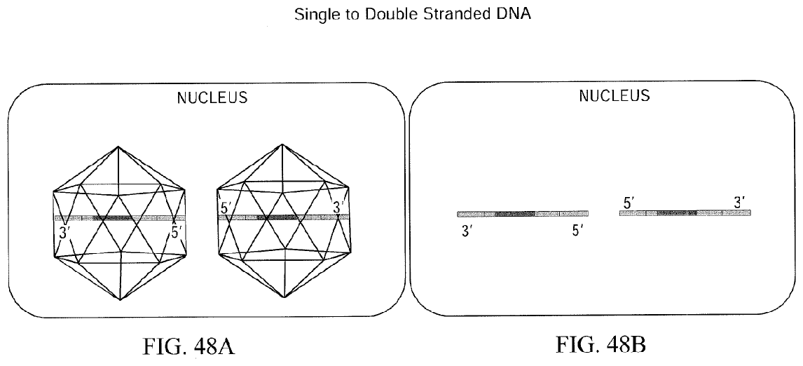

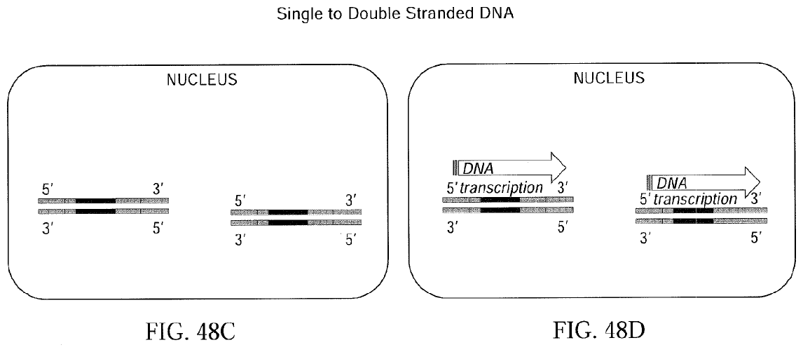

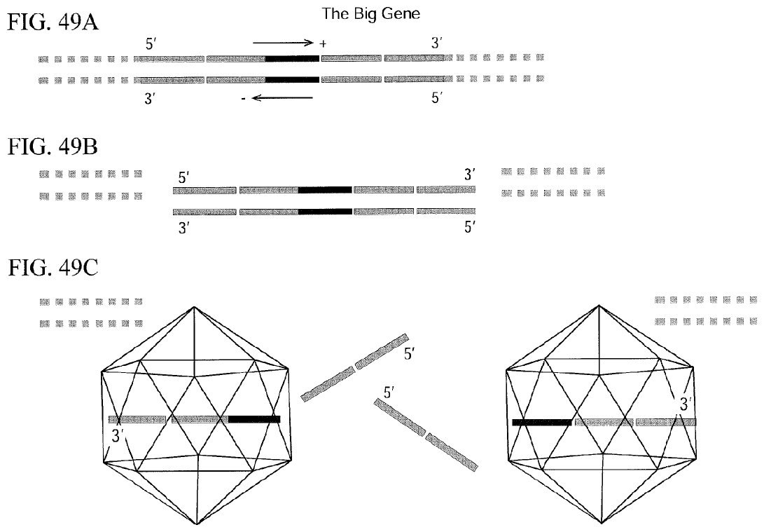

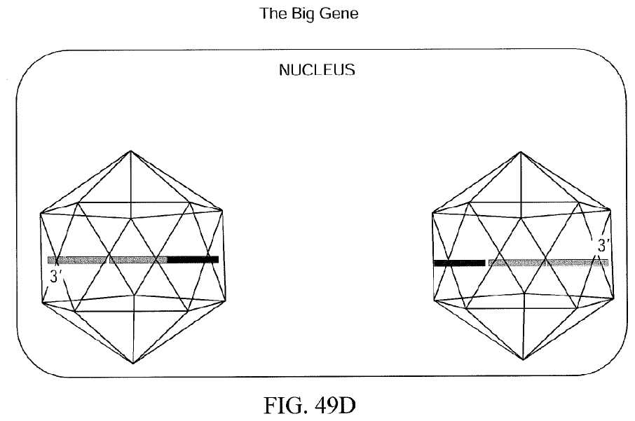

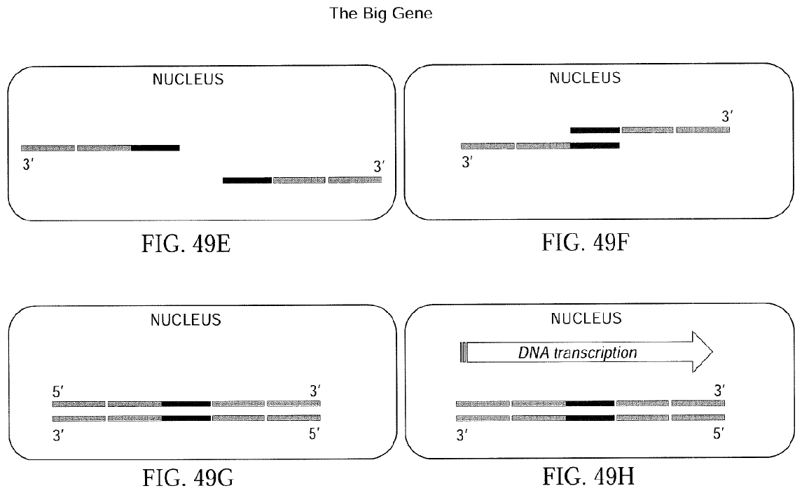

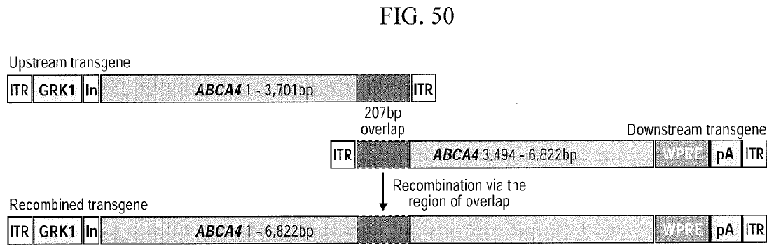

Elements may be included in both the upstream and downstream vectors of the disclosure to increase expression of ABCA4 protein. For example, the inclusion of an intron in a vector, such as the upstream vector of the disclosure, can increase the expression of an RNA or protein of interest from that vector. An intron is a nucleotide sequence within a gene that is removed by RNA splicing during RNA maturation. Introns can vary in length from tens of base pairs to multiple megabases. However, spliceosomal introns (i.e. introns that are spliced by the eukaryotic spliceosome) may comprise a splice donor (SD) site at the 5′ end of the intron, a branch site in the intron near the 3′ end, and a splice acceptor (SA) site at the 3′ end. These intron elements facilitate proper intron splicing. SD sites may comprise a consensus GU at the 5′ end of the intron and the SA site at the 3′ end of the intron may terminate with “AG.” Upstream of the SA site, introns often contain a region high in pyrimidines, which is between the branch point adenine nucleotide and the SA. Without wishing to be bound by any particular theory, the presence of an intron can affect the rate of RNA transcription, nuclear export or RNA transcript stability. Further, the presence of an intron may also increase the efficiency of mRNA translation, yielding more of a protein of interest (e.g. ABCA4). FIGS. 33 and 34 describe two exemplary introns (and accompanying exons) for use with ABCA4 dual vectors, IntEx and RBG SA/SD. However, the disclosure encompasses the use in a construct of the disclosure any intron that boosts gene expression and facilitates splicing in a eukaryotic cell.

In some embodiments of the vectors of the disclosure, the intron, the IntEx or the SA/SD (including a RBD SA/SD) may be one of several elements that function to increase protein expression from the vector. For example, the promoter and, optionally, an enhancer, can affect not just cell or tissue specificity of gene expression, but also the levels of mRNA that are transcribed from the vector. Promoters are regions of DNA that initiate RNA transcription. Depending on the specific sequence elements of the promoter, promoters may vary in strength and tissue specificity. Enhancers are DNA sequences that regulate transcription from promoters by affecting the ability of the promoter to recruit RNA polymerase and initiate transcription. Therefore, the choice of promoter, and optionally, the inclusion of an enhancer and/or the choice of the enhancer itself, in a vector can significantly affect the expression of a gene encoded by the vector. Exemplary promoters, such as the rhodopsin kinase promoter or chicken beta actin promoter, optionally combined with a CMV enhancer, are shown in FIGS. 33 and 34. In some embodiments, vectors of the disclosure comprise an exemplary promoter, such as the rhodopsin kinase promoter or chicken beta actin promoter, while excluding the use of an enhancer element. In some embodiments, vectors of the disclosure comprise an exemplary promoter, such as the chicken beta actin promoter, while excluding the use of an enhancer element, such as a CMV enhancer element. In some embodiments, vectors of the disclosure comprise an exemplary promoter, such as the rhodopsin kinase promoter or chicken beta actin promoter, while excluding the use of an enhancer element and while including an intron, an IntEx or an SD/SA. In some embodiments, vectors of the disclosure comprise an exemplary promoter, such as the chicken beta actin promoter, while excluding the use of an enhancer element, such as a CMV enhancer element and while including an intron, an IntEx or an SD/SA.

Elements in the non-coding sequences of the mRNA transcript itself can also affect protein levels of a sequence encoded in a vector. Without wishing to be limited by any particular theory, sequence elements in the mRNA untranslated regions (UTRs) can effect mRNA stability, which, in turn, affects levels of protein translation. An exemplary sequence element is a Posttranscriptional Regulatory Element (PRE) (e.g. a Woodchuck Hepatitis PRE (WPRE)), which increases mRNA stability. Exemplary promoters, enhancers, PREs, and the arrangement of these elements in vectors of the disclosure, are shown in FIGS. 33 and 34.

In some embodiments of the first AAV vector of the disclosure, the promoter may be operably linked with an intron and an exon sequence. In some embodiments of the first AAV vector of the disclosure, a nucleic acid sequence may comprise the promoter, an intron and an exon sequence. The intron and the exon sequence may be downstream of the promoter sequence. The intron and the exon sequence may be positioned between the promoter sequence and the upstream ABCA4 nucleic acid sequence (US-ABCA4). The presence of an intron and an exon may increase levels of protein expression. In some embodiments, the intron is positioned between the promoter and the exon. In some embodiments, including those embodiments wherein the intron is positioned between the promoter and the exon, the exon is positioned 5′ of the US-ABCA4 sequence. In some embodiments, the promoter comprises a promoter isolated or derived from a vertebrate gene. In some embodiments, the promoter is GRK1 promoter or a chicken beta actin (CBA) promoter.

The exon may comprise a coding sequence, a non-coding sequence, or a combination of both. In some embodiments, the exon comprises a non-coding sequence. In some embodiments, the exon is isolated or derived from a mammalian gene. In embodiments, the mammal is a rabbit (Oryctolagus cuniculus). In some embodiments, the mammalian gene comprises a rabbit beta globin gene or a portion thereof. In some embodiments, the exon comprises or consists of a nucleic acid sequence having at least 80%, at least 90%, at least 95%, at least 96%, at least 97%, at least 98%, or at least 99% identity to the nucleic acid sequence of:

(SEQ ID NO: 14)
CTCCTGGGCA ACGTGCTGGT TATTGTGCTG TCTCATCATT
TTGGCAAAGA ATT.

In some embodiments, the exon comprises or consists of a nucleic acid sequence having 100% identity to the nucleic acid sequence of:

(SEQ ID NO: 14)
CTCCTGGGCA ACGTGCTGGT TATTGTGCTG TCTCATCATT
TTGGCAAAGA ATT.

Introns may comprise a splice donor site, a splice acceptor site or a branch point. Introns may comprise a splice donor site, a splice acceptor site and a branch point. Exemplary splice acceptor sites comprise nucleotides “GT” (“GU” in the pre-mRNA) at the 5′ end of the intron. Exemplary splice acceptor sites comprise an “AG” at the 3′ end of the intron. In some embodiments, the branch point comprises an adenosine (A) between 20 and 40 nucleotides, inclusive of the endpoints, upstream of the 3′ end of the intron. The intron may comprise an artificial or non-naturally occurring sequence. Alternatively, the intron may be isolated or derived from a vertebrate gene. The intron may comprise a sequence encoding a fusion of two sequences, each of which may be isolated or derived from a vertebrate gene. In some embodiments, a vertebrate gene from which the intron nucleic acid sequence or a portion thereof is derived comprises a chicken (Gallus gallus) gene. In some embodiments, the chicken gene comprises a chicken beta actin gene. In some embodiments, a vertebrate gene from which the intron nucleic acid sequence or a portion thereof is derived comprises a rabbit (Oryctolagus cuniculus) gene. In some embodiments, the rabbit gene comprises a rabbit beta globin gene or a portion thereof. In some embodiments, the intron comprises or consists of a nucleic acid sequence having at least 80%, at least 90%, at least 95%, at least 96%, at least 97%, at least 98%, or at least 99% identity to the nucleic acid sequence of:

| (SEQ ID NO: 13) | |
|---|---|
| 1 | GTGCCGCAGG GGGACGGCTG CCTTCGGGGG GGACGGGGCA GGGCGGGGTT CGGCTTCTGG |
| 61 | CGTGTGACCG GCGGCTCTAG AGCCTCTGCT AACCATGTTC ATGCCTTCTT CTTTTTCCTA |
| 121 | CAG. |

In some embodiments, the intron comprises or consists of a nucleic acid sequence having 100% identity to the nucleic acid sequence of:

| (SEQ ID NO: 13) | |
|---|---|
| 1 | GTGCCGCAGG GGGACGGCTG CCTTCGGGGG GGACGGGGCA GGGCGGGGTT CGGCTTCTGG |
| 61 | CGTGTGACCG GCGGCTCTAG AGCCTCTGCT AACCATGTTC ATGCCTTCTT CTTTTTCCTA |
| 121 | CAG. |

In some embodiments of the first (or upstream) AAV vector, the promoter comprises a hybrid promoter (a Cytomegalovirus (CMV) enhancer with a chicken beta actin (CBA) promoter). In some embodiments, the CMV enhancer sequence comprises or consists of a nucleic acid sequence having at least 80%, at least 90%, at least 95%, at least 96%, at least 97%, at least 98%, at least 99%, or at least any percentage identity in between to the nucleic acid sequence of:

| (SEQ ID NO: 15) | |
|---|---|
| 1 | CCATTGACGT CAATAATGAC GTATGTTCCC ATAGTAACGC CAATAGGGAC TTTCCATTGA |
| 61 | CGTCAATGGG TGGAGTATTT ACGGTAAACT GCCCACTTGG CAGTACATCA AGTGTATCAT |
| 121 | ATGCCAAGTA CGCCCCCTAT TGACGTCAAT GACGGTAAAT GGCCCGCCTG GCATTATGCC |
| 181 | CAGTACATGA CCTTATGGGA CTTTCCTACT TGGCAGTACA TCTACGTATT AGTCA. |

In some embodiments, the sequence encoding the first (or upstream) AAV vector comprises a sequence encoding a CBA promoter (without a CMV enhancer element), a sequence encoding an intron and a sequence encoding an exon. In some embodiments, the CBA promoter sequence comprises or consists of a nucleic acid sequence having at least 80%, at least 90%, at least 95%, at least 96%, at least 97%, at least 98%, at least 99%, or at least any percentage identity in between to the nucleic acid sequence of:

| (SEQ ID NO: 16) | |
|---|---|
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCGG GAGTCGCTGC GCGCTGCCTT |
| 301 | CGCCCCGTGC CCCGCTCCGC CGCCGCCTCG CGCCGCCCGC CCCGGCTCTG ACTGACCGCG |
| 361 | TTACTCCCAC AG. |

In some embodiments, the CBA promoter sequence comprises or consists of a nucleic acid sequence having at least 80%, at least 90%, at least 95%, at least 96%, at least 97%, at least 98%, at least 99%, or at least any percentage identity in between to the nucleic acid sequence of:

| (SEQ ID NO: 24) | |
|---|---|
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCG. |

In some embodiments, the sequence encoding the intron comprises or consists of the nucleic acid sequence of SEQ ID NO: 13. In some embodiments, the sequence encoding the exon comprises or consists of the nucleic acid sequence of SEQ ID NO: 14.

The first AAV vector may comprise an untranslated region (UTR) located between the promoter and the upstream ABCA4 nucleic acid sequence (i.e. a 5′ UTR).

Any suitable UTR sequence may be used, the selection of which may be readily made by the skilled person.

The UTR may comprise or consist of one or more of the following elements: a Gallus 3-actin (CBA) intron 1 or a portion thereof, an Oryctolagus cuniculus β-globin (RBG) intron 2 or a portion thereof, and an Oryctolagus cuniculus β-globin exon 3 or a portion thereof.

The UTR may comprise a Kozak consensus sequence. Any suitable Kozak consensus sequence may be used.

In certain preferred embodiments, the UTR comprises the nucleic acid sequence specified in SEQ ID NO: 6,a variant or a portion thereof having at least 90% (e.g. at least 90%, 95%, 96%, 97%, 98%, 99%, 99.1%, 99.2%, 99.3%, 99.4%, 99.5%, 99.6%, 99.7%, 99.8% or 99.9%) sequence identity.

The UTR of SEQ ID NO: 6 is 186 nucleotides in length and includes a Gallus β-actin (CBA) intron 1 fragment (with predicted splice donor site), Oryctolagus cuniculus β-globin (RBG) intron 2 fragment (including predicted branch point and splice acceptor site) and Oryctolagus cuniculus β-globin exon 3 fragment immediately prior to a Kozak consensus sequence.

The presence of a UTR as described above, in particular a UTR sequence as specified in SEQ ID NO: 6 or a variant thereof having at least 90% sequence identity, may increase translational yield from the ABCA4 transgene.

The second (“downstream”) AAV vector of the AAV vector system of the disclosure may comprise a post-transcriptional response element (also known as post-transcriptional regulatory element) or PRE. Any suitable PRE may be used, the selection of which may be readily made by the skilled person. In certain embodiments, the presence of a suitable PRE may enhance expression of the ABCA4 transgene.

In certain preferred embodiments, the PRE is a Woodchuck Hepatitis Virus PRE (WPRE). In certain particularly preferred embodiments, the WPRE has a sequence as specified in SEQ ID NO: 7 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

The second AAV vector may comprise a poly-adenylation sequence located 3′ to the downstream ABCA4 nucleic acid sequence. Any suitable poly-adenylation sequence may be used, the selection of which may be readily made by the skilled person.

In certain preferred embodiments, the poly-adenylation sequence is a bovine Growth Hormone (bGH) poly-adenylation sequence. In a particularly preferred embodiment, the bGH poly-adenylation sequence has a sequence as specified in SEQ ID NO: 8 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity. In certain embodiments, the sequence encoding the polyadenylation sequence comprises or consists of a nucleic acid sequence having at least 80%, at least 90%, at least 95%, at least 96%, at least 97%, at least 98%, at least 99%, or at least any percentage identity in between to the nucleic acid sequence of:

| (SEQ ID NO: 25) | |
|---|---|
| 1 | CGCTGATCAG CCTCGACTGT GCCTTCTAGT TGCCAGCCAT CTGTTGTTTG CCCCTCCCCC |
| 61 | GTGCCTTCCT TGACCCTGGA AGGTGCCACT CCCACTGTCC TTTCCTAATA AAATGAGGAA |
| 121 | ATTGCATCGC ATTGTCTGAG TAGGTGTCAT TCTATTCTGG GGGGTGGGGT GGGGCAGGAC |
| 181 | AGCAAGGGGG AGGATTGGGA AGACAATAGC AGGCATGCTG GGGATGCGGT GGGCTCTATG |
| 241 | GCTTCTGAGG CGGAAAGAAC CAG. |

In certain preferred embodiments of the AAV vector system of the disclosure, the first AAV vector comprises the nucleic acid sequence of SEQ ID NO: 9, and the second AAV vector comprises the nucleic acid sequence of SEQ ID NO: 10.

In certain preferred embodiments of the AAV vector system of the disclosure, the first AAV vector comprises the nucleic acid sequence of SEQ ID NO: 3, and the second AAV vector comprises the nucleic acid sequence of SEQ ID NO: 4.

The AAV vector system of the disclosure may be suitable for expressing a human ABCA4 protein in a target cell.

The disclosure provides a method for expressing a human ABCA4 protein in a target cell, the method comprising the steps of: transducing the target cell with the first AAV vector and the second AAV vector as described above, such that a functional ABCA4 protein is expressed in the target cell.

Expression of human ABCA4 protein requires that the target cell be transduced with both the first AAV vector and the second AAV vector. In certain embodiments, the target cell may be transduced with the first AAV vector and the second AAV vector in any order (first AAV vector followed by second AAV vector, or second AAV vector followed by first AAV vector) or simultaneously.

Methods for transducing target cells with AAV vectors are known in the art and will be familiar to a skilled person.

The target cell is may be a cell of the eye, preferably a retinal cell (e.g. a neuronal photoreceptor cell, a rod cell, a cone cell, or a retinal pigment epithelium cell).

The disclosure also provides the first AAV vector, as defined above. There is also provided the second AAV vector, as defined above.

The disclosure provides an AAV vector, comprising a nucleic acid sequence comprising a 5′ end portion of an ABCA4 CDS, wherein the 5′ end portion of an ABCA4 CDS consists of a sequence of contiguous nucleotides corresponding to nucleotides 105 to 3805 of SEQ ID NO: 1. In certain embodiments, this AAV vector does not comprise any additional ABCA4 CDS beyond said sequence of contiguous nucleotides.

The first AAV vector may comprise 5′ and 3′ ITRs, preferably AAV ITRs; a promoter, for example a GRK1 promoter; and/or a UTR; said elements being as described above in relation to the AAV vector system of the disclosure.

In some embodiments, the first AAV vector comprises the nucleic acid sequence of SEQ ID NO: 9.

In some embodiments, the first AAV vector comprises the nucleic acid sequence of SEQ ID NO: 9 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the first AAV vector comprises the nucleic acid sequence of SEQ ID NO: 9 with the proviso that the nucleotide at the position corresponding to nucleotide 1640 of SEQ ID NO: 1 is G, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the first AAV vector comprises the nucleic acid sequence of SEQ ID NO: 3.

In some embodiments, the first AAV vector comprises the nucleic acid sequence of SEQ ID NO: 3 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the first AAV vector comprises the nucleic acid sequence of SEQ ID NO: 3 with the proviso that the nucleotide at the position corresponding to nucleotide 1640 of SEQ ID NO: 1 is G, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

The disclosure provides an AAV vector, comprising a nucleic acid sequence comprising a 3′ end portion of an ABCA4 CDS, wherein the 3′ end portion of an ABCA4 CDS consists of a sequence of contiguous nucleotides corresponding to nucleotides 3598 to 6926 of SEQ ID NO: 1. In some embodiments, this AAV vector does not comprise any additional ABCA4 CDS beyond said sequence of contiguous nucleotides.

The second vector may comprise 5′ and 3′ ITRs, preferably AAV ITRs; a PRE, preferably a WPRE; and/or a poly-adenylation sequence, preferably a bGH poly-adenylation sequence; said elements being as described above in relation to the AAV vector system of the disclosure.

In some embodiments, the second AAV vector comprises the nucleic acid sequence of SEQ ID NO: 10.

In some embodiments, the second AAV vector comprises the nucleic acid sequence of SEQ ID NO: 10 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the second AAV vector comprises the nucleic acid sequence of SEQ ID NO: 10 with the proviso that the nucleotide at the position corresponding to nucleotide 5279 of SEQ ID NO: 1 is G and the nucleotide at the position corresponding to nucleotide 6173 of SEQ ID NO: 1 is T, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the second AAV vector comprises the nucleic acid sequence of SEQ ID NO: 4.

In some embodiments, the second AAV vector comprises the nucleic acid sequence of SEQ ID NO: 4 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the second AAV vector comprises the nucleic acid sequence of SEQ ID NO: 4 with the proviso that the nucleotide at the position corresponding to nucleotide 5279 of SEQ ID NO: 1 is G and the nucleotide at the position corresponding to nucleotide 6173 of SEQ ID NO: 1 is T, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

The disclosure also provides nucleic acids comprising the nucleic acid sequences described above. The disclosure also provides an AAV vector genome derivable from an AAV vector as described above.

Also provided is a kit comprising the first AAV vector and the second AAV vector as described above. The AAV vectors may be provided in the kits in the form of AAV particles.

Further provided is a kit comprising a nucleic acid comprising the first nucleic acid sequence and a nucleic acid comprising the second nucleic acid sequence, as described above.

The disclosure also provides a pharmaceutical composition comprising the AAV vector system as described above and a pharmaceutically acceptable excipient.

The AAV vector system of the disclosure, the kit of the disclosure, and the pharmaceutical composition of the disclosure, may be used in gene therapy. For example, AAV vector system of the disclosure, the kit of the disclosure, and the pharmaceutical composition of the disclosure, may be used in preventing or treating disease.

In some embodiments, use of the compositions and methods of the disclosure to prevent or treat disease comprises administration of the first AAV vector and second AAV vector to a target cell, to provide expression of ABCA4 protein.

In some embodiments, the disease to be prevented or treated is characterized by degradation of retinal cells. An example of such a disease is Stargardt disease. In some embodiments, the first and second AAV vectors of the disclosure may be administered to an eye of a patient, for example to retinal tissue of the eye, such that functional ABCA4 protein is expressed to compensate for the mutation(s) present in the disease.

The AAV vectors of the disclosure may be formulated as pharmaceutical compositions or medicaments.

An example AAV vector system of the disclosure comprises a first AAV vector and a second AAV vector; wherein the first AAV vector comprises the nucleic acid sequence of SEQ ID NO: 9; and the second AAV vector comprises the nucleic acid sequence of SEQ ID NO: 10.

A further exemplary AAV vector system of the disclosure comprises a first AAV vector and a second AAV vector; wherein the first AAV vector comprises the nucleic acid sequence of SEQ ID NO: 9 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity; and the second AAV vector comprises the nucleic acid sequence of SEQ ID NO: 10 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the methods and uses of the disclosure may also be performed where SEQ ID NO: 2 is used as a reference sequence in place of SEQ ID NO: 1.

In this regard, SEQ ID NO: 2 is identical to SEQ ID NO: 1 with the exception of the following mutations: nucleotide 1640 G>T, nucleotide 5279 G>A, nucleotide 6173 T>C. These mutations do not alter the encoded amino acid sequence, and thus the ABCA4 protein encoded by SEQ ID NO: 2 is identical to the ABCA4 protein encoded by SEQ ID NO:1.

Thus, in alternative embodiments of the disclosure, references above to SEQ ID NO: 1 may be replaced with references to SEQ ID NO:2.

### Sequence Correspondence

As used herein, the term “corresponding to” when used with regard to the nucleotides in a given nucleic acid sequence defines nucleotide positions by reference to a particular SEQ ID NO. However, when such references are made, it will be understood that the disclosure is not to be limited to the exact sequence as set out in the particular SEQ ID NO referred to but includes variant sequences thereof. The nucleotides corresponding to the nucleotide positions in SEQ ID NO: 1 can be readily determined by sequence alignment, such as by using sequence alignment programs, the use of which is well known in the art. In this regard, a skilled person would readily appreciate that the degenerate nature of the genetic code means that variations in a nucleic acid sequence encoding a given polypeptide may be present without changing the amino acid sequence of the encoded polypeptide. Thus, identification of nucleotide locations in other ABCA4 coding sequences is contemplated (i.e. nucleotides at positions which the skilled person would consider correspond to the positions identified in, for example, SEQ ID NO: 1).

By way of example, SEQ ID NO: 2 is identical to SEQ ID NO: 1 with the exception of three specific mutations, as described above (these three mutations do not alter the amino acid sequence of the encoded ABCA4 polypeptide). In this case, a skilled person would therefore consider that a given nucleotide position in SEQ ID NO: 2 corresponded to the equivalent numbered nucleotide position in SEQ ID NO: 1.

### AAV Vectors

The viral vectors of the disclosure comprise adeno-associated viral (AAV) vectors. An AAV vector of the disclosure may be in the form of a mature AAV particle or virion, i.e. nucleic acid surrounded by an AAV protein capsid.

The AAV vector may comprise an AAV genome or a derivative thereof.

An AAV genome is a polynucleotide sequence, which may, in some embodiments, encode functions for the production of an AAV particle. These functions include, for example, those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, an AAV genome of a vector of the disclosure may be replication-deficient.

The AAV genome may be in single-stranded form, either positive or negative-sense, or alternatively in double-stranded form. In some embodiments, the use of a double-stranded form allows bypass of the DNA replication step in the target cell and so can accelerate transgene expression.

In some embodiments, the AAV genome of a vector of the disclosure may be in single-stranded form.

The AAV genome may be from any naturally derived serotype, isolate or clade of AAV. Thus, the AAV genome may be the full genome of a naturally occurring AAV. As is known to the skilled person, AAVs occurring in nature may be classified according to various biological systems.

AAVs are referred to in terms of their serotype. A serotype corresponds to a variant subspecies of AAV which, owing to its profile of expression of capsid surface antigens, has a distinctive reactivity which can be used to distinguish it from other variant subspecies. A virus having a particular AAV serotype does not efficiently cross-react with neutralizing antibodies specific for any other AAV serotype.

AAV serotypes include AAV1, AAV2, AAV3, AAV4, AAV5, AAV6, AAV7, AAV8, AAV9, AAV10 and AAV11, and also recombinant serotypes, such as Rec2 and Rec3, recently identified from primate brain. Any of these AAV serotypes may be used in the disclosure. Thus, in one embodiment of the disclosure, an AAV vector of the disclosure may be derived from an AAV1, AAV2, AAV3, AAV4, AAV5, AAV6, AAV7, AAV8, AAV9, AAV10, AAV11, Rec2 or Rec3 AAV.

Reviews of AAV serotypes may be found in Choi et al. (2005) Curr. Gene Ther. 5: 299-310 and Wu et al. (2006) Molecular Therapy 14: 316-27. The sequences of AAV genomes or of elements of AAV genomes including ITR sequences, rep or cap genes may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC 001862; Avian AAV ATCC VR-865 AY186198, AY629583, NC_004828; Avian AAV strain DA-1 NC_006263, AY629583; Bovine AAV NC_005889, AY388617.

AAV may also be referred to in terms of clades or clones. This refers, for example, to the phylogenetic relationship of naturally derived AAVs, or to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognizably distinct population at a genetic level.

The skilled person can select an appropriate serotype, clade, clone or isolate of AAV for use in the disclosure on the basis of their common general knowledge. For instance, the AAV5 capsid has been shown to transduce primate cone photoreceptors efficiently as evidenced by the successful correction of an inherited color vision defect (Mancuso et al. (2009) Nature 461: 784-7).

The AAV serotype can determine the tissue specificity of infection (or tropism) of an AAV virus. Accordingly, in some preferred embodiments the AAV serotypes for use in AAVs administered to patients of the disclosure are those which have natural tropism for or a high efficiency of infection of target cells within the eye. In one embodiment, AAV serotypes for use in the disclosure are those which infect cells of the neurosensory retina, retinal pigment epithelium and/or choroid.

In some embodiments, the AAV genome of a naturally derived serotype, isolate or clade of AAV comprises at least one inverted terminal repeat sequence (ITR). An ITR sequence may act in cis to provide a functional origin of replication and allows for integration and excision of the vector from the genome of a cell. The AAV genome may also comprise packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins m ay make up the capsid of an AAV particle. Capsid variants are discussed below.

In some embodiments, a promoter can be operably linked to each of the packaging genes. Specific examples of such promoters include the p5, p19 and p40 promoters (Laughlin et al. (1979) Proc. Natl. Acad. Sci. USA 76: 5567-5571). For example, the p5 and p19 promoters may be used to express the rep gene, while the p40 promoter may be used to express the cap gene.

In some embodiments, the AAV genome used in a vector of the disclosure may therefore be the full genome of a naturally occurring AAV. For example, a vector comprising a full AAV genome may be used to prepare an AAV vector in vitro. In some embodiments, such a vector may in principle be administered to patients. In some preferred embodiments, the AAV genome will be derivative for the purpose of administration to patients. Such derivatization is known in the art and the disclosure encompasses the use of any known derivative of an AAV genome, and derivatives which could be generated by applying techniques known in the art. Derivatization of the AAV genome and of the AAV capsid are reviewed in Coura and Nardi (2007) Virology Journal 4: 99, and in Choi et al. and Wu et al., referenced above.

Derivatives of an AAV genome include any truncated or modified forms of an AAV genome which allow for expression of a transgene from a vector of the disclosure in vivo. In some embodiments, it is possible to truncate the AAV genome to include minimal viral sequence yet retain the above function. This may contribute to the safety of the AAV genome, by example reducing the risk of recombination of the vector with wild-type virus, and also avoiding triggering a cellular immune response by the presence of viral gene proteins in the target cell.

A derivative of an AAV genome may include at least one inverted terminal repeat sequence (ITR). In some embodiments, a derivative of an AAV genome may include more than one ITR, such as two ITRs or more. One or more of the ITRs may be derived from AAV genomes having different serotypes, or may be a chimeric or mutant ITR. An exemplary mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The inclusion of one or more ITRs may aid concatamer formation of a vector of the disclosure in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In some preferred embodiments, ITR elements will be the only sequences retained from the native AAV genome in the derivative. Thus, a derivative may not include the rep and/or cap genes of the native genome and any other sequences of the native genome. This may also reduce the possibility of integration of the vector into the host cell genome. Additionally, reducing the size of the AAV genome allows for increased flexibility in incorporating other sequence elements (such as regulatory elements) within the vector in addition to the transgene.

The following portions may be removed in a derivative of the disclosure: one inverted terminal repeat (ITR) sequence, the replication (rep) and capsid (cap) genes. However, in some embodiments, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the vector may be tolerated in a therapeutic setting.

Where a derivative comprises capsid proteins i.e. VP1, VP2 and/or VP3, the derivative may be a chimeric, shuffled or capsid-modified derivative of one or more naturally occurring AAVs. The disclosure encompasses the provision of capsid protein sequences from different serotypes, clades, clones, or isolates of AAV within the same vector (i.e. a pseudotyped vector).

Chimeric, shuffled or capsid-modified derivatives may be selected to provide one or more functionalities for the viral vector. For example, these derivatives may display increased efficiency of gene delivery, decreased immunogenicity (humoral or cellular), an altered tropism range and/or improved targeting of a particular cell type compared to an AAV vector comprising a naturally occurring AAV genome, such as that of AAV2. Increased efficiency of gene delivery may be effected by improved receptor or co-receptor binding at the cell surface, improved internalization, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed, for example, by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins of the disclosure also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. For example, capsid genes may be modified by the insertion of a sequence of an unrelated protein or peptide within an open reading frame of a capsid coding sequence, or at the N- and/or C-terminus of a capsid coding sequence.

The unrelated protein or peptide may be one which acts as a ligand for a particular cell type, thereby conferring improved binding to a target cell or improving the specificity of targeting of the vector to a particular cell population. The unrelated protein may also be one which assists purification of the viral particle as part of the production process, i.e. an epitope or affinity tag. The site of insertion may be selected so as not to interfere with other functions of the viral particle e.g. internalization, trafficking of the viral particle. The skilled person can identify suitable sites for insertion based on their common general knowledge. Particular sites are disclosed in Choi et al., referenced above.

The disclosure additionally encompasses the provision of sequences of an AAV genome in a different order and configuration to that of a native AAV genome. The disclosure also encompasses the replacement of one or more AAV sequences or genes with sequences from another virus or with chimeric genes composed of sequences from more than one virus. Such chimeric genes may be composed of sequences from two or more related viral proteins of different viral species.

AAV vectors of the disclosure include transcapsidated forms wherein an AAV genome or derivative having an ITR of one serotype is packaged in the capsid of a different serotype. AAV vectors of the disclosure also include mosaic forms wherein a mixture of unmodified capsid proteins from two or more different serotypes makes up the viral capsid. An AAV vector may also include chemically modified forms bearing ligands adsorbed to the capsid surface. For example, such ligands may include antibodies for targeting a particular cell surface receptor.

Thus, for example, AAV vectors of the disclosure may include those with an AAV2 genome and AAV2 capsid proteins (AAV2/2), those with an AAV2 genome and AAV5 capsid proteins (AAV2/5) and those with an AAV2 genome and AAV8 capsid proteins (AAV2/8).

An AAV vector of the disclosure may comprise a mutant AAV capsid protein. In one embodiment, an AAV vector of the disclosure comprises a mutant AAV8 capsid protein. In some embodiments, the mutant AAV8 capsid protein is an AAV8 Y733F capsid protein. In some embodiments, the AAV8 Y733F mutant capsid protein comprises an amino acid sequence with at least 95% identity to SEQ ID NO: 12 with a substitution of phenylalanine for tyrosine at position 733 of SEQ ID NO: 12. In some embodiments, the AAV8 Y733F mutant capsid protein comprises an amino acid sequence of SEQ ID NO: 12 with a substitution of phenylalanine for tyrosine at position 733 of SEQ ID NO: 12.

### Methods of Administration

The viral vectors of the disclosure may be administered to the eye of a subject by subretinal, direct retinal, suprachoroidal or intravitreal injection.

A skilled person will be familiar with and well able to carry out individual subretinal, direct retinal, suprachoroidal or intravitreal injections.

### Subretinal Injection

Subretinal injections are injections into the subretinal space, i.e. underneath the neurosensory retina. During a subretinal injection, the injected material is directed into, and creates a space between, the photoreceptor cell and retinal pigment epithelial (RPE) layers.

When the injection is carried out through a small retinotomy, a retinal detachment may be created. The detached, raised layer of the retina that is generated by the injected material is referred to as a “bleb”.

The hole created by the subretinal injection may be sufficiently small that the injected solution does not significantly reflux back into the vitreous cavity after administration. Such reflux would be problematic when a medicament is injected, because the effects of the medicament would be directed away from the target zone. Preferably, the injection creates a self-sealing entry point in the neurosensory retina, i.e. once the injection needle is removed, the hole created by the needle reseals such that very little or substantially no injected material is released through the hole.

To facilitate this process, specialist subretinal injection needles are commercially available (e.g. DORC 41G Teflon subretinal injection needle, Dutch Ophthalmic Research Center International BV, Zuidland, The Netherlands). These are needles designed to carry out subretinal injections.

In some embodiments, subretinal injection comprises a scleral tunnel approach through the posterior pole to the superior retina with a Hamilton syringe and 34-gauge needle (ESS labs, UK). Alternatively, or in addition, subretinal injections can comprise performing an anterior chamber paracentesis with a 33G needle prior to the sub-retinal injection using a WPI syringe and a bevelled 35G-needle system (World Precision Instruments, UK).

Animal subjects, can be anaesthetized, for example, by intraperitoneal injection containing ketamine (80 mg/kg) and xylazine (10 mg/kg) and pupils fully dilated with tropicamide eye drops (Mydriaticum 1%, Bausch & Lomb, UK) and phenylephrine eye drops (phenylephrine hydrochloride 2.5%, Bausch & Lomb, UK). Proxymetacaine eye drops (proxymetacaine hydrochloride 0.5%, Bausch & Lomb, UK) can also be applied prior to sub-retinal injection. Post-injection, chloramphenicol eye drops can be applied (chloramphenicol 0.5%, Bausch & Lomb, UK), anaesthesia reversed with atipamezole (2 mg/kg), and carbomer gel applied (Viscotears, Novartis, UK) to prevent cataract formation.

Unless damage to the retina occurs during the injection, and as long as a sufficiently small needle is used, injected material remains localized between the detached neurosensory retina and the RPE at the site of the localized retinal detachment (i.e. does not reflux into the vitreous cavity). Indeed, the persistence of the bleb over a short time frame indicates that there may be little escape of the injected material into the vitreous. The bleb may dissipate over a longer time frame as the injected material is absorbed.

Visualizations of the eye, for example the retina, for example using optical coherence tomography, may be made pre-operatively.

### Two-Step Subretinal Injection

In some embodiments, the AAV vectors of the disclosure may be delivered with accuracy and safety by using a two-step method in which a localized retinal detachment is created by the subretinal injection of a first solution. The first solution does not comprise the vector. A second subretinal injection is then used to deliver the medicament comprising the vector into the subretinal fluid of the bleb created by the first subretinal injection. Because the injection delivering the medicament is not being used to detach the retina, a specific volume of solution may be injected in this second step.

An AAV vector of the disclosure may be delivered by:

(a) administering a solution to the subject by subretinal injection in an amount effective to at least partially detach the retina to form a subretinal bleb, wherein the solution does not comprise the vector; and

(b) administering a medicament composition by subretinal injection into the bleb formed by step (a), wherein the medicament comprises the vector.

The volume of solution injected in step (a) to at least partially detach the retina may be, for example, about 10-1000 μL, for example about 50-1000, 100-1000, 250-1000, 500-1000, 10-500, 50-500, 100-500, 250-500 μL. The volume may be, for example, about 10, 50, 100, 200, 300, 400, 500, 600, 700, 800, 900 or 1000 μL.

The volume of the medicament composition injected in step (b) may be, for example, about 10-500 μL, for example about 50-500, 100-500, 200-500, 300-500, 400-500, 50-250, 100-250, 200-250 or 50-150 μL. The volume may be, for example, about 10, 50, 100, 150, 200, 250, 300, 350, 400, 450 or 500 μL. In some preferred embodiments, y, the volume of the medicament composition injected in step (b) is 100 μL. Larger volumes may increase the risk of stretching the retina, while smaller volumes may be difficult to see.

The solution that does not comprise the medicament (i.e. the “first solution” of step (a)) may be similarly formulated to the solution that does comprise the medicament, as described below. An exemplary solution that does not comprise the medicament is balanced saline solution (BSS), TMN 200 or a similar buffer solution matched to the pH and osmolality of the subretinal space.

### Visualizing the Retina During Surgery

In some embodiments, for example during end-stage retinal degenerations, identifying the retina is difficult because it is thin, transparent and difficult to see against the disrupted and heavily pigmented epithelium on which it sits. The use of a blue vital dye (e.g. Brilliant Peel©, Geuder; MembraneBlue-Dual©, Dorc) may facilitate the identification of the retinal hole made for the retinal detachment procedure (i.e. step (a) in the two-step subretinal injection method of the disclosure) so that the medicament can be administered through the same hole without the risk of reflux back into the vitreous cavity.

The use of the blue vital dye may also identify any regions of the retina where there is a thickened internal limiting membrane or epiretinal membrane, as injection through either of these structures may hinder clean access into the subretinal space. Furthermore, contraction of either of these structures in the immediate post-operative period may lead to stretching of the retinal entry hole, which may lead to reflux of the medicament into the vitreous cavity.

### Suprachoroidal Injection

Vectors or compositions of the disclosure may be administered via suprachoroidal injection. Any means of suprachoroidal injection is envisaged as a potential delivery system for a vector or a composition of the disclosure. Suprachoroidal injections are injections into the suprachoroidal space, which is the space between the choroid and the sclera. Injection into the suprachoroidal space is thus a potential route of administration for the delivery of compositions to proximate eye structures such as the retina, retinal pigment epithelium (RPE) or macula. In some embodiments, injection into the suprachoroidal space is done in an anterior portion of the eye using a microneedle, microcannula, or microcatheter. An anterior portion of the eye may comprise or consist of an area anterior to the equator of the eye. The vector composition or AAV viral particles may diffuse posteriorly from an injection site via a suprachoroidal route. In some embodiments, the suprachoroidal space in the posterior eye is injected directly using a catheter system. In this embodiment, the suprachoroidal space may be catheterized via an incision in the pars plana. In some embodiments, an injection or an infusion via a suprachoroidal route traverses the choroid, Bruch's membrane and/or RPE layer to deliver a vector or a composition of the disclosure to a subretinal space. In some embodiments, including those in which a vector or a composition of the disclosure is delivered to a subretinal space via a suprachoroidal route, one or more injections is made into at least one of the sclera, the pars plana, the choroid, the Bruch's membrane, and the RPE layer. In some embodiments, including those in which a vector or a composition of the disclosure is delivered to a subretinal space via a suprachoroidal route, a two-step procedure is used to create a bleb in a suprachoroidal or a subretinal space prior to delivery of a vector or a composition of the disclosure.

### Pharmaceutical Compositions and Injected Solutions

The AAV vectors and AAV vector system of the disclosure may be formulated into pharmaceutical compositions. These compositions may comprise, in addition to the medicament, a pharmaceutically acceptable carrier, diluent, excipient, buffer, stabilizer or other materials well known in the art. Such materials should be non-toxic and should not interfere with the efficacy of the active ingredient. The precise nature of the carrier or other material may be determined by the skilled person according to the route of administration, e.g. subretinal, direct retinal, suprachoroidal or intravitreal injection.

The pharmaceutical composition may be in liquid form. Liquid pharmaceutical compositions include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included.

For injection at the site of affliction, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection or TMN 200. Preservatives, stabilisers, buffers, antioxidants and/or other additives may be included as required.

Buffers may have an effect on the stability and biocompatibity of the viral vectors and vector particles of the disclosure following storage and passage through injection devices for AAV gene therapy. In some embodiments, the viral vectors and vector particles of the disclosure may be diluted in TMN 200 buffer to maintain biocompatibility and stability. TMN 200 buffer comprises 20 mM Tris (pH adjusted to 8.0), 1 mM MgCl₂ and 200 mM NaCl at pH 8.

The determination of the physical viral genome titer comprises part of the characterization of the viral vector or viral particle. In some embodiments, determination of the physical viral genome titre comprises a step in ensuring the potency and safety of viral vectors and viral particles during gene therapy. In some embodiments, a method to determine the AAV titer comprises quantitative PCR (qPCR). There are different variables that can influence the results, such as the conformation of the DNA used as standard or the enzymatic digestion during the sample preparation. The viral vector or particle preparation whose titer is to be measured may be compared against a standard dilution curve generated using a plasmid. In some embodiments, the plasmid DNA used in the standard curve is in the supercoiled conformation. In some embodiments, the plasmid DNA used in the standard curve is in the linear conformation. Linearized plasmid can be prepared, for example by digestion with HindIII restriction enzyme, visualized by agarose gel electrophoresis and purified using the QIAquick Gel Extraction Kit (Qiagen) following manufacturer's instructions. Other restriction enzymes that cut within the plasmid used to generate the standard curve may also be appropriate. In some embodiments, the use of supercoiled plasmid as the standard increased the titre of the AAV vector compared to the use of linearized plasmid.

To extract the DNA from purified AAV vectors for quantification of AAV genome titer, two enzymatic methods can be used. In some embodiments, the AAV vector may be singly digested with DNase I. In some embodiments, the AAV vector may be and double digested with DNase I and an additional proteinase K treatment. QPCR can then performed with the CFX Connect Real-Time PCR Detection System (BioRad) using primers and Taqman probe specific to the transgene sequence.

For delayed release, the medicament may be included in a pharmaceutical composition which is formulated for slow release, such as in microcapsules formed from biocompatible polymers or in liposomal carrier systems according to methods known in the art.

### Method of Treatment

It is to be appreciated that all references herein to treatment include curative, palliative and prophylactic treatment; although in the context of the disclosure references to preventing are associated with prophylactic treatment. Treatment may also include arresting progression in the severity of a disease.

The treatment of all mammals, including humans, is envisaged. However, both human and veterinary treatments are within the scope of the disclosure.

### Variants, Derivatives, Analogues, Homologues and Fragments

In addition to the specific proteins and nucleotides mentioned herein, the disclosure also encompasses the use of variants, derivatives, analogues, homologues and fragments thereof. In the context of the disclosure, a variant of any given sequence is a sequence in which the specific sequence of residues (whether amino acid or nucleic acid residues) has been modified in such a manner that the polypeptide or polynucleotide in question substantially retains its function. A variant sequence can be obtained, for example, by addition, deletion, substitution, modification, replacement and/or variation of at least one residue present in the naturally-occurring protein.

The term “derivative” as used herein, in relation to proteins or polypeptides of the disclosure includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Amino acid substitutions may be made, for example from 1, 2 or 3 to 10, 20 or more substitutions provided that the modified sequence substantially retains the required activity or ability. Amino acid substitutions may include the use of non-naturally occurring analogues. Proteins used in the disclosure may also have deletions, insertions or substitutions of amino acid residues which produce a silent change and result in a functionally equivalent protein.

Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence and the wild type nucleotide sequence. The term “homology” can be equated with “identity”.

A homologous sequence may include an amino acid sequence which may be at least 50%, 55%, 60%, 65%, 70%, 75%, 80%, 85% or 90% identical, preferably at least 95% or 97% or 99% identical to the subject sequence. For example, the homologues will comprise the same active sites etc. as the subject amino acid sequence. Although homology can also be considered in terms of similarity (i.e. amino acid residues having similar chemical properties/functions), in the context of the disclosure homology can also be expressed in terms of sequence identity.

A homologous sequence may include a nucleotide sequence which may be at least 50%, 55%, 60%, 65%, 70%, 75%, 80%, 85% or 90% identical, preferably at least 95% or 97% or 99% identical to the subject sequence. Although homology can also be considered in terms of similarity, in the context of the disclosure homology can also be expressed in terms of sequence identity.

Reference to a sequence which has a percent identity to any one of the SEQ ID NOs detailed herein refers to a sequence which has the stated percent identity over the entire length of the SEQ ID NO referred to.

Homology comparisons can be conducted by eye or with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Such ungapped alignments may be performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalizing unduly the overall homology score. This is achieved by inserting “gaps” in the sequence alignment to try to maximize local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” may be used that charge a relatively high cost for the existence of a gap and a smaller penalty for each subsequent residue in the gap. This is the most commonly used gap scoring system. High gap penalties will of course produce optimized alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly comprises the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid-Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, the GCG Bestfit program can be used. Another tool, called BLAST 2 Sequences is also available for comparing protein and nucleotide sequences (see FEMS Microbiol. Lett. (1999) 174: 247-50; FEMS Microbiol. Lett. (1999) 177: 187-8). Although the final percent homology can be measured in terms of identity, the alignment process itself may not be based on an all-or-nothing pair comparison. Instead, a scaled similarity score matrix may be used that assigns scores to each pairwise comparison based on chemical similarity or evolutionary distance. An example of such a matrix is the BLOSUM62 matrix—the default matrix for the BLAST suite of programs. GCG Wisconsin programs may use either the public default values or a custom symbol comparison table if supplied (see the user manual for further details). Some applications, use the public default values for the GCG package, or in the case of other software, the default matrix, such as BLOSUM62.

Once the software has produced an optimal alignment, it is possible to calculate percent homology, preferably percent sequence identity. The software may do this as part of the sequence comparison and generates a numerical result.

“Fragments” are also variants and the term may refer to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the disclosure to make the encoded protein. These methods are only illustrative of the numerous standard techniques known in the art for manipulation of DNA sequences and other known techniques may also be used.

### Codon Optimization

The present disclosure encompasses codon optimized variants of the nucleic acid sequences described herein.

Codon optimization takes advantage of redundancies in the genetic code to enable a nucleotide sequence to be altered while maintaining the same amino acid sequence of the encoded protein.

Codon optimization may be carried out to facilitate an increase or decrease in the expression of an encoded protein. This may be effected by tailoring codon usage in a nucleotide sequence to that of a specific cell type, thus taking advantage of cellular codon bias corresponding to a bias in the relative abundance of particular tRNAs in the cell type. By altering the codons in the nucleotide sequence so that they are tailored to match the relative abundance of corresponding tRNAs, it is possible to increase expression. Conversely, it is possible to decrease expression by selecting codons for which the corresponding tRNAs are known to be rare in the particular cell type. Methods for codon optimization of nucleic acid sequences are known in the art and will be familiar to a skilled person.

### Near Darkness Agility Maze

The baseline or improved visual acuity of a subject of the disclosure may be measured by having the subject navigate through an enclosure characterized by low light or dark conditions and including one or more obstacles for the subject to avoid. The subject may be in need of a composition of the disclosure, optionally, provided by a method of treating of the disclosure. The subject may have received a composition of the disclosure, optionally, provided by a method of treating of the disclosure in one or both eyes and in one or more doses and/or procedures/injections. The enclosure may be indoors or outdoors. The enclosure is characterized by a controlled light level ranging from a level that recapitulates daylight to a level that simulates complete darkness. Within this range, the controlled light level of the enclosure may be preferably set to recapitulate natural dusk or evening light levels at which a subject of the disclosure prior to receiving a composition of the disclosure may have decreased visual acuity. Following administration of a composition of the disclosure, the subject may have improved visual acuity at all light levels, but the improvement is preferably measured at lower light levels, including those that recapitulate natural dusk or evening light levels (indoors or outdoors).

In some embodiments of the enclosure, the one or more obstacles are aligned with one or more designated paths and/or courses within the enclosure. A successful passage through the enclosure by a subject may include traversing a designated path and avoiding traversal of a non-designated path. A successful passage through the enclosure by a subject may include traversing any path, including a designated path, while avoiding contact with one or more obstacles positioned either within a path or in proximity to a path. A successful or improved passage through the enclosure by a subject may include traversing any path, including a designated path, while avoiding contact with one or more obstacles positioned either within a path or in proximity to a path with a decreased time required to traverse the path from a designated start position to a designated end position (e.g. when compared to a healthy individual with normal visual acuity or when compared to a prior traversal by the subject). In some embodiments, an enclosure may comprise at least 1, 2, 3, 4, 5, 6, 7, 8, 9 or 10 paths or designated paths. A designated path may differ from a non-designated path by the identification of the designated path by the experimenter as containing an intended start position and an intended end position.

In some embodiments of the enclosure, the one or more obstacles are not fixed to a surface of the disclosure. In some embodiments, the one or more obstacles are fixed to a surface of the disclosure. In some embodiments, the one or more obstacles are fixed to an internal surface of the enclosure, including, but not limited to, a floor, a wall and a ceiling of the enclosure. In some embodiments, the one or more obstacles comprise a solid object. In some embodiments, the one or more obstacles comprise a liquid object (e.g. a “water hazard”). In some embodiments, the one or more obstacles comprise in any combination or sequence along at least one path or in close proximity to a path, an object to be circumvented by a subject; an object to be stepped over by a subject; an object to be balanced upon by walking or standing; an object having an incline, a decline or a combination thereof; an object to be touched (for example, to determine a subject's ability to see and/or judge depth perception); and an object to be traversed by walking or standing beneath it (e.g., including bending one or more directions to avoid the object). In some embodiments of the enclosure, the one or more obstacles must be encountered by the subject in a designated order.

In certain embodiments, baseline or improved visual acuity of a subject may be measured by having the subject navigate through a course or enclosure characterized by low light or dark conditions and including one or more obstacles for the subject to avoid, wherein the course or enclosure is present in an installation. In particular embodiments, the installation includes a modular lighting system and a series of different mobility course floor layouts. In certain embodiments, one room houses all mobility courses with one set of lighting rigs. For example, a single course may be set up at a time during mobility testing, and the same room/lighting rigs may be used for mobility testing independent of the course (floor layout) in use. In particular embodiments, the different mobility courses provided for testing are designed to vary in difficulty, with harder courses featuring low contrast pathways and hard to see obstacles, and easier courses featuring high contrast pathways and easy to see obstacles.

In some embodiments of the enclosure, the subject may be tested prior to administration of a composition of the disclosure to establish, for example, a baseline measurement of accuracy and/or speed or to diagnose a subject as having a retinal disease or at risk of developing a retinal disease. In some embodiments, the subject may be tested following administration of a composition of the disclosure to determine a change from a baseline measurement or a comparison to a score from a healthy individual (e.g. for monitoring/testing the efficacy of the composition to improve visual acuity).

### Adaptive Optics and Scanning Laser Ophthalmoscopy (AOSLO)

The baseline or improved measurement of retinal cell viability of a subject of the disclosure may be measured by one or more AOSLO techniques. Scanning Laser Ophthalmoscopy (SLO) may be used to view a distinct layer of a retina of an eye of a subject. Preferably, adaptive optics (AO) are incorporated in SLO (AOSLO), to correct for artifacts in images from SLO alone typically caused by structure of the anterior eye, including, but not limited to the cornea and the lens of the eye. Artifacts produced by using SLO alone decrease resolution of the resultant image. Adaptive optics allow for the resolution of a single cell of a layer of the retina and detect directionally backscattered light (waveguided light) from normal or intact retinal cells (e.g. normal or intact photoreceptor cells).

In some embodiments of the disclosure, using an AOSLO technique, an intact cell produce a waveguided and/or detectable signal. In some embodiments a non-intact cell does not produce a waveguided and/or detectable signal.

AOSLO may be used to image and, preferably, evaluate the retina or a portion thereof in a subject. In some embodiments, the subject has one or both retinas imaged using an AOSLO technique. In some embodiments, the subject has one or both retinas imaged using an AOSLO technique prior to administration of a composition of the disclosure (e.g. to determine a baseline measurement for subsequent comparison following treatment and/or to determine the presence and/or the severity of retinal disease). In some embodiments, the subject has one or both retinas imaged using an AOSLO technique following an administration of a composition of the disclosure (e.g. to determine an efficacy of the composition and/or to monitor the subject following administration for improvement resulting from treatment).

In some embodiments of the disclosure, the retina is imaged by either confocal or non-confocal (split-detector) AOSLO to evaluate a density of one or more retinal cells. In some embodiments, the one or more retinal cells include, but are not limited to a photoreceptor cell. In some embodiments, the one or more retinal cells include, but are not limited to a cone photoreceptor cell. In some embodiments, the one or more retinal cells include, but are not limited to a rod photoreceptor cell. In some embodiments, the density is measured as number of cells per millimeter. In some embodiments, the density is measured as number of live or viable cells per millimeter. In some embodiments, the density is measured as number of intact cells per millimeter (cells comprising an AAV particle or a transgene sequence of the disclosure). In some embodiments, the density is measured as number of responsive cells per millimeter. In some embodiments, a responsive cell is a functional cell.

In some embodiments, AOSLO may be used to capture an image of a mosaic of photoreceptor cells within a retina of the subject. In some embodiments, the mosaic includes intact cells, non-intact cells or a combination thereof. In some embodiments, a mosaic comprises a composite or montage of images representing an entire retina, an inner segment, an outer segment, or a portion thereof. In some embodiments, the image of a mosaic comprises a portion of a retina comprising or contacting a composition of the disclosure. In some embodiments, the image of a mosaic comprises a portion of a retina juxtaposed to a portion of the retina comprising or contacting a composition of the disclosure. In some embodiments, the image of a mosaic comprises a treated area and an untreated area, wherein the treated area comprises or contacts a composition of the disclosure and the untreated area does not comprise or contact a composition of the disclosure.

In some embodiments, AOSLO may be used alone or in combination with optical coherence tomography (OCT) to visualize directly a retinal, a portion of a retinal or a retinal cell of a subject. In some embodiments, adaptive optics may be used in combination with OCT (AO-OCT) to visualize directly a retinal, a portion of a retinal or a retinal cell of a subject.

In some embodiments of the disclosure, the outer or inner segment is imaged by either confocal or non-confocal (split-detector) AOSLO to evaluate a density of cells therein or a level of integrity of the outer segment, the inner segment or a combination thereof. In some embodiments, AOSLO may be used to detect a diameter of an inner segment, an outer segment or a combination thereof.

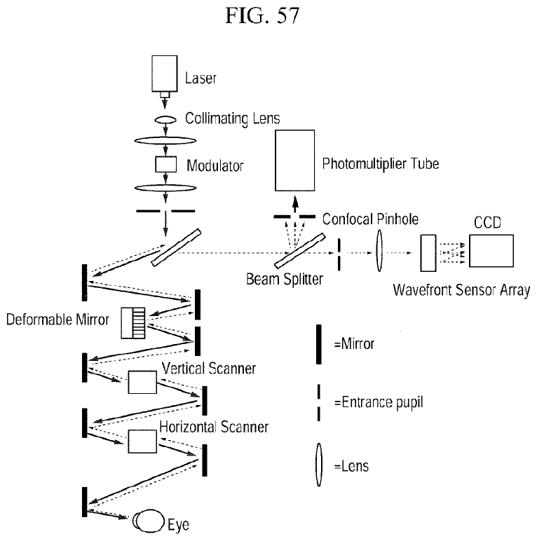

An exemplary AOSLO system is shown in FIG. 57.

Additional description of AOSLO and various techniques may be described at least in Georgiou et al. Br J Opthalmol 2017; 0:1-8; Scoles et al. Invest Opthalmol Vis Sci. 2014; 55:4244-4251; and Tanna et al. Invest Opthalmol Vis Sci. 2017; 58:3608-3615.

SEQUENCES

SEQ ID NO: 1

| | |
|---|---|
| 1 | AGGACACAGC GTCCGGAGCC AGAGGCGCTC TTAACGGCGT TTATGTCCTT TGCTGTCTGA |
| 61 | GGGGCCTCAG CTCTGACCAA TCTGGTCTTC GTGTGGTCAT TAGCATGGGC TTCGTGAGAC |
| 121 | AGATACAGCT TTTGCTCTGG AAGAACTGGA CCCTGCGGAA AAGGCAAAAG ATTCGCTTTG |
| 181 | TGGTGGAACT CGTGTGGCCT TTATCTTTAT TTCTGGTCTT GATCTGGTTA AGGAATGCCA |
| 241 | ACCCGCTCTA CAGCCATCAT GAATGCCATT TCCCCAACAA GGCGATGCCC TCAGCAGGAA |
| 301 | TGCTGCCGTG GCTCCAGGGG ATCTTCTGCA ATGTGAACAA TCCCTGTTTT CAAAGCCCCA |
| 361 | CCCCAGGAGA ATCTCCTGGA ATTGTGTCAA ACTATAACAA CTCCATCTTG GCAAGGGTAT |
| 421 | ATCGAGATTT TCAAGAACTC CTCATGAATG CACCAGAGAG CCAGCACCTT GGCCGTATTT |
| 481 | GGACAGAGCT ACACATCTTG TCCCAATTCA TGGACACCCT CCGGACTCAC CCGGAGAGAA |
| 541 | TTGCAGGAAG AGGAATACGA ATAAGGGATA TCTTGAAAGA TGAAGAAACA CTGACACTAT |
| 601 | TTCTCATTAA AAACATCGGC CTGTCTGACT CAGTGGTCTA CCTTCTGATC AACTCTCAAG |
| 661 | TCCGTCCAGA GCAGTTCGCT CATGGAGTCC CGGACCTGGC GCTGAAGGAC ATCGCCTGCA |
| 721 | GCGAGGCCCT CCTGGAGCGC TTCATCATCT TCAGCCAGAG ACGCGGGGCA AAGACGGTGC |
| 781 | GCTATGCCCT GTGCTCCCTC TCCCAGGGCA CCCTACAGTG GATAGAAGAC ACTCTGTATG |
| 841 | CCAACGTGGA CTTCTTCAAG CTCTTCCGTG TGCTTCCCAC ACTCCTAGAC AGCCGTTCTC |
| 901 | AAGGTATCAA TCTGAGATCT TGGGGAGGAA TATTATCTGA TATGTCACCA AGAATTCAAG |
| 961 | AGTTTATCCA TCGGCCGAGT ATGCAGGACT TGCTGTGGGT GACCAGGCCC CTCATGCAGA |
| 1021 | ATGGTGGTCC AGAGACCTTT ACAAAGCTGA TGGGCATCCT GTCTGACCTC CTGTGTGGCT |
| 1081 | ACCCCGAGGG AGGTGGCTCT CGGGTGCTCT CCTTCAACTG GTATGAAGAC AATAACTATA |
| 1141 | AGGCCTTTCT GGGGATTGAC TCCACAAGGA AGGATCCTAT CTATTCTTAT GACAGAAGAA |
| 1201 | CAACATCCTT TTGTAATGCA TTGATCCAGA GCCTGGAGTC AAATCCTTTA ACCAAAATCG |
| 1261 | CTTGGAGGGC GGCAAAGCCT TTGCTGATGG GAAAAATCCT GTACACTCCT GATTCACCTG |
| 1321 | CAGCACGAAG GATACTGAAG AATGCCAACT CAACTTTTGA AGAACTGGAA CACGTTAGGA |
| 1381 | AGTTGGTCAA AGCCTGGGAA GAAGTAGGGC CCCAGATCTG GTACTTCTTT GACAACAGCA |
| 1441 | CACAGATGAA CATGATCAGA GATACCCTGG GGAACCCAAC AGTAAAAGAC TTTTTGAATA |
| 1501 | GGCAGCTTGG TGAAGAAGGT ATTACTGCTG AAGCCATCCT AAACTTCCTC TACAAGGGCC |
| 1561 | CTCGGGAAAG CCAGGCTGAC GACATGGCCA ACTTCGACTG GAGGGACATA TTTAACATCA |
| 1621 | CTGATCGCAC CCTCCGCCTG GTCAATCAAT ACCTGGAGTG CTTGGTCCTG GATAAGTTTG |
| 1681 | AAAGCTACAA TGATGAAACT CAGCTCACCC AACGTGCCCT CTCTCTACTG GAGGAAAACA |
| 1741 | TGTTCTGGGC CGGAGTGGTA TTCCCTGACA TGTATCCCTG GACCAGCTCT CTACCACCCC |
| 1801 | ACGTGAAGTA TAAGATCCGA ATGGACATAG ACGTGGTGGA GAAAACCAAT AAGATTAAAG |
| 1861 | ACAGGTATTG GGATTCTGGT CCCAGAGCTG ATCCCGTGGA AGATTTCCGG TACATCTGGG |
| 1921 | GCGGGTTTGC CTATCTGCAG GACATGGTTG AACAGGGGAT CACAAGGAGC CAGGTGCAGG |
| 1981 | CGGAGGCTCC AGTTGGAATC TACCTCCAGC AGATGCCCTA CCCCTGCTTC GTGGACGATT |
| 2041 | CTTTCATGAT CATCCTGAAC CGCTGTTTCC CTATCTTCAT GGTGCTGGCA TGGATCTACT |
| 2101 | CTGTCTCCAT GACTGTGAAG AGCATCGTCT TGGAGAAGGA GTTGCGACTG AAGGAGACCT |
| 2161 | TGAAAAATCA GGGTGTCTCC AATGCAGTGA TTTGGTGTAC CTGGTTCCTG GACAGCTTCT |
| 2221 | CCATCATGTC GATGAGCATC TTCCTCCTGA CGATATTCAT CATGCATGGA AGAATCCTAC |
| 2281 | ATTACAGCGA CCCATTCATC CTCTTCCTGT TCTTGTTGGC TTTCTCCACT GCCACCATCA |
| 2341 | TGCTGTGCTT TCTGCTCAGC ACCTTCTTCT CCAAGGCCAG TCTGGCAGCA GCCTGTAGTG |
| 2401 | GTGTCATCTA TTTCACCCTC TACCTGCCAC ACATCCTGTG CTTCGCCTGG CAGGACCGCA |
| 2461 | TGACCGCTGA GCTGAAGAAG GCTGTGAGCT TACTGTCTCC GGTGGCATTT GGATTTGGCA |
| 2521 | CTGAGTACCT GGTTCGCTTT GAAGAGCAAG GCCTGGGGCT GCAGTGGAGC AACATCGGGA |
| 2581 | ACAGTCCCAC GGAAGGGGAC GAATTCAGCT TCCTGCTGTC CATGCAGATG ATGCTCCTTG |
| 2641 | ATGCTGCTGT CTATGGCTTA CTCGCTTGGT ACCTTGATCA GGTGTTTCCA GGAGACTATG |
| 2701 | GAACCCCACT TCCTTGGTAC TTTCTTCTAC AAGAGTCGTA TTGGCTTGGC GGTGAAGGGT |
| 2761 | GTTCAACCAG AGAAGAAAGA GCCCTGGAAA AGACCGAGCC CCTAACAGAG GAAACGGAGG |
| 2821 | ATCCAGAGCA CCCAGAAGGA ATACACGACT CCTTCTTTGA ACGTGAGCAT CCAGGGTGGG |
| 2881 | TTCCTGGGGT ATGCGTGAAG AATCTGGTAA AGATTTTTGA GCCCTGTGGC CGGCCAGCTG |
| 2941 | TGGACCGTCT GAACATCACC TTCTACGAGA ACCAGATCAC CGCATTCCTG GGCCACAATG |
| 3001 | GAGCTGGGAA AACCACCACC TTGTCCATCC TGACGGGTCT GTTGCCACCA ACCTCTGGGA |
| 3061 | CTGTGCTCGT TGGGGGAAGG GACATTGAAA CCAGCCTGGA TGCAGTCCGG CAGAGCCTTG |
| 3121 | GCATGTGTCC ACAGCACAAC ATCCTGTTCC ACCACCTCAC GGTGGCTGAG CACATGCTGT |
| 3181 | TCTATGCCCA GCTGAAAGGA AAGTCCCAGG AGGAGGCCCA GCTGGAGATG GAAGCCATGT |
| 3241 | TGGAGGACAC AGGCCTCCAC CACAAGCGGA ATGAAGAGGC TCAGGACCTA TCAGGTGGCA |
| 3301 | TGCAGAGAAA GCTGTCGGTT GCCATTGCCT TTGTGGGAGA TGCCAAGGTG GTGATTCTGG |
| 3361 | ACGAACCCAC CTCTGGGGTG GACCCTTACT CGAGACGCTC AATCTGGGAT CTGCTCCTGA |
| 3421 | AGTATCGCTC AGGCAGAACC ATCATCATGT CCACTCACCA CATGGACGAG GCCGACCTCC |
| 3481 | TTGGGGACCG CATTGCCATC ATTGCCCAGG GAAGGCTCTA CTGCTCAGGC ACCCCACTCT |
| 3541 | TCCTGAAGAA CTGCTTTGGC ACAGGCTTGT ACTTAACCTT GGTGCGCAAG ATGAAAAACA |
| 3601 | TCCAGAGCCA AAGGAAAGGC AGTGAGGGGA CCTGCAGCTG CTCGTCTAAG GGTTTCTCCA |
| 3661 | CCACGTGTCC AGCCCACGTC GATGACCTAA CTCCAGAACA AGTCCTGGAT GGGGATGTAA |
| 3721 | ATGAGCTGAT GGATGTAGTT CTCCACCATG TTCCAGAGGC AAAGCTGGTG GAGTGCATTG |
| 3781 | GTCAAGAACT TATCTTCCTT CTTCCAAATA AGAACTTCAA GCACAGAGCA TATGCCAGCC |
| 3841 | TTTTCAGAGA GCTGGAGGAG ACGCTGGCTG ACCTTGGTCT CAGCAGTTTT GGAATTTCTG |
| 3901 | ACACTCCCCT GGAAGAGATT TTTCTGAAGG TCACGGAGGA TTCTGATTCA GGACCTCTGT |
| 3961 | TTGCGGGTGG CGCTCAGCAG AAAAGAGAAA ACGTCAACCC CCGACACCCC TGCTTGGGTC |
| 4021 | CCAGAGAGAA GGCTGGACAG ACACCCCAGG ACTCCAATGT CTGCTCCCCA GGGGCGCCGG |
| 4061 | CTGCTCACCC AGAGGGCCAG CCTCCCCCAG AGCCAGAGTG CCCAGGCCCG CAGCTCAACA |
| 4121 | CGGGGACACA GCTGGTCCTC CAGCATGTGC AGGCGCTGCT GGTCAAGAGA TTCCAACACA |
| 4181 | CCATCCGCAG CCACAAGGAC TTCCTGGCGC AGATCGTGCT CCCGGCTACC TTTGTGTTTT |
| 4241 | TGGCTCTGAT GCTTTCTATT GTTATCCCTC CTTTTGGCGA ATACCCCGCT TTGACCCTTC |
| 4301 | ACCCCTGGAT ATATGGGCAG CAGTACACCT TCTTCAGCAT GGATGAACCA GGCAGTGAGC |
| 4361 | AGTTCACGGT ACTTGCAGAC GTCCTCCTGA ATAAGCCAGG CTTTGGCAAC CGCTGCCTGA |
| 4421 | AGGAAGGGTG GCTTCCGGAG TACCCCTGTG GCAACTCAAC ACCCTGGAAG ACTCCTTCTG |
| 4481 | TGTCCCCAAA CATCACCCAG CTGTTCCAGA AGCAGAAATG GACACAGGTC AACCCTTCAC |
| 4541 | CATCCTGCAG GTGCAGCACC AGGGAGAAGC TCACCATGCT GCCAGAGTGC CCCGAGGGTG |
| 4601 | CCGGGGGCCT CCCGCCCCCC CAGAGAACAC AGCGCAGCAC GGAAATTCTA CAAGACCTGA |
| 4661 | CGGACAGGAA CATCTCCGAC TTCTTGGTAA AAACGTATCC TGCTCTTATA AGAAGCAGCT |
| 4721 | TAAAGAGCAA ATTCTGGGTC AATGAACAGA GGTATGGAGG AATTTCCATT GGAGGAAAGC |
| 4781 | TCCCAGTCGT CCCCATCACG GGGGAAGCAC TTGTTGGGTT TTTAAGCGAC CTTGGCCGGA |
| 4841 | TCATGAATGT GAGCGGGGGC CCTATCACTA GAGAGGCCTC TAAAGAAATA CCTGATTTCC |
| 4901 | TTAAACATCT AGAAACTGAA GACAACATTA AGGTGTGGTT TAATAACAAA GGCTGGCATG |
| 4961 | CCCTGGTCAG CTTTCTCAAT GTGGCCCACA ACGCCATCTT ACGGGCCAGC CTGCCTAAGG |
| 5021 | ACAGGAGCCC CGAGGAGTAT GGAATCACCG TCATTAGCCA ACCCCTGAAC CTGACCAAGG |
| 5081 | AGCAGCTCTC AGAGATTACA GTGCTGACCA CTTCAGTGGA TGCTGTGGTT GCCATCTGCG |
| 5141 | TGATTTTCTC CATGTCCTTC GTCCCAGCCA GCTTTGTCCT TTATTTGATC CAGGAGCGGG |
| 5201 | TGAACAAATC CAAGCACCTC CAGTTTATCA GTGGAGTGAG CCCCACCACC TACTGGGTGA |
| 5261 | CCAACTTCCT CTGGGACATC ATGAATTATT CCGTGAGTGC TGGGCTGGTG GTGGGCATCT |
| 5321 | TCATCGGGTT TCAGAAGAAA GCCTACACTT CTCCAGAAAA CCTTCCTGCC CTTGTGGCAC |
| 5381 | TGCTCCTGCT GTATGGATGG GCGGTCATTC CCATGATGTA CCCAGCATCC TTCCTGTTTG |
| 5441 | ATGTCCCCAG CACAGCCTAT GTGGCTTTAT CTTGTGCTAA TCTGTTCATC GGCATCAACA |
| 5501 | GCAGTGCTAT TACCTTCATC TTGGAATTAT TTGAGAATAA CCGGACGCTG CTCAGGTTCA |
| 5561 | ACGCCGTGCT GAGGAAGCTG CTCATTGTCT TCCCCCACTT CTGCCTGGGC CGGGGCCTCA |
| 5621 | TTGACCTTGC ACTGAGCCAG GCTGTGACAG ATGTCTATGC CCGGTTTGGT GAGGAGCACT |
| 5681 | CTGCAAATCC GTTCCACTGG GACCTGATTG GGAAGAACCT GTTTGCCATG GTGGTGGAAG |
| 5741 | GGGTGGTGTA CTTCCTCCTG ACCCTGCTGG TCCAGCGCCA CTTCTTCCTC TCCCAATGGA |
| 5801 | TTGCCGAGCC CACTAAGGAG CCCATTGTTG ATGAAGATGA TGATGTGGCT GAAGAAAGAC |
| 5861 | AAAGAATTAT TACTGGTGGA AATAAAACTG ACATCTTAAG GCTACATGAA CTAACCAAGA |
| 5921 | TTTATCCAGG CACCTCCAGC CCAGCAGTGG ACAGGCTGTG TGTCGGAGTT CGCCCTGGAG |
| 5981 | AGTGCTTTGG CCTCCTGGGA GTGAATGGTG CCGGCAAAAC AACCACATTC AAGATGCTCA |
| 6041 | CTGGGGACAC CACAGTGACC TCAGGGGATG CCACCGTAGC AGGCAAGAGT ATTTTAACCA |
| 6101 | ATATTTCTGA AGTCCATCAA AATATGGGCT ACTGTCCTCA GTTTGATGCA ATTGATGAGC |
| 6161 | TGCTCACAGG ACGAGAACAT CTTTACCTTT ATGCCCGGCT TCGAGGTGTA CCAGCAGAAG |
| 6221 | AAATCGAAAA GGTTGCAAAC TGGAGTATTA AGAGCCTGGG CCTGACTGTC TACGCCGACT |
| 6281 | GCCTGGCTGG CACGTACAGT GGGGGCAACA AGCGGAAACT CTCCACAGCC ATCGCACTCA |
| 6341 | TTGGCTGCCC ACCGCTGGTG CTGCTGGATG AGCCCACCAC AGGGATGGAC CCCCAGGCAC |
| 6401 | GCCGCATGCT GTGGAACGTC ATCGTGAGCA TCATCAGAGA AGGGAGGGCT GTGGTCCTCA |
| 6461 | CATCCCACAG CATGGAAGAA TGTGAGGCAC TGTGTACCCG GCTGGCCATC ATGGTAAAGG |
| 6521 | GCGCCTTTCG ATGTATGGGC ACCATTCAGC ATCTCAAGTC CAAATTTGGA GATGGCTATA |
| 6581 | TCGTCACAAT GAAGATCAAA TCCCCGAAGG ACGACCTGCT TCCTGACCTG AACCCTGTGG |
| 6641 | AGCAGTTCTT CCAGGGGAAC TTCCCAGGCA GTGTGCAGAG GGAGAGGCAC TACAACATGC |
| 6701 | TCCAGTTCCA GGTCTCCTCC TCCTCCCTGG CGAGGATCTT CCAGCTCCTC CTCTCCCACA |
| 6761 | AGGACAGCCT GCTCATCGAG GAGTACTCAG TCACACAGAC CACACTGGAC CAGGTGTTTG |
| 6821 | TAAATTTTGC TAAACAGCAG ACTGAAAGTC ATGACCTCCC TCTGCACCCT CGAGCTGCTG |
| 6881 | GAGCCAGTCG ACAAGCCCAG GACTGATCTT TCACACCGCT CGTTCCTGCA GCCAGAAAGG |
| 6941 | AACTCTGGGC AGCTGGAGGC GCAGGAGCCT GTGCCCATAT GGTCATCCAA ATGGACTGGC |
| 7001 | CAGCGTAAAT GACCCCACTG CAGCAGAAAA CAAACACACG AGGAGCATGC AGCGAATTCA |
| 7061 | GAAAGAGGTC TTTCAGAAGG AAACCGAAAC TGACTTGCTC ACCTGGAACA CCTGATGGTG |
| 7121 | AAACCAAACA AATACAAAAT CCTTCTCCAG ACCCCAGAAC TAGAAACCCC GGGCCATCCC |
| 7181 | ACTAGCAGCT TTGGCCTCCA TATTGCTCTC ATTTCAAGCA GATCTGCTTT TCTGCATGTT |
| 7241 | TGTCTGTGTG TCTGCGTTGT GTGTGATTTT CATGGAAAAA TAAAATGCAA ATGCACTCAT |
| 7301 | CACAAA |

SEQ ID NO: 2

| | |
|---|---|
| 1 | AGGACACAGC GTCCGGAGCC AGAGGCGCTC TTAACGGCGT TTATGTCCTT TGCTGTCTGA |
| 61 | GGGGCCTCAG CTCTGACCAA TCTGGTCTTC GTGTGGTCAT TAGCATGGGC TTCGTGAGAC |
| 121 | AGATACAGCT TTTGCTCTGG AAGAACTGGA CCCTGCGGAA AAGGCAAAAG ATTCGCTTTG |
| 181 | TGGTGGAACT CGTGTGGCCT TTATCTTTAT TTCTGGTCTT GATCTGGTTA AGGAATGCCA |
| 241 | ACCCGCTCTA CAGCCATCAT GAATGCCATT TCCCCAACAA GGCGATGCCC TCAGCAGGAA |
| 301 | TGCTGCCGTG GCTCCAGGGG ATCTTCTGCA ATGTGAACAA TCCCTGTTTT CAAAGCCCCA |
| 361 | CCCCAGGAGA ATCTCCTGGA ATTGTGTCAA ACTATAACAA CTCCATCTTG GCAAGGGTAT |
| 421 | ATCGAGATTT TCAAGAACTC CTCATGAATG CACCAGAGAG CCAGCACCTT GGCCGTATTT |
| 481 | GGACAGAGCT ACACATCTTG TCCCAATTCA TGGACACCCT CCGGACTCAC CCGGAGAGAA |
| 541 | TTGCAGGAAG AGGAATACGA ATAAGGGATA TCTTGAAAGA TGAAGAAACA CTGACACTAT |
| 601 | TTCTCATTAA AAACATCGGC CTGTCTGACT CAGTGGTCTA CCTTCTGATC AACTCTCAAG |
| 661 | TCCGTCCAGA GCAGTTCGCT CATGGAGTCC CGGACCTGGC GCTGAAGGAC ATCGCCTGCA |
| 721 | GCGAGGCCCT CCTGGAGCGC TTCATCATCT TCAGCCAGAG ACGCGGGGCA AAGACGGTGC |
| 781 | GCTATGCCCT GTGCTCCCTC TCCCAGGGCA CCCTACAGTG GATAGAAGAC ACTCTGTATG |
| 841 | CCAACGTGGA CTTCTTCAAG CTCTTCCGTG TGCTTCCCAC ACTCCTAGAC AGCCGTTCTC |
| 901 | AAGGTATCAA TCTGAGATCT TGGGGAGGAA TATTATCTGA TATGTCACCA AGAATTCAAG |
| 961 | AGTTTATCCA TCGGCCGAGT ATGCAGGACT TGCTGTGGGT GACCAGGCCC CTCATGCAGA |
| 1021 | ATGGTGGTCC AGAGACCTTT ACAAAGCTGA TGGGCATCCT GTCTGACCTC CTGTGTGGCT |
| 1081 | ACCCCGAGGG AGGTGGCTCT CGGGTGCTCT CCTTCAACTG GTATGAAGAC AATAACTATA |
| 1141 | AGGCCTTTCT GGGGATTGAC TCCACAAGGA AGGATCCTAT CTATTCTTAT GACAGAAGAA |
| 1201 | CAACATCCTT TTGTAATGCA TTGATCCAGA GCCTGGAGTC AAATCCTTTA ACCAAAATCG |
| 1261 | CTTGGAGGGC GGCAAAGCCT TTGCTGATGG GAAAAATCCT GTACACTCCT GATTCACCTG |
| 1321 | CAGCACGAAG GATACTGAAG AATGCCAACT CAACTTTTGA AGAACTGGAA CACGTTAGGA |
| 1381 | AGTTGGTCAA AGCCTGGGAA GAAGTAGGGC CCCAGATCTG GTACTTCTTT GACAACAGCA |
| 1441 | CACAGATGAA CATGATCAGA GATACCCTGG GGAACCCAAC AGTAAAAGAC TTTTTGAATA |
| 1501 | GGCAGCTTGG TGAAGAAGGT ATTACTGCTG AAGCCATCCT AAACTTCCTC TACAAGGGCC |
| 1561 | CTCGGGAAAG CCAGGCTGAC GACATGGCCA ACTTCGACTG GAGGGACATA TTTAACATCA |
| 1621 | CTGATCGCAC CCTCCGCCTT GTCAATCAAT ACCTGGAGTG CTTGGTCCTG GATAAGTTTG |
| 1681 | AAAGCTACAA TGATGAAACT CAGCTCACCC AACGTGCCCT CTCTCTACTG GAGGAAAACA |
| 1741 | TGTTCTGGGC CGGAGTGGTA TTCCCTGACA TGTATCCCTG GACCAGCTCT CTACCACCCC |
| 1801 | ACGTGAAGTA TAAGATCCGA ATGGACATAG ACGTGGTGGA GAAAACCAAT AAGATTAAAG |
| 1861 | ACAGGTATTG GGATTCTGGT CCCAGAGCTG ATCCCGTGGA AGATTTCCGG TACATCTGGG |
| 1921 | GCGGGTTTGC CTATCTGCAG GACATGGTTG AACAGGGGAT CACAAGGAGC CAGGTGCAGG |
| 1981 | CGGAGGCTCC AGTTGGAATC TACCTCCAGC AGATGCCCTA CCCCTGCTTC GTGGACGATT |
| 2041 | CTTTCATGAT CATCCTGAAC CGCTGTTTCC CTATCTTCAT GGTGCTGGCA TGGATCTACT |
| 2101 | CTGTCTCCAT GACTGTGAAG AGCATCGTCT TGGAGAAGGA GTTGCGACTG AAGGAGACCT |
| 2161 | TGAAAAATCA GGGTGTCTCC AATGCAGTGA TTTGGTGTAC CTGGTTCCTG GACAGCTTCT |
| 2221 | CCATCATGTC GATGAGCATC TTCCTCCTGA CGATATTCAT CATGCATGGA AGAATCCTAC |
| 2281 | ATTACAGCGA CCCATTCATC CTCTTCCTGT TCTTGTTGGC TTTCTCCACT GCCACCATCA |
| 2341 | TGCTGTGCTT TCTGCTCAGC ACCTTCTTCT CCAAGGCCAG TCTGGCAGCA GCCTGTAGTG |
| 2401 | GTGTCATCTA TTTCACCCTC TACCTGCCAC ACATCCTGTG CTTCGCCTGG CAGGACCGCA |
| 2461 | TGACCGCTGA GCTGAAGAAG GCTGTGAGCT TACTGTCTCC GGTGGCATTT GGATTTGGCA |
| 2521 | CTGAGTACCT GGTTCGCTTT GAAGAGCAAG GCCTGGGGCT GCAGTGGAGC AACATCGGGA |
| 2581 | ACAGTCCCAC GGAAGGGGAC GAATTCAGCT TCCTGCTGTC CATGCAGATG ATGCTCCTTG |
| 2641 | ATGCTGCTGT CTATGGCTTA CTCGCTTGGT ACCTTGATCA GGTGTTTCCA GGAGACTATG |
| 2701 | GAACCCCACT TCCTTGGTAC TTTCTTCTAC AAGAGTCGTA TTGGCTTGGC GGTGAAGGGT |
| 2761 | GTTCAACCAG AGAAGAAAGA GCCCTGGAAA AGACCGAGCC CCTAACAGAG GAAACGGAGG |
| 2821 | ATCCAGAGCA CCCAGAAGGA ATACACGACT CCTTCTTTGA ACGTGAGCAT CCAGGGTGGG |
| 2881 | TTCCTGGGGT ATGCGTGAAG AATCTGGTAA AGATTTTTGA GCCCTGTGGC CGGCCAGCTG |
| 2941 | TGGACCGTCT GAACATCACC TTCTACGAGA ACCAGATCAC CGCATTCCTG GGCCACAATG |
| 3001 | GAGCTGGGAA AACCACCACC TTGTCCATCC TGACGGGTCT GTTGCCACCA ACCTCTGGGA |
| 3061 | CTGTGCTCGT TGGGGGAAGG GACATTGAAA CCAGCCTGGA TGCAGTCCGG CAGAGCCTTG |
| 3121 | GCATGTGTCC ACAGCACAAC ATCCTGTTCC ACCACCTCAC GGTGGCTGAG CACATGCTGT |
| 3181 | TCTATGCCCA GCTGAAAGGA AAGTCCCAGG AGGAGGCCCA GCTGGAGATG GAAGCCATGT |
| 3241 | TGGAGGACAC AGGCCTCCAC CACAAGCGGA ATGAAGAGGC TCAGGACCTA TCAGGTGGCA |
| 3301 | TGCAGAGAAA GCTGTCGGTT GCCATTGCCT TTGTGGGAGA TGCCAAGGTG GTGATTCTGG |
| 3361 | ACGAACCCAC CTCTGGGGTG GACCCTTACT CGAGACGCTC AATCTGGGAT CTGCTCCTGA |
| 3421 | AGTATCGCTC AGGCAGAACC ATCATCATGT CCACTCACCA CATGGACGAG GCCGACCTCC |
| 3481 | TTGGGGACCG CATTGCCATC ATTGCCCAGG GAAGGCTCTA CTGCTCAGGC ACCCCACTCT |
| 3541 | TCCTGAAGAA CTGCTTTGGC ACAGGCTTGT ACTTAACCTT GGTGCGCAAG ATGAAAAACA |
| 3601 | TCCAGAGCCA AAGGAAAGGC AGTGAGGGGA CCTGCAGCTG CTCGTCTAAG GGTTTCTCCA |
| 3661 | CCACGTGTCC AGCCCACGTC GATGACCTAA CTCCAGAACA AGTCCTGGAT GGGGATGTAA |
| 3721 | ATGAGCTGAT GGATGTAGTT CTCCACCATG TTCCAGAGGC AAAGCTGGTG GAGTGCATTG |
| 3781 | GTCAAGAACT TATCTTCCTT CTTCCAAATA AGAACTTCAA GCACAGAGCA TATGCCAGCC |
| 3841 | TTTTCAGAGA GCTGGAGGAG ACGCTGGCTG ACCTTGGTCT CAGCAGTTTT GGAATTTCTG |
| 3901 | ACACTCCCCT GGAAGAGATT TTTCTGAAGG TCACGGAGGA TTCTGATTCA GGACCTCTGT |
| 3961 | TTGCGGGTGG CGCTCAGCAG AAAAGAGAAA ACGTCAACCC CCGACACCCC TGCTTGGGTC |
| 4021 | CCAGAGAGAA GGCTGGACAG ACACCCCAGG ACTCCAATGT CTGCTCCCCA GGGGCGCCGG |
| 4081 | CTGCTCACCC AGAGGGCCAG CCTCCCCCAG AGCCAGAGTG CCCAGGCCCG CAGCTCAACA |
| 4141 | CGGGGACACA GCTGGTCCTC CAGCATGTGC AGGCGCTGCT GGTCAAGAGA TTCCAACACA |
| 4201 | CCATCCGCAG CCACAAGGAC TTCCTGGCGC AGATCGTGCT CCCGGCTACC TTTGTGTTTT |
| 4261 | TGGCTCTGAT GCTTTCTATT GTTATCCCTC CTTTTGGCGA ATACCCCGCT TTGACCCTTC |
| 4321 | ACCCCTGGAT ATATGGGCAG CAGTACACCT TCTTCAGCAT GGATGAACCA GGCAGTGAGC |
| 4381 | AGTTCACGGT ACTTGCAGAC GTCCTCCTGA ATAAGCCAGG CTTTGGCAAC CGCTGCCTGA |
| 4441 | AGGAAGGGTG GCTTCCGGAG TACCCCTGTG GCAACTCAAC ACCCTGGAAG ACTCCTTCTG |
| 4501 | TGTCCCCAAA CATCACCCAG CTGTTCCAGA AGCAGAAATG GACACAGGTC AACCCTTCAC |
| 4561 | CATCCTGCAG GTGCAGCACC AGGGAGAAGC TCACCATGCT GCCAGAGTGC CCCGAGGGTG |
| 4621 | CCGGGGGCCT CCCGCCCCCC CAGAGAACAC AGCGCAGCAC GGAAATTCTA CAAGACCTGA |
| 4681 | CGGACAGGAA CATCTCCGAC TTCTTGGTAA AAACGTATCC TGCTCTTATA AGAAGCAGCT |
| 4741 | TAAAGAGCAA ATTCTGGGTC AATGAACAGA GGTATGGAGG AATTTCCATT GGAGGAAAGC |
| 4801 | TCCCAGTCGT CCCCATCACG GGGGAAGCAC TTGTTGGGTT TTTAAGCGAC CTTGGCCGGA |
| 4861 | TCATGAATGT GAGCGGGGGC CCTATCACTA GAGAGGCCTC TAAAGAAATA CCTGATTTCC |
| 4921 | TTAAACATCT AGAAACTGAA GACAACATTA AGGTGTGGTT TAATAACAAA GGCTGGCATG |
| 4981 | CCCTGGTCAG CTTTCTCAAT GTGGCCCACA ACGCCATCTT ACGGGCCAGC CTGCCTAAGG |
| 5041 | ACAGGAGCCC CGAGGAGTAT GGAATCACCG TCATTAGCCA ACCCCTGAAC CTGACCAAGG |
| 5101 | AGCAGCTCTC AGAGATTACA GTGCTGACCA CTTCAGTGGA TGCTGTGGTT GCCATCTGCG |
| 5161 | TGATTTTCTC CATGTCCTTC GTCCCAGCCA GCTTTGTCCT TTATTTGATC CAGGAGCGGG |
| 5221 | TGAACAAATC CAAGCACCTC CAGTTTATCA GTGGAGTGAG CCCCACCACC TACTGGGTAA |
| 5281 | CCAACTTCCT CTGGGACATC ATGAATTATT CCGTGAGTGC TGGGCTGGTG GTGGGCATCT |
| 5341 | TCATCGGGTT TCAGAAGAAA GCCTACACTT CTCCAGAAAA CCTTCCTGCC CTTGTGGCAC |
| 5401 | TGCTCCTGCT GTATGGATGG GCGGTCATTC CCATGATGTA CCCAGCATCC TTCCTGTTTG |
| 5461 | ATGTCCCCAG CACAGCCTAT GTGGCTTTAT CTTGTGCTAA TCTGTTCATC GGCATCAACA |
| 5521 | GCAGTGCTAT TACCTTCATC TTGGAATTAT TTGAGAATAA CCGGACGCTG CTCAGGTTCA |
| 5581 | ACGCCGTGCT GAGGAAGCTG CTCATTGTCT TCCCCCACTT CTGCCTGGGC CGGGGCCTCA |
| 5641 | TTGACCTTGC ACTGAGCCAG GCTGTGACAG ATGTCTATGC CCGGTTTGGT GAGGAGCACT |
| 5701 | CTGCAAATCC GTTCCACTGG GACCTGATTG GGAAGAACCT GTTTGCCATG GTGGTGGAAG |
| 5761 | GGGTGGTGTA CTTCCTCCTG ACCCTGCTGG TCCAGCGCCA CTTCTTCCTC TCCCAATGGA |
| 5821 | TTGCCGAGCC CACTAAGGAG CCCATTGTTG ATGAAGATGA TGATGTGGCT GAAGAAAGAC |
| 5881 | AAAGAATTAT TACTGGTGGA AATAAAACTG ACATCTTAAG GCTACATGAA CTAACCAAGA |
| 5941 | TTTATCCAGG CACCTCCAGC CCAGCAGTGG ACAGGCTGTG TGTCGGAGTT CGCCCTGGAG |
| 6001 | AGTGCTTTGG CCTCCTGGGA GTGAATGGTG CCGGCAAAAC AACCACATTC AAGATGCTCA |
| 6061 | CTGGGGACAC CACAGTGACC TCAGGGGATG CCACCGTAGC AGGCAAGAGT ATTTTAACCA |
| 6121 | ATATTTCTGA AGTCCATCAA AATATGGGCT ACTGTCCTCA GTTTGATGCA ATCGATGAGC |
| 6181 | TGCTCACAGG ACGAGAACAT CTTTACCTTT ATGCCCGGCT TCGAGGTGTA CCAGCAGAAG |
| 6241 | AAATCGAAAA GGTTGCAAAC TGGAGTATTA AGAGCCTGGG CCTGACTGTC TACGCCGACT |
| 6301 | GCCTGGCTGG CACGTACAGT GGGGGCAACA AGCGGAAACT CTCCACAGCC ATCGCACTCA |
| 6361 | TTGGCTGCCC ACCGCTGGTG CTGCTGGATG AGCCCACCAC AGGGATGGAC CCCCAGGCAC |
| 6421 | GCCGCATGCT GTGGAACGTC ATCGTGAGCA TCATCAGAGA AGGGAGGGCT GTGGTCCTCA |
| 6481 | CATCCCACAG CATGGAAGAA TGTGAGGCAC TGTGTACCCG GCTGGCCATC ATGGTAAAGG |
| 6541 | GCGCCTTTCG ATGTATGGGC ACCATTCAGC ATCTCAAGTC CAAATTTGGA GATGGCTATA |
| 6601 | TCGTCACAAT GAAGATCAAA TCCCCGAAGG ACGACCTGCT TCCTGACCTG AACCCTGTGG |
| 6661 | AGCAGTTCTT CCAGGGGAAC TTCCCAGGCA GTGTGCAGAG GGAGAGGCAC TACAACATGC |
| 6721 | TCCAGTTCCA GGTCTCCTCC TCCTCCCTGG CGAGGATCTT CCAGCTCCTC CTCTCCCACA |
| 6781 | AGGACAGCCT GCTCATCGAG GAGTACTCAG TCACACAGAC CACACTGGAC CAGGTGTTTG |
| 6841 | TAAATTTTGC TAAACAGCAG ACTGAAAGTC ATGACCTCCC TCTGCACCCT CGAGCTGCTG |
| 6901 | GAGCCAGTCG ACAAGCCCAG GACTGATCTT TCACACCGCT CGTTCCTGCA GCCAGAAAGG |
| 6961 | AACTCTGGGC AGCTGGAGGC GCAGGAGCCT GTGCCCATAT GGTCATCCAA ATGGACTGGC |
| 7021 | CAGCGTAAAT GACCCCACTG CAGCAGAAAA CAAACACACG AGGAGCATGC AGCGAATTCA |
| 7081 | GAAAGAGGTC TTTCAGAAGG AAACCGAAAC TGACTTGCTC ACCTGGAACA CCTGATGGTG |
| 7141 | AAACCAAACA AATACAAAAT CCTTCTCCAG ACCCCAGAAC TAGAAACCCC GGGCCATCCC |
| 7201 | ACTAGCAGCT TTGGCCTCCA TATTGCTCTC ATTTCAAGCA GATCTGCTTT TCTGCATGTT |
| 7261 | TGTCTGTGTG TCTGCGTTGT GTGTGATTTT CATGGAAAAA TAAAATGCAA ATGCACTCAT |
| 7321 | CACAAA |

SEQ ID NO: 3

| | |
|---|---|
| 1 | TTGGCCACTC CCTCTCTGCG CGCTCGCTCG CTCACTGAGG CCGGGCGACC AAAGGTCGCC |
| 61 | CGACGCCCGG GCTTTGCCCG GGCGGCCTCA GTGAGCGAGC GAGCGCGCAG AGAGGGAGTG |
| 121 | GCCAACTCCA TCACTAGGGG TTCCTGCGGC AATTCAGTCG ATAACTATAA CGGTCCTAAG |
| 181 | GTAGCGATTT AAATGGTACC GGGCCCCAGA AGCCTGGTGG TTGTTTGTCC TTCTCAGGGG |
| 241 | AAAAGTGAGG CGGCCCCTTG GAGGAAGGGG CCGGGCAGAA TGATCTAATC GGATTCCAAG |
| 301 | CAGCTCAGGG GATTGTCTTT TTCTAGCACC TTCTTGCCAC TCCTAAGCGT CCTCCGTGAC |
| 361 | CCCGGCTGGG ATTTAGCCTG GTGCTGTGTC AGCCCCGGGT GCCGCAGGGG GACGGCTGCC |
| 421 | TTCGGGGGGG ACGGGGCAGG GCGGGGTTCG GCTTCTGGCG TGTGACCGGC GGCTCTAGAG |
| 481 | CCTCTGCTAA CCATGTTCAT GCCTTCTTCT TTTTCCTACA GCTCCTGGGC AACGTGCTGG |
| 541 | TTATTGTGCT GTCTCATCAT TTTGGCAAAG AATTACCACC ATGGGCTTCG TGAGACAGAT |
| 601 | ACAGCTTTTG CTCTGGAAGA ACTGGACCCT GCGGAAAAGG CAAAAGATTC GCTTTGTGGT |
| 661 | GGAACTCGTG TGGCCTTTAT CTTTATTTCT GGTCTTGATC TGGTTAAGGA ATGCCAACCC |
| 721 | GCTCTACAGC CATCATGAAT GCCATTTCCC CAACAAGGCG ATGCCCTCAG CAGGAATGCT |
| 781 | GCCGTGGCTC CAGGGGATCT TCTGCAATGT GAACAATCCC TGTTTTCAAA GCCCCACCCC |
| 841 | AGGAGAATCT CCTGGAATTG TGTCAAACTA TAACAACTCC ATCTTGGCAA GGGTATATCG |
| 901 | AGATTTTCAA GAACTCCTCA TGAATGCACC AGAGAGCCAG CACCTTGGCC GTATTTGGAC |
| 961 | AGAGCTACAC ATCTTGTCCC AATTCATGGA CACCCTCCGG ACTCACCCGG AGAGAATTGC |
| 1021 | AGGAAGAGGA ATACGAATAA GGGATATCTT GAAAGATGAA GAAACACTGA CACTATTTCT |
| 1081 | CATTAAAAAC ATCGGCCTGT CTGACTCAGT GGTCTACCTT CTGATCAACT CTCAAGTCCG |
| 1141 | TCCAGAGCAG TTCGCTCATG GAGTCCCGGA CCTGGCGCTG AAGGACATCG CCTGCAGCGA |
| 1201 | GGCCCTCCTG GAGCGCTTCA TCATCTTCAG CCAGAGACGC GGGGCAAAGA CGGTGCGCTA |
| 1261 | TGCCCTGTGC TCCCTCTCCC AGGGCACCCT ACAGTGGATA GAAGACACTC TGTATGCCAA |
| 1321 | CGTGGACTTC TTCAAGCTCT TCCGTGTGCT TCCCACACTC CTAGACAGCC GTTCTCAAGG |
| 1381 | TATCAATCTG AGATCTTGGG GAGGAATATT ATCTGATATG TCACCAAGAA TTCAAGAGTT |
| 1441 | TATCCATCGG CCGAGTATGC AGGACTTGCT GTGGGTGACC AGGCCCCTCA TGCAGAATGG |
| 1501 | TGGTCCAGAG ACCTTTACAA AGCTGATGGG CATCCTGTCT GACCTCCTGT GTGGCTACCC |
| 1561 | CGAGGGAGGT GGCTCTCGGG TGCTCTCCTT CAACTGGTAT GAAGACAATA ACTATAAGGC |
| 1621 | CTTTCTGGGG ATTGACTCCA CAAGGAAGGA TCCTATCTAT TCTTATGACA GAAGAACAAC |
| 1681 | ATCCTTTTGT AATGCATTGA TCCAGAGCCT GGAGTCAAAT CCTTTAACCA AAATCGCTTG |
| 1741 | GAGGGCGGCA AAGCCTTTGC TGATGGGAAA AATCCTGTAC ACTCCTGATT CACCTGCAGC |
| 1801 | ACGAAGGATA CTGAAGAATG CCAACTCAAC TTTTGAAGAA CTGGAACACG TTAGGAAGTT |
| 1861 | GGTCAAAGCC TGGGAAGAAG TAGGGCCCCA GATCTGGTAC TTCTTTGACA ACAGCACACA |
| 1921 | GATGAACATG ATCAGAGATA CCCTGGGGAA CCCAACAGTA AAAGACTTTT TGAATAGGCA |
| 1981 | GCTTGGTGAA GAAGGTATTA CTGCTGAAGC CATCCTAAAC TTCCTCTACA AGGGCCCTCG |
| 2041 | GGAAAGCCAG GCTGACGACA TGGCCAACTT CGACTGGAGG GACATATTTA ACATCACTGA |
| 2101 | TCGCACCCTC CGCCTTGTCA ATCAATACCT GGAGTGCTTG GTCCTGGATA AGTTTGAAAG |
| 2161 | CTACAATGAT GAAACTCAGC TCACCCAACG TGCCCTCTCT CTACTGGAGG AAAACATGTT |
| 2221 | CTGGGCCGGA GTGGTATTCC CTGACATGTA TCCCTGGACC AGCTCTCTAC CACCCCACGT |
| 2281 | GAAGTATAAG ATCCGAATGG ACATAGACGT GGTGGAGAAA ACCAATAAGA TTAAAGACAG |
| 2341 | GTATTGGGAT TCTGGTCCCA GAGCTGATCC CGTGGAAGAT TTCCGGTACA TCTGGGGCGG |
| 2401 | GTTTGCCTAT CTGCAGGACA TGGTTGAACA GGGGATCACA AGGAGCCAGG TGCAGGCGGA |
| 2461 | GGCTCCAGTT GGAATCTACC TCCAGCAGAT GCCCTACCCC TGCTTCGTGG ACGATTCTTT |
| 2521 | CATGATCATC CTGAACCGCT GTTTCCCTAT CTTCATGGTG CTGGCATGGA TCTACTCTGT |
| 2581 | CTCCATGACT GTGAAGAGCA TCGTCTTGGA GAAGGAGTTG CGACTGAAGG AGACCTTGAA |
| 2641 | AAATCAGGGT GTCTCCAATG CAGTGATTTG GTGTACCTGG TTCCTGGACA GCTTCTCCAT |
| 2701 | CATGTCGATG AGCATCTTCC TCCTGACGAT ATTCATCATG CATGGAAGAA TCCTACATTA |
| 2761 | CAGCGACCCA TTCATCCTCT TCCTGTTCTT GTTGGCTTTC TCCACTGCCA CCATCATGCT |
| 2821 | GTGCTTTCTG CTCAGCACCT TCTTCTCCAA GGCCAGTCTG GCAGCAGCCT GTAGTGGTGT |
| 2881 | CATCTATTTC ACCCTCTACC TGCCACACAT CCTGTGCTTC GCCTGGCAGG ACCGCATGAC |
| 2941 | CGCTGAGCTG AAGAAGGCTG TGAGCTTACT GTCTCCGGTG GCATTTGGAT TTGGCACTGA |
| 3001 | GTACCTGGTT CGCTTTGAAG AGCAAGGCCT GGGGCTGCAG TGGAGCAACA TCGGGAACAG |
| 3061 | TCCCACGGAA GGGGACGAAT TCAGCTTCCT GCTGTCCATG CAGATGATGC TCCTTGATGC |
| 3121 | TGCTGTCTAT GGCTTACTCG CTTGGTACCT TGATCAGGTG TTTCCAGGAG ACTATGGAAC |
| 3181 | CCCACTTCCT TGGTACTTTC TTCTACAAGA GTCGTATTGG CTTGGCGGTG AAGGGTGTTC |
| 3241 | AACCAGAGAA GAAAGAGCCC TGGAAAAGAC CGAGCCCCTA ACAGAGGAAA CGGAGGATCC |
| 3301 | AGAGCACCCA GAAGGAATAC ACGACTCCTT CTTTGAACGT GAGCATCCAG GGTGGGTTCC |
| 3361 | TGGGGTATGC GTGAAGAATC TGGTAAAGAT TTTTGAGCCC TGTGGCCGGC CAGCTGTGGA |
| 3421 | CCGTCTGAAC ATCACCTTCT ACGAGAACCA GATCACCGCA TTCCTGGGCC ACAATGGAGC |
| 3481 | TGGGAAAACC ACCACCTTGT CCATCCTGAC GGGTCTGTTG CCACCAACCT CTGGGACTGT |
| 3541 | GCTCGTTGGG GGAAGGGACA TTGAAACCAG CCTGGATGCA GTCCGGCAGA GCCTTGGCAT |
| 3601 | GTGTCCACAG CACAACATCC TGTTCCACCA CCTCACGGTG GCTGAGCACA TGCTGTTCTA |
| 3661 | TGCCCAGCTG AAAGGAAAGT CCCAGGAGGA GGCCCAGCTG GAGATGGAAG CCATGTTGGA |
| 3721 | GGACACAGGC CTCCACCACA AGCGGAATGA AGAGGCTCAG GACCTATCAG GTGGCATGCA |
| 3781 | GAGAAAGCTG TCGGTTGCCA TTGCCTTTGT GGGAGATGCC AAGGTGGTGA TTCTGGACGA |
| 3841 | ACCCACCTCT GGGGTGGACC CTTACTCGAG ACGCTCAATC TGGGATCTGC TCCTGAAGTA |
| 3901 | TCGCTCAGGC AGAACCATCA TCATGTCCAC TCACCACATG GACGAGGCCG ACCTCCTTGG |
| 3961 | GGACCGCATT GCCATCATTG CCCAGGGAAG GCTCTACTGC TCAGGCACCC CACTCTTCCT |
| 4021 | GAAGAACTGC TTTGGCACAG GCTTGTACTT AACCTTGGTG CGCAAGATGA AAAACATCCA |
| 4081 | GAGCCAAAGG AAAGGCAGTG AGGGGACCTG CAGCTGCTCG TCTAAGGGTT TCTCCACCAC |
| 4141 | GTGTCCAGCC CACGTCGATG ACCTAACTCC AGAACAAGTC CTGGATGGGG ATGTAAATGA |
| 4201 | GCTGATGGAT GTAGTTCTCC ACCATGTTCC AGAGGCAAAG CTGGTGGAGT GCATTGGTCA |
| 4261 | AGAACTTATC TTCCTTCTTC CATTTAAATT AGGGATAACA GGGTAATGGC GCGGGCCGCA |
| 4321 | GGAACCCCTA GTGATGGAGT TGGCCACTCC CTCTCTGCGC GCTCGCTCGC TCACTGAGGC |
| 4381 | CGCCCGGGCA AAGCCCGGGC GTCGGGCGAC CTTTGGTCGC CCGGCCTCAG TGAGCGAGCG |
| 4441 | AGCGCGCAGA GAGGGAGTGG CCAA |

SEQ ID NO: 4

| | |
|---|---|
| 1 | TTGGCCACTC CCTCTCTGCG CGCTCGCTCG CTCACTGAGG CCGGGCGACC AAAGGTCGCC |
| 61 | CGACGCCCGG GCTTTGCCCG GGCGGCCTCA GTGAGCGAGC GAGCGCGCAG AGAGGGAGTG |
| 121 | GCCAACTCCA TCACTAGGGG TTCCTGCGGC AATTCAGTCG ATAACTATAA CGGTCCTAAG |
| 181 | GTAGCGATTT AAATAACATC CAGAGCCAAA GGAAAGGCAG TGAGGGGACC TGCAGCTGCT |
| 241 | CGTCTAAGGG TTTCTCCACC ACGTGTCCAG CCCACGTCGA TGACCTAACT CCAGAACAAG |
| 301 | TCCTGGATGG GGATGTAAAT GAGCTGATGG ATGTAGTTCT CCACCATGTT CCAGAGGCAA |
| 361 | AGCTGGTGGA GTGCATTGGT CAAGAACTTA TCTTCCTTCT TCCAAATAAG AACTTCAAGC |
| 421 | ACAGAGCATA TGCCAGCCTT TTCAGAGAGC TGGAGGAGAC GCTGGCTGAC CTTGGTCTCA |
| 481 | GCAGTTTTGG AATTTCTGAC ACTCCCCTGG AAGAGATTTT TCTGAAGGTC ACGGAGGATT |
| 541 | CTGATTCAGG ACCTCTGTTT GCGGGTGGCG CTCAGCAGAA AAGAGAAAAC GTCAACCCCC |
| 601 | GACACCCCTG CTTGGGTCCC AGAGAGAAGG CTGGACAGAC ACCCCAGGAC TCCAATGTCT |
| 661 | GCTCCCCAGG GGCGCCGGCT GCTCACCCAG AGGGCCAGCC TCCCCCAGAG CCAGAGTGCC |
| 721 | CAGGCCCGCA GCTCAACACG GGGACACAGC TGGTCCTCCA GCATGTGCAG GCGCTGCTGG |
| 781 | TCAAGAGATT CCAACACACC ATCCGCAGCC ACAAGGACTT CCTGGCGCAG ATCGTGCTCC |
| 841 | CGGCTACCTT TGTGTTTTTG GCTCTGATGC TTTCTATTGT TATCCCTCCT TTTGGCGAAT |
| 901 | ACCCCGCTTT GACCCTTCAC CCCTGGATAT ATGGGCAGCA GTACACCTTC TTCAGCATGG |
| 961 | ATGAACCAGG CAGTGAGCAG TTCACGGTAC TTGCAGACGT CCTCCTGAAT AAGCCAGGCT |
| 1021 | TTGGCAACCG CTGCCTGAAG GAAGGGTGGC TTCCGGAGTA CCCCTGTGGC AACTCAACAC |
| 1081 | CCTGGAAGAC TCCTTCTGTG TCCCCAAACA TCACCCAGCT GTTCCAGAAG CAGAAATGGA |
| 1141 | CACAGGTCAA CCCTTCACCA TCCTGCAGGT GCAGCACCAG GGAGAAGCTC ACCATGCTGC |
| 1201 | CAGAGTGCCC CGAGGGTGCC GGGGGCCTCC CGCCCCCCCA GAGAACACAG CGCAGCACGG |
| 1261 | AAATTCTACA AGACCTGACG GACAGGAACA TCTCCGACTT CTTGGTAAAA ACGTATCCTG |
| 1321 | CTCTTATAAG AAGCAGCTTA AAGAGCAAAT TCTGGGTCAA TGAACAGAGG TATGGAGGAA |
| 1381 | TTTCCATTGG AGGAAAGCTC CCAGTCGTCC CCATCACGGG GGAAGCACTT GTTGGGTTTT |
| 1441 | TAAGCGACCT TGGCCGGATC ATGAATGTGA GCGGGGGCCC TATCACTAGA GAGGCCTCTA |
| 1501 | AAGAAATACC TGATTTCCTT AAACATCTAG AAACTGAAGA CAACATTAAG GTGTGGTTTA |
| 1561 | ATAACAAAGG CTGGCATGCC CTGGTCAGCT TTCTCAATGT GGCCCACAAC GCCATCTTAC |
| 1621 | GGGCCAGCCT GCCTAAGGAC AGGAGCCCCG AGGAGTATGG AATCACCGTC ATTAGCCAAC |
| 1681 | CCCTGAACCT GACCAAGGAG CAGCTCTCAG AGATTACAGT GCTGACCACT TCAGTGGATG |
| 1741 | CTGTGGTTGC CATCTGCGTG ATTTTCTCCA TGTCCTTCGT CCCAGCCAGC TTTGTCCTTT |
| 1801 | ATTTGATCCA GGAGCGGGTG AACAAATCCA AGCACCTCCA GTTTATCAGT GGAGTGAGCC |
| 1861 | CCACCACCTA CTGGGTAACC AACTTCCTCT GGGACATCAT GAATTATTCC GTGAGTGCTG |
| 1921 | GGCTGGTGGT GGGCATCTTC ATCGGGTTTC AGAAGAAAGC CTACACTTCT CCAGAAAACC |
| 1981 | TTCCTGCCCT TGTGGCACTG CTCCTGCTGT ATGGATGGGC GGTCATTCCC ATGATGTACC |
| 2041 | CAGCATCCTT CCTGTTTGAT GTCCCCAGCA CAGCCTATGT GGCTTTATCT TGTGCTAATC |
| 2101 | TGTTCATCGG CATCAACAGC AGTGCTATTA CCTTCATCTT GGAATTATTT GAGAATAACC |
| 2161 | GGACGCTGCT CAGGTTCAAC GCCGTGCTGA GGAAGCTGCT CATTGTCTTC CCCCACTTCT |
| 2221 | GCCTGGGCCG GGGCCTCATT GACCTTGCAC TGAGCCAGGC TGTGACAGAT GTCTATGCCC |
| 2281 | GGTTTGGTGA GGAGCACTCT GCAAATCCGT TCCACTGGGA CCTGATTGGG AAGAACCTGT |
| 2341 | TTGCCATGGT GGTGGAAGGG GTGGTGTACT TCCTCCTGAC CCTGCTGGTC CAGCGCCACT |
| 2401 | TCTTCCTCTC CCAATGGATT GCCGAGCCCA CTAAGGAGCC CATTGTTGAT GAAGATGATG |
| 2461 | ATGTGGCTGA AGAAAGACAA AGAATTATTA CTGGTGGAAA TAAAACTGAC ATCTTAAGGC |
| 2521 | TACATGAACT AACCAAGATT TATCCAGGCA CCTCCAGCCC AGCAGTGGAC AGGCTGTGTG |
| 2581 | TCGGAGTTCG CCCTGGAGAG TGCTTTGGCC TCCTGGGAGT GAATGGTGCC GGCAAAACAA |
| 2641 | CCACATTCAA GATGCTCACT GGGGACACCA CAGTGACCTC AGGGGATGCC ACCGTAGCAG |
| 2701 | GCAAGAGTAT TTTAACCAAT ATTTCTGAAG TCCATCAAAA TATGGGCTAC TGTCCTCAGT |
| 2761 | TTGATGCAAT CGATGAGCTG CTCACAGGAC GAGAACATCT TTACCTTTAT GCCCGGCTTC |
| 2821 | GAGGTGTACC AGCAGAAGAA ATCGAAAAGG TTGCAAACTG GAGTATTAAG AGCCTGGGCC |
| 2881 | TGACTGTCTA CGCCGACTGC CTGGCTGGCA CGTACAGTGG GGGCAACAAG CGGAAACTCT |
| 2941 | CCACAGCCAT CGCACTCATT GGCTGCCCAC CGCTGGTGCT GCTGGATGAG CCCACCACAG |
| 3001 | GGATGGACCC CCAGGCACGC CGCATGCTGT GGAACGTCAT CGTGAGCATC ATCAGAGAAG |
| 3061 | GGAGGGCTGT GGTCCTCACA TCCCACAGCA TGGAAGAATG TGAGGCACTG TGTACCCGGC |
| 3121 | TGGCCATCAT GGTAAAGGGC GCCTTTCGAT GTATGGGCAC CATTCAGCAT CTCAAGTCCA |
| 3181 | AATTTGGAGA TGGCTATATC GTCACAATGA AGATCAAATC CCCGAAGGAC GACCTGCTTC |
| 3241 | CTGACCTGAA CCCTGTGGAG CAGTTCTTCC AGGGGAACTT CCCAGGCAGT GTGCAGAGGG |
| 3301 | AGAGGCACTA CAACATGCTC CAGTTCCAGG TCTCCTCCTC CTCCCTGGCG AGGATCTTCC |
| 3361 | AGCTCCTCCT CTCCCACAAG GACAGCCTGC TCATCGAGGA GTACTCAGTC ACACAGACCA |
| 3421 | CACTGGACCA GGTGTTTGTA AATTTTGCTA AACAGCAGAC TGAAAGTCAT GACCTCCCTC |
| 3481 | TGCACCCTCG AGCTGCTGGA GCCAGTCGAC AAGCCCAGGA CTGAAAGCTT ATCGATAATC |
| 3541 | AACCTCTGGA TTACAAAATT TGTGAAAGAT TGACTGGTAT TCTTAACTAT GTTGCTCCTT |
| 3601 | TTACGCTATG TGGATACGCT GCTTTAATGC CTTTGTATCA TGCTATTGCT TCCCGTATGG |
| 3661 | CTTTCATTTT CTCCTCCTTG TATAAATCCT GGTTGCTGTC TCTTTATGAG GAGTTGTGGC |
| 3721 | CCGTTGTCAG GCAACGTGGC GTGGTGTGCA CTGTGTTTGC TGACGCAACC CCCACTGGTT |
| 3781 | GGGGCATTGC CACCACCTGT CAGCTCCTTT CCGGGACTTT CGCTTTCCCC CTCCCTATTG |
| 3841 | CCACGGCGGA ACTCATCGCC GCCTGCCTTG CCCGCTGCTG GACAGGGGCT CGGCTGTTGG |
| 3901 | GCACTGACAA TTCCGTGGTG TTGTCGGGGA AATCATCGTC CTTTCCTTGG CTGCTCGCCT |
| 3961 | GTGTTGCCAC CTGGATTCTG CGCGGGACGT CCTTCTGCTA CGTCCCTTCG GCCCTCAATC |
| 4021 | CAGCGGACCT TCCTTCCCGC GGCCTGCTGC CGGCTCTGCG GCCTCTTCCG CGTCTTCGCC |
| 4081 | TTCGCCCTCA GACGAGTCGG ATCTCCCTTT GGGCCGCCTC CCCGCATGCC GCTGATCAGC |
| 4141 | CTCGACTGTG CCTTCTAGTT GCCAGCCATC TGTTGTTTGC CCCTCCCCCG TGCCTTCCTT |
| 4201 | GACCCTGGAA GGTGCCACTC CCACTGTCCT TTCCTAATAA AATGAGGAAA TTGCATCGCA |
| 4261 | TTGTCTGAGT AGGTGTCATT CTATTCTGGG GGGTGGGGTG GGGCAGGACA GCAAGGGGGA |
| 4321 | GGATTGGGAA GACAATAGCA GGCATGCTGG GGATGCGGTG GGCTCTATGG CTTCTGAGGC |
| 4381 | GGAAAGAACC AGCTGGGGAT TTAAATTAGG GATAACAGGG TAATGGCGCG GGCCGCAGGA |
| 4441 | ACCCCTAGTG ATGGAGTTGG CCACTCCCTC TCTGCGCGCT CGCTCGCTCA CTGAGGCCGC |
| 4501 | CCGGGCAAAG CCCGGGCGTC GGGCGACCTT TGGTCGCCCG GCCTCAGTGA GCGAGCGAGC |
| 4561 | GCGCAGAGAG GGAGTGGCCA A |

SEQ ID NO: 5

| | |
|---|---|
| 1 | GGGCCCCAGA AGCCTGGTGG TTGTTTGTCC TTCTCAGGGG AAAAGTGAGG CGGCCCCTTG |
| 61 | GAGGAAGGGG CCGGGCAGAA TGATCTAATC GGATTCCAAG CAGCTCAGGG GATTGTCTTT |
| 121 | TTCTAGCACC TTCTTGCCAC TCCTAAGCGT CCTCCGTGAC CCCGGCTGGG ATTTAGCCTG |
| 181 | GTGCTGTGTC AGCCCCGGG |

SEQ ID NO: 6

| | |
|---|---|
| 1 | GTGCCGCAGG GGGACGGCTG CCTTCGGGGG GGACGGGGCA GGGCGGGGTT CGGCTTCTGG |
| 61 | CGTGTGACCG GCGGCTCTAG AGCCTCTGCT AACCATGTTC ATGCCTTCTT CTTTTTCCTA |
| 121 | CAGCTCCTGG GCAACGTGCT GGTTATTGTG CTGTCTCATC ATTTTGGCAA AGAATTACCA |
| 181 | CCATGG |

SEQ ID NO: 7

| | |
|---|---|
| 1 | ATCGATAATC AACCTCTGGA TTACAAAATT TGTGAAAGAT TGACTGGTAT TCTTAACTAT |
| 61 | GTTGCTCCTT TTACGCTATG TGGATACGCT GCTTTAATGC CTTTGTATCA TGCTATTGCT |
| 121 | TCCCGTATGG CTTTCATTTT CTCCTCCTTG TATAAATCCT GGTTGCTGTC TCTTTATGAG |
| 181 | GAGTTGTGGC CCGTTGTCAG GCAACGTGGC GTGGTGTGCA CTGTGTTTGC TGACGCAACC |
| 241 | CCCACTGGTT GGGGCATTGC CACCACCTGT CAGCTCCTTT CCGGGACTTT CGCTTTCCCC |
| 301 | CTCCCTATTG CCACGGCGGA ACTCATCGCC GCCTGCCTTG CCCGCTGCTG GACAGGGGCT |
| 361 | CGGCTGTTGG GCACTGACAA TTCCGTGGTG TTGTCGGGGA AATCATCGTC CTTTCCTTGG |
| 421 | CTGCTCGCCT GTGTTGCCAC CTGGATTCTG CGCGGGACGT CCTTCTGCTA CGTCCCTTCG |
| 481 | GCCCTCAATC CAGCGGACCT TCCTTCCCGC GGCCTGCTGC CGGCTCTGCG GCCTCTTCCG |
| 541 | CGTCTTCGCC TTCGCCCTCA GACGAGTCGG ATCTCCCTTT GGGCCGCCTC CCC |

SEQ ID NO: 8

| | |
|---|---|
| 1 | CGCTGATCAG CCTCGACTGT GCCTTCTAGT TGCCAGCCAT CTGTTGTTTG CCCCTCCCCC |
| 61 | GTGCCTTCCT TGACCCTGGA AGGTGCCACT CCCACTGTCC TTTCCTAATA AAATGAGGAA |
| 121 | ATTGCATCGC ATTGTCTGAG TAGGTGTCAT TCTATTCTGG GGGGTGGGGT GGGGCAGGAC |
| 181 | AGCAAGGGGG AGGATTGGGA AGACAATAGC AGGCATGCTG GGGATGCGGT GGGCTCTATG |
| 241 | GCTTCTGAGG CGGAAAGAAC CAGCTGGGG |

SEQ ID NO: 9

| | |
|---|---|
| 1 | GGTACCGGGC CCCAGAAGCC TGGTGGTTGT TTGTCCTTCT CAGGGGAAAA GTGAGGCGGC |
| 61 | CCCTTGGAGG AAGGGGCCGG GCAGAATGAT CTAATCGGAT TCCAAGCAGC TCAGGGGATT |
| 121 | GTCTTTTTCT AGCACCTTCT TGCCACTCCT AAGCGTCCTC CGTGACCCCG GCTGGGATTT |
| 181 | AGCCTGGTGC TGTGTCAGCC CCGGGTGCCG CAGGGGGACG GCTGCCTTCG GGGGGGACGG |
| 241 | GGCAGGGCGG GGTTCGGCTT CTGGCGTGTG ACCGGCGGCT CTAGAGCCTC TGCTAACCAT |
| 301 | GTTCATGCCT TCTTCTTTTT CCTACAGCTC CTGGGCAACG TGCTGGTTAT TGTGCTGTCT |
| 361 | CATCATTTTG GCAAAGAATT ACCACCATGG GCTTCGTGAG ACAGATACAG CTTTTGCTCT |
| 421 | GGAAGAACTG GACCCTGCGG AAAAGGCAAA AGATTCGCTT TGTGGTGGAA CTCGTGTGGC |
| 481 | CTTTATCTTT ATTTCTGGTC TTGATCTGGT TAAGGAATGC CAACCCGCTC TACAGCCATC |
| 541 | ATGAATGCCA TTTCCCCAAC AAGGCGATGC CCTCAGCAGG AATGCTGCCG TGGCTCCAGG |
| 601 | GGATCTTCTG CAATGTGAAC AATCCCTGTT TTCAAAGCCC CACCCCAGGA GAATCTCCTG |
| 661 | GAATTGTGTC AAACTATAAC AACTCCATCT TGGCAAGGGT ATATCGAGAT TTTCAAGAAC |
| 721 | TCCTCATGAA TGCACCAGAG AGCCAGCACC TTGGCCGTAT TTGGACAGAG CTACACATCT |
| 781 | TGTCCCAATT CATGGACACC CTCCGGACTC ACCCGGAGAG AATTGCAGGA AGAGGAATAC |
| 841 | GAATAAGGGA TATCTTGAAA GATGAAGAAA CACTGACACT ATTTCTCATT AAAAACATCG |
| 901 | GCCTGTCTGA CTCAGTGGTC TACCTTCTGA TCAACTCTCA AGTCCGTCCA GAGCAGTTCG |
| 961 | CTCATGGAGT CCCGGACCTG GCGCTGAAGG ACATCGCCTG CAGCGAGGCC CTCCTGGAGC |
| 1021 | GCTTCATCAT CTTCAGCCAG AGACGCGGGG CAAAGACGGT GCGCTATGCC CTGTGCTCCC |
| 1081 | TCTCCCAGGG CACCCTACAG TGGATAGAAG ACACTCTGTA TGCCAACGTG GACTTCTTCA |
| 1141 | AGCTCTTCCG TGTGCTTCCC ACACTCCTAG ACAGCCGTTC TCAAGGTATC AATCTGAGAT |
| 1201 | CTTGGGGAGG AATATTATCT GATATGTCAC CAAGAATTCA AGAGTTTATC CATCGGCCGA |
| 1261 | GTATGCAGGA CTTGCTGTGG GTGACCAGGC CCCTCATGCA GAATGGTGGT CCAGAGACCT |
| 1321 | TTACAAAGCT GATGGGCATC CTGTCTGACC TCCTGTGTGG CTACCCCGAG GGAGGTGGCT |
| 1381 | CTCGGGTGCT CTCCTTCAAC TGGTATGAAG ACAATAACTA TAAGGCCTTT CTGGGGATTG |
| 1441 | ACTCCACAAG GAAGGATCCT ATCTATTCTT ATGACAGAAG AACAACATCC TTTTGTAATG |
| 1501 | CATTGATCCA GAGCCTGGAG TCAAATCCTT TAACCAAAAT CGCTTGGAGG GCGGCAAAGC |
| 1561 | CTTTGCTGAT GGGAAAAATC CTGTACACTC CTGATTCACC TGCAGCACGA AGGATACTGA |
| 1621 | AGAATGCCAA CTCAACTTTT GAAGAACTGG AACACGTTAG GAAGTTGGTC AAAGCCTGGG |
| 1681 | AAGAAGTAGG GCCCCAGATC TGGTACTTCT TTGACAACAG CACACAGATG AACATGATCA |
| 1741 | GAGATACCCT GGGGAACCCA ACAGTAAAAG ACTTTTTGAA TAGGCAGCTT GGTGAAGAAG |
| 1801 | GTATTACTGC TGAAGCCATC CTAAACTTCC TCTACAAGGG CCCTCGGGAA AGCCAGGCTG |
| 1861 | ACGACATGGC CAACTTCGAC TGGAGGGACA TATTTAACAT CACTGATCGC ACCCTCCGCC |
| 1921 | TTGTCAATCA ATACCTGGAG TGCTTGGTCC TGGATAAGTT TGAAAGCTAC AATGATGAAA |
| 1981 | CTCAGCTCAC CCAACGTGCC CTCTCTCTAC TGGAGGAAAA CATGTTCTGG GCCGGAGTGG |
| 2041 | TATTCCCTGA CATGTATCCC TGGACCAGCT CTCTACCACC CCACGTGAAG TATAAGATCC |
| 2101 | GAATGGACAT AGACGTGGTG GAGAAAACCA ATAAGATTAA AGACAGGTAT TGGGATTCTG |
| 2161 | GTCCCAGAGC TGATCCCGTG GAAGATTTCC GGTACATCTG GGGCGGGTTT GCCTATCTGC |
| 2221 | AGGACATGGT TGAACAGGGG ATCACAAGGA GCCAGGTGCA GGCGGAGGCT CCAGTTGGAA |
| 2281 | TCTACCTCCA GCAGATGCCC TACCCCTGCT TCGTGGACGA TTCTTTCATG ATCATCCTGA |
| 2341 | ACCGCTGTTT CCCTATCTTC ATGGTGCTGG CATGGATCTA CTCTGTCTCC ATGACTGTGA |
| 2401 | AGAGCATCGT CTTGGAGAAG GAGTTGCGAC TGAAGGAGAC CTTGAAAAAT CAGGGTGTCT |
| 2461 | CCAATGCAGT GATTTGGTGT ACCTGGTTCC TGGACAGCTT CTCCATCATG TCGATGAGCA |
| 2521 | TCTTCCTCCT GACGATATTC ATCATGCATG GAAGAATCCT ACATTACAGC GACCCATTCA |
| 2581 | TCCTCTTCCT GTTCTTGTTG GCTTTCTCCA CTGCCACCAT CATGCTGTGC TTTCTGCTCA |
| 2641 | GCACCTTCTT CTCCAAGGCC AGTCTGGCAG CAGCCTGTAG TGGTGTCATC TATTTCACCC |
| 2701 | TCTACCTGCC ACACATCCTG TGCTTCGCCT GGCAGGACCG CATGACCGCT GAGCTGAAGA |
| 2761 | AGGCTGTGAG CTTACTGTCT CCGGTGGCAT TTGGATTTGG CACTGAGTAC CTGGTTCGCT |
| 2821 | TTGAAGAGCA AGGCCTGGGG CTGCAGTGGA GCAACATCGG GAACAGTCCC ACGGAAGGGG |
| 2881 | ACGAATTCAG CTTCCTGCTG TCCATGCAGA TGATGCTCCT TGATGCTGCT GTCTATGGCT |
| 2941 | TACTCGCTTG GTACCTTGAT CAGGTGTTTC CAGGAGACTA TGGAACCCCA CTTCCTTGGT |
| 3001 | ACTTTCTTCT ACAAGAGTCG TATTGGCTTG GCGGTGAAGG GTGTTCAACC AGAGAAGAAA |
| 3061 | GAGCCCTGGA AAAGACCGAG CCCCTAACAG AGGAAACGGA GGATCCAGAG CACCCAGAAG |
| 3121 | GAATACACGA CTCCTTCTTT GAACGTGAGC ATCCAGGGTG GGTTCCTGGG GTATGCGTGA |
| 3181 | AGAATCTGGT AAAGATTTTT GAGCCCTGTG GCCGGCCAGC TGTGGACCGT CTGAACATCA |
| 3241 | CCTTCTACGA GAACCAGATC ACCGCATTCC TGGGCCACAA TGGAGCTGGG AAAACCACCA |
| 3301 | CCTTGTCCAT CCTGACGGGT CTGTTGCCAC CAACCTCTGG GACTGTGCTC GTTGGGGGAA |
| 3361 | GGGACATTGA AACCAGCCTG GATGCAGTCC GGCAGAGCCT TGGCATGTGT CCACAGCACA |
| 3421 | ACATCCTGTT CCACCACCTC ACGGTGGCTG AGCACATGCT GTTCTATGCC CAGCTGAAAG |
| 3481 | GAAAGTCCCA GGAGGAGGCC CAGCTGGAGA TGGAAGCCAT GTTGGAGGAC ACAGGCCTCC |
| 3541 | ACCACAAGCG GAATGAAGAG GCTCAGGACC TATCAGGTGG CATGCAGAGA AAGCTGTCGG |
| 3601 | TTGCCATTGC CTTTGTGGGA GATGCCAAGG TGGTGATTCT GGACGAACCC ACCTCTGGGG |
| 3661 | TGGACCCTTA CTCGAGACGC TCAATCTGGG ATCTGCTCCT GAAGTATCGC TCAGGCAGAA |
| 3721 | CCATCATCAT GTCCACTCAC CACATGGACG AGGCCGACCT CCTTGGGGAC CGCATTGCCA |
| 3781 | TCATTGCCCA GGGAAGGCTC TACTGCTCAG GCACCCCACT CTTCCTGAAG AACTGCTTTG |
| 3841 | GCACAGGCTT GTACTTAACC TTGGTGCGCA AGATGAAAAA CATCCAGAGC CAAAGGAAAG |
| 3901 | GCAGTGAGGG GACCTGCAGC TGCTCGTCTA AGGGTTTCTC CACCACGTGT CCAGCCCACG |
| 3961 | TCGATGACCT AACTCCAGAA CAAGTCCTGG ATGGGGATGT AAATGAGCTG ATGGATGTAG |
| 4021 | TTCTCCACCA TGTTCCAGAG GCAAAGCTGG TGGAGTGCAT TGGTCAAGAA CTTATCTTCC |
| 4081 | TTCTTCC |

SEQ ID NO: 10

| | |
|---|---|
| 1 | ACATCCAGAG CCAAAGGAAA GGCAGTGAGG GGACCTGCAG CTGCTCGTCT AAGGGTTTCT |
| 61 | CCACCACGTG TCCAGCCCAC GTCGATGACC TAACTCCAGA ACAAGTCCTG GATGGGGATG |
| 121 | TAAATGAGCT GATGGATGTA GTTCTCCACC ATGTTCCAGA GGCAAAGCTG GTGGAGTGCA |
| 181 | TTGGTCAAGA ACTTATCTTC CTTCTTCCAA ATAAGAACTT CAAGCACAGA GCATATGCCA |
| 241 | GCCTTTTCAG AGAGCTGGAG GAGACGCTGG CTGACCTTGG TCTCAGCAGT TTTGGAATTT |
| 301 | CTGACACTCC CCTGGAAGAG ATTTTTCTGA AGGTCACGGA GGATTCTGAT TCAGGACCTC |
| 361 | TGTTTGCGGG TGGCGCTCAG CAGAAAAGAG AAAACGTCAA CCCCCGACAC CCCTGCTTGG |
| 421 | GTCCCAGAGA GAAGGCTGGA CAGACACCCC AGGACTCCAA TGTCTGCTCC CCAGGGGCGC |
| 481 | CGGCTGCTCA CCCAGAGGGC CAGCCTCCCC CAGAGCCAGA GTGCCCAGGC CCGCAGCTCA |
| 541 | ACACGGGGAC ACAGCTGGTC CTCCAGCATG TGCAGGCGCT GCTGGTCAAG AGATTCCAAC |
| 601 | ACACCATCCG CAGCCACAAG GACTTCCTGG CGCAGATCGT GCTCCCGGCT ACCTTTGTGT |
| 661 | TTTTGGCTCT GATGCTTTCT ATTGTTATCC CTCCTTTTGG CGAATACCCC GCTTTGACCC |
| 721 | TTCACCCCTG GATATATGGG CAGCAGTACA CCTTCTTCAG CATGGATGAA CCAGGCAGTG |
| 781 | AGCAGTTCAC GGTACTTGCA GACGTCCTCC TGAATAAGCC AGGCTTTGGC AACCGCTGCC |
| 841 | TGAAGGAAGG GTGGCTTCCG GAGTACCCCT GTGGCAACTC AACACCCTGG AAGACTCCTT |
| 901 | CTGTGTCCCC AAACATCACC CAGCTGTTCC AGAAGCAGAA ATGGACACAG GTCAACCCTT |
| 961 | CACCATCCTG CAGGTGCAGC ACCAGGGAGA AGCTCACCAT GCTGCCAGAG TGCCCCGAGG |
| 1021 | GTGCCGGGGG CCTCCCGCCC CCCCAGAGAA CACAGCGCAG CACGGAAATT CTACAAGACC |
| 1081 | TGACGGACAG GAACATCTCC GACTTCTTGG TAAAAACGTA TCCTGCTCTT ATAAGAAGCA |
| 1141 | GCTTAAAGAG CAAATTCTGG GTCAATGAAC AGAGGTATGG AGGAATTTCC ATTGGAGGAA |
| 1201 | AGCTCCCAGT CGTCCCCATC ACGGGGGAAG CACTTGTTGG GTTTTTAAGC GACCTTGGCC |
| 1261 | GGATCATGAA TGTGAGCGGG GGCCCTATCA CTAGAGAGGC CTCTAAAGAA ATACCTGATT |
| 1321 | TCCTTAAACA TCTAGAAACT GAAGACAACA TTAAGGTGTG GTTTAATAAC AAAGGCTGGC |
| 1381 | ATGCCCTGGT CAGCTTTCTC AATGTGGCCC ACAACGCCAT CTTACGGGCC AGCCTGCCTA |
| 1441 | AGGACAGGAG CCCCGAGGAG TATGGAATCA CCGTCATTAG CCAACCCCTG AACCTGACCA |
| 1501 | AGGAGCAGCT CTCAGAGATT ACAGTGCTGA CCACTTCAGT GGATGCTGTG GTTGCCATCT |
| 1561 | GCGTGATTTT CTCCATGTCC TTCGTCCCAG CCAGCTTTGT CCTTTATTTG ATCCAGGAGC |
| 1621 | GGGTGAACAA ATCCAAGCAC CTCCAGTTTA TCAGTGGAGT GAGCCCCACC ACCTACTGGG |
| 1681 | TAACCAACTT CCTCTGGGAC ATCATGAATT ATTCCGTGAG TGCTGGGCTG GTGGTGGGCA |
| 1741 | TCTTCATCGG GTTTCAGAAG AAAGCCTACA CTTCTCCAGA AAACCTTCCT GCCCTTGTGG |
| 1801 | CACTGCTCCT GCTGTATGGA TGGGCGGTCA TTCCCATGAT GTACCCAGCA TCCTTCCTGT |
| 1861 | TTGATGTCCC CAGCACAGCC TATGTGGCTT TATCTTGTGC TAATCTGTTC ATCGGCATCA |
| 1921 | ACAGCAGTGC TATTACCTTC ATCTTGGAAT TATTTGAGAA TAACCGGACG CTGCTCAGGT |
| 1981 | TCAACGCCGT GCTGAGGAAG CTGCTCATTG TCTTCCCCCA CTTCTGCCTG GGCCGGGGCC |
| 2041 | TCATTGACCT TGCACTGAGC CAGGCTGTGA CAGATGTCTA TGCCCGGTTT GGTGAGGAGC |
| 2101 | ACTCTGCAAA TCCGTTCCAC TGGGACCTGA TTGGGAAGAA CCTGTTTGCC ATGGTGGTGG |
| 2161 | AAGGGGTGGT GTACTTCCTC CTGACCCTGC TGGTCCAGCG CCACTTCTTC CTCTCCCAAT |
| 2221 | GGATTGCCGA GCCCACTAAG GAGCCCATTG TTGATGAAGA TGATGATGTG GCTGAAGAAA |
| 2281 | GACAAAGAAT TATTACTGGT GGAAATAAAA CTGACATCTT AAGGCTACAT GAACTAACCA |
| 2341 | AGATTTATCC AGGCACCTCC AGCCCAGCAG TGGACAGGCT GTGTGTCGGA GTTCGCCCTG |
| 2401 | GAGAGTGCTT TGGCCTCCTG GGAGTGAATG GTGCCGGCAA AACAACCACA TTCAAGATGC |
| 2461 | TCACTGGGGA CACCACAGTG ACCTCAGGGG ATGCCACCGT AGCAGGCAAG AGTATTTTAA |
| 2521 | CCAATATTTC TGAAGTCCAT CAAAATATGG GCTACTGTCC TCAGTTTGAT GCAATCGATG |
| 2581 | AGCTGCTCAC AGGACGAGAA CATCTTTACC TTTATGCCCG GCTTCGAGGT GTACCAGCAG |
| 2641 | AAGAAATCGA AAAGGTTGCA AACTGGAGTA TTAAGAGCCT GGGCCTGACT GTCTACGCCG |
| 2701 | ACTGCCTGGC TGGCACGTAC AGTGGGGGCA ACAAGCGGAA ACTCTCCACA GCCATCGCAC |
| 2761 | TCATTGGCTG CCCACCGCTG GTGCTGCTGG ATGAGCCCAC CACAGGGATG GACCCCCAGG |
| 2821 | CACGCCGCAT GCTGTGGAAC GTCATCGTGA GCATCATCAG AGAAGGGAGG GCTGTGGTCC |
| 2881 | TCACATCCCA CAGCATGGAA GAATGTGAGG CACTGTGTAC CCGGCTGGCC ATCATGGTAA |
| 2941 | AGGGCGCCTT TCGATGTATG GGCACCATTC AGCATCTCAA GTCCAAATTT GGAGATGGCT |
| 3001 | ATATCGTCAC AATGAAGATC AAATCCCCGA AGGACGACCT GCTTCCTGAC CTGAACCCTG |
| 3061 | TGGAGCAGTT CTTCCAGGGG AACTTCCCAG GCAGTGTGCA GAGGGAGAGG CACTACAACA |
| 3121 | TGCTCCAGTT CCAGGTCTCC TCCTCCTCCC TGGCGAGGAT CTTCCAGCTC CTCCTCTCCC |
| 3181 | ACAAGGACAG CCTGCTCATC GAGGAGTACT CAGTCACACA GACCACACTG GACCAGGTGT |
| 3241 | TTGTAAATTT TGCTAAACAG CAGACTGAAA GTCATGACCT CCCTCTGCAC CCTCGAGCTG |
| 3301 | CTGGAGCCAG TCGACAAGCC CAGGACTGAA AGCTTATCGA TAATCAACCT CTGGATTACA |
| 3361 | AAATTTGTGA AAGATTGACT GGTATTCTTA ACTATGTTGC TCCTTTTACG CTATGTGGAT |
| 3421 | ACGCTGCTTT AATGCCTTTG TATCATGCTA TTGCTTCCCG TATGGCTTTC ATTTTCTCCT |
| 3481 | CCTTGTATAA ATCCTGGTTG CTGTCTCTTT ATGAGGAGTT GTGGCCCGTT GTCAGGCAAC |
| 3541 | GTGGCGTGGT GTGCACTGTG TTTGCTGACG CAACCCCCAC TGGTTGGGGC ATTGCCACCA |
| 3601 | CCTGTCAGCT CCTTTCCGGG ACTTTCGCTT TCCCCCTCCC TATTGCCACG GCGGAACTCA |
| 3661 | TCGCCGCCTG CCTTGCCCGC TGCTGGACAG GGGCTCGGCT GTTGGGCACT GACAATTCCG |
| 3721 | TGGTGTTGTC GGGGAAATCA TCGTCCTTTC CTTGGCTGCT CGCCTGTGTT GCCACCTGGA |
| 3781 | TTCTGCGCGG GACGTCCTTC TGCTACGTCC CTTCGGCCCT CAATCCAGCG GACCTTCCTT |
| 3841 | CCCGCGGCCT GCTGCCGGCT CTGCGGCCTC TTCCGCGTCT TCGCCTTCGC CCTCAGACGA |
| 3901 | GTCGGATCTC CCTTTGGGCC GCCTCCCCGC ATGCCGCTGA TCAGCCTCGA CTGTGCCTTC |
| 3961 | TAGTTGCCAG CCATCTGTTG TTTGCCCCTC CCCCGTGCCT TCCTTGACCC TGGAAGGTGC |
| 4021 | CACTCCCACT GTCCTTTCCT AATAAAATGA GGAAATTGCA TCGCATTGTC TGAGTAGGTG |
| 4081 | TCATTCTATT CTGGGGGGTG GGGTGGGGCA GGACAGCAAG GGGGAGGATT GGGAAGACAA |
| 4141 | TAGCAGGCAT GCTGGGGATG CGGTGGGCTC TATGGCTTCT GAGGCGGAAA GAACCAGCTG |
| 4201 | GGG |

SEQ ID NO: 11

| | |
|---|---|
| 1 | ATGGGCTTCG TGAGACAGAT ACAGCTTTTG CTCTGGAAGA ACTGGACCCT GCGGAAAAGG |
| 61 | CAAAAGATTC GCTTTGTGGT GGAACTCGTG TGGCCTTTAT CTTTATTTCT GGTCTTGATC |
| 121 | TGGTTAAGGA ATGCCAACCC GCTCTACAGC CATCATGAAT GCCATTTCCC CAACAAGGCG |
| 181 | ATGCCCTCAG CAGGAATGCT GCCGTGGCTC CAGGGGATCT TCTGCAATGT GAACAATCCC |
| 241 | TGTTTTCAAA GCCCCACCCC AGGAGAATCT CCTGGAATTG TGTCAAACTA TAACAACTCC |
| 301 | ATCTTGGCAA GGGTATATCG AGATTTTCAA GAACTCCTCA TGAATGCACC AGAGAGCCAG |
| 361 | CACCTTGGCC GTATTTGGAC AGAGCTACAC ATCTTGTCCC AATTCATGGA CACCCTCCGG |
| 421 | ACTCACCCGG AGAGAATTGC AGGAAGAGGA ATACGAATAA GGGATATCTT GAAAGATGAA |
| 481 | GAAACACTGA CACTATTTCT CATTAAAAAC ATCGGCCTGT CTGACTCAGT GGTCTACCTT |
| 541 | CTGATCAACT CTCAAGTCCG TCCAGAGCAG TTCGCTCATG GAGTCCCGGA CCTGGCGCTG |
| 601 | AAGGACATCG CCTGCAGCGA GGCCCTCCTG GAGCGCTTCA TCATCTTCAG CCAGAGACGC |
| 661 | GGGGCAAAGA CGGTGCGCTA TGCCCTGTGC TCCCTCTCCC AGGGCACCCT ACAGTGGATA |
| 721 | GAAGACACTC TGTATGCCAA CGTGGACTTC TTCAAGCTCT TCCGTGTGCT TCCCACACTC |
| 781 | CTAGACAGCC GTTCTCAAGG TATCAATCTG AGATCTTGGG GAGGAATATT ATCTGATATG |
| 841 | TCACCAAGAA TTCAAGAGTT TATCCATCGG CCGAGTATGC AGGACTTGCT GTGGGTGACC |
| 901 | AGGCCCCTCA TGCAGAATGG TGGTCCAGAG ACCTTTACAA AGCTGATGGG CATCCTGTCT |
| 961 | GACCTCCTGT GTGGCTACCC CGAGGGAGGT GGCTCTCGGG TGCTCTCCTT CAACTGGTAT |
| 1021 | GAAGACAATA ACTATAAGGC CTTTCTGGGG ATTGACTCCA CAAGGAAGGA TCCTATCTAT |
| 1081 | TCTTATGACA GAAGAACAAC ATCCTTTTGT AATGCATTGA TCCAGAGCCT GGAGTCAAAT |
| 1141 | CCTTTAACCA AAATCGCTTG GAGGGCGGCA AAGCCTTTGC TGATGGGAAA AATCCTGTAC |
| 1201 | ACTCCTGATT CACCTGCAGC ACGAAGGATA CTGAAGAATG CCAACTCAAC TTTTGAAGAA |
| 1261 | CTGGAACACG TTAGGAAGTT GGTCAAAGCC TGGGAAGAAG TAGGGCCCCA GATCTGGTAC |
| 1321 | TTCTTTGACA ACAGCACACA GATGAACATG ATCAGAGATA CCCTGGGGAA CCCAACAGTA |
| 1381 | AAAGACTTTT TGAATAGGCA GCTTGGTGAA GAAGGTATTA CTGCTGAAGC CATCCTAAAC |
| 1441 | TTCCTCTACA AGGGCCCTCG GGAAAGCCAG GCTGACGACA TGGCCAACTT CGACTGGAGG |
| 1501 | GACATATTTA ACATCACTGA TCGCACCCTC CGCCTGGTCA ATCAATACCT GGAGTGCTTG |
| 1561 | GTCCTGGATA AGTTTGAAAG CTACAATGAT GAAACTCAGC TCACCCAACG TGCCCTCTCT |
| 1621 | CTACTGGAGG AAAACATGTT CTGGGCCGGA GTGGTATTCC CTGACATGTA TCCCTGGACC |
| 1681 | AGCTCTCTAC CACCCCACGT GAAGTATAAG ATCCGAATGG ACATAGACGT GGTGGAGAAA |
| 1741 | ACCAATAAGA TTAAAGACAG GTATTGGGAT TCTGGTCCCA GAGCTGATCC CGTGGAAGAT |
| 1801 | TTCCGGTACA TCTGGGGCGG GTTTGCCTAT CTGCAGGACA TGGTTGAACA GGGGATCACA |
| 1861 | AGGAGCCAGG TGCAGGCGGA GGCTCCAGTT GGAATCTACC TCCAGCAGAT GCCCTACCCC |
| 1921 | TGCTTCGTGG ACGATTCTTT CATGATCATC CTGAACCGCT GTTTCCCTAT CTTCATGGTG |
| 1981 | CTGGCATGGA TCTACTCTGT CTCCATGACT GTGAAGAGCA TCGTCTTGGA GAAGGAGTTG |
| 2041 | CGACTGAAGG AGACCTTGAA AAATCAGGGT GTCTCCAATG CAGTGATTTG GTGTACCTGG |
| 2101 | TTCCTGGACA GCTTCTCCAT CATGTCGATG AGCATCTTCC TCCTGACGAT ATTCATCATG |
| 2161 | CATGGAAGAA TCCTACATTA CAGCGACCCA TTCATCCTCT TCCTGTTCTT GTTGGCTTTC |
| 2221 | TCCACTGCCA CCATCATGCT GTGCTTTCTG CTCAGCACCT TCTTCTCCAA GGCCAGTCTG |
| 2281 | GCAGCAGCCT GTAGTGGTGT CATCTATTTC ACCCTCTACC TGCCACACAT CCTGTGCTTC |
| 2341 | GCCTGGCAGG ACCGCATGAC CGCTGAGCTG AAGAAGGCTG TGAGCTTACT GTCTCCGGTG |
| 2401 | GCATTTGGAT TTGGCACTGA GTACCTGGTT CGCTTTGAAG AGCAAGGCCT GGGGCTGCAG |
| 2461 | TGGAGCAACA TCGGGAACAG TCCCACGGAA GGGGACGAAT TCAGCTTCCT GCTGTCCATG |
| 2521 | CAGATGATGC TCCTTGATGC TGCTGTCTAT GGCTTACTCG CTTGGTACCT TGATCAGGTG |
| 2581 | TTTCCAGGAG ACTATGGAAC CCCACTTCCT TGGTACTTTC TTCTACAAGA GTCGTATTGG |
| 2641 | CTTGGCGGTG AAGGGTGTTC AACCAGAGAA GAAAGAGCCC TGGAAAAGAC CGAGCCCCTA |
| 2701 | ACAGAGGAAA CGGAGGATCC AGAGCACCCA GAAGGAATAC ACGACTCCTT CTTTGAACGT |
| 2761 | GAGCATCCAG GGTGGGTTCC TGGGGTATGC GTGAAGAATC TGGTAAAGAT TTTTGAGCCC |
| 2821 | TGTGGCCGGC CAGCTGTGGA CCGTCTGAAC ATCACCTTCT ACGAGAACCA GATCACCGCA |
| 2881 | TTCCTGGGCC ACAATGGAGC TGGGAAAACC ACCACCTTGT CCATCCTGAC GGGTCTGTTG |
| 2941 | CCACCAACCT CTGGGACTGT GCTCGTTGGG GGAAGGGACA TTGAAACCAG CCTGGATGCA |
| 3001 | GTCCGGCAGA GCCTTGGCAT GTGTCCACAG CACAACATCC TGTTCCACCA CCTCACGGTG |
| 3061 | GCTGAGCACA TGCTGTTCTA TGCCCAGCTG AAAGGAAAGT CCCAGGAGGA GGCCCAGCTG |
| 3121 | GAGATGGAAG CCATGTTGGA GGACACAGGC CTCCACCACA AGCGGAATGA AGAGGCTCAG |
| 3181 | GACCTATCAG GTGGCATGCA GAGAAAGCTG TCGGTTGCCA TTGCCTTTGT GGGAGATGCC |
| 3241 | AAGGTGGTGA TTCTGGACGA ACCCACCTCT GGGGTGGACC CTTACTCGAG ACGCTCAATC |
| 3301 | TGGGATCTGC TCCTGAAGTA TCGCTCAGGC AGAACCATCA TCATGTCCAC TCACCACATG |
| 3361 | GACGAGGCCG ACCTCCTTGG GGACCGCATT GCCATCATTG CCCAGGGAAG GCTCTACTGC |
| 3421 | TCAGGCACCC CACTCTTCCT GAAGAACTGC TTTGGCACAG GCTTGTACTT AACCTTGGTG |
| 3481 | CGCAAGATGA AAAACATCCA GAGCCAAAGG AAAGGCAGTG AGGGGACCTG CAGCTGCTCG |
| 3541 | TCTAAGGGTT TCTCCACCAC GTGTCCAGCC CACGTCGATG ACCTAACTCC AGAACAAGTC |
| 3601 | CTGGATGGGG ATGTAAATGA GCTGATGGAT GTAGTTCTCC ACCATGTTCC AGAGGCAAAG |
| 3661 | CTGGTGGAGT GCATTGGTCA AGAACTTATC TTCCTTCTTC CAAATAAGAA CTTCAAGCAC |
| 3721 | AGAGCATATG CCAGCCTTTT CAGAGAGCTG GAGGAGACGC TGGCTGACCT TGGTCTCAGC |
| 3781 | AGTTTTGGAA TTTCTGACAC TCCCCTGGAA GAGATTTTTC TGAAGGTCAC GGAGGATTCT |
| 3841 | GATTCAGGAC CTCTGTTTGC GGGTGGCGCT CAGCAGAAAA GAGAAAACGT CAACCCCCGA |
| 3901 | CACCCCTGCT TGGGTCCCAG AGAGAAGGCT GGACAGACAC CCCAGGACTC CAATGTCTGC |
| 3961 | TCCCCAGGGG CGCCGGCTGC TCACCCAGAG GGCCAGCCTC CCCCAGAGCC AGAGTGCCCA |
| 4021 | GGCCCGCAGC TCAACACGGG GACACAGCTG GTCCTCCAGC ATGTGCAGGC GCTGCTGGTC |
| 4081 | AAGAGATTCC AACACACCAT CCGCAGCCAC AAGGACTTCC TGGCGCAGAT CGTGCTCCCG |
| 4141 | GCTACCTTTG TGTTTTTGGC TCTGATGCTT TCTATTGTTA TCCCTCCTTT TGGCGAATAC |
| 4201 | CCCGCTTTGA CCCTTCACCC CTGGATATAT GGGCAGCAGT ACACCTTCTT CAGCATGGAT |
| 4261 | GAACCAGGCA GTGAGCAGTT CACGGTACTT GCAGACGTCC TCCTGAATAA GCCAGGCTTT |
| 4321 | GGCAACCGCT GCCTGAAGGA AGGGTGGCTT CCGGAGTACC CCTGTGGCAA CTCAACACCC |
| 4381 | TGGAAGACTC CTTCTGTGTC CCCAAACATC ACCCAGCTGT TCCAGAAGCA GAAATGGACA |
| 4441 | CAGGTCAACC CTTCACCATC CTGCAGGTGC AGCACCAGGG AGAAGCTCAC CATGCTGCCA |
| 4501 | GAGTGCCCCG AGGGTGCCGG GGGCCTCCCG CCCCCCCAGA GAACACAGCG CAGCACGGAA |
| 4561 | ATTCTACAAG ACCTGACGGA CAGGAACATC TCCGACTTCT TGGTAAAAAC GTATCCTGCT |
| 4621 | CTTATAAGAA GCAGCTTAAA GAGCAAATTC TGGGTCAATG AACAGAGGTA TGGAGGAATT |
| 4681 | TCCATTGGAG GAAAGCTCCC AGTCGTCCCC ATCACGGGGG AAGCACTTGT TGGGTTTTTA |
| 4741 | AGCGACCTTG GCCGGATCAT GAATGTGAGC GGGGGCCCTA TCACTAGAGA GGCCTCTAAA |
| 4801 | GAAATACCTG ATTTCCTTAA ACATCTAGAA ACTGAAGACA ACATTAAGGT GTGGTTTAAT |
| 4861 | AACAAAGGCT GGCATGCCCT GGTCAGCTTT CTCAATGTGG CCCACAACGC CATCTTACGG |
| 4921 | GCCAGCCTGC CTAAGGACAG GAGCCCCGAG GAGTATGGAA TCACCGTCAT TAGCCAACCC |
| 4981 | CTGAACCTGA CCAAGGAGCA GCTCTCAGAG ATTACAGTGC TGACCACTTC AGTGGATGCT |
| 5041 | GTGGTTGCCA TCTGCGTGAT TTTCTCCATG TCCTTCGTCC CAGCCAGCTT TGTCCTTTAT |
| 5101 | TTGATCCAGG AGCGGGTGAA CAAATCCAAG CACCTCCAGT TTATCAGTGG AGTGAGCCCC |
| 5161 | ACCACCTACT GGGTGACCAA CTTCCTCTGG GACATCATGA ATTATTCCGT GAGTGCTGGG |
| 5221 | CTGGTGGTGG GCATCTTCAT CGGGTTTCAG AAGAAAGCCT ACACTTCTCC AGAAAACCTT |
| 5281 | CCTGCCCTTG TGGCACTGCT CCTGCTGTAT GGATGGGCGG TCATTCCCAT GATGTACCCA |
| 5341 | GCATCCTTCC TGTTTGATGT CCCCAGCACA GCCTATGTGG CTTTATCTTG TGCTAATCTG |
| 5401 | TTCATCGGCA TCAACAGCAG TGCTATTACC TTCATCTTGG AATTATTTGA GAATAACCGG |
| 5461 | ACGCTGCTCA GGTTCAACGC CGTGCTGAGG AAGCTGCTCA TTGTCTTCCC CCACTTCTGC |
| 5521 | CTGGGCCGGG GCCTCATTGA CCTTGCACTG AGCCAGGCTG TGACAGATGT CTATGCCCGG |
| 5581 | TTTGGTGAGG AGCACTCTGC AAATCCGTTC CACTGGGACC TGATTGGGAA GAACCTGTTT |
| 5641 | GCCATGGTGG TGGAAGGGGT GGTGTACTTC CTCCTGACCC TGCTGGTCCA GCGCCACTTC |
| 5701 | TTCCTCTCCC AATGGATTGC CGAGCCCACT AAGGAGCCCA TTGTTGATGA AGATGATGAT |
| 5761 | GTGGCTGAAG AAAGACAAAG AATTATTACT GGTGGAAATA AAACTGACAT CTTAAGGCTA |
| 5821 | CATGAACTAA CCAAGATTTA TCCAGGCACC TCCAGCCCAG CAGTGGACAG GCTGTGTGTC |
| 5881 | GGAGTTCGCC CTGGAGAGTG CTTTGGCCTC CTGGGAGTGA ATGGTGCCGG CAAAACAACC |
| 5941 | ACATTCAAGA TGCTCACTGG GGACACCACA GTGACCTCAG GGGATGCCAC CGTAGCAGGC |
| 6001 | AAGAGTATTT TAACCAATAT TTCTGAAGTC CATCAAAATA TGGGCTACTG TCCTCAGTTT |
| 6061 | GATGCAATTG ATGAGCTGCT CACAGGACGA GAACATCTTT ACCTTTATGC CCGGCTTCGA |
| 6121 | GGTGTACCAG CAGAAGAAAT CGAAAAGGTT GCAAACTGGA GTATTAAGAG CCTGGGCCTG |
| 6181 | ACTGTCTACG CCGACTGCCT GGCTGGCACG TACAGTGGGG GCAACAAGCG GAAACTCTCC |
| 6241 | ACAGCCATCG CACTCATTGG CTGCCCACCG CTGGTGCTGC TGGATGAGCC CACCACAGGG |
| 6301 | ATGGACCCCC AGGCACGCCG CATGCTGTGG AACGTCATCG TGAGCATCAT CAGAGAAGGG |
| 6361 | AGGGCTGTGG TCCTCACATC CCACAGCATG GAAGAATGTG AGGCACTGTG TACCCGGCTG |
| 6421 | GCCATCATGG TAAAGGGCGC CTTTCGATGT ATGGGCACCA TTCAGCATCT CAAGTCCAAA |
| 6481 | TTTGGAGATG GCTATATCGT CACAATGAAG ATCAAATCCC CGAAGGACGA CCTGCTTCCT |
| 6541 | GACCTGAACC CTGTGGAGCA GTTCTTCCAG GGGAACTTCC CAGGCAGTGT GCAGAGGGAG |
| 6601 | AGGCACTACA ACATGCTCCA GTTCCAGGTC TCCTCCTCCT CCCTGGCGAG GATCTTCCAG |
| 6661 | CTCCTCCTCT CCCACAAGGA CAGCCTGCTC ATCGAGGAGT ACTCAGTCAC ACAGACCACA |
| 6721 | CTGGACCAGG TGTTTGTAAA TTTTGCTAAA CAGCAGACTG AAAGTCATGA CCTCCCTCTG |
| 6781 | CACCCTCGAG CTGCTGGAGC CAGTCGACAA GCCCAGGACT GA |

SEQ ID NO: 12

| | |
|---|---|
| 1 | MAADGYLPDW LEDNLSEGIR EWWALKPGAP KPKANQQKQD DGRGLVLPGY KYLGPFNGLD |
| 61 | KGEPVNAADA AALEHDKAYD QQLQAGDNPY LRYNHADAEF QERLQEDTSF GGNLGRAVFQ |
| 121 | AKKRVLEPLG LVEEGAKTAP GKKRPVEPSP QRSPDSSTGI GKKGQQPARK RLNFGQTGDS |
| 181 | ESVPDPQPLG EPPAAPSGVG PNTMAAGGGA PMADNNEGAD GVGSSSGNWH CDSTWLGDRV |
| 241 | ITTSTRTWAL PTYNNHLYKQ ISNGTSGGAT NDNTYFGYST PWGYFDFNRF HCHFSPRDWQ |
| 301 | RLINNNWGFR PKRLSFKLFN IQVKEVTQNE GTKTIANNLT STIQVFTDSE YQLPYVLGSA |
| 361 | HQGCLPPFPA DVFMIPQYGY LTLNNGSQAV GRSSFYCLEY FPSQMLRTGN NFQFTYTFED |
| 421 | VPFHSSYAHS QSLDRLMNPL IDQYLYYLSR TQTTGGTANT QTLGFSQGGP NTMANQAKNW |
| 481 | LPGPCYRQQR VSTTTGQNNN SNFAWTAGTK YHLNGRNSLA NPGIAMATHK DDEERFFPSN |
| 541 | GILIFGKQNA ARDNADYSDV MLTSEEEIKT TNPVATEEYG IVADNLQQQN TAPQIGTVNS |
| 601 | QGALPGMVWQ NRDVYLQGPI WAKIPHTDGN FHPSPLMGGF GLKHPPPQIL IKNTPVPADP |
| 661 | PTTFNQSKLN SFITQYSTGQ VSVEIEWELQ KENSKRWNPE IQYTSNYYKS TSVDFAVNTE |
| 721 | GVYSEPRPIG TRYLTRN |

SEQ ID NO: 13

| | |
|---|---|
| 1 | GTGCCGCAGG GGGACGGCTG CCTTCGGGGG GGACGGGGCA GGGCGGGGTT CGGCTTCTGG |
| 61 | CGTGTGACCG GCGGCTCTAG AGCCTCTGCT AACCATGTTC ATGCCTTCTT CTTTTTCCTA |
| 121 | CAG |

SEQ ID NO: 14

CTCCTGGGCA ACGTGCTGGT TATTGTGCTG TCTCATCATT TTGGCAAAGA ATT

SEQ ID NO: 15

| | |
|---|---|
| 1 | CCATTGACGT CAATAATGAC GTATGTTCCC ATAGTAACGC CAATAGGGAC TTTCCATTGA |
| 61 | CGTCAATGGG TGGAGTATTT ACGGTAAACT GCCCACTTGG CAGTACATCA AGTGTATCAT |
| 121 | ATGCCAAGTA CGCCCCCTAT TGACGTCAAT GACGGTAAAT GGCCCGCCTG GCATTATGCC |
| 181 | CAGTACATGA CCTTATGGGA CTTTCCTACT TGGCAGTACA TCTACGTATT AGTCA. |

SEQ ID NO: 16

| | |
|---|---|
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCGG GAGTCGCTGC GCGCTGCCTT |
| 301 | CGCCCCGTGC CCCGCTCCGC CGCCGCCTCG CGCCGCCCGC CCCGGCTCTG ACTGACCGCG |
| 361 | TTACTCCCAC AG |

SEQ ID NO: 24

| | |
|---|---|
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCG |

SEQ ID NO: 25

| | |
|---|---|
| 1 | CGCTGATCAG CCTCGACTGT GCCTTCTAGT TGCCAGCCAT CTGTTGTTTG CCCCTCCCCC |
| 61 | GTGCCTTCCT TGACCCTGGA AGGTGCCACT CCCACTGTCC TTTCCTAATA AAATGAGGAA |
| 121 | ATTGCATCGC ATTGTCTGAG TAGGTGTCAT TCTATTCTGG GGGGTGGGGT GGGGCAGGAC |
| 181 | AGCAAGGGGG AGGATTGGGA AGACAATAGC AGGCATGCTG GGGATGCGGT GGGCTCTATG |
| 241 | GCTTCTGAGG CGGAAAGAAC CAG |

SEQ ID NO: 40

| | |
|---|---|
| 1 | CTGCGCGCTC GCTCGCTCAC TGAGGCCGCC CGGGCAAAGC CCGGGCGTCG GGCGACCTTT |
| 61 | GGTCGCCCGG CCTCAGTGAG CGAGCGAGCG CGCAGAGAGG GAGTGGCCAA CTCCATCACT |
| 121 | AGGGGTTCCT GCGGCAATTC AGTCGATAAC TATAACGGTC CTAAGGTAGC GATTTAAATA |
| 181 | ACATCCAGAG CCAAAGGAAA GGCAGTGAGG GGACCTGCAG CTGCTCGTCT AAGGGTTTCT |
| 241 | CCACCACGTG TCCAGCCCAC GTCGATGACC TAACTCCAGA ACAAGTCCTG GATGGGGATG |
| 301 | TAAATGAGCT GATGGATGTA GTTCTCCACC ATGTTCCAGA GGCAAAGCTG GTGGAGTGCA |
| 361 | TTGGTCAAGA ACTTATCTTC CTTCTTCCAA ATAAGAACTT CAAGCACAGA GCATATGCCA |
| 421 | GCCTTTTCAG AGAGCTGGAG GAGACGCTGG CTGACCTTGG TCTCAGCAGT TTTGGAATTT |
| 481 | CTGACACTCC CCTGGAAGAG ATTTTTCTGA AGGTCACGGA GGATTCTGAT TCAGGACCTC |
| 541 | TGTTTGCGGG TGGCGCTCAG CAGAAAAGAG AAAACGTCAA CCCCCGACAC CCCTGCTTGG |
| 601 | GTCCCAGAGA GAAGGCTGGA CAGACACCCC AGGACTCCAA TGTCTGCTCC CCAGGGGCGC |
| 661 | CGGCTGCTCA CCCAGAGGGC CAGCCTCCCC CAGAGCCAGA GTGCCCAGGC CCGCAGCTCA |
| 721 | ACACGGGGAC ACAGCTGGTC CTCCAGCATG TGCAGGCGCT GCTGGTCAAG AGATTCCAAC |
| 781 | ACACCATCCG CAGCCACAAG GACTTCCTGG CGCAGATCGT GCTCCCGGCT ACCTTTGTGT |
| 841 | TTTTGGCTCT GATGCTTTCT ATTGTTATCC CTCCTTTTGG CGAATACCCC GCTTTGACCC |
| 901 | TTCACCCCTG GATATATGGG CAGCAGTACA CCTTCTTCAG CATGGATGAA CCAGGCAGTG |
| 961 | AGCAGTTCAC GGTACTTGCA GACGTCCTCC TGAATAAGCC AGGCTTTGGC AACCGCTGCC |
| 1021 | TGAAGGAAGG GTGGCTTCCG GAGTACCCCT GTGGCAACTC AACACCCTGG AAGACTCCTT |
| 1081 | CTGTGTCCCC AAACATCACC CAGCTGTTCC AGAAGCAGAA ATGGACACAG GTCAACCCTT |
| 1141 | CACCATCCTG CAGGTGCAGC ACCAGGGAGA AGCTCACCAT GCTGCCAGAG TGCCCCGAGG |
| 1201 | GTGCCGGGGG CCTCCCGCCC CCCCAGAGAA CACAGCGCAG CACGGAAATT CTACAAGACC |
| 1261 | TGACGGACAG GAACATCTCC GACTTCTTGG TAAAAACGTA TCCTGCTCTT ATAAGAAGCA |
| 1321 | GCTTAAAGAG CAAATTCTGG GTCAATGAAC AGAGGTATGG AGGAATTTCC ATTGGAGGAA |
| 1381 | AGCTCCCAGT CGTCCCCATC ACGGGGGAAG CACTTGTTGG GTTTTTAAGC GACCTTGGCC |
| 1441 | GGATCATGAA TGTGAGCGGG GGCCCTATCA CTAGAGAGGC CTCTAAAGAA ATACCTGATT |
| 1501 | TCCTTAAACA TCTAGAAACT GAAGACAACA TTAAGGTGTG GTTTAATAAC AAAGGCTGGC |
| 1561 | ATGCCCTGGT CAGCTTTCTC AATGTGGCCC ACAACGCCAT CTTACGGGCC AGCCTGCCTA |
| 1621 | AGGACAGGAG CCCCGAGGAG TATGGAATCA CCGTCATTAG CCAACCCCTG AACCTGACCA |
| 1681 | AGGAGCAGCT CTCAGAGATT ACAGTGCTGA CCACTTCAGT GGATGCTGTG GTTGCCATCT |
| 1741 | GCGTGATTTT CTCCATGTCC TTCGTCCCAG CCAGCTTTGT CCTTTATTTG ATCCAGGAGC |
| 1801 | GGGTGAACAA ATCCAAGCAC CTCCAGTTTA TCAGTGGAGT GAGCCCCACC ACCTACTGGG |
| 1861 | TAACCAACTT CCTCTGGGAC ATCATGAATT ATTCCGTGAG TGCTGGGCTG GTGGTGGGCA |
| 1921 | TCTTCATCGG GTTTCAGAAG AAAGCCTACA CTTCTCCAGA AAACCTTCCT GCCCTTGTGG |
| 1981 | CACTGCTCCT GCTGTATGGA TGGGCGGTCA TTCCCATGAT GTACCCAGCA TCCTTCCTGT |
| 2041 | TTGATGTCCC CAGCACAGCC TATGTGGCTT TATCTTGTGC TAATCTGTTC ATCGGCATCA |
| 2101 | ACAGCAGTGC TATTACCTTC ATCTTGGAAT TATTTGAGAA TAACCGGACG CTGCTCAGGT |
| 2161 | TCAACGCCGT GCTGAGGAAG CTGCTCATTG TCTTCCCCCA CTTCTGCCTG GGCCGGGGCC |
| 2221 | TCATTGACCT TGCACTGAGC CAGGCTGTGA CAGATGTCTA TGCCCGGTTT GGTGAGGAGC |
| 2281 | ACTCTGCAAA TCCGTTCCAC TGGGACCTGA TTGGGAAGAA CCTGTTTGCC ATGGTGGTGG |
| 2341 | AAGGGGTGGT GTACTTCCTC CTGACCCTGC TGGTCCAGCG CCACTTCTTC CTCTCCCAAT |
| 2401 | GGATTGCCGA GCCCACTAAG GAGCCCATTG TTGATGAAGA TGATGATGTG GCTGAAGAAA |
| 2461 | GACAAAGAAT TATTACTGGT GGAAATAAAA CTGACATCTT AAGGCTACAT GAACTAACCA |
| 2521 | AGATTTATCC AGGCACCTCC AGCCCAGCAG TGGACAGGCT GTGTGTCGGA GTTCGCCCTG |
| 2581 | GAGAGTGCTT TGGCCTCCTG GGAGTGAATG GTGCCGGCAA AACAACCACA TTCAAGATGC |
| 2641 | TCACTGGGGA CACCACAGTG ACCTCAGGGG ATGCCACCGT AGCAGGCAAG AGTATTTTAA |
| 2701 | CCAATATTTC TGAAGTCCAT CAAAATATGG GCTACTGTCC TCAGTTTGAT GCAATCGATG |
| 2761 | AGCTGCTCAC AGGACGAGAA CATCTTTACC TTTATGCCCG GCTTCGAGGT GTACCAGCAG |
| 2821 | AAGAAATCGA AAAGGTTGCA AACTGGAGTA TTAAGAGCCT GGGCCTGACT GTCTACGCCG |
| 2881 | ACTGCCTGGC TGGCACGTAC AGTGGGGGCA ACAAGCGGAA ACTCTCCACA GCCATCGCAC |
| 2941 | TCATTGGCTG CCCACCGCTG GTGCTGCTGG ATGAGCCCAC CACAGGGATG GACCCCCAGG |
| 3001 | CACGCCGCAT GCTGTGGAAC GTCATCGTGA GCATCATCAG AGAAGGGAGG GCTGTGGTCC |
| 3061 | TCACATCCCA CAGCATGGAA GAATGTGAGG CACTGTGTAC CCGGCTGGCC ATCATGGTAA |
| 3121 | AGGGCGCCTT TCGATGTATG GGCACCATTC AGCATCTCAA GTCCAAATTT GGAGATGGCT |
| 3181 | ATATCGTCAC AATGAAGATC AAATCCCCGA AGGACGACCT GCTTCCTGAC CTGAACCCTG |
| 3241 | TGGAGCAGTT CTTCCAGGGG AACTTCCCAG GCAGTGTGCA GAGGGAGAGG CACTACAACA |
| 3301 | TGCTCCAGTT CCAGGTCTCC TCCTCCTCCC TGGCGAGGAT CTTCCAGCTC CTCCTCTCCC |
| 3361 | ACAAGGACAG CCTGCTCATC GAGGAGTACT CAGTCACACA GACCACACTG GACCAGGTGT |
| 3421 | TTGTAAATTT TGCTAAACAG CAGACTGAAA GTCATGACCT CCCTCTGCAC CCTCGAGCTG |
| 3481 | CTGGAGCCAG TCGACAAGCC CAGGACTGAA AGCTTATCGA TAATCAACCT CTGGATTACA |
| 3541 | AAATTTGTGA AAGATTGACT GGTATTCTTA ACTATGTTGC TCCTTTTACG CTATGTGGAT |
| 3601 | ACGCTGCTTT AATGCCTTTG TATCATGCTA TTGCTTCCCG TATGGCTTTC ATTTTCTCCT |
| 3661 | CCTTGTATAA ATCCTGGTTG CTGTCTCTTT ATGAGGAGTT GTGGCCCGTT GTCAGGCAAC |
| 3721 | GTGGCGTGGT GTGCACTGTG TTTGCTGACG CAACCCCCAC TGGTTGGGGC ATTGCCACCA |
| 3781 | CCTGTCAGCT CCTTTCCGGG ACTTTCGCTT TCCCCCTCCC TATTGCCACG GCGGAACTCA |
| 3841 | TCGCCGCCTG CCTTGCCCGC TGCTGGACAG GGGCTCGGCT GTTGGGCACT GACAATTCCG |
| 3901 | TGGTGTTGTC GGGGAAATCA TCGTCCTTTC CTTGGCTGCT CGCCTGTGTT GCCACCTGGA |
| 3961 | TTCTGCGCGG GACGTCCTTC TGCTACGTCC CTTCGGCCCT CAATCCAGCG GACCTTCCTT |
| 4021 | CCCGCGGCCT GCTGCCGGCT CTGCGGCCTC TTCCGCGTCT TCGCCTTCGC CCTCAGACGA |
| 4081 | GTCGGATCTC CCTTTGGGCC GCCTCCCCGC ATGCCGCTGA TCAGCCTCGA CTGTGCCTTC |
| 4141 | TAGTTGCCAG CCATCTGTTG TTTGCCCCTC CCCCGTGCCT TCCTTGACCC TGGAAGGTGC |
| 4201 | CACTCCCACT GTCCTTTCCT AATAAAATGA GGAAATTGCA TCGCATTGTC TGAGTAGGTG |
| 4261 | TCATTCTATT CTGGGGGGTG GGGTGGGGCA GGACAGCAAG GGGGAGGATT GGGAAGACAA |
| 4321 | TAGCAGGCAT GCTGGGGATG CGGTGGGCTC TATGGCTTCT GAGGCGGAAA GAACCAGCTG |
| 4381 | GGGATTTAAA TTAGGGATAA CAGGGTAATG GCGCGGGCCG CAGGAACCCC TAGTGATGGA |
| 4441 | GTTGGCCACT CCCTCTCTGC GCGCTCGCTC GCTCACTGAG GCCGGGCGAC CAAAGGTCGC |
| 4501 | CCGACGCCCG GGCGGCCTCA GTGAGCGAGC GAGCGCGCAG AGCTAGAATT AATTCCGTGT |
| 4561 | ATTCTATAGT GTCACCTAAA TCGTATGTGT ATGATACATA AGGTTATGTA TTAATTGTAG |
| 4621 | CCGCGTTCTA ACGACAATAT GTACAAGCCT AATTGTGTAG CATCTGGCTT AGCGGCCGCC |
| 4681 | TACCGTCAAA CAGTCAATCC CGTTCTACGC CATTTGACAC ATAACGCCCG GGATAACAGA |
| 4741 | GCTGAATTTG ACGGACTACG ATATTGCTTA TGTGCCACCA ATCAACAGTT AACGAACACG |
| 4801 | TGGCGGCGCG GAACGCCTCC GGCCAGGCCG CGCGCTTCGC ATATTTACTT CGAGCAGTGT |
| 4861 | AGGTGTGACA ACGTAGCATG CAGCCACATC CCTAGCTTGA ACCGGAGATA AAGGTCTACG |
| 4921 | CGCGCGACGT CCACATTCAC ACGGTTCAGA TTCCTGGTGC TACCCAAAAC AAAGTCCATA |
| 4981 | GGTTTTTCAT TGGGACTACG GCGCGAAGCT AAGTGGTTTC ACACCTACAA GGGAAACATG |
| 5041 | CCCAAACTAT GAGGACAACA TCGTCCGCAG AAACAATCGG CCGCGATAGG GGTTGCACGT |
| 5101 | TGTCAGATGA AAGAGCCACA CTCGGGGAGC AGTCCGCGGA CGCCACCTCG TGCAACTTCG |
| 5161 | GCTAACCATA TAATCTAAAA AAGTTGAGGT TTGCAGTTGT CGGGGCGAGA TCAAACCCAA |
| 5221 | GTATATAGTC CTGTCCGGAG CCTTAGTTCA CGTACTCGCG ACCCTTGAAA GCGCGTCAAG |
| 5281 | CTTATCGCTC ACTGACTAGC TCAATGTGTG GCAATCTAAG TAGGAGGTCT GTCGCAAGGC |
| 5341 | AAAAATGCTA ATTATTGGTA GCAAGCTTAG ATAAGGTGGA GGGATTGCAC AATTCAGAAG |
| 5401 | GCGTCTTCTC TGCTACACCC GAGCGGGGTG CTTTATCAAG GGGAAGCTTG ATGTCCCACG |
| 5461 | GGATGAACGA GAGCCTCCAT GGCATCTCAC GACCTACTTA ACTTCGGGGG ATGGGTAGAA |
| 5521 | GTTAGCTGAA CATACAAATG GGAATAGGAT TGTGCCCTCG GACGAGACTG AACGGATCGC |
| 5581 | AGTCAACCCG CGCAAAGTTT ACATATTAAT TCTTACGGCG TGTCAGAGAG GCAATGGCTT |
| 5641 | GACTTGTGGT GGATCACAGT TTGTGAGTAA CGGCAAGATG CGGTAAACAC TGTAATGCGA |
| 5701 | GCTTCATTGA CTCGGCTTAA AGTTCCTGGT ACCATAATGA ATACACGGTG GTTAGTTGTC |
| 5761 | AATTGCTTGT GCACCGCCGC ACCTTGCGGT CCTCGGTCCA GCCTGCGCAG GGTATAAATG |
| 5821 | AAGCACGTCC CACCCAGACT GTTCCATCGT ACCTCCAAAT ACGGATTCAA CCTGGCGTCT |
| 5881 | ATTTCCAGAT ATGGGCCCTA GGGGTGATAG ACTCCCAAGT CTAAGGACTA CCATGGGATA |
| 5941 | TGTTTCACGT ATCCAAAAAG TAACCATAAT ACTGCGTTTC CGTTCACCCA AGTGAGGATG |
| 6001 | TTGCCTTTGT ACTGGTTTCA TAGTCCTGCC GTACCAGGCG TCTTCCTTAG CCGGCGCTAC |
| 6061 | TTCCAGCCCG GAACTGTCTT GTTTCTCGAT GTGAGACCCT TGTCAGCCGC CCGCGGTGGT |
| 6121 | GCACGTAAAA GCCGATTGGA GTATTAAGTA TTTACAACTC CGAATCTTAA GAGCCCTGCT |
| 6181 | CTAGTTTGGA TTCATATATC AGCATAGGCT TCGCAACCTA GTGAATGAGC GGTACGAACT |
| 6241 | TTCGCGGAGT GCGAAAAGCG ACCGAGCAAT CGAGATACGT ACCGTTAGAT TCACGCTCCA |
| 6301 | GACAGCACTC TGAGTCTTTG ATTTATAACC ATCGAAGGAA TCGACTTCAC GTCCCTAGCG |
| 6361 | TGTTGAGTCA TCCGCAGAAG AGACGATGAG GGCTCGCCCC CCGAAATAGT TCTGCTTCAA |
| 6421 | ACTATAGGCT GCCCTACTTG GTCTCCGAGG TACTATGGGG TCCTCGACGG TTCGAGGCCC |
| 6481 | CCAACCCATG TTCAATCAGC TCGTATGTCT ACCCTCGAGC TAACACAGGA ACCAGCTGAG |
| 6541 | ACTTGCCTGG CGTCACTTGG GCACGTTCCA TATACATAAT GAAGTACGCC GCAGGGTCTC |
| 6601 | TCCGTTACCG AACTGTGCTC GACCTAAAGT CCGGTACCCA TCGGCGTCCT GTCACATTTG |
| 6661 | TGGCATTAGG TATGAACTAA CTCTGGGGGG CTTCTACGAC CATGGTAAAA GTTTTGTGCT |
| 6721 | GCCAGACAAC TGTTAATAAA CATGTCGCTG CGTAGAACGC CAAGAACCAG CTGGGATGAG |
| 6781 | TGCCTTATTT ACCCCGCGCG AGGTGGGTCT GAGTAGGTAG CATCGAGGTT TACGCCTAAG |
| 6841 | TTGGACCGCA AATATAGGCC CTTTGCCGGG ATCCCCACTA TCTGTGAATT GTGAAACCCG |
| 6901 | TTGGCACCCT GTACAAAGTG CATAGCTACA TCATTGGTAA CAAGACGTAA ACGGAGGTTC |
| 6961 | GCTCACTCCC ACTTCGGAAA GATAACCGGG GAACTAGGAG GGTATGGTGC GCGCATGGAA |
| 7021 | AGGGCCGGGA AGTAACTCTG GCCTTCACGG AACGATAAGT TACAATTTGG GAACAGTCGG |
| 7081 | AGAGCGCCAC TACGTGCTTT TTTGGCTTAC CTCATATCTC GTAGTTGGTG AGGGTTAAAA |
| 7141 | TTCGCGGGAG AAGATCCAGC CTAAGTATAT GGTTACATCG CGGCCGCCTG AAGCAGACCC |
| 7201 | TATCATCTCT CTCGTAAACT GCCGTCAGAG TCGGTTTGGT TGGACGAACC TTCTGAGTTT |
| 7261 | CTGGTAACGC CGTCCCGCAC CCGGAAATGG TCAGCGAACC AATCAGCAGG GTCATCGCTA |
| 7321 | GCCAGATCCT CTACGCCGGA CGCATCGTGG CCGGCATCAC CGGCGCCACA GGTGCGGTTG |
| 7381 | CTGGCGCCTA TATCGCCGAC ATCACCGATG GGGAAGATCG GGCTCGCCAC TTCGGGCTCA |
| 7441 | TGAGCGCTTG TTTCGGCGTG GGTATGGTGG CAGGCCGCCC TTAGAAAAAC TCATCGAGCA |
| 7501 | TCAAATGAAA CTGCAATTTA TTCATATCAG GATTATCAAT ACCATATTTT TGAAAAAGCC |
| 7561 | GTTTCTGTAA TGAAGGAGAA AACTCACCGA GGCAGTTCCA TAGGATGGCA AGATCCTGGT |
| 7621 | ATCGGTCTGC GATTCCGACT CGTCCAACAT CAATACAACC TATTAATTTC CCCTCGTCAA |
| 7681 | AAATAAGGTT ATCAAGTGAG AAATCACCAT GAGTGACGAC TGAATCCGGT GAGAATGGCA |
| 7741 | AAAGCTTATG CATTTCTTTC CAGACTTGTT CAACAGGCCA GCCATTACGC TCGTCATCAA |
| 7801 | AATCACTCGC ATCAACCAAA CCGTTATTCA TTCGTGATTG CGCCTGAGCG AGACGAAATA |
| 7861 | CGCGATCGCT GTTAAAAGGA CAATTACAAA CAGGAATCGA ATGCAACCGG CGCAGGAACA |
| 7921 | CTGCCAGCGC ATCAACAATA TTTTCACCTG AATCAGGATA TTCTTCTAAT ACCTGGAATG |
| 7981 | CTGTTTTCCC GGGGATCGCA GTGGTGAGTA ACCATGCATC ATCAGGAGTA CGGATAAAAT |
| 8041 | GCTTGATGGT CGGAAGAGGC ATAAATTCCG TCAGCCAGTT TAGTCTGACC ATCTCATCTG |
| 8101 | TAACATCATT GGCAACGCTA CCTTTGCCAT GTTTCAGAAA CAACTCTGGC GCATCGGGCT |
| 8161 | TCCCATACAA TCGATAGATT GTCGCACCTG ATTGCCCGAC ATTATCGCGA GCCCATTTAT |
| 8221 | ACCCATATAA ATCAGCATCC ATGTTGGAAT TTAATCGCGG CCTCGAGCAA GACGTTTCCC |
| 8281 | GTTGAATATG GCTCATAACA CCCCTTGTAT TACTGTTTAT GTAAGCAGAC AGTTTTATTG |
| 8341 | TTCATGATGA TATATTTTTA TCTTGTGCAA TGTAACATCA GAGATTTTGA GACACAACGT |
| 8401 | GGTTTGCAGG AGTCAGGCAA CTATGGATGA ACGAAATAGA CAGATCGCTG AGATAGGTGC |
| 8461 | CTCACTGATT AAGCATTGGT AACTGTCAGA CCAAGTTTAC TCATATATAC TTTAGATTGA |
| 8521 | TTTAAAACTT CATTTTTAAT TTAAAAGGAT CTAGGTGAAG ATCCTTTTTG ATAATCTCAT |
| 8581 | GACCAAAATC CCTTAACGTG AGTTTTCGTT CCACTGAGCG TCAGACCCCG TAGAAAAGAT |
| 8641 | CAAAGGATCT TCTTGAGATC CTTTTTTTCT GCGCGTAATC TGCTGCTTGC AAACAAAAAA |
| 8701 | ACCACCGCTA CCAGCGGTGG TTTGTTTGCC GGATCAAGAG CTACCAACTC TTTTTCCGAA |
| 8761 | GGTAACTGGC TTCAGCAGAG CGCAGATACC AAATACTGTT CTTCTAGTGT AGCCGTAGTT |
| 8821 | AGGCCACCAC TTCAAGAACT CTGTAGCACC GCCTACATAC CTCGCTCTGC TAATCCTGTT |
| 8881 | ACCAGTGGCT GCTGCCAGTG GCGATAAGTC GTGTCTTACC GGGTTGGACT CAAGACGATA |
| 8941 | GTTACCGGAT AAGGCGCAGC GGTCGGGCTG AACGGGGGGT TCGTGCACAC AGCCCAGCTT |
| 9001 | GGAGCGAACG ACCTACACCG AACTGAGATA CCTACAGCGT GAGCTATGAG AAAGCGCCAC |
| 9061 | GCTTCCCGAA GGGAGAAAGG CGGACAGGTA TCCGGTAAGC GGCAGGGTCG GAACAGGAGA |
| 9121 | GCGCACGAGG GAGCTTCCAG GGGGAAACGC CTGGTATCTT TATAGTCCTG TCGGGTTTCG |
| 9181 | CCACCTCTGA CTTGAGCGTC GATTTTTGTG ATGCTCGTCA GGGGGGCGGA GCCTATGGAA |
| 9241 | AAACGCCAGC AACGCGGCCT TTTTACGGTT CCTGGCCTTT TGCTGGCCTT TTGCTCACAT |
| 9301 | GTTCTTTCCT GCGTTATCCC CTGATTCTGT GGATAACCGT ATTACCGCCT TTGAGTGAGC |
| 9361 | TGATACCGCT CGCCGCAGCC GAACGACCGA GCGCAGCGAG TCAGTGAGCG AGGAAGCGGA |
| 9421 | AGAGCGCCCA ATACGCAAAC CGCCTCTCCC CGCGCGTTGG CCGATTCATT AATGCAGCTG |
| 9481 | TGGAATGTGT GTCAGTTAGG GTGTGGAAAG TCCCCAGGCT CCCCAGCAGG CAGAAGTATG |
| 9541 | CAAAGCATGC ATCTCAATTA GTCAGCAACC AGGTGTGGAA AGTCCCCAGG CTCCCCAGCA |
| 9601 | GGCAGAAGTA TGCAAAGCAT GCATCTCAAT TAGTCAGCAA CCATAGTCCC GCCCCTAACT |
| 9661 | CCGCCCATCC CGCCCCTAAC TCCGCCCAGT TCCGCCCATT CTCCGCCCCA TGGCTGACTA |
| 9721 | ATTTTTTTTA TTTATGCAGA GGCCGAGGCC GCCTCGGCCT CTGAGCTATT CCAGAAGTAG |
| 9781 | TGAGGAGGCT TTTTTGGAGG CCTAGGCTTT TGCAAAAAG |

SEQ ID NO: 46

| | |
|---|---|
| 1 | CTGCGCGCTC GCTCGCTCAC TGAGGCCGCC CGGGCAAAGC CCGGGCGTCG GGCGACCTTT |
| 61 | GGTCGCCCGG CCTCAGTGAG CGAGCGAGCG CGCAGAGAGG GAGTGGCCAA CTCCATCACT |
| 121 | AGGGGTTCCT GCGGCAATTC AGTCGATAAC TATAACGGTC CTAAGGTAGC GATTTAAATG |
| 181 | GTACCCATGG TCGAGGTGAG CCCCACGTTC TGCTTCACTC TCCCCATCTC CCCCCCCTCC |
| 241 | CCACCCCCAA TTTTGTATTT ATTTATTTTT TAATTATTTT GTGCAGCGAT GGGGGCGGGG |
| 301 | GGGGGGGGGG GGCGCGCGCC AGGCGGGGCG GGGCGGGGCG AGGGGCGGGG CGGGGCGAGG |
| 361 | CGGAGAGGTG CGGCGGCAGC CAATCAGAGC GGCGCGCTCC GAAAGTTTCC TTTTATGGCG |
| 421 | AGGCGGCGGC GGCGGCGGCC CTATAAAAAG CGAAGCGCGC GGCGGGCGTG CCGCAGGGGG |
| 481 | ACGGCTGCCT TCGGGGGGGA CGGGGCAGGG CGGGGTTCGG CTTCTGGCGT GTGACCGGCG |
| 541 | GCTCTAGAGC CTCTGCTAAC CATGTTCATG CCTTCTTCTT TTTCCTACAG CTCCTGGGCA |
| 601 | ACGTGCTGGT TATTGTGCTG TCTCATCATT TTGGCAAAGA ATTACCACCA TGGGCTTCGT |
| 661 | GAGACAGATA CAGCTTTTGC TCTGGAAGAA CTGGACCCTG CGGAAAAGGC AAAAGATTCG |
| 721 | CTTTGTGGTG GAACTCGTGT GGCCTTTATC TTTATTTCTG GTCTTGATCT GGTTAAGGAA |
| 781 | TGCCAACCCG CTCTACAGCC ATCATGAATG CCATTTCCCC AACAAGGCGA TGCCCTCAGC |
| 841 | AGGAATGCTG CCGTGGCTCC AGGGGATCTT CTGCAATGTG AACAATCCCT GTTTTCAAAG |
| 901 | CCCCACCCCA GGAGAATCTC CTGGAATTGT GTCAAACTAT AACAACTCCA TCTTGGCAAG |
| 961 | GGTATATCGA GATTTTCAAG AACTCCTCAT GAATGCACCA GAGAGCCAGC ACCTTGGCCG |
| 1021 | TATTTGGACA GAGCTACACA TCTTGTCCCA ATTCATGGAC ACCCTCCGGA CTCACCCGGA |
| 1081 | GAGAATTGCA GGAAGAGGAA TACGAATAAG GGATATCTTG AAAGATGAAG AAACACTGAC |
| 1141 | ACTATTTCTC ATTAAAAACA TCGGCCTGTC TGACTCAGTG GTCTACCTTC TGATCAACTC |
| 1201 | TCAAGTCCGT CCAGAGCAGT TCGCTCATGG AGTCCCGGAC CTGGCGCTGA AGGACATCGC |
| 1261 | CTGCAGCGAG GCCCTCCTGG AGCGCTTCAT CATCTTCAGC CAGAGACGCG GGGCAAAGAC |
| 1321 | GGTGCGCTAT GCCCTGTGCT CCCTCTCCCA GGGCACCCTA CAGTGGATAG AAGACACTCT |
| 1381 | GTATGCCAAC GTGGACTTCT TCAAGCTCTT CCGTGTGCTT CCCACACTCC TAGACAGCCG |
| 1441 | TTCTCAAGGT ATCAATCTGA GATCTTGGGG AGGAATATTA TCTGATATGT CACCAAGAAT |
| 1501 | TCAAGAGTTT ATCCATCGGC CGAGTATGCA GGACTTGCTG TGGGTGACCA GGCCCCTCAT |
| 1561 | GCAGAATGGT GGTCCAGAGA CCTTTACAAA GCTGATGGGC ATCCTGTCTG ACCTCCTGTG |
| 1621 | TGGCTACCCC GAGGGAGGTG GCTCTCGGGT GCTCTCCTTC AACTGGTATG AAGACAATAA |
| 1681 | CTATAAGGCC TTTCTGGGGA TTGACTCCAC AAGGAAGGAT CCTATCTATT CTTATGACAG |
| 1741 | AAGAACAACA TCCTTTTGTA ATGCATTGAT CCAGAGCCTG GAGTCAAATC CTTTAACCAA |
| 1801 | AATCGCTTGG AGGGCGGCAA AGCCTTTGCT GATGGGAAAA ATCCTGTACA CTCCTGATTC |
| 1861 | ACCTGCAGCA CGAAGGATAC TGAAGAATGC CAACTCAACT TTTGAAGAAC TGGAACACGT |
| 1921 | TAGGAAGTTG GTCAAAGCCT GGGAAGAAGT AGGGCCCCAG ATCTGGTACT TCTTTGACAA |
| 1981 | CAGCACACAG ATGAACATGA TCAGAGATAC CCTGGGGAAC CCAACAGTAA AAGACTTTTT |
| 2041 | GAATAGGCAG CTTGGTGAAG AAGGTATTAC TGCTGAAGCC ATCCTAAACT TCCTCTACAA |
| 2101 | GGGCCCTCGG GAAAGCCAGG CTGACGACAT GGCCAACTTC GACTGGAGGG ACATATTTAA |
| 2161 | CATCACTGAT CGCACCCTCC GCCTTGTCAA TCAATACCTG GAGTGCTTGG TCCTGGATAA |
| 2221 | GTTTGAAAGC TACAATGATG AAACTCAGCT CACCCAACGT GCCCTCTCTC TACTGGAGGA |
| 2281 | AAACATGTTC TGGGCCGGAG TGGTATTCCC TGACATGTAT CCCTGGACCA GCTCTCTACC |
| 2341 | ACCCCACGTG AAGTATAAGA TCCGAATGGA CATAGACGTG GTGGAGAAAA CCAATAAGAT |
| 2401 | TAAAGACAGG TATTGGGATT CTGGTCCCAG AGCTGATCCC GTGGAAGATT TCCGGTACAT |
| 2461 | CTGGGGCGGG TTTGCCTATC TGCAGGACAT GGTTGAACAG GGGATCACAA GGAGCCAGGT |
| 2521 | GCAGGCGGAG GCTCCAGTTG GAATCTACCT CCAGCAGATG CCCTACCCCT GCTTCGTGGA |
| 2581 | CGATTCTTTC ATGATCATCC TGAACCGCTG TTTCCCTATC TTCATGGTGC TGGCATGGAT |
| 2641 | CTACTCTGTC TCCATGACTG TGAAGAGCAT CGTCTTGGAG AAGGAGTTGC GACTGAAGGA |
| 2701 | GACCTTGAAA AATCAGGGTG TCTCCAATGC AGTGATTTGG TGTACCTGGT TCCTGGACAG |
| 2761 | CTTCTCCATC ATGTCGATGA GCATCTTCCT CCTGACGATA TTCATCATGC ATGGAAGAAT |
| 2821 | CCTACATTAC AGCGACCCAT TCATCCTCTT CCTGTTCTTG TTGGCTTTCT CCACTGCCAC |
| 2881 | CATCATGCTG TGCTTTCTGC TCAGCACCTT CTTCTCCAAG GCCAGTCTGG CAGCAGCCTG |
| 2941 | TAGTGGTGTC ATCTATTTCA CCCTCTACCT GCCACACATC CTGTGCTTCG CCTGGCAGGA |
| 3001 | CCGCATGACC GCTGAGCTGA AGAAGGCTGT GAGCTTACTG TCTCCGGTGG CATTTGGATT |
| 3061 | TGGCACTGAG TACCTGGTTC GCTTTGAAGA GCAAGGCCTG GGGCTGCAGT GGAGCAACAT |
| 3121 | CGGGAACAGT CCCACGGAAG GGGACGAATT CAGCTTCCTG CTGTCCATGC AGATGATGCT |
| 3181 | CCTTGATGCT GCTGTCTATG GCTTACTCGC TTGGTACCTT GATCAGGTGT TTCCAGGAGA |
| 3241 | CTATGGAACC CCACTTCCTT GGTACTTTCT TCTACAAGAG TCGTATTGGC TTGGCGGTGA |
| 3301 | AGGGTGTTCA ACCAGAGAAG AAAGAGCCCT GGAAAAGACC GAGCCCCTAA CAGAGGAAAC |
| 3361 | GGAGGATCCA GAGCACCCAG AAGGAATACA CGACTCCTTC TTTGAACGTG AGCATCCAGG |
| 3421 | GTGGGTTCCT GGGGTATGCG TGAAGAATCT GGTAAAGATT TTTGAGCCCT GTGGCCGGCC |
| 3481 | AGCTGTGGAC CGTCTGAACA TCACCTTCTA CGAGAACCAG ATCACCGCAT TCCTGGGCCA |
| 3541 | CAATGGAGCT GGGAAAACCA CCACCTTGTC CATCCTGACG GGTCTGTTGC CACCAACCTC |
| 3601 | TGGGACTGTG CTCGTTGGGG GAAGGGACAT TGAAACCAGC CTGGATGCAG TCCGGCAGAG |
| 3661 | CCTTGGCATG TGTCCACAGC ACAACATCCT GTTCCACCAC CTCACGGTGG CTGAGCACAT |
| 3721 | GCTGTTCTAT GCCCAGCTGA AAGGAAAGTC CCAGGAGGAG GCCCAGCTGG AGATGGAAGC |
| 3781 | CATGTTGGAG GACACAGGCC TCCACCACAA GCGGAATGAA GAGGCTCAGG ACCTATCAGG |
| 3841 | TGGCATGCAG AGAAAGCTGT CGGTTGCCAT TGCCTTTGTG GGAGATGCCA AGGTGGTGAT |
| 3901 | TCTGGACGAA CCCACCTCTG GGGTGGACCC TTACTCGAGA CGCTCAATCT GGGATCTGCT |
| 3961 | CCTGAAGTAT CGCTCAGGCA GAACCATCAT CATGTCCACT CACCACATGG ACGAGGCCGA |
| 4021 | CCTCCTTGGG GACCGCATTG CCATCATTGC CCAGGGAAGG CTCTACTGCT CAGGCACCCC |
| 4081 | ACTCTTCCTG AAGAACTGCT TTGGCACAGG CTTGTACTTA ACCTTGGTGC GCAAGATGAA |
| 4141 | AAACATCCAG AGCCAAAGGA AAGGCAGTGA GGGGACCTGC AGCTGCTCGT CTAAGGGTTT |
| 4201 | CTCCACCACG TGTCCAGCCC ACGTCGATGA CCTAACTCCA GAACAAGTCC TGGATGGGGA |
| 4261 | TGTAAATGAG CTGATGGATG TAGTTCTCCA CCATGTTCCA GAGGCAAAGC TGGTGGAGTG |
| 4321 | CATTGGTCAA GAACTTATCT TCCTTCTTCC ATTTAAATTA GGGATAACAG GGTGGTGGCG |
| 4381 | CGGGCCGCAG GAACCCCTAG TGATGGAGTT GGCCACTCCC TCTCTGCGCG CTCGCTCGCT |
| 4441 | CACTGAGGCC GGGCGACCAA AGGTCGCCCG ACGCCCGGGC GGCCTCAGTG AGCGAGCGAG |
| 4501 | CGCGCAGAGC TAGAATTAAT TCCGTGTATT CTATAGTGTC ACCTAAATCG TATGTGTATG |
| 4561 | ATACATAAGG TTATGTATTA ATTGTAGCCG CGTTCTAACG ACAATATGTA CAAGCCTAAT |
| 4621 | TGTGTAGCAT CTGGCTTAGC GGCCGCCTAC CGTCAAACAG TCAATCCCGT TCTACGCCAT |
| 4681 | TTGACACATA ACGCCCGGGA TAACAGAGCT GAATTTGACG GACTACGATA TTGCTTATGT |
| 4741 | GCCACCAATC AACAGTTAAC GAACACGTGG CGGCGCGGAA CGCCTCCGGC CAGGCCGCGC |
| 4801 | GCTTCGCATA TTTACTTCGA GCAGTGTAGG TGTGACAACG TAGCATGCAG CCACATCCCT |
| 4861 | AGCTTGAACC GGAGATAAAG GTCTACGCGC GCGACGTCCA CATTCACACG GTTCAGATTC |
| 4921 | CTGGTGCTAC CCAAAACAAA GTCCATAGGT TTTTCATTGG GACTACGGCG CGAAGCTAAG |
| 4981 | TGGTTTCACA CCTACAAGGG AAACATGCCC AAACTATGAG GACAACATCG TCCGCAGAAA |
| 5041 | CAATCGGCCG CGATAGGGGT TGCACGTTGT CAGATGAAAG AGCCACACTC GGGGAGCAGT |
| 5101 | CCGCGGACGC CACCTCGTGC AACTTCGGCT AACCATATAA TCTAAAAAAG TTGAGGTTTG |
| 5161 | CAGTTGTCGG GGCGAGATCA AACCCAAGTA TATAGTCCTG TCCGGAGCCT TAGTTCACGT |
| 5221 | ACTCGCGACC CTTGAAAGCG CGTCAAGCTT ATCGCTCACT GACTAGCTCA ATGTGTGGCA |
| 5281 | ATCTAAGTAG GAGGTCTGTC GCAAGGCAAA AATGCTAATT ATTGGTAGCA AGCTTAGATA |
| 5341 | AGGTGGAGGG ATTGCACAAT TCAGAAGGCG TCTTCTCTGC TACACCCGAG CGGGGTGCTT |
| 5401 | TATCAAGGGG AAGCTTGATG TCCCACGGGA TGAACGAGAG CCTCCATGGC ATCTCACGAC |
| 5461 | CTACTTAACT TCGGGGGATG GGTAGAAGTT AGCTGAACAT ACAAATGGGA ATAGGATTGT |
| 5521 | GCCCTCGGAC GAGACTGAAC GGATCGCAGT CAACCCGCGC AAAGTTTACA TATTAATTCT |
| 5581 | TACGGCGTGT CAGAGAGGCA ATGGCTTGAC TTGTGGTGGA TCACAGTTTG TGAGTAACGG |
| 5641 | CAAGATGCGG TAAACACTGT AATGCGAGCT TCATTGACTC GGCTTAAAGT TCCTGGTACC |
| 5701 | ATAATGAATA CACGGTGGTT AGTTGTCAAT TGCTTGTGCA CCGCCGCACC TTGCGGTCCT |
| 5761 | CGGTCCAGCC TGCGCAGGGT ATAAATGAAG CACGTCCCAC CCAGACTGTT CCATCGTACC |
| 5821 | TCCAAATACG GATTCAACCT GGCGTCTATT TCCAGATATG GGCCCTAGGG GTGATAGACT |
| 5881 | CCCAAGTCTA AGGACTACCA TGGGATATGT TTCACGTATC CAAAAAGTAA CCATAATACT |
| 5941 | GCGTTTCCGT TCACCCAAGT GAGGATGTTG CCTTTGTACT GGTTTCATAG TCCTGCCGTA |
| 6001 | CCAGGCGTCT TCCTTAGCCG GCGCTACTTC CAGCCCGGAA CTGTCTTGTT TCTCGATGTG |
| 6061 | AGACCCTTGT CAGCCGCCCG CGGTGGTGCA CGTAAAAGCC GATTGGAGTA TTAAGTATTT |
| 6121 | ACAACTCCGA ATCTTAAGAG CCCTGCTCTA GTTTGGATTC ATATATCAGC ATAGGCTTCG |
| 6181 | CAACCTAGTG AATGAGCGGT ACGAACTTTC GCGGAGTGCG AAAAGCGACC GAGCAATCGA |
| 6241 | GATACGTACC GTTAGATTCA CGCTCCAGAC AGCACTCTGA GTCTTTGATT TATAACCATC |
| 6301 | GAAGGAATCG ACTTCACGTC CCTAGCGTGT TGAGTCATCC GCAGAAGAGA CGATGAGGGC |
| 6361 | TCGCCCCCCG AAATAGTTCT GCTTCAAACT ATAGGCTGCC CTACTTGGTC TCCGAGGTAC |
| 6421 | TATGGGGTCC TCGACGGTTC GAGGCCCCCA ACCCATGTTC AATCAGCTCG TATGTCTACC |
| 6481 | CTCGAGCTAA CACAGGAACC AGCTGAGACT TGCCTGGCGT CACTTGGGCA CGTTCCATAT |
| 6541 | ACATAATGAA GTACGCCGCA GGGTCTCTCC GTTACCGAAC TGTGCTCGAC CTAAAGTCCG |
| 6601 | GTACCCATCG GCGTCCTGTC ACATTTGTGG CATTAGGTAT GAACTAACTC TGGGGGGCTT |
| 6661 | CTACGACCAT GGTAAAAGTT TTGTGCTGCC AGACAACTGT TAATAAACAT GTCGCTGCGT |
| 6721 | AGAACGCCAA GAACCAGCTG GGATGAGTGC CTTATTTACC CCGCGCGAGG TGGGTCTGAG |
| 6781 | TAGGTAGCAT CGAGGTTTAC GCCTAAGTTG GACCGCAAAT ATAGGCCCTT TGCCGGGATC |
| 6841 | CCCACTATCT GTGAATTGTG AAACCCGTTG GCACCCTGTA CAAAGTGCAT AGCTACATCA |
| 6901 | TTGGTAACAA GACGTAAACG GAGGTTCGCT CACTCCCACT TCGGAAAGAT AACCGGGGAA |
| 6961 | CTAGGAGGGT ATGGTGCGCG CATGGAAAGG GCCGGGAAGT AACTCTGGCC TTCACGGAAC |
| 7021 | GATAAGTTAC AATTTGGGAA CAGTCGGAGA GCGCCACTAC GTGCTTTTTT GGCTTACCTC |
| 7081 | ATATCTCGTA GTTGGTGAGG GTTAAAATTC GCGGGAGAAG ATCCAGCCTA AGTATATGGT |
| 7141 | TACATCGCGG CCGCCTGAAG CAGACCCTAT CATCTCTCTC GTAAACTGCC GTCAGAGTCG |
| 7201 | GTTTGGTTGG ACGAACCTTC TGAGTTTCTG GTAACGCCGT CCCGCACCCG GAAATGGTCA |
| 7261 | GCGAACCAAT CAGCAGGGTC ATCGCTAGCC AGATCCTCTA CGCCGGACGC ATCGTGGCCG |
| 7321 | GCATCACCGG CGCCACAGGT GCGGTTGCTG GCGCCTATAT CGCCGACATC ACCGATGGGG |
| 7381 | AAGATCGGGC TCGCCACTTC GGGCTCATGA GCGCTTGTTT CGGCGTGGGT ATGGTGGCAG |
| 7441 | GCCGCCCTTA GAAAAACTCA TCGAGCATCA AATGAAACTG CAATTTATTC ATATCAGGAT |
| 7501 | TATCAATACC ATATTTTTGA AAAAGCCGTT TCTGTAATGA AGGAGAAAAC TCACCGAGGC |
| 7561 | AGTTCCATAG GATGGCAAGA TCCTGGTATC GGTCTGCGAT TCCGACTCGT CCAACATCAA |
| 7621 | TACAACCTAT TAATTTCCCC TCGTCAAAAA TAAGGTTATC AAGTGAGAAA TCACCATGAG |
| 7681 | TGACGACTGA ATCCGGTGAG AATGGCAAAA GCTTATGCAT TTCTTTCCAG ACTTGTTCAA |
| 7741 | CAGGCCAGCC ATTACGCTCG TCATCAAAAT CACTCGCATC AACCAAACCG TTATTCATTC |
| 7801 | GTGATTGCGC CTGAGCGAGA CGAAATACGC GATCGCTGTT AAAAGGACAA TTACAAACAG |
| 7861 | GAATCGAATG CAACCGGCGC AGGAACACTG CCAGCGCATC AACAATATTT TCACCTGAAT |
| 7921 | CAGGATATTC TTCTAATACC TGGAATGCTG TTTTCCCGGG GATCGCAGTG GTGAGTAACC |
| 7981 | ATGCATCATC AGGAGTACGG ATAAAATGCT TGATGGTCGG AAGAGGCATA AATTCCGTCA |
| 8041 | GCCAGTTTAG TCTGACCATC TCATCTGTAA CATCATTGGC AACGCTACCT TTGCCATGTT |
| 8101 | TCAGAAACAA CTCTGGCGCA TCGGGCTTCC CATACAATCG ATAGATTGTC GCACCTGATT |
| 8161 | GCCCGACATT ATCGCGAGCC CATTTATACC CATATAAATC AGCATCCATG TTGGAATTTA |
| 8221 | ATCGCGGCCT CGAGCAAGAC GTTTCCCGTT GAATATGGCT CATAACACCC CTTGTATTAC |
| 8281 | TGTTTATGTA AGCAGACAGT TTTATTGTTC ATGATGATAT ATTTTTATCT TGTGCAATGT |
| 8341 | AACATCAGAG ATTTTGAGAC ACAACGTGGT TTGCAGGAGT CAGGCAACTA TGGATGAACG |
| 8401 | AAATAGACAG ATCGCTGAGA TAGGTGCCTC ACTGATTAAG CATTGGTAAC TGTCAGACCA |
| 8461 | AGTTTACTCA TATATACTTT AGATTGATTT AAAACTTCAT TTTTAATTTA AAAGGATCTA |
| 8521 | GGTGAAGATC CTTTTTGATA ATCTCATGAC CAAAATCCCT TAACGTGAGT TTTCGTTCCA |
| 8581 | CTGAGCGTCA GACCCCGTAG AAAAGATCAA AGGATCTTCT TGAGATCCTT TTTTTCTGCG |
| 8641 | CGTAATCTGC TGCTTGCAAA CAAAAAAACC ACCGCTACCA GCGGTGGTTT GTTTGCCGGA |
| 8701 | TCAAGAGCTA CCAACTCTTT TTCCGAAGGT AACTGGCTTC AGCAGAGCGC AGATACCAAA |
| 8761 | TACTGTTCTT CTAGTGTAGC CGTAGTTAGG CCACCACTTC AAGAACTCTG TAGCACCGCC |
| 8821 | TACATACCTC GCTCTGCTAA TCCTGTTACC AGTGGCTGCT GCCAGTGGCG ATAAGTCGTG |
| 8881 | TCTTACCGGG TTGGACTCAA GACGATAGTT ACCGGATAAG GCGCAGCGGT CGGGCTGAAC |
| 8941 | GGGGGGTTCG TGCACACAGC CCAGCTTGGA GCGAACGACC TACACCGAAC TGAGATACCT |
| 9001 | ACAGCGTGAG CTATGAGAAA GCGCCACGCT TCCCGAAGGG AGAAAGGCGG ACAGGTATCC |
| 9061 | GGTAAGCGGC AGGGTCGGAA CAGGAGAGCG CACGAGGGAG CTTCCAGGGG GAAACGCCTG |
| 9121 | GTATCTTTAT AGTCCTGTCG GGTTTCGCCA CCTCTGACTT GAGCGTCGAT TTTTGTGATG |
| 9181 | CTCGTCAGGG GGGCGGAGCC TATGGAAAAA CGCCAGCAAC GCGGCCTTTT TACGGTTCCT |
| 9241 | GGCCTTTTGC TGGCCTTTTG CTCACATGTT CTTTCCTGCG TTATCCCCTG ATTCTGTGGA |
| 9301 | TAACCGTATT ACCGCCTTTG AGTGAGCTGA TACCGCTCGC CGCAGCCGAA CGACCGAGCG |
| 9361 | CAGCGAGTCA GTGAGCGAGG AAGCGGAAGA GCGCCCAATA CGCAAACCGC CTCTCCCCGC |
| 9421 | GCGTTGGCCG ATTCATTAAT GCAGCTGTGG AATGTGTGTC AGTTAGGGTG TGGAAAGTCC |
| 9481 | CCAGGCTCCC CAGCAGGCAG AAGTATGCAA AGCATGCATC TCAATTAGTC AGCAACCAGG |
| 9541 | TGTGGAAAGT CCCCAGGCTC CCCAGCAGGC AGAAGTATGC AAAGCATGCA TCTCAATTAG |
| 9601 | TCAGCAACCA TAGTCCCGCC CCTAACTCCG CCCATCCCGC CCCTAACTCC GCCCAGTTCC |
| 9661 | GCCCATTCTC CGCCCCATGG CTGACTAATT TTTTTTATTT ATGCAGAGGC CGAGGCCGCC |
| 9721 | TCGGCCTCTG AGCTATTCCA GAAGTAGTGA GGAGGCTTTT TTGGAGGCCT AGGCTTTTGC |
| 9781 | AAAAAG |

SEQ ID NO: 53

| | |
|---|---|
| 1 | CTGCGCGCTC GCTCGCTCAC TGAGGCCGCC CGGGCAAAGC CCGGGCGTCG GGCGACCTTT |
| 61 | GGTCGCCCGG CCTCAGTGAG CGAGCGAGCG CGCAGAGAGG GAGTGGCCAA CTCCATCACT |
| 121 | AGGGGTTCCT GCGGCAATTC AGTCGATAAC TATAACGGTC CTAAGGTAGC GATTTAAATG |
| 181 | GTACCCATGG TCGAGGTGAG CCCCACGTTC TGCTTCACTC TCCCCATCTC CCCCCCCTCC |
| 241 | CCACCCCCAA TTTTGTATTT ATTTATTTTT TAATTATTTT GTGCAGCGAT GGGGGCGGGG |
| 301 | GGGGGGGGGG GGCGCGCGCC AGGCGGGGCG GGGCGGGGCG AGGGGCGGGG CGGGGCGAGG |
| 361 | CGGAGAGGTG CGGCGGCAGC CAATCAGAGC GGCGCGCTCC GAAAGTTTCC TTTTATGGCG |
| 421 | AGGCGGCGGC GGCGGCGGCC CTATAAAAAG CGAAGCGCGC GGCGGGCGGG AGTCGCTGCG |
| 481 | CGCTGCCTTC GCCCCGTGCC CCGCTCCGCC GCCGCCTCGC GCCGCCCGCC CCGGCTCTGA |
| 541 | CTGACCGCGT TACTCCCACA GGTGAGCGGG CGGGACGGCC CTTCTCCTCC GGGCTGTAAT |
| 601 | TAGCGCTTGG TTTAATGACG GCTTGTTTCT TTTCTGTGGC TGCGTGAAAG CCTTGAGGGG |
| 661 | CTCCGGGAGG GCCCTTTGTG CGGGGGGAGC GGCTCGGGGC TGTCCGCGGG GGGACGGCTG |
| 721 | CCTTCGGGGG GGACGGGGCA GGGCGGGGTT CGGCTTCTGG CGTGTGACCG GCGGCTCTAG |
| 781 | AGCCTCTGCT AACCATGTTC ATGCCTTCTT CTTTTTCCTA CAGCTCCTGG GCAACGTGCT |
| 841 | GGTTATTGTG CTGTCTCATC ATTTTGGCAA AGAATTGGAT CCTAGCTTGA TATCGAATTC |
| 901 | CTGCAGCCCG GCACCACCAT GGGCTTCGTG AGACAGATAC AGCTTTTGCT CTGGAAGAAC |
| 961 | TGGACCCTGC GGAAAAGGCA AAAGATTCGC TTTGTGGTGG AACTCGTGTG GCCTTTATCT |
| 1021 | TTATTTCTGG TCTTGATCTG GTTAAGGAAT GCCAACCCGC TCTACAGCCA TCATGAATGC |
| 1081 | CATTTCCCCA ACAAGGCGAT GCCCTCAGCA GGAATGCTGC CGTGGCTCCA GGGGATCTTC |
| 1141 | TGCAATGTGA ACAATCCCTG TTTTCAAAGC CCCACCCCAG GAGAATCTCC TGGAATTGTG |
| 1201 | TCAAACTATA ACAACTCCAT CTTGGCAAGG GTATATCGAG ATTTTCAAGA ACTCCTCATG |
| 1261 | AATGCACCAG AGAGCCAGCA CCTTGGCCGT ATTTGGACAG AGCTACACAT CTTGTCCCAA |
| 1321 | TTCATGGACA CCCTCCGGAC TCACCCGGAG AGAATTGCAG GAAGAGGAAT ACGAATAAGG |
| 1381 | GATATCTTGA AAGATGAAGA AACACTGACA CTATTTCTCA TTAAAAACAT CGGCCTGTCT |
| 1441 | GACTCAGTGG TCTACCTTCT GATCAACTCT CAAGTCCGTC CAGAGCAGTT CGCTCATGGA |
| 1501 | GTCCCGGACC TGGCGCTGAA GGACATCGCC TGCAGCGAGG CCCTCCTGGA GCGCTTCATC |
| 1561 | ATCTTCAGCC AGAGACGCGG GGCAAAGACG GTGCGCTATG CCCTGTGCTC CCTCTCCCAG |
| 1621 | GGCACCCTAC AGTGGATAGA AGACACTCTG TATGCCAACG TGGACTTCTT CAAGCTCTTC |
| 1681 | CGTGTGCTTC CCACACTCCT AGACAGCCGT TCTCAAGGTA TCAATCTGAG ATCTTGGGGA |
| 1741 | GGAATATTAT CTGATATGTC ACCAAGAATT CAAGAGTTTA TCCATCGGCC GAGTATGCAG |
| 1801 | GACTTGCTGT GGGTGACCAG GCCCCTCATG CAGAATGGTG GTCCAGAGAC CTTTACAAAG |
| 1861 | CTGATGGGCA TCCTGTCTGA CCTCCTGTGT GGCTACCCCG AGGGAGGTGG CTCTCGGGTG |
| 1921 | CTCTCCTTCA ACTGGTATGA AGACAATAAC TATAAGGCCT TTCTGGGGAT TGACTCCACA |
| 1981 | AGGAAGGATC CTATCTATTC TTATGACAGA AGAACAACAT CCTTTTGTAA TGCATTGATC |
| 2041 | CAGAGCCTGG AGTCAAATCC TTTAACCAAA ATCGCTTGGA GGGCGGCAAA GCCTTTGCTG |
| 2101 | ATGGGAAAAA TCCTGTACAC TCCTGATTCA CCTGCAGCAC GAAGGATACT GAAGAATGCC |
| 2161 | AACTCAACTT TTGAAGAACT GGAACACGTT AGGAAGTTGG TCAAAGCCTG GGAAGAAGTA |
| 2221 | GGGCCCCAGA TCTGGTACTT CTTTGACAAC AGCACACAGA TGAACATGAT CAGAGATACC |
| 2281 | CTGGGGAACC CAACAGTAAA AGACTTTTTG AATAGGCAGC TTGGTGAAGA AGGTATTACT |
| 2341 | GCTGAAGCCA TCCTAAACTT CCTCTACAAG GGCCCTCGGG AAAGCCAGGC TGACGACATG |
| 2401 | GCCAACTTCG ACTGGAGGGA CATATTTAAC ATCACTGATC GCACCCTCCG CCTTGTCAAT |
| 2461 | CAATACCTGG AGTGCTTGGT CCTGGATAAG TTTGAAAGCT ACAATGATGA AACTCAGCTC |
| 2521 | ACCCAACGTG CCCTCTCTCT ACTGGAGGAA AACATGTTCT GGGCCGGAGT GGTATTCCCT |
| 2581 | GACATGTATC CCTGGACCAG CTCTCTACCA CCCCACGTGA AGTATAAGAT CCGAATGGAC |
| 2641 | ATAGACGTGG TGGAGAAAAC CAATAAGATT AAAGACAGGT ATTGGGATTC TGGTCCCAGA |
| 2701 | GCTGATCCCG TGGAAGATTT CCGGTACATC TGGGGCGGGT TTGCCTATCT GCAGGACATG |
| 2761 | GTTGAACAGG GGATCACAAG GAGCCAGGTG CAGGCGGAGG CTCCAGTTGG AATCTACCTC |
| 2821 | CAGCAGATGC CCTACCCCTG CTTCGTGGAC GATTCTTTCA TGATCATCCT GAACCGCTGT |
| 2881 | TTCCCTATCT TCATGGTGCT GGCATGGATC TACTCTGTCT CCATGACTGT GAAGAGCATC |
| 2941 | GTCTTGGAGA AGGAGTTGCG ACTGAAGGAG ACCTTGAAAA ATCAGGGTGT CTCCAATGCA |
| 3001 | GTGATTTGGT GTACCTGGTT CCTGGACAGC TTCTCCATCA TGTCGATGAG CATCTTCCTC |
| 3061 | CTGACGATAT TCATCATGCA TGGAAGAATC CTACATTACA GCGACCCATT CATCCTCTTC |
| 3121 | CTGTTCTTGT TGGCTTTCTC CACTGCCACC ATCATGCTGT GCTTTCTGCT CAGCACCTTC |
| 3181 | TTCTCCAAGG CCAGTCTGGC AGCAGCCTGT AGTGGTGTCA TCTATTTCAC CCTCTACCTG |
| 3241 | CCACACATCC TGTGCTTCGC CTGGCAGGAC CGCATGACCG CTGAGCTGAA GAAGGCTGTG |
| 3301 | AGCTTACTGT CTCCGGTGGC ATTTGGATTT GGCACTGAGT ACCTGGTTCG CTTTGAAGAG |
| 3361 | CAAGGCCTGG GGCTGCAGTG GAGCAACATC GGGAACAGTC CCACGGAAGG GGACGAATTC |
| 3421 | AGCTTCCTGC TGTCCATGCA GATGATGCTC CTTGATGCTG CTGTCTATGG CTTACTCGCT |
| 3481 | TGGTACCTTG ATCAGGTGTT TCCAGGAGAC TATGGAACCC CACTTCCTTG GTACTTTCTT |
| 3541 | CTACAAGAGT CGTATTGGCT TGGCGGTGAA GGGTGTTCAA CCAGAGAAGA AAGAGCCCTG |
| 3601 | GAAAAGACCG AGCCCCTAAC AGAGGAAACG GAGGATCCAG AGCACCCAGA AGGAATACAC |
| 3661 | GACTCCTTCT TTGAACGTGA GCATCCAGGG TGGGTTCCTG GGGTATGCGT GAAGAATCTG |
| 3721 | GTAAAGATTT TTGAGCCCTG TGGCCGGCCA GCTGTGGACC GTCTGAACAT CACCTTCTAC |
| 3781 | GAGAACCAGA TCACCGCATT CCTGGGCCAC AATGGAGCTG GGAAAACCAC CACCTTGTCC |
| 3841 | ATCCTGACGG GTCTGTTGCC ACCAACCTCT GGGACTGTGC TCGTTGGGGG AAGGGACATT |
| 3901 | GAAACCAGCC TGGATGCAGT CCGGCAGAGC CTTGGCATGT GTCCACAGCA CAACATCCTG |
| 3961 | TTCCACCACC TCACGGTGGC TGAGCACATG CTGTTCTATG CCCAGCTGAA AGGAAAGTCC |
| 4021 | CAGGAGGAGG CCCAGCTGGA GATGGAAGCC ATGTTGGAGG ACACAGGCCT CCACCACAAG |
| 4081 | CGGAATGAAG AGGCTCAGGA CCTATCAGGT GGCATGCAGA GAAAGCTGTC GGTTGCCATT |
| 4141 | GCCTTTGTGG GAGATGCCAA GGTGGTGATT CTGGACGAAC CCACCTCTGG GGTGGACCCT |
| 4201 | TACTCGAGAC GCTCAATCTG GGATCTGCTC CTGAAGTATC GCTCAGGCAG AACCATCATC |
| 4261 | ATGTCCACTC ACCACATGGA CGAGGCCGAC CTCCTTGGGG ACCGCATTGC CATCATTGCC |
| 4321 | CAGGGAAGGC TCTACTGCTC AGGCACCCCA CTCTTCCTGA AGAACTGCTT TGGCACAGGC |
| 4381 | TTGTACTTAA CCTTGGTGCG CAAGATGAAA AACATCCAGA GCCAAAGGAA AGGCAGTGAG |
| 4441 | GGGACCTGCA GCTGCTCGTC TAAGGGTTTC TCCACCACGT GTCCAGCCCA CGTCGATGAC |
| 4501 | CTAACTCCAG AACAAGTCCT GGATGGGGAT GTAAATGAGC TGATGGATGT AGTTCTCCAC |
| 4561 | CATGTTCCAG AGGCAAAGCT GGTGGAGTGC ATTGGTCAAG AACTTATCTT CCTTCTTCCA |
| 4621 | TTTAAATTAG GGATAACAGG GTGGTGGCGC GGGCCGCAGG AACCCCTAGT GATGGAGTTG |
| 4681 | GCCACTCCCT CTCTGCGCGC TCGCTCGCTC ACTGAGGCCG GGCGACCAAA GGTCGCCCGA |
| 4741 | CGCCCGGGCG GCCTCAGTGA GCGAGCGAGC GCGCAGAGCT AGAATTAATT CCGTGTATTC |
| 4801 | TATAGTGTCA CCTAAATCGT ATGTGTATGA TACATAAGGT TATGTATTAA TTGTAGCCGC |
| 4861 | GTTCTAACGA CAATATGTAC AAGCCTAATT GTGTAGCATC TGGCTTAGCG GCCGCCTACC |
| 4921 | GTCAAACAGT CAATCCCGTT CTACGCCATT TGACACATAA CGCCCGGGAT AACAGAGCTG |
| 4981 | AATTTGACGG ACTACGATAT TGCTTATGTG CCACCAATCA ACAGTTAACG AACACGTGGC |
| 5041 | GGCGCGGAAC GCCTCCGGCC AGGCCGCGCG CTTCGCATAT TTACTTCGAG CAGTGTAGGT |
| 5101 | GTGACAACGT AGCATGCAGC CACATCCCTA GCTTGAACCG GAGATAAAGG TCTACGCGCG |
| 5161 | CGACGTCCAC ATTCACACGG TTCAGATTCC TGGTGCTACC CAAAACAAAG TCCATAGGTT |
| 5221 | TTTCATTGGG ACTACGGCGC GAAGCTAAGT GGTTTCACAC CTACAAGGGA AACATGCCCA |
| 5281 | AACTATGAGG ACAACATCGT CCGCAGAAAC AATCGGCCGC GATAGGGGTT GCACGTTGTC |
| 5341 | AGATGAAAGA GCCACACTCG GGGAGCAGTC CGCGGACGCC ACCTCGTGCA ACTTCGGCTA |
| 5401 | ACCATATAAT CTAAAAAAGT TGAGGTTTGC AGTTGTCGGG GCGAGATCAA ACCCAAGTAT |
| 5461 | ATAGTCCTGT CCGGAGCCTT AGTTCACGTA CTCGCGACCC TTGAAAGCGC GTCAAGCTTA |
| 5521 | TCGCTCACTG ACTAGCTCAA TGTGTGGCAA TCTAAGTAGG AGGTCTGTCG CAAGGCAAAA |
| 5581 | ATGCTAATTA TTGGTAGCAA GCTTAGATAA GGTGGAGGGA TTGCACAATT CAGAAGGCGT |
| 5641 | CTTCTCTGCT ACACCCGAGC GGGGTGCTTT ATCAAGGGGA AGCTTGATGT CCCACGGGAT |
| 5701 | GAACGAGAGC CTCCATGGCA TCTCACGACC TACTTAACTT CGGGGGATGG GTAGAAGTTA |
| 5761 | GCTGAACATA CAAATGGGAA TAGGATTGTG CCCTCGGACG AGACTGAACG GATCGCAGTC |
| 5821 | AACCCGCGCA AAGTTTACAT ATTAATTCTT ACGGCGTGTC AGAGAGGCAA TGGCTTGACT |
| 5881 | TGTGGTGGAT CACAGTTTGT GAGTAACGGC AAGATGCGGT AAACACTGTA ATGCGAGCTT |
| 5941 | CATTGACTCG GCTTAAAGTT CCTGGTACCA TAATGAATAC ACGGTGGTTA GTTGTCAATT |
| 6001 | GCTTGTGCAC CGCCGCACCT TGCGGTCCTC GGTCCAGCCT GCGCAGGGTA TAAATGAAGC |
| 6061 | ACGTCCCACC CAGACTGTTC CATCGTACCT CCAAATACGG ATTCAACCTG GCGTCTATTT |
| 6121 | CCAGATATGG GCCCTAGGGG TGATAGACTC CCAAGTCTAA GGACTACCAT GGGATATGTT |
| 6181 | TCACGTATCC AAAAAGTAAC CATAATACTG CGTTTCCGTT CACCCAAGTG AGGATGTTGC |
| 6241 | CTTTGTACTG GTTTCATAGT CCTGCCGTAC CAGGCGTCTT CCTTAGCCGG CGCTACTTCC |
| 6301 | AGCCCGGAAC TGTCTTGTTT CTCGATGTGA GACCCTTGTC AGCCGCCCGC GGTGGTGCAC |
| 6361 | GTAAAAGCCG ATTGGAGTAT TAAGTATTTA CAACTCCGAA TCTTAAGAGC CCTGCTCTAG |
| 6421 | TTTGGATTCA TATATCAGCA TAGGCTTCGC AACCTAGTGA ATGAGCGGTA CGAACTTTCG |
| 6481 | CGGAGTGCGA AAAGCGACCG AGCAATCGAG ATACGTACCG TTAGATTCAC GCTCCAGACA |
| 6541 | GCACTCTGAG TCTTTGATTT ATAACCATCG AAGGAATCGA CTTCACGTCC CTAGCGTGTT |
| 6601 | GAGTCATCCG CAGAAGAGAC GATGAGGGCT CGCCCCCCGA AATAGTTCTG CTTCAAACTA |
| 6661 | TAGGCTGCCC TACTTGGTCT CCGAGGTACT ATGGGGTCCT CGACGGTTCG AGGCCCCCAA |
| 6721 | CCCATGTTCA ATCAGCTCGT ATGTCTACCC TCGAGCTAAC ACAGGAACCA GCTGAGACTT |
| 6781 | GCCTGGCGTC ACTTGGGCAC GTTCCATATA CATAATGAAG TACGCCGCAG GGTCTCTCCG |
| 6841 | TTACCGAACT GTGCTCGACC TAAAGTCCGG TACCCATCGG CGTCCTGTCA CATTTGTGGC |
| 6901 | ATTAGGTATG AACTAACTCT GGGGGGCTTC TACGACCATG GTAAAAGTTT TGTGCTGCCA |
| 6961 | GACAACTGTT AATAAACATG TCGCTGCGTA GAACGCCAAG AACCAGCTGG GATGAGTGCC |
| 7021 | TTATTTACCC CGCGCGAGGT GGGTCTGAGT AGGTAGCATC GAGGTTTACG CCTAAGTTGG |
| 7081 | ACCGCAAATA TAGGCCCTTT GCCGGGATCC CCACTATCTG TGAATTGTGA AACCCGTTGG |
| 7141 | CACCCTGTAC AAAGTGCATA GCTACATCAT TGGTAACAAG ACGTAAACGG AGGTTCGCTC |
| 7201 | ACTCCCACTT CGGAAAGATA ACCGGGGAAC TAGGAGGGTA TGGTGCGCGC ATGGAAAGGG |
| 7261 | CCGGGAAGTA ACTCTGGCCT TCACGGAACG ATAAGTTACA ATTTGGGAAC AGTCGGAGAG |
| 7321 | CGCCACTACG TGCTTTTTTG GCTTACCTCA TATCTCGTAG TTGGTGAGGG TTAAAATTCG |
| 7381 | CGGGAGAAGA TCCAGCCTAA GTATATGGTT ACATCGCGGC CGCCTGAAGC AGACCCTATC |
| 7441 | ATCTCTCTCG TAAACTGCCG TCAGAGTCGG TTTGGTTGGA CGAACCTTCT GAGTTTCTGG |
| 7501 | TAACGCCGTC CCGCACCCGG AAATGGTCAG CGAACCAATC AGCAGGGTCA TCGCTAGCCA |
| 7561 | GATCCTCTAC GCCGGACGCA TCGTGGCCGG CATCACCGGC GCCACAGGTG CGGTTGCTGG |
| 7621 | CGCCTATATC GCCGACATCA CCGATGGGGA AGATCGGGCT CGCCACTTCG GGCTCATGAG |
| 7681 | CGCTTGTTTC GGCGTGGGTA TGGTGGCAGG CCGCCCTTAG AAAAACTCAT CGAGCATCAA |
| 7741 | ATGAAACTGC AATTTATTCA TATCAGGATT ATCAATACCA TATTTTTGAA AAAGCCGTTT |
| 7801 | CTGTAATGAA GGAGAAAACT CACCGAGGCA GTTCCATAGG ATGGCAAGAT CCTGGTATCG |
| 7861 | GTCTGCGATT CCGACTCGTC CAACATCAAT ACAACCTATT AATTTCCCCT CGTCAAAAAT |
| 7921 | AAGGTTATCA AGTGAGAAAT CACCATGAGT GACGACTGAA TCCGGTGAGA ATGGCAAAAG |
| 7981 | CTTATGCATT TCTTTCCAGA CTTGTTCAAC AGGCCAGCCA TTACGCTCGT CATCAAAATC |
| 8041 | ACTCGCATCA ACCAAACCGT TATTCATTCG TGATTGCGCC TGAGCGAGAC GAAATACGCG |
| 8101 | ATCGCTGTTA AAAGGACAAT TACAAACAGG AATCGAATGC AACCGGCGCA GGAACACTGC |
| 8161 | CAGCGCATCA ACAATATTTT CACCTGAATC AGGATATTCT TCTAATACCT GGAATGCTGT |
| 8221 | TTTCCCGGGG ATCGCAGTGG TGAGTAACCA TGCATCATCA GGAGTACGGA TAAAATGCTT |
| 8281 | GATGGTCGGA AGAGGCATAA ATTCCGTCAG CCAGTTTAGT CTGACCATCT CATCTGTAAC |
| 8341 | ATCATTGGCA ACGCTACCTT TGCCATGTTT CAGAAACAAC TCTGGCGCAT CGGGCTTCCC |
| 8401 | ATACAATCGA TAGATTGTCG CACCTGATTG CCCGACATTA TCGCGAGCCC ATTTATACCC |
| 8461 | ATATAAATCA GCATCCATGT TGGAATTTAA TCGCGGCCTC GAGCAAGACG TTTCCCGTTG |
| 8521 | AATATGGCTC ATAACACCCC TTGTATTACT GTTTATGTAA GCAGACAGTT TTATTGTTCA |
| 8581 | TGATGATATA TTTTTATCTT GTGCAATGTA ACATCAGAGA TTTTGAGACA CAACGTGGTT |
| 8641 | TGCAGGAGTC AGGCAACTAT GGATGAACGA AATAGACAGA TCGCTGAGAT AGGTGCCTCA |
| 8701 | CTGATTAAGC ATTGGTAACT GTCAGACCAA GTTTACTCAT ATATACTTTA GATTGATTTA |
| 8761 | AAACTTCATT TTTAATTTAA AAGGATCTAG GTGAAGATCC TTTTTGATAA TCTCATGACC |
| 8821 | AAAATCCCTT AACGTGAGTT TTCGTTCCAC TGAGCGTCAG ACCCCGTAGA AAAGATCAAA |
| 8881 | GGATCTTCTT GAGATCCTTT TTTTCTGCGC GTAATCTGCT GCTTGCAAAC AAAAAAACCA |
| 8941 | CCGCTACCAG CGGTGGTTTG TTTGCCGGAT CAAGAGCTAC CAACTCTTTT TCCGAAGGTA |
| 9001 | ACTGGCTTCA GCAGAGCGCA GATACCAAAT ACTGTTCTTC TAGTGTAGCC GTAGTTAGGC |
| 9061 | CACCACTTCA AGAACTCTGT AGCACCGCCT ACATACCTCG CTCTGCTAAT CCTGTTACCA |
| 9121 | GTGGCTGCTG CCAGTGGCGA TAAGTCGTGT CTTACCGGGT TGGACTCAAG ACGATAGTTA |
| 9181 | CCGGATAAGG CGCAGCGGTC GGGCTGAACG GGGGGTTCGT GCACACAGCC CAGCTTGGAG |
| 9241 | CGAACGACCT ACACCGAACT GAGATACCTA CAGCGTGAGC TATGAGAAAG CGCCACGCTT |
| 9301 | CCCGAAGGGA GAAAGGCGGA CAGGTATCCG GTAAGCGGCA GGGTCGGAAC AGGAGAGCGC |
| 9361 | ACGAGGGAGC TTCCAGGGGG AAACGCCTGG TATCTTTATA GTCCTGTCGG GTTTCGCCAC |
| 9421 | CTCTGACTTG AGCGTCGATT TTTGTGATGC TCGTCAGGGG GGCGGAGCCT ATGGAAAAAC |
| 9481 | GCCAGCAACG CGGCCTTTTT ACGGTTCCTG GCCTTTTGCT GGCCTTTTGC TCACATGTTC |
| 9541 | TTTCCTGCGT TATCCCCTGA TTCTGTGGAT AACCGTATTA CCGCCTTTGA GTGAGCTGAT |
| 9601 | ACCGCTCGCC GCAGCCGAAC GACCGAGCGC AGCGAGTCAG TGAGCGAGGA AGCGGAAGAG |
| 9661 | CGCCCAATAC GCAAACCGCC TCTCCCCGCG CGTTGGCCGA TTCATTAATG CAGCTGTGGA |
| 9721 | ATGTGTGTCA GTTAGGGTGT GGAAAGTCCC CAGGCTCCCC AGCAGGCAGA AGTATGCAAA |
| 9781 | GCATGCATCT CAATTAGTCA GCAACCAGGT GTGGAAAGTC CCCAGGCTCC CCAGCAGGCA |
| 9841 | GAAGTATGCA AAGCATGCAT CTCAATTAGT CAGCAACCAT AGTCCCGCCC CTAACTCCGC |
| 9901 | CCATCCCGCC CCTAACTCCG CCCAGTTCCG CCCATTCTCC GCCCCATGGC TGACTAATTT |
| 9961 | TTTTTATTTA TGCAGAGGCC GAGGCCGCCT CGGCCTCTGA GCTATTCCAG AAGTAGTGAG |
| 10021 | GAGGCTTTTT TGGAGGCCTA GGCTTTTGCA AAAAG |

SEQ ID NO: 59

| | |
|---|---|
| 1 | CTGCGCGCTC GCTCGCTCAC TGAGGCCGCC CGGGCAAAGC CCGGGCGTCG GGCGACCTTT |
| 61 | GGTCGCCCGG CCTCAGTGAG CGAGCGAGCG CGCAGAGAGG GAGTGGCCAA CTCCATCACT |
| 121 | AGGGGTTCCT GCGGCAATTC AGTCGATAAC TATAACGGTC CTAAGGTAGC GATTTAAATG |
| 181 | GTACCCTCAG ATCTGAATTC GGTACCTAGT TATTAATAGT AATCAATTAC GGGGTCATTA |
| 241 | GTTCATAGCC CATATATGGA GTTCCGCGTT ACATAACTTA CGGTAAATGG CCCGCCTGGC |
| 301 | TGACCGCCCA ACGACCCCCG CCCATTGACG TCAATAATGA CGTATGTTCC CATAGTAACG |
| 361 | CCAATAGGGA CTTTCCATTG ACGTCAATGG GTGGAGTATT TACGGTAAAC TGCCCACTTG |
| 421 | GCAGTACATC AAGTGTATCA TATGCCAAGT ACGCCCCCTA TTGACGTCAA TGACGGTAAA |
| 481 | TGGCCCGCCT GGCATTATGC CCAGTACATG ACCTTATGGG ACTTTCCTAC TTGGCAGTAC |
| 541 | ATCTACGTAT TAGTCATCGC TATTACCATG GTCGAGGTGA GCCCCACGTT CTGCTTCACT |
| 601 | CTCCCCATCT CCCCCCCCTC CCCACCCCCA ATTTTGTATT TATTTATTTT TTAATTATTT |
| 661 | TGTGCAGCGA TGGGGGCGGG GGGGGGGGGG GGGCGCGCGC CAGGCGGGGC GGGGCGGGGC |
| 721 | GAGGGGCGGG GCGGGGCGAG GCGGAGAGGT GCGGCGGCAG CCAATCAGAG CGGCGCGCTC |
| 781 | CGAAAGTTTC CTTTTATGGC GAGGCGGCGG CGGCGGCGGC CCTATAAAAA GCGAAGCGCG |
| 841 | CGGCGGGCGA CCACCATGGG CTTCGTGAGA CAGATACAGC TTTTGCTCTG GAAGAACTGG |
| 901 | ACCCTGCGGA AAAGGCAAAA GATTCGCTTT GTGGTGGAAC TCGTGTGGCC TTTATCTTTA |
| 961 | TTTCTGGTCT TGATCTGGTT AAGGAATGCC AACCCGCTCT ACAGCCATCA TGAATGCCAT |
| 1021 | TTCCCCAACA AGGCGATGCC CTCAGCAGGA ATGCTGCCGT GGCTCCAGGG GATCTTCTGC |
| 1081 | AATGTGAACA ATCCCTGTTT TCAAAGCCCC ACCCCAGGAG AATCTCCTGG AATTGTGTCA |
| 1141 | AACTATAACA ACTCCATCTT GGCAAGGGTA TATCGAGATT TTCAAGAACT CCTCATGAAT |
| 1201 | GCACCAGAGA GCCAGCACCT TGGCCGTATT TGGACAGAGC TACACATCTT GTCCCAATTC |
| 1261 | ATGGACACCC TCCGGACTCA CCCGGAGAGA ATTGCAGGAA GAGGAATACG AATAAGGGAT |
| 1321 | ATCTTGAAAG ATGAAGAAAC ACTGACACTA TTTCTCATTA AAAACATCGG CCTGTCTGAC |
| 1381 | TCAGTGGTCT ACCTTCTGAT CAACTCTCAA GTCCGTCCAG AGCAGTTCGC TCATGGAGTC |
| 1441 | CCGGACCTGG CGCTGAAGGA CATCGCCTGC AGCGAGGCCC TCCTGGAGCG CTTCATCATC |
| 1501 | TTCAGCCAGA GACGCGGGGC AAAGACGGTG CGCTATGCCC TGTGCTCCCT CTCCCAGGGC |
| 1561 | ACCCTACAGT GGATAGAAGA CACTCTGTAT GCCAACGTGG ACTTCTTCAA GCTCTTCCGT |
| 1621 | GTGCTTCCCA CACTCCTAGA CAGCCGTTCT CAAGGTATCA ATCTGAGATC TTGGGGAGGA |
| 1681 | ATATTATCTG ATATGTCACC AAGAATTCAA GAGTTTATCC ATCGGCCGAG TATGCAGGAC |
| 1741 | TTGCTGTGGG TGACCAGGCC CCTCATGCAG AATGGTGGTC CAGAGACCTT TACAAAGCTG |
| 1801 | ATGGGCATCC TGTCTGACCT CCTGTGTGGC TACCCCGAGG GAGGTGGCTC TCGGGTGCTC |
| 1861 | TCCTTCAACT GGTATGAAGA CAATAACTAT AAGGCCTTTC TGGGGATTGA CTCCACAAGG |
| 1921 | AAGGATCCTA TCTATTCTTA TGACAGAAGA ACAACATCCT TTTGTAATGC ATTGATCCAG |
| 1981 | AGCCTGGAGT CAAATCCTTT AACCAAAATC GCTTGGAGGG CGGCAAAGCC TTTGCTGATG |
| 2041 | GGAAAAATCC TGTACACTCC TGATTCACCT GCAGCACGAA GGATACTGAA GAATGCCAAC |
| 2101 | TCAACTTTTG AAGAACTGGA ACACGTTAGG AAGTTGGTCA AAGCCTGGGA AGAAGTAGGG |
| 2161 | CCCCAGATCT GGTACTTCTT TGACAACAGC ACACAGATGA ACATGATCAG AGATACCCTG |
| 2221 | GGGAACCCAA CAGTAAAAGA CTTTTTGAAT AGGCAGCTTG GTGAAGAAGG TATTACTGCT |
| 2281 | GAAGCCATCC TAAACTTCCT CTACAAGGGC CCTCGGGAAA GCCAGGCTGA CGACATGGCC |
| 2341 | AACTTCGACT GGAGGGACAT ATTTAACATC ACTGATCGCA CCCTCCGCCT TGTCAATCAA |
| 2401 | TACCTGGAGT GCTTGGTCCT GGATAAGTTT GAAAGCTACA ATGATGAAAC TCAGCTCACC |
| 2461 | CAACGTGCCC TCTCTCTACT GGAGGAAAAC ATGTTCTGGG CCGGAGTGGT ATTCCCTGAC |
| 2521 | ATGTATCCCT GGACCAGCTC TCTACCACCC CACGTGAAGT ATAAGATCCG AATGGACATA |
| 2581 | GACGTGGTGG AGAAAACCAA TAAGATTAAA GACAGGTATT GGGATTCTGG TCCCAGAGCT |
| 2641 | GATCCCGTGG AAGATTTCCG GTACATCTGG GGCGGGTTTG CCTATCTGCA GGACATGGTT |
| 2701 | GAACAGGGGA TCACAAGGAG CCAGGTGCAG GCGGAGGCTC CAGTTGGAAT CTACCTCCAG |
| 2761 | CAGATGCCCT ACCCCTGCTT CGTGGACGAT TCTTTCATGA TCATCCTGAA CCGCTGTTTC |
| 2821 | CCTATCTTCA TGGTGCTGGC ATGGATCTAC TCTGTCTCCA TGACTGTGAA GAGCATCGTC |
| 2881 | TTGGAGAAGG AGTTGCGACT GAAGGAGACC TTGAAAAATC AGGGTGTCTC CAATGCAGTG |
| 2941 | ATTTGGTGTA CCTGGTTCCT GGACAGCTTC TCCATCATGT CGATGAGCAT CTTCCTCCTG |
| 3001 | ACGATATTCA TCATGCATGG AAGAATCCTA CATTACAGCG ACCCATTCAT CCTCTTCCTG |
| 3061 | TTCTTGTTGG CTTTCTCCAC TGCCACCATC ATGCTGTGCT TTCTGCTCAG CACCTTCTTC |
| 3121 | TCCAAGGCCA GTCTGGCAGC AGCCTGTAGT GGTGTCATCT ATTTCACCCT CTACCTGCCA |
| 3181 | CACATCCTGT GCTTCGCCTG GCAGGACCGC ATGACCGCTG AGCTGAAGAA GGCTGTGAGC |
| 3241 | TTACTGTCTC CGGTGGCATT TGGATTTGGC ACTGAGTACC TGGTTCGCTT TGAAGAGCAA |
| 3301 | GGCCTGGGGC TGCAGTGGAG CAACATCGGG AACAGTCCCA CGGAAGGGGA CGAATTCAGC |
| 3361 | TTCCTGCTGT CCATGCAGAT GATGCTCCTT GATGCTGCTG TCTATGGCTT ACTCGCTTGG |
| 3421 | TACCTTGATC AGGTGTTTCC AGGAGACTAT GGAACCCCAC TTCCTTGGTA CTTTCTTCTA |
| 3481 | CAAGAGTCGT ATTGGCTTGG CGGTGAAGGG TGTTCAACCA GAGAAGAAAG AGCCCTGGAA |
| 3541 | AAGACCGAGC CCCTAACAGA GGAAACGGAG GATCCAGAGC ACCCAGAAGG AATACACGAC |
| 3601 | TCCTTCTTTG AACGTGAGCA TCCAGGGTGG GTTCCTGGGG TATGCGTGAA GAATCTGGTA |
| 3661 | AAGATTTTTG AGCCCTGTGG CCGGCCAGCT GTGGACCGTC TGAACATCAC CTTCTACGAG |
| 3721 | AACCAGATCA CCGCATTCCT GGGCCACAAT GGAGCTGGGA AAACCACCAC CTTGTCCATC |
| 3781 | CTGACGGGTC TGTTGCCACC AACCTCTGGG ACTGTGCTCG TTGGGGGAAG GGACATTGAA |
| 3841 | ACCAGCCTGG ATGCAGTCCG GCAGAGCCTT GGCATGTGTC CACAGCACAA CATCCTGTTC |
| 3901 | CACCACCTCA CGGTGGCTGA GCACATGCTG TTCTATGCCC AGCTGAAAGG AAAGTCCCAG |
| 3961 | GAGGAGGCCC AGCTGGAGAT GGAAGCCATG TTGGAGGACA CAGGCCTCCA CCACAAGCGG |
| 4021 | AATGAAGAGG CTCAGGACCT ATCAGGTGGC ATGCAGAGAA AGCTGTCGGT TGCCATTGCC |
| 4081 | TTTGTGGGAG ATGCCAAGGT GGTGATTCTG GACGAACCCA CCTCTGGGGT GGACCCTTAC |
| 4141 | TCGAGACGCT CAATCTGGGA TCTGCTCCTG AAGTATCGCT CAGGCAGAAC CATCATCATG |
| 4201 | TCCACTCACC ACATGGACGA GGCCGACCTC CTTGGGGACC GCATTGCCAT CATTGCCCAG |
| 4261 | GGAAGGCTCT ACTGCTCAGG CACCCCACTC TTCCTGAAGA ACTGCTTTGG CACAGGCTTG |
| 4321 | TACTTAACCT TGGTGCGCAA GATGAAAAAC ATCCAGAGCC AAAGGAAAGG CAGTGAGGGG |
| 4381 | ACCTGCAGCT GCTCGTCTAA GGGTTTCTCC ACCACGTGTC CAGCCCACGT CGATGACCTA |
| 4441 | ACTCCAGAAC AAGTCCTGGA TGGGGATGTA AATGAGCTGA TGGATGTAGT TCTCCACCAT |
| 4501 | GTTCCAGAGG CAAAGCTGGT GGAGTGCATT GGTCAAGAAC TTATCTTCCT TCTTCCATTT |
| 4561 | AAATTAGGGA TAACAGGGTG GTGGCGCGGG CCGCAGGAAC CCCTAGTGAT GGAGTTGGCC |
| 4621 | ACTCCCTCTC TGCGCGCTCG CTCGCTCACT GAGGCCGGGC GACCAAAGGT CGCCCGACGC |
| 4681 | CCGGGCGGCC TCAGTGAGCG AGCGAGCGCG CAGAGCTAGA ATTAATTCCG TGTATTCTAT |
| 4741 | AGTGTCACCT AAATCGTATG TGTATGATAC ATAAGGTTAT GTATTAATTG TAGCCGCGTT |
| 4801 | CTAACGACAA TATGTACAAG CCTAATTGTG TAGCATCTGG CTTAGCGGCC GCCTACCGTC |
| 4861 | AAACAGTCAA TCCCGTTCTA CGCCATTTGA CACATAACGC CCGGGATAAC AGAGCTGAAT |
| 4921 | TTGACGGACT ACGATATTGC TTATGTGCCA CCAATCAACA GTTAACGAAC ACGTGGCGGC |
| 4981 | GCGGAACGCC TCCGGCCAGG CCGCGCGCTT CGCATATTTA CTTCGAGCAG TGTAGGTGTG |
| 5041 | ACAACGTAGC ATGCAGCCAC ATCCCTAGCT TGAACCGGAG ATAAAGGTCT ACGCGCGCGA |
| 5101 | CGTCCACATT CACACGGTTC AGATTCCTGG TGCTACCCAA AACAAAGTCC ATAGGTTTTT |
| 5161 | CATTGGGACT ACGGCGCGAA GCTAAGTGGT TTCACACCTA CAAGGGAAAC ATGCCCAAAC |
| 5221 | TATGAGGACA ACATCGTCCG CAGAAACAAT CGGCCGCGAT AGGGGTTGCA CGTTGTCAGA |
| 5281 | TGAAAGAGCC ACACTCGGGG AGCAGTCCGC GGACGCCACC TCGTGCAACT TCGGCTAACC |
| 5341 | ATATAATCTA AAAAAGTTGA GGTTTGCAGT TGTCGGGGCG AGATCAAACC CAAGTATATA |
| 5401 | GTCCTGTCCG GAGCCTTAGT TCACGTACTC GCGACCCTTG AAAGCGCGTC AAGCTTATCG |
| 5461 | CTCACTGACT AGCTCAATGT GTGGCAATCT AAGTAGGAGG TCTGTCGCAA GGCAAAAATG |
| 5521 | CTAATTATTG GTAGCAAGCT TAGATAAGGT GGAGGGATTG CACAATTCAG AAGGCGTCTT |
| 5581 | CTCTGCTACA CCCGAGCGGG GTGCTTTATC AAGGGGAAGC TTGATGTCCC ACGGGATGAA |
| 5641 | CGAGAGCCTC CATGGCATCT CACGACCTAC TTAACTTCGG GGGATGGGTA GAAGTTAGCT |
| 5701 | GAACATACAA ATGGGAATAG GATTGTGCCC TCGGACGAGA CTGAACGGAT CGCAGTCAAC |
| 5761 | CCGCGCAAAG TTTACATATT AATTCTTACG GCGTGTCAGA GAGGCAATGG CTTGACTTGT |
| 5821 | GGTGGATCAC AGTTTGTGAG TAACGGCAAG ATGCGGTAAA CACTGTAATG CGAGCTTCAT |
| 5881 | TGACTCGGCT TAAAGTTCCT GGTACCATAA TGAATACACG GTGGTTAGTT GTCAATTGCT |
| 5941 | TGTGCACCGC CGCACCTTGC GGTCCTCGGT CCAGCCTGCG CAGGGTATAA ATGAAGCACG |
| 6001 | TCCCACCCAG ACTGTTCCAT CGTACCTCCA AATACGGATT CAACCTGGCG TCTATTTCCA |
| 6061 | GATATGGGCC CTAGGGGTGA TAGACTCCCA AGTCTAAGGA CTACCATGGG ATATGTTTCA |
| 6121 | CGTATCCAAA AAGTAACCAT AATACTGCGT TTCCGTTCAC CCAAGTGAGG ATGTTGCCTT |
| 6181 | TGTACTGGTT TCATAGTCCT GCCGTACCAG GCGTCTTCCT TAGCCGGCGC TACTTCCAGC |
| 6241 | CCGGAACTGT CTTGTTTCTC GATGTGAGAC CCTTGTCAGC CGCCCGCGGT GGTGCACGTA |
| 6301 | AAAGCCGATT GGAGTATTAA GTATTTACAA CTCCGAATCT TAAGAGCCCT GCTCTAGTTT |
| 6361 | GGATTCATAT ATCAGCATAG GCTTCGCAAC CTAGTGAATG AGCGGTACGA ACTTTCGCGG |
| 6421 | AGTGCGAAAA GCGACCGAGC AATCGAGATA CGTACCGTTA GATTCACGCT CCAGACAGCA |
| 6481 | CTCTGAGTCT TTGATTTATA ACCATCGAAG GAATCGACTT CACGTCCCTA GCGTGTTGAG |
| 6541 | TCATCCGCAG AAGAGACGAT GAGGGCTCGC CCCCCGAAAT AGTTCTGCTT CAAACTATAG |
| 6601 | GCTGCCCTAC TTGGTCTCCG AGGTACTATG GGGTCCTCGA CGGTTCGAGG CCCCCAACCC |
| 6661 | ATGTTCAATC AGCTCGTATG TCTACCCTCG AGCTAACACA GGAACCAGCT GAGACTTGCC |
| 6721 | TGGCGTCACT TGGGCACGTT CCATATACAT AATGAAGTAC GCCGCAGGGT CTCTCCGTTA |
| 6781 | CCGAACTGTG CTCGACCTAA AGTCCGGTAC CCATCGGCGT CCTGTCACAT TTGTGGCATT |
| 6841 | AGGTATGAAC TAACTCTGGG GGGCTTCTAC GACCATGGTA AAAGTTTTGT GCTGCCAGAC |
| 6901 | AACTGTTAAT AAACATGTCG CTGCGTAGAA CGCCAAGAAC CAGCTGGGAT GAGTGCCTTA |
| 6961 | TTTACCCCGC GCGAGGTGGG TCTGAGTAGG TAGCATCGAG GTTTACGCCT AAGTTGGACC |
| 7021 | GCAAATATAG GCCCTTTGCC GGGATCCCCA CTATCTGTGA ATTGTGAAAC CCGTTGGCAC |
| 7081 | CCTGTACAAA GTGCATAGCT ACATCATTGG TAACAAGACG TAAACGGAGG TTCGCTCACT |
| 7141 | CCCACTTCGG AAAGATAACC GGGGAACTAG GAGGGTATGG TGCGCGCATG GAAAGGGCCG |
| 7201 | GGAAGTAACT CTGGCCTTCA CGGAACGATA AGTTACAATT TGGGAACAGT CGGAGAGCGC |
| 7261 | CACTACGTGC TTTTTTGGCT TACCTCATAT CTCGTAGTTG GTGAGGGTTA AAATTCGCGG |
| 7321 | GAGAAGATCC AGCCTAAGTA TATGGTTACA TCGCGGCCGC CTGAAGCAGA CCCTATCATC |
| 7381 | TCTCTCGTAA ACTGCCGTCA GAGTCGGTTT GGTTGGACGA ACCTTCTGAG TTTCTGGTAA |
| 7441 | CGCCGTCCCG CACCCGGAAA TGGTCAGCGA ACCAATCAGC AGGGTCATCG CTAGCCAGAT |
| 7501 | CCTCTACGCC GGACGCATCG TGGCCGGCAT CACCGGCGCC ACAGGTGCGG TTGCTGGCGC |
| 7561 | CTATATCGCC GACATCACCG ATGGGGAAGA TCGGGCTCGC CACTTCGGGC TCATGAGCGC |
| 7621 | TTGTTTCGGC GTGGGTATGG TGGCAGGCCG CCCTTAGAAA AACTCATCGA GCATCAAATG |
| 7681 | AAACTGCAAT TTATTCATAT CAGGATTATC AATACCATAT TTTTGAAAAA GCCGTTTCTG |
| 7741 | TAATGAAGGA GAAAACTCAC CGAGGCAGTT CCATAGGATG GCAAGATCCT GGTATCGGTC |
| 7801 | TGCGATTCCG ACTCGTCCAA CATCAATACA ACCTATTAAT TTCCCCTCGT CAAAAATAAG |
| 7861 | GTTATCAAGT GAGAAATCAC CATGAGTGAC GACTGAATCC GGTGAGAATG GCAAAAGCTT |
| 7921 | ATGCATTTCT TTCCAGACTT GTTCAACAGG CCAGCCATTA CGCTCGTCAT CAAAATCACT |
| 7981 | CGCATCAACC AAACCGTTAT TCATTCGTGA TTGCGCCTGA GCGAGACGAA ATACGCGATC |
| 8041 | GCTGTTAAAA GGACAATTAC AAACAGGAAT CGAATGCAAC CGGCGCAGGA ACACTGCCAG |
| 8101 | CGCATCAACA ATATTTTCAC CTGAATCAGG ATATTCTTCT AATACCTGGA ATGCTGTTTT |
| 8161 | CCCGGGGATC GCAGTGGTGA GTAACCATGC ATCATCAGGA GTACGGATAA AATGCTTGAT |
| 8221 | GGTCGGAAGA GGCATAAATT CCGTCAGCCA GTTTAGTCTG ACCATCTCAT CTGTAACATC |
| 8281 | ATTGGCAACG CTACCTTTGC CATGTTTCAG AAACAACTCT GGCGCATCGG GCTTCCCATA |
| 8341 | CAATCGATAG ATTGTCGCAC CTGATTGCCC GACATTATCG CGAGCCCATT TATACCCATA |
| 8401 | TAAATCAGCA TCCATGTTGG AATTTAATCG CGGCCTCGAG CAAGACGTTT CCCGTTGAAT |
| 8461 | ATGGCTCATA ACACCCCTTG TATTACTGTT TATGTAAGCA GACAGTTTTA TTGTTCATGA |
| 8521 | TGATATATTT TTATCTTGTG CAATGTAACA TCAGAGATTT TGAGACACAA CGTGGTTTGC |
| 8581 | AGGAGTCAGG CAACTATGGA TGAACGAAAT AGACAGATCG CTGAGATAGG TGCCTCACTG |
| 8641 | ATTAAGCATT GGTAACTGTC AGACCAAGTT TACTCATATA TACTTTAGAT TGATTTAAAA |
| 8701 | CTTCATTTTT AATTTAAAAG GATCTAGGTG AAGATCCTTT TTGATAATCT CATGACCAAA |
| 8761 | ATCCCTTAAC GTGAGTTTTC GTTCCACTGA GCGTCAGACC CCGTAGAAAA GATCAAAGGA |
| 8821 | TCTTCTTGAG ATCCTTTTTT TCTGCGCGTA ATCTGCTGCT TGCAAACAAA AAAACCACCG |
| 8881 | CTACCAGCGG TGGTTTGTTT GCCGGATCAA GAGCTACCAA CTCTTTTTCC GAAGGTAACT |
| 8941 | GGCTTCAGCA GAGCGCAGAT ACCAAATACT GTTCTTCTAG TGTAGCCGTA GTTAGGCCAC |
| 9001 | CACTTCAAGA ACTCTGTAGC ACCGCCTACA TACCTCGCTC TGCTAATCCT GTTACCAGTG |
| 9061 | GCTGCTGCCA GTGGCGATAA GTCGTGTCTT ACCGGGTTGG ACTCAAGACG ATAGTTACCG |
| 9121 | GATAAGGCGC AGCGGTCGGG CTGAACGGGG GGTTCGTGCA CACAGCCCAG CTTGGAGCGA |
| 9181 | ACGACCTACA CCGAACTGAG ATACCTACAG CGTGAGCTAT GAGAAAGCGC CACGCTTCCC |
| 9241 | GAAGGGAGAA AGGCGGACAG GTATCCGGTA AGCGGCAGGG TCGGAACAGG AGAGCGCACG |
| 9301 | AGGGAGCTTC CAGGGGGAAA CGCCTGGTAT CTTTATAGTC CTGTCGGGTT TCGCCACCTC |
| 9361 | TGACTTGAGC GTCGATTTTT GTGATGCTCG TCAGGGGGGC GGAGCCTATG GAAAAACGCC |
| 9421 | AGCAACGCGG CCTTTTTACG GTTCCTGGCC TTTTGCTGGC CTTTTGCTCA CATGTTCTTT |
| 9481 | CCTGCGTTAT CCCCTGATTC TGTGGATAAC CGTATTACCG CCTTTGAGTG AGCTGATACC |
| 9541 | GCTCGCCGCA GCCGAACGAC CGAGCGCAGC GAGTCAGTGA GCGAGGAAGC GGAAGAGCGC |
| 9601 | CCAATACGCA AACCGCCTCT CCCCGCGCGT TGGCCGATTC ATTAATGCAG CTGTGGAATG |
| 9661 | TGTGTCAGTT AGGGTGTGGA AAGTCCCCAG GCTCCCCAGC AGGCAGAAGT ATGCAAAGCA |
| 9721 | TGCATCTCAA TTAGTCAGCA ACCAGGTGTG GAAAGTCCCC AGGCTCCCCA GCAGGCAGAA |
| 9781 | GTATGCAAAG CATGCATCTC AATTAGTCAG CAACCATAGT CCCGCCCCTA ACTCCGCCCA |
| 9841 | TCCCGCCCCT AACTCCGCCC AGTTCCGCCC ATTCTCCGCC CCATGGCTGA CTAATTTTTT |
| 9901 | TTATTTATGC AGAGGCCGAG GCCGCCTCGG CCTCTGAGCT ATTCCAGAAG TAGTGAGGAG |
| 9961 | GCTTTTTTGG AGGCCTAGGC TTTTGCAAAA AG |

SEQ ID NO: 65

| | |
|---|---|
| 1 | CTGCGCGCTC GCTCGCTCAC TGAGGCCGCC CGGGCAAAGC CCGGGCGTCG GGCGACCTTT |
| 61 | GGTCGCCCGG CCTCAGTGAG CGAGCGAGCG CGCAGAGAGG GAGTGGCCAA CTCCATCACT |
| 121 | AGGGGTTCCT GCGGCAATTC AGTCGATAAC TATAACGGTC CTAAGGTAGC GATTTAAATG |
| 181 | GTACCGGGCC CCAGAAGCCT GGTGGTTGTT TGTCCTTCTC AGGGGAAAAG TGAGGCGGCC |
| 241 | CCTTGGAGGA AGGGGCCGGG CAGAATGATC TAATCGGATT CCAAGCAGCT CAGGGGATTG |
| 301 | TCTTTTTCTA GCACCTTCTT GCCACTCCTA AGCGTCCTCC GTGACCCCGG CTGGGATTTA |
| 361 | GCCTGGTGCT GTGTCAGCCC CGGGTGCCGC AGGGGGACGG CTGCCTTCGG GGGGGACGGG |
| 421 | GCAGGGCGGG GTTCGGCTTC TGGCGTGTGA CCGGCGGCTC TAGAGCCTCT GCTAACCATG |
| 481 | TTCATGCCTT CTTCTTTTTC CTACAGCTCC TGGGCAACGT GCTGGTTATT GTGCTGTCTC |
| 541 | ATCATTTTGG CAAAGAATTA CCACCATGGG CTTCGTGAGA CAGATACAGC TTTTGCTCTG |
| 601 | GAAGAACTGG ACCCTGCGGA AAAGGCAAAA GATTCGCTTT GTGGTGGAAC TCGTGTGGCC |
| 661 | TTTATCTTTA TTTCTGGTCT TGATCTGGTT AAGGAATGCC AACCCGCTCT ACAGCCATCA |
| 721 | TGAATGCCAT TTCCCCAACA AGGCGATGCC CTCAGCAGGA ATGCTGCCGT GGCTCCAGGG |
| 781 | GATCTTCTGC AATGTGAACA ATCCCTGTTT TCAAAGCCCC ACCCCAGGAG AATCTCCTGG |
| 841 | AATTGTGTCA AACTATAACA ACTCCATCTT GGCAAGGGTA TATCGAGATT TTCAAGAACT |
| 901 | CCTCATGAAT GCACCAGAGA GCCAGCACCT TGGCCGTATT TGGACAGAGC TACACATCTT |
| 961 | GTCCCAATTC ATGGACACCC TCCGGACTCA CCCGGAGAGA ATTGCAGGAA GAGGAATACG |
| 1021 | AATAAGGGAT ATCTTGAAAG ATGAAGAAAC ACTGACACTA TTTCTCATTA AAAACATCGG |
| 1081 | CCTGTCTGAC TCAGTGGTCT ACCTTCTGAT CAACTCTCAA GTCCGTCCAG AGCAGTTCGC |
| 1141 | TCATGGAGTC CCGGACCTGG CGCTGAAGGA CATCGCCTGC AGCGAGGCCC TCCTGGAGCG |
| 1201 | CTTCATCATC TTCAGCCAGA GACGCGGGGC AAAGACGGTG CGCTATGCCC TGTGCTCCCT |
| 1261 | CTCCCAGGGC ACCCTACAGT GGATAGAAGA CACTCTGTAT GCCAACGTGG ACTTCTTCAA |
| 1321 | GCTCTTCCGT GTGCTTCCCA CACTCCTAGA CAGCCGTTCT CAAGGTATCA ATCTGAGATC |
| 1381 | TTGGGGAGGA ATATTATCTG ATATGTCACC AAGAATTCAA GAGTTTATCC ATCGGCCGAG |
| 1441 | TATGCAGGAC TTGCTGTGGG TGACCAGGCC CCTCATGCAG AATGGTGGTC CAGAGACCTT |
| 1501 | TACAAAGCTG ATGGGCATCC TGTCTGACCT CCTGTGTGGC TACCCCGAGG GAGGTGGCTC |
| 1561 | TCGGGTGCTC TCCTTCAACT GGTATGAAGA CAATAACTAT AAGGCCTTTC TGGGGATTGA |
| 1621 | CTCCACAAGG AAGGATCCTA TCTATTCTTA TGACAGAAGA ACAACATCCT TTTGTAATGC |
| 1681 | ATTGATCCAG AGCCTGGAGT CAAATCCTTT AACCAAAATC GCTTGGAGGG CGGCAAAGCC |
| 1741 | TTTGCTGATG GGAAAAATCC TGTACACTCC TGATTCACCT GCAGCACGAA GGATACTGAA |
| 1801 | GAATGCCAAC TCAACTTTTG AAGAACTGGA ACACGTTAGG AAGTTGGTCA AAGCCTGGGA |
| 1861 | AGAAGTAGGG CCCCAGATCT GGTACTTCTT TGACAACAGC ACACAGATGA ACATGATCAG |
| 1921 | AGATACCCTG GGGAACCCAA CAGTAAAAGA CTTTTTGAAT AGGCAGCTTG GTGAAGAAGG |
| 1981 | TATTACTGCT GAAGCCATCC TAAACTTCCT CTACAAGGGC CCTCGGGAAA GCCAGGCTGA |
| 2041 | CGACATGGCC AACTTCGACT GGAGGGACAT ATTTAACATC ACTGATCGCA CCCTCCGCCT |
| 2101 | TGTCAATCAA TACCTGGAGT GCTTGGTCCT GGATAAGTTT GAAAGCTACA ATGATGAAAC |
| 2161 | TCAGCTCACC CAACGTGCCC TCTCTCTACT GGAGGAAAAC ATGTTCTGGG CCGGAGTGGT |
| 2221 | ATTCCCTGAC ATGTATCCCT GGACCAGCTC TCTACCACCC CACGTGAAGT ATAAGATCCG |
| 2281 | AATGGACATA GACGTGGTGG AGAAAACCAA TAAGATTAAA GACAGGTATT GGGATTCTGG |
| 2341 | TCCCAGAGCT GATCCCGTGG AAGATTTCCG GTACATCTGG GGCGGGTTTG CCTATCTGCA |
| 2401 | GGACATGGTT GAACAGGGGA TCACAAGGAG CCAGGTGCAG GCGGAGGCTC CAGTTGGAAT |
| 2461 | CTACCTCCAG CAGATGCCCT ACCCCTGCTT CGTGGACGAT TCTTTCATGA TCATCCTGAA |
| 2521 | CCGCTGTTTC CCTATCTTCA TGGTGCTGGC ATGGATCTAC TCTGTCTCCA TGACTGTGAA |
| 2581 | GAGCATCGTC TTGGAGAAGG AGTTGCGACT GAAGGAGACC TTGAAAAATC AGGGTGTCTC |
| 2641 | CAATGCAGTG ATTTGGTGTA CCTGGTTCCT GGACAGCTTC TCCATCATGT CGATGAGCAT |
| 2701 | CTTCCTCCTG ACGATATTCA TCATGCATGG AAGAATCCTA CATTACAGCG ACCCATTCAT |
| 2761 | CCTCTTCCTG TTCTTGTTGG CTTTCTCCAC TGCCACCATC ATGCTGTGCT TTCTGCTCAG |
| 2821 | CACCTTCTTC TCCAAGGCCA GTCTGGCAGC AGCCTGTAGT GGTGTCATCT ATTTCACCCT |
| 2881 | CTACCTGCCA CACATCCTGT GCTTCGCCTG GCAGGACCGC ATGACCGCTG AGCTGAAGAA |
| 2941 | GGCTGTGAGC TTACTGTCTC CGGTGGCATT TGGATTTGGC ACTGAGTACC TGGTTCGCTT |
| 3001 | TGAAGAGCAA GGCCTGGGGC TGCAGTGGAG CAACATCGGG AACAGTCCCA CGGAAGGGGA |
| 3061 | CGAATTCAGC TTCCTGCTGT CCATGCAGAT GATGCTCCTT GATGCTGCTG TCTATGGCTT |
| 3121 | ACTCGCTTGG TACCTTGATC AGGTGTTTCC AGGAGACTAT GGAACCCCAC TTCCTTGGTA |
| 3181 | CTTTCTTCTA CAAGAGTCGT ATTGGCTTGG CGGTGAAGGG TGTTCAACCA GAGAAGAAAG |
| 3241 | AGCCCTGGAA AAGACCGAGC CCCTAACAGA GGAAACGGAG GATCCAGAGC ACCCAGAAGG |
| 3301 | AATACACGAC TCCTTCTTTG AACGTGAGCA TCCAGGGTGG GTTCCTGGGG TATGCGTGAA |
| 3361 | GAATCTGGTA AAGATTTTTG AGCCCTGTGG CCGGCCAGCT GTGGACCGTC TGAACATCAC |
| 3421 | CTTCTACGAG AACCAGATCA CCGCATTCCT GGGCCACAAT GGAGCTGGGA AAACCACCAC |
| 3481 | CTTGTCCATC CTGACGGGTC TGTTGCCACC AACCTCTGGG ACTGTGCTCG TTGGGGGAAG |
| 3541 | GGACATTGAA ACCAGCCTGG ATGCAGTCCG GCAGAGCCTT GGCATGTGTC CACAGCACAA |
| 3601 | CATCCTGTTC CACCACCTCA CGGTGGCTGA GCACATGCTG TTCTATGCCC AGCTGAAAGG |
| 3661 | AAAGTCCCAG GAGGAGGCCC AGCTGGAGAT GGAAGCCATG TTGGAGGACA CAGGCCTCCA |
| 3721 | CCACAAGCGG AATGAAGAGG CTCAGGACCT ATCAGGTGGC ATGCAGAGAA AGCTGTCGGT |
| 3781 | TGCCATTGCC TTTGTGGGAG ATGCCAAGGT GGTGATTCTG GACGAACCCA CCTCTGGGGT |
| 3841 | GGACCCTTAC TCGAGACGCT CAATCTGGGA TCTGCTCCTG AAGTATCGCT CAGGCAGAAC |
| 3901 | CATCATCATG TCCACTCACC ACATGGACGA GGCCGACCTC CTTGGGGACC GCATTGCCAT |
| 3961 | CATTGCCCAG GGAAGGCTCT ACTGCTCAGG CACCCCACTC TTCCTGAAGA ACTGCTTTGG |
| 4021 | CACAGGCTTG TACTTAACCT TGGTGCGCAA GATGAAAAAC ATCCAGAGCC AAAGGAAAGG |
| 4081 | CAGTGAGGGG ACCTGCAGCT GCTCGTCTAA GGGTTTCTCC ACCACGTGTC CAGCCCACGT |
| 4141 | CGATGACCTA ACTCCAGAAC AAGTCCTGGA TGGGGATGTA AATGAGCTGA TGGATGTAGT |
| 4201 | TCTCCACCAT GTTCCAGAGG CAAAGCTGGT GGAGTGCATT GGTCAAGAAC TTATCTTCCT |
| 4261 | TCTTCCATTT AAATTAGGGA TAACAGGGTG GTGGCGCGGG CCGCAGGAAC CCCTAGTGAT |
| 4321 | GGAGTTGGCC ACTCCCTCTC TGCGCGCTCG CTCGCTCACT GAGGCCGGGC GACCAAAGGT |
| 4381 | CGCCCGACGC CCGGGCGGCC TCAGTGAGCG AGCGAGCGCG CAGAGCTAGA ATTAATTCCG |
| 4441 | TGTATTCTAT AGTGTCACCT AAATCGTATG TGTATGATAC ATAAGGTTAT GTATTAATTG |
| 4501 | TAGCCGCGTT CTAACGACAA TATGTACAAG CCTAATTGTG TAGCATCTGG CTTAGCGGCC |
| 4561 | GCCTACCGTC AAACAGTCAA TCCCGTTCTA CGCCATTTGA CACATAACGC CCGGGATAAC |
| 4621 | AGAGCTGAAT TTGACGGACT ACGATATTGC TTATGTGCCA CCAATCAACA GTTAACGAAC |
| 4681 | ACGTGGCGGC GCGGAACGCC TCCGGCCAGG CCGCGCGCTT CGCATATTTA CTTCGAGCAG |
| 4741 | TGTAGGTGTG ACAACGTAGC ATGCAGCCAC ATCCCTAGCT TGAACCGGAG ATAAAGGTCT |
| 4801 | ACGCGCGCGA CGTCCACATT CACACGGTTC AGATTCCTGG TGCTACCCAA AACAAAGTCC |
| 4861 | ATAGGTTTTT CATTGGGACT ACGGCGCGAA GCTAAGTGGT TTCACACCTA CAAGGGAAAC |
| 4921 | ATGCCCAAAC TATGAGGACA ACATCGTCCG CAGAAACAAT CGGCCGCGAT AGGGGTTGCA |
| 4981 | CGTTGTCAGA TGAAAGAGCC ACACTCGGGG AGCAGTCCGC GGACGCCACC TCGTGCAACT |
| 5041 | TCGGCTAACC ATATAATCTA AAAAAGTTGA GGTTTGCAGT TGTCGGGGCG AGATCAAACC |
| 5101 | CAAGTATATA GTCCTGTCCG GAGCCTTAGT TCACGTACTC GCGACCCTTG AAAGCGCGTC |
| 5161 | AAGCTTATCG CTCACTGACT AGCTCAATGT GTGGCAATCT AAGTAGGAGG TCTGTCGCAA |
| 5221 | GGCAAAAATG CTAATTATTG GTAGCAAGCT TAGATAAGGT GGAGGGATTG CACAATTCAG |
| 5281 | AAGGCGTCTT CTCTGCTACA CCCGAGCGGG GTGCTTTATC AAGGGGAAGC TTGATGTCCC |
| 5341 | ACGGGATGAA CGAGAGCCTC CATGGCATCT CACGACCTAC TTAACTTCGG GGGATGGGTA |
| 5401 | GAAGTTAGCT GAACATACAA ATGGGAATAG GATTGTGCCC TCGGACGAGA CTGAACGGAT |
| 5461 | CGCAGTCAAC CCGCGCAAAG TTTACATATT AATTCTTACG GCGTGTCAGA GAGGCAATGG |
| 5521 | CTTGACTTGT GGTGGATCAC AGTTTGTGAG TAACGGCAAG ATGCGGTAAA CACTGTAATG |
| 5581 | CGAGCTTCAT TGACTCGGCT TAAAGTTCCT GGTACCATAA TGAATACACG GTGGTTAGTT |
| 5641 | GTCAATTGCT TGTGCACCGC CGCACCTTGC GGTCCTCGGT CCAGCCTGCG CAGGGTATAA |
| 5701 | ATGAAGCACG TCCCACCCAG ACTGTTCCAT CGTACCTCCA AATACGGATT CAACCTGGCG |
| 5761 | TCTATTTCCA GATATGGGCC CTAGGGGTGA TAGACTCCCA AGTCTAAGGA CTACCATGGG |
| 5821 | ATATGTTTCA CGTATCCAAA AAGTAACCAT AATACTGCGT TTCCGTTCAC CCAAGTGAGG |
| 5881 | ATGTTGCCTT TGTACTGGTT TCATAGTCCT GCCGTACCAG GCGTCTTCCT TAGCCGGCGC |
| 5941 | TACTTCCAGC CCGGAACTGT CTTGTTTCTC GATGTGAGAC CCTTGTCAGC CGCCCGCGGT |
| 6001 | GGTGCACGTA AAAGCCGATT GGAGTATTAA GTATTTACAA CTCCGAATCT TAAGAGCCCT |
| 6061 | GCTCTAGTTT GGATTCATAT ATCAGCATAG GCTTCGCAAC CTAGTGAATG AGCGGTACGA |
| 6121 | ACTTTCGCGG AGTGCGAAAA GCGACCGAGC AATCGAGATA CGTACCGTTA GATTCACGCT |
| 6181 | CCAGACAGCA CTCTGAGTCT TTGATTTATA ACCATCGAAG GAATCGACTT CACGTCCCTA |
| 6241 | GCGTGTTGAG TCATCCGCAG AAGAGACGAT GAGGGCTCGC CCCCCGAAAT AGTTCTGCTT |
| 6301 | CAAACTATAG GCTGCCCTAC TTGGTCTCCG AGGTACTATG GGGTCCTCGA CGGTTCGAGG |
| 6361 | CCCCCAACCC ATGTTCAATC AGCTCGTATG TCTACCCTCG AGCTAACACA GGAACCAGCT |
| 6421 | GAGACTTGCC TGGCGTCACT TGGGCACGTT CCATATACAT AATGAAGTAC GCCGCAGGGT |
| 6481 | CTCTCCGTTA CCGAACTGTG CTCGACCTAA AGTCCGGTAC CCATCGGCGT CCTGTCACAT |
| 6541 | TTGTGGCATT AGGTATGAAC TAACTCTGGG GGGCTTCTAC GACCATGGTA AAAGTTTTGT |
| 6601 | GCTGCCAGAC AACTGTTAAT AAACATGTCG CTGCGTAGAA CGCCAAGAAC CAGCTGGGAT |
| 6661 | GAGTGCCTTA TTTACCCCGC GCGAGGTGGG TCTGAGTAGG TAGCATCGAG GTTTACGCCT |
| 6721 | AAGTTGGACC GCAAATATAG GCCCTTTGCC GGGATCCCCA CTATCTGTGA ATTGTGAAAC |
| 6781 | CCGTTGGCAC CCTGTACAAA GTGCATAGCT ACATCATTGG TAACAAGACG TAAACGGAGG |
| 6841 | TTCGCTCACT CCCACTTCGG AAAGATAACC GGGGAACTAG GAGGGTATGG TGCGCGCATG |
| 6901 | GAAAGGGCCG GGAAGTAACT CTGGCCTTCA CGGAACGATA AGTTACAATT TGGGAACAGT |
| 6961 | CGGAGAGCGC CACTACGTGC TTTTTTGGCT TACCTCATAT CTCGTAGTTG GTGAGGGTTA |
| 7021 | AAATTCGCGG GAGAAGATCC AGCCTAAGTA TATGGTTACA TCGCGGCCGC CTGAAGCAGA |
| 7081 | CCCTATCATC TCTCTCGTAA ACTGCCGTCA GAGTCGGTTT GGTTGGACGA ACCTTCTGAG |
| 7141 | TTTCTGGTAA CGCCGTCCCG CACCCGGAAA TGGTCAGCGA ACCAATCAGC AGGGTCATCG |
| 7201 | CTAGCCAGAT CCTCTACGCC GGACGCATCG TGGCCGGCAT CACCGGCGCC ACAGGTGCGG |
| 7261 | TTGCTGGCGC CTATATCGCC GACATCACCG ATGGGGAAGA TCGGGCTCGC CACTTCGGGC |
| 7321 | TCATGAGCGC TTGTTTCGGC GTGGGTATGG TGGCAGGCCG CCCTTAGAAA AACTCATCGA |
| 7381 | GCATCAAATG AAACTGCAAT TTATTCATAT CAGGATTATC AATACCATAT TTTTGAAAAA |
| 7441 | GCCGTTTCTG TAATGAAGGA GAAAACTCAC CGAGGCAGTT CCATAGGATG GCAAGATCCT |
| 7501 | GGTATCGGTC TGCGATTCCG ACTCGTCCAA CATCAATACA ACCTATTAAT TTCCCCTCGT |
| 7561 | CAAAAATAAG GTTATCAAGT GAGAAATCAC CATGAGTGAC GACTGAATCC GGTGAGAATG |
| 7621 | GCAAAAGCTT ATGCATTTCT TTCCAGACTT GTTCAACAGG CCAGCCATTA CGCTCGTCAT |
| 7681 | CAAAATCACT CGCATCAACC AAACCGTTAT TCATTCGTGA TTGCGCCTGA GCGAGACGAA |
| 7741 | ATACGCGATC GCTGTTAAAA GGACAATTAC AAACAGGAAT CGAATGCAAC CGGCGCAGGA |
| 7801 | ACACTGCCAG CGCATCAACA ATATTTTCAC CTGAATCAGG ATATTCTTCT AATACCTGGA |
| 7861 | ATGCTGTTTT CCCGGGGATC GCAGTGGTGA GTAACCATGC ATCATCAGGA GTACGGATAA |
| 7921 | AATGCTTGAT GGTCGGAAGA GGCATAAATT CCGTCAGCCA GTTTAGTCTG ACCATCTCAT |
| 7981 | CTGTAACATC ATTGGCAACG CTACCTTTGC CATGTTTCAG AAACAACTCT GGCGCATCGG |
| 8041 | GCTTCCCATA CAATCGATAG ATTGTCGCAC CTGATTGCCC GACATTATCG CGAGCCCATT |
| 8101 | TATACCCATA TAAATCAGCA TCCATGTTGG AATTTAATCG CGGCCTCGAG CAAGACGTTT |
| 8161 | CCCGTTGAAT ATGGCTCATA ACACCCCTTG TATTACTGTT TATGTAAGCA GACAGTTTTA |
| 8221 | TTGTTCATGA TGATATATTT TTATCTTGTG CAATGTAACA TCAGAGATTT TGAGACACAA |
| 8281 | CGTGGTTTGC AGGAGTCAGG CAACTATGGA TGAACGAAAT AGACAGATCG CTGAGATAGG |
| 8341 | TGCCTCACTG ATTAAGCATT GGTAACTGTC AGACCAAGTT TACTCATATA TACTTTAGAT |
| 8401 | TGATTTAAAA CTTCATTTTT AATTTAAAAG GATCTAGGTG AAGATCCTTT TTGATAATCT |
| 8461 | CATGACCAAA ATCCCTTAAC GTGAGTTTTC GTTCCACTGA GCGTCAGACC CCGTAGAAAA |
| 8521 | GATCAAAGGA TCTTCTTGAG ATCCTTTTTT TCTGCGCGTA ATCTGCTGCT TGCAAACAAA |
| 8581 | AAAACCACCG CTACCAGCGG TGGTTTGTTT GCCGGATCAA GAGCTACCAA CTCTTTTTCC |
| 8641 | GAAGGTAACT GGCTTCAGCA GAGCGCAGAT ACCAAATACT GTTCTTCTAG TGTAGCCGTA |
| 8701 | GTTAGGCCAC CACTTCAAGA ACTCTGTAGC ACCGCCTACA TACCTCGCTC TGCTAATCCT |
| 8761 | GTTACCAGTG GCTGCTGCCA GTGGCGATAA GTCGTGTCTT ACCGGGTTGG ACTCAAGACG |
| 8821 | ATAGTTACCG GATAAGGCGC AGCGGTCGGG CTGAACGGGG GGTTCGTGCA CACAGCCCAG |
| 8881 | CTTGGAGCGA ACGACCTACA CCGAACTGAG ATACCTACAG CGTGAGCTAT GAGAAAGCGC |
| 8941 | CACGCTTCCC GAAGGGAGAA AGGCGGACAG GTATCCGGTA AGCGGCAGGG TCGGAACAGG |
| 9001 | AGAGCGCACG AGGGAGCTTC CAGGGGGAAA CGCCTGGTAT CTTTATAGTC CTGTCGGGTT |
| 9061 | TCGCCACCTC TGACTTGAGC GTCGATTTTT GTGATGCTCG TCAGGGGGGC GGAGCCTATG |
| 9121 | GAAAAACGCC AGCAACGCGG CCTTTTTACG GTTCCTGGCC TTTTGCTGGC CTTTTGCTCA |
| 9181 | CATGTTCTTT CCTGCGTTAT CCCCTGATTC TGTGGATAAC CGTATTACCG CCTTTGAGTG |
| 9241 | AGCTGATACC GCTCGCCGCA GCCGAACGAC CGAGCGCAGC GAGTCAGTGA GCGAGGAAGC |
| 9301 | GGAAGAGCGC CCAATACGCA AACCGCCTCT CCCCGCGCGT TGGCCGATTC ATTAATGCAG |
| 9361 | CTGTGGAATG TGTGTCAGTT AGGGTGTGGA AAGTCCCCAG GCTCCCCAGC AGGCAGAAGT |
| 9421 | ATGCAAAGCA TGCATCTCAA TTAGTCAGCA ACCAGGTGTG GAAAGTCCCC AGGCTCCCCA |
| 9481 | GCAGGCAGAA GTATGCAAAG CATGCATCTC AATTAGTCAG CAACCATAGT CCCGCCCCTA |
| 9541 | ACTCCGCCCA TCCCGCCCCT AACTCCGCCC AGTTCCGCCC ATTCTCCGCC CCATGGCTGA |
| 9601 | CTAATTTTTT TTATTTATGC AGAGGCCGAG GCCGCCTCGG CCTCTGAGCT ATTCCAGAAG |
| 9661 | TAGTGAGGAG GCTTTTTTGG AGGCCTAGGC TTTTGCAAAA AG |

SEQ ID NO: 70

| | |
|---|---|
| 1 | MGFVRQIQLL LWKNWTLRKR QKIRFVVELV WPLSLFLVLI WLRNANPLYS HHECHFPNKA |
| 61 | MPSAGMLPWL QGIFCNVNNP CFQSPTPGES PGIVSNYNNS ILARVYRDFQ ELLMNAPESQ |
| 121 | HLGRIWTELH ILSQFMDTLR THPERIAGRG IRIRDILKDE ETLTLFLIKN IGLSDSVVYL |
| 181 | LINSQVRPEQ FAHGVPDLAL KDIACSEALL ERFIIFSQRR GAKTVRYALC SLSQGTLQWI |
| 241 | EDTLYANVDF FKLFRVLPTL LDSRSQGINL RSWGGILSDM SPRIQEFIHR PSMQDLLWVT |
| 301 | RPLMQNGGPE TFTKLMGILS DLLCGYPEGG GSRVLSFNWY EDNNYKAFLG IDSTRKDPIY |
| 361 | SYDRRTTSFC NALIQSLESN PLTKIAWRAA KPLLMGKILY TPDSPAARRI LKNANSTFEE |
| 421 | LEHVRKLVKA WEEVGPQIWY FFDNSTQMNM IRDTLGNPTV KDFLNRQLGE EGITAEAILN |
| 481 | FLYKGPRESQ ADDMANFDWR DIFNITDRTL RLVNQYLECL VLDKFESYND ETQLTQRALS |
| 541 | LLEENMFWAG VVFPDMYPWT SSLPPHVKYK IRMDIDVVEK TNKIKDRYWD SGPRADPVED |
| 601 | FRYIWGGFAY LQDMVEQGIT RSQVQAEAPV GIYLQQMPYP CFVDDSFMII LNRCFPIFMV |
| 661 | LAWIYSVSMT VKSIVLEKEL RLKETLKNQG VSNAVIWCTW FLDSFSIMSM SIFLLTIFIM |
| 721 | HGRILHYSDP FILFLFLLAF STATIMLCFL LSTFFSKASL AAACSGVIYF TLYLPHILCF |
| 781 | AWQDRMTAEL KKAVSLLSPV AFGFGTEYLV RFEEQGLGLQ WSNIGNSPTE GDEFSFLLSM |
| 841 | QMMLLDAAVY GLLAWYLDQV FPGDYGTPLP WYFLLQESYW LGGEGCSTRE ERALEKTEPL |
| 901 | TEETEDPEHP EGIHDSFFER EHPGWVPGVC VKNLVKIFEP CGRPAVDRLN ITFYENQITA |
| 961 | FLGHNGAGKT TTLSILTGLL PPTSGTVLVG GRDIETSLDA VRQSLGMCPQ HNILFHHLTV |
| 1021 | AEHMLFYAQL KGKSQEEAQL EMEAMLEDTG LHHKRNEEAQ DLSGGMQRKL SVAIAFVGDA |
| 1081 | KVVILDEPTS GVDPYSRRSI WDLLLKYRSG RTIIMSTHHM DEADLLGDRI AIIAQGRLYC |
| 1141 | SGTPLFLKNC FGTGLYLTLV RKMKNIQSQR KGSEGTCSCS SKGFSTTCPA HVDDLTPEQV |
| 1201 | LDGDVNELMD VVLHHVPEAK LVECIGQELI FLLPNKNFKH RAYASLFREL EETLADLGLS |
| 1261 | SFGISDTPLE EIFLKVTEDS DSGPLFAGGA QQKRENVNPR HPCLGPREKA GQTPQDSNVC |
| 1321 | SPGAPAAHPE GQPPPEPECP GPQLNTGTQL VLQHVQALLV KRFQHTIRSH KDFLAQIVLP |
| 1381 | ATFVFLALML SIVIPPFGEY PALTLHPWIY GQQYTFFSMD EPGSEQFTVL ADVLLNKPGF |
| 1441 | GNRCLKEGWL PEYPCGNSTP WKTPSVSPNI TQLFQKQKWT QVNPSPSCRC STREKLTMLP |
| 1501 | ECPEGAGGLP PPQRTQRSTE ILQDLTDRNI SDFLVKTYPA LIRSSLKSKF WVNEQRYGGI |
| 1561 | SIGGKLPVVP ITGEALVGFL SDLGRIMNVS GGPITREASK EIPDFLKHLE TEDNIKVWFN |
| 1621 | NKGWHALVSF LNVAHNAILR ASLPKDRSPE EYGITVISQP LNLTKEQLSE ITVLTTSVDA |
| 1681 | VVAICVIFSM SFVPASFVLY LIQERVNKSK HLQFISGVSP TTYWVTNFLW DIMNYSVSAG |
| 1741 | LVVGIFIGFQ KKAYTSPENL PALVALLLLY GWAVIPMMYP ASFLFDVPST AYVALSCANL |
| 1801 | FIGINSSAIT FILELFENNR TLLRFNAVLR KLLIVFPHFC LGRGLIDLAL SQAVTDVYAR |
| 1861 | FGEEHSANPF HWDLIGKNLF AMVVEGVVYF LLTLLVQRHF FLSQWIAEPT KEPIVDEDDD |
| 1921 | VAEERQRIIT GGNKTDILRL HELTKIYPGT SSPAVDRLCV GVRPGECFGL LGVNGAGKTT |
| 1981 | TFKMLTGDTT VTSGDATVAG KSILTNISEV HQNMGYCPQF DAIDELLTGR EHLYLYARLR |
| 2041 | GVPAEEIEKV ANWSIKSLGL TVYADCLAGT YSGGNKRKLS TAIALIGCPP LVLLDEPTTG |
| 2101 | MDPQARRMLW NVIVSIIREG RAVVLTSHSM EECEALCTRL AIMVKGAFRC MGTIQHLKSK |
| 2161 | FGDGYIVTMK IKSPKDDLLP DLNPVEQFFQ GNFPGSVQRE RHYNMLQFQV SSSSLARIFQ |
| 2221 | LLLSHKDSLL IEEYSVTQTT LDQVFVNFAK QQTESHDLPL HPRAAGASRQ AQD |

### EXAMPLES

### Example 1: Preparation of Upstream and Downstream AAV Vectors

The generation of a given AAV vector comprised three plasmids: pTransgene, pRepCap and pHelper. pTransgene contains either the upstream or downstream ABCA4 transgene as detailed below (ITR integrity confirmed). pRepCap contains the rep and cap genes of the AAV genome. The rep genes are from the AAV2 genome whereas the cap genes varies depending on serotype requirement. pHelper contains the required adenoviral genes necessary for successful AAV generation. The plasmids are complexed with polyethylenimine (PEI) for a triple transfection mix that is applied to HEK293T cells, and HEK293T cells were transfected using a typical PEI protocol to deliver the required plasmids: pRepCap, pHelper (pDeltaAdF6) and pTransgene. HEK293T cells were grown in HYPERFlasks (SLS, UK) and transfected using a typical PEI protocol to deliver a total of 500 pg of the required plasmids: pRepCap, pHelper (pDeltaAdF6) and pTransgene. Cells were harvested three days post-transfection, lysed and the AAV population isolated by ultracentrifugation with an iodixanol gradient followed by purification in Amicon Ultra-15 100K filter units (MerckMillipore, UK). Three days post-transfection, the cells were collected and lysed. The lysate was treated with Benzonase and clarified before applying to an iodixanol gradient comprised of 15%, 25%, 40% and 60% phases. The gradients were spun at 59,000 rpm for 1 hour 30 minutes and the 40% fraction was then withdrawn. This AAV phase was then purified and concentrated using an Amicon Ultra-15 100K filter unit. Following this step, 100-200 μl of purified AAV is obtained in PBS. The final preparations were collected in PBS. SDS-PAGE analysis was used to confirm good purification of each preparation and qPCR titres were determined using primers targeting either the upstream (FW 5′GCACCTTGGCCGTATTTGGACAG, REV 5′TGAGTCAGACAGGCCGATGT) or downstream (FW 5′TGGCGCAGATCGTGCT, REV 5′ACAGAAGGAGTCTTCCA) portion of ABCA4 coding sequence. Primer sets were confirmed to have 95-105% efficiency.

### Example 2—Structure and Cloning of Exemplary AAV Vectors

Adeno-associated virus (AAV) is the current vector of choice for retinal gene therapy due to its ability to diffuse through the various cell layers within the retinal structure, low immunogenicity, excellent tropism for photoreceptor cells and extensive proof of concept in a variety of pre-clinical models. Human clinical trials have shown safety and efficacy with AAV vectors in the retina and gene therapy trials for multiple conditions have been reported in the past decade with more currently ongoing. For some disorders such as Stargardt disease, the therapeutic genes are too large to fit within a transgene that can be packaged into a single AAV capsid. Gene therapy replacement for these disorders is therefore an intriguing challenge. Given the restricted packaging capacity of AAV, its potential to treat “large gene” diseases initially seemed limited, yet more recent studies have indicated that AAV gene therapy delivery of genes over 3.5 kb in size using two or more AAV particles is a distinct possibility.

Different AAV dual vector systems exist: 1. fragmented AAV (fAAV); 2. trans-splicing dual vectors; 3. overlapping dual vectors; and 4. hybrid dual vectors. However, the unpredictability of both the fAAV and trans-splicing methods is likely to raise regulatory concerns. The original dual vector approaches using fragmented transgenes have fallen out of favor due to concerns relating to random truncation and recombination. The alternative hybrid and overlapping dual vector systems rely on a region of homologous overlap between two transgenes. The overlapping approach is the least explored of these strategies yet it is the simplest dual vector design. Dual vector strategies that rely on a region of homologous overlap between two transgenes can be precisely predicted and replicated.

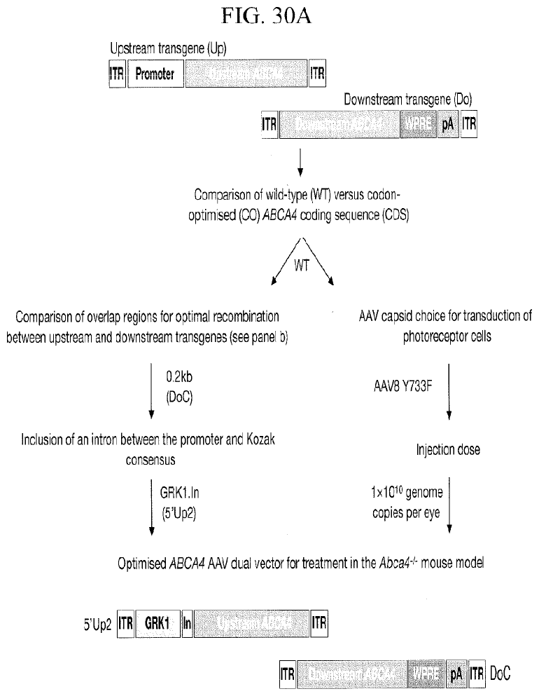

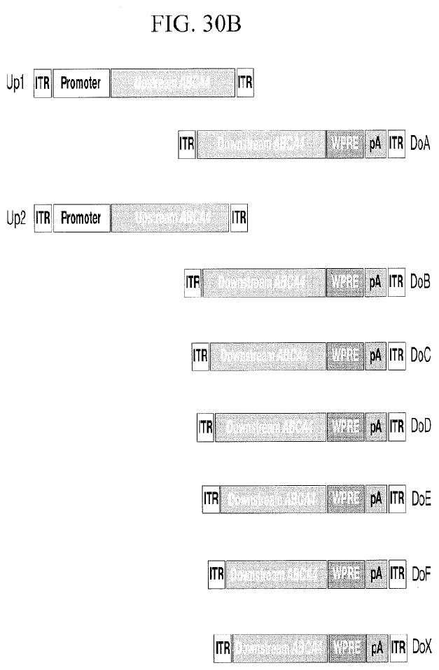

Both the hybrid and overlapping dual vector systems rely on a region of homologous overlap between two transgenes. The region of overlap has been shown to influence the success of transgene reformation. Previous studies have suggested that the success of the overlapping approach relies on homologous recombination (HR), of which there are different forms involved in DNA repair mechanisms, and through one of these sub-pathways the two overlapping dual vector transgenes (on plus and minus strands) may be recombined. The effectiveness of these molecular mechanisms may be tissue-dependent. In the case of Stargardt disease, the target cells are terminally differentiated photoreceptors and both non-homologous end-joining (NHEJ) and homologous recombination (HR) mechanisms are active in mouse rod photoreceptor cells. If consistently correct reformation of the larger transgene occurs then it is an indicator of a Homologous Recombination (HR) pathway being involved. Through systematic vector design variations assessing different overlap regions, codon-optimization of the coding sequence and inclusion of untranslated genetic elements (FIG. 30) we achieved therapeutic levels of our target protein and reduced the production of truncated protein forms that are a known problem in dual vector strategies. Systematic design variations of an overlapping dual vector system achieved therapeutic levels of the target protein, ATP-binding cassette transporter protein family member 4 (ABCA4) and reduced the production of truncated protein forms that are a known side effect of dual vector strategies.

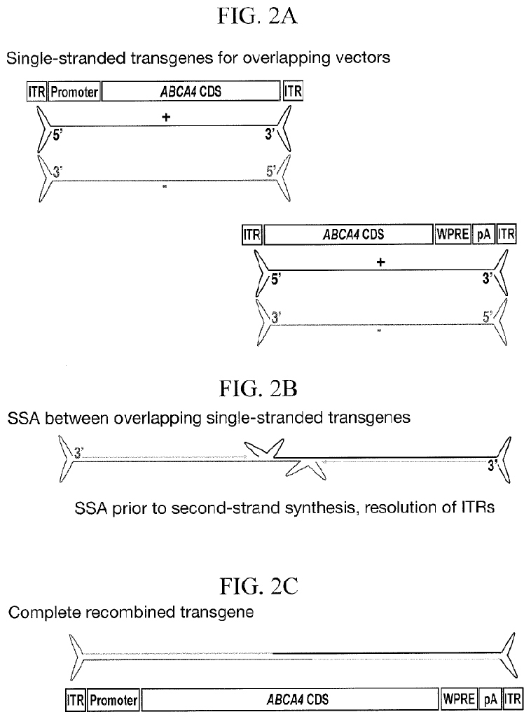

In a normal recombinant AAV scenario, double-stranded transgenes are formed either by recruitment of the corresponding plus and minus single-stranded DNA (ssDNA) transgene forms by single-strand annealing (SSA) or by second-strand synthesis. Mechanisms of recombination between two overlapping transgenes could therefore also occur by SSA of complementary regions from opposing transgenes (FIG. 2). The resulting structures would mimic a situation requiring the HR DNA repair RAD51-independent mechanism, although a RAD50-dependent mechanism would theoretically also be possible, as has been implicated in the fAAV approach.

The disclosure provides compositions and methods for increasing the levels of ABCA4 protein by, for example, exploring different lengths of the overlap region when delivered by dual overlapping vectors. Additionally, the disclosure aims to increase expression from successfully recombined transgenes through the use of codon-optimized coding sequence and inclusion of untranslated regions (UTRs). Codon-optimization can increase the rate of translation of a given coding sequence and recent pre-clinical studies have indicated potential benefits of such in gene therapy transgenes. 5′UTR structures, in particular spliceable introns between the promoter and coding sequence, can enhance expression from transgenes. Spliced mRNAs may have enhanced translational efficiency compared to identical mRNAs not generated through splicing, and addition of intron 2 of the rabbit β-globin (RBG) gene in the 5′UTR of a transgene previously led to a 500-fold increase in protein expression. The outcome of including a 5′UTR containing spliceable intron/exon elements in combination with the 3′UTR Woodchuck hepatitis virus post-transcriptional regulatory element (WPRE) was assessed. The WPRE has previously been shown to increase transcript stability and enhance transgene expression in pre-clinical studies.

There are various elements that can enhance the success of an overlapping dual vector system for the treatment of Stargardt disease. Mutations in ATP-binding cassette transporter protein family member 4 (ABCA4) prevent the transport of retinoids from photoreceptor cell disc outer membranes to the retinal pigment epithelium (RPE), which leads to a build-up of undesired retinoid derivatives in the photoreceptor outer segments. Due to constant generation of photoreceptor outer segments, as older discs become more terminal they are consumed by the RPE. In photoreceptor cells carrying mutant, non-functional ABCA4, bisretinoids retained in the disc membranes build up in the RPE cells with further biochemical processes taking place that lead to formation of the toxicity compound A2E, a key element of lipofuscin. The outcome of this accumulation may be death of the RPE cells with subsequent degeneration and death of the photoreceptor cells, which rely on the RPE cells for survival. There are previously characterized the fundal changes in the pigmented Abca4^{-/-} mouse model, and deuterised vitamin A has a positive effect on fundus fluorescence and bisretinoid accumulation. Hence this mouse is an appropriate model in which to assess therapeutic effects that are relevant to the human condition. Delivering functional ABCA4 to the photoreceptor outer segments of Abca4^{-/-} mice to a level of efficacy that would reduce accumulation of bisretinoids/A2E/lipofuscin, may, in a patient with Stargardt disease, prevent death of the RPE cells and the degeneration of the photoreceptor cells they support.

Described below are embodiments of an overlapping dual AAV vector system that utilizes the endogenous DNA repair pathways of the targeted cell to reconstitute a functional, large ABCA4 transgene for gene therapy.

The ABCA4 coding sequence NM_000350 was used as the WT form with the exception of the following nucleotide changes that did not influence the amino acid sequence: 1,536 G>T; 5,175 G>A; 6,069 T>C (numbered herein according to the ABCA4 coding sequence, SEQ ID NO: 11). Codon-optimization of this ABCA4 coding sequence was performed and generated by GenScript (Piscataway, N.J., US). WT and CO ABCA4 full length coding sequence (6,822 nucleotides) were inserted into plasmids containing AAV2 ITRs to generate CAG.ABCA4.pA and CAG.coABCA4.pA. Upstream transgenes for dual vector in vitro comparisons contained a shortened version of CAG using only the CMV. CBA enhancer/promoter elements prior to the ABCA4 coding sequence fragment. These constructs were generated by amplifying the CMV. CBA elements of CAG and attaching them to the desired ABCA4 coding sequence fragment (see Table 2) by PCR before cloning the entire fragment in between the AAV2 ITRs using SwaI restriction sites. Upstream transgenes for in vivo experiments were prepared in the same way except the GRK1 promoter was amplified and attached to the desired ABCA4 coding sequence fragment by PCR before being ligated (inserted) into the AAV plasmid. For the optimized upstream transgene, 176 nucleotides of the CAG intron/exon region were amplified and attached to the end of the GRK1 promoter by PCR technique. This amplicon was then attached using PCR to the desired ABCA4 coding sequence and inserted between the ITRs using SwaI. Downstream transgenes were identical for in vitro and in vivo use, the desired fragments of ABCA4 coding sequence (see Table 2) were amplified and attached to WPRE and bovine growth hormone polyA signal by PCR before being inserted into ITR containing plasmids using SwaI restriction sites.

### Upstream Vector

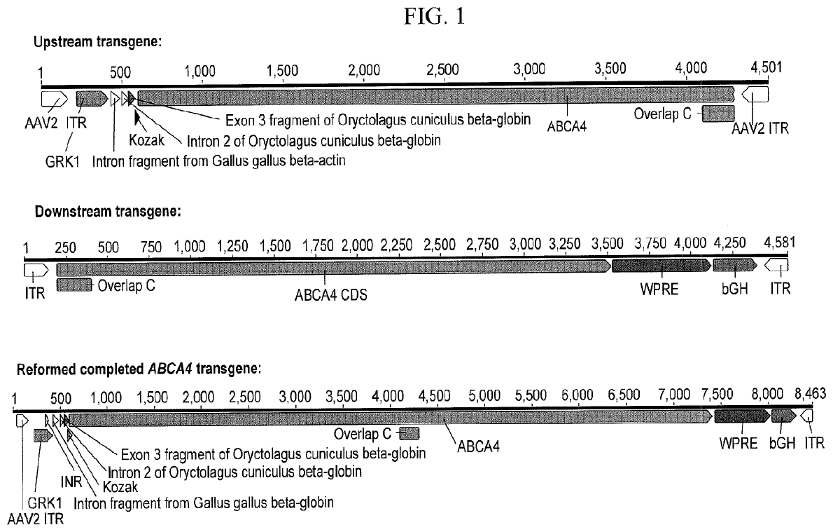

This vector contains a promoter, untranslated region (UTR) and upstream segment of ABCA4 CDS with an AAV2 ITR at each end of the transgene (FIG. 1). ABCA4 is expressed in photoreceptor cells of the retina and therefore a human rhodopsin kinase (GRK1) promoter element has been incorporated. The specific GRK1 promoter sequence contained in the upstream vector is as described by Khani et al. (Investigative Ophthalmology and Visual Science, 48(9), 3954-3961, 2007) comprising of nucleotides−112 to +87 of the GRK1 gene and has been used in pre-clinical studies for gene therapy targeting the photoreceptor cells.

The 199 nucleotides of the GRK1 promoter are followed by an untranslated region (UTR) 186 nucleotides in length. This nucleotide sequence was selected from the larger UTR (443 nucleotides) contained in the REP1 clinical trial vector (MacLaren et al., 2014). Specifically, the selected sequence includes a Gallus β-actin (CBA) intron 1 fragment (with predicted splice donor site), Oryctolagus cuniculus β-globin (RBG) intron 2 fragment (including predicted branch point and splice acceptor site) and Oryctolagus cuniculus β-globin exon 3 fragment immediately prior to the Kozak consensus, which leads into the ABCA4 CDS. This UTR fragment has been added to the original GRK1 promoter element to increase translational yield (Rafiq et al., 1997; Chatterjee et al., 2009). By itself, the GRK1 promoter has shown very good gene expression capabilities in photoreceptor cells.

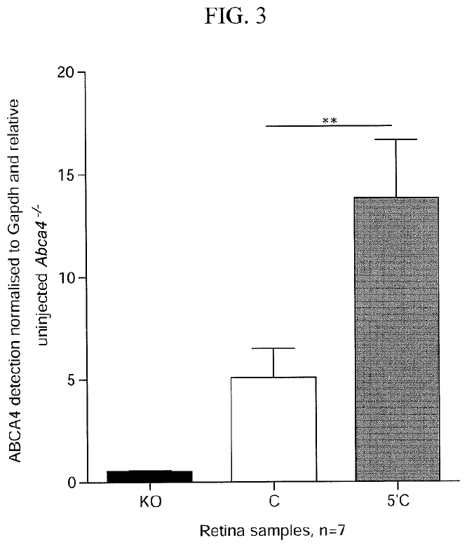

Comparison of dual vector injected Abca4^{-/-} retinae reveals more ABCA4 protein is generated from eyes in which the upstream vector carries the GRK1.5′UTR element compared to the GRK1 promoter element alone (FIG. 3).

Having determined the optimal overlap sequence within the human ABCA4 coding sequence that both improved recombination efficiency and limited production of tABCA4, the dual vector system was optimized to further to increase full length ABCA4 expression levels from successfully recombined transgenes. Original transgene designs for in vivo assessments included the GRK1 promoter element with a portion of the ABCA4 coding sequence in the upstream transgene and ABCA4 coding sequence, WPRE and polyA signal in the downstream transgene. While the GRK1 promoter drives good expression in mouse photoreceptor cells, various elements could improve yield. Inclusion of an intron within a transgene has been shown to improve translational yield and in the case of a dual vector system, this could contribute to achieving the level of target protein required to elicit a therapeutic effect. To investigate the influence of a 5′UTR sequence containing a spliceable element, a region from the 5′UTR similar to an intron used in an AAV2/2 CAG vector was inserted.

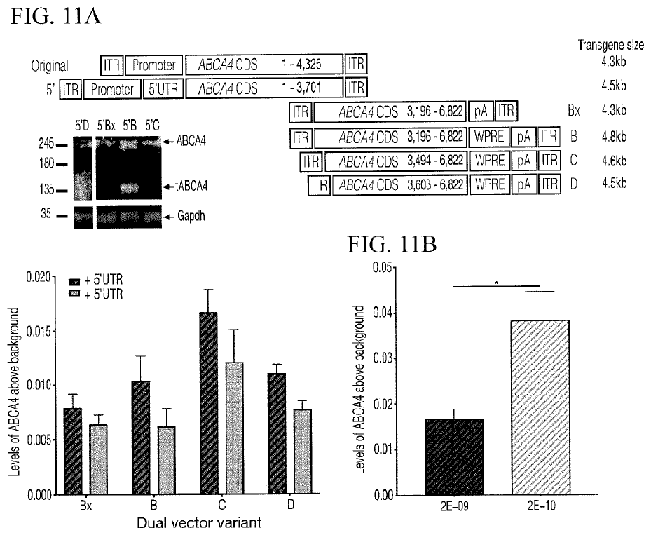

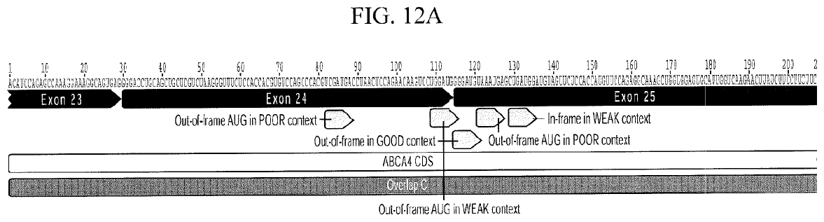

The effect of the 5′ UTR can be seen in FIG. 11. 176 nucleotides were inserted between the GRK1 promoter and Kozak consensus of upstream transgene variant B. The selected sequence contained a chicken (Gallus gallus) β-actin (CBA) intron 1 fragment with predicted splice donor site; a rabbit (Oryctolagus cuniculus) β-globin (RBG) intron 2 fragment including a predicted branch point and splice acceptor site and a rabbit (Oryctolagus cuniculus) β-globin exon 3 fragment immediately prior to the Kozak consensus and human ABCA4 coding sequence. The influence of this additional feature was tested in vivo by comparing the ABCA4 expression achieved following treatment with four dual vector variants with and without the 5′UTR in the upstream transgene: Overlap B (B), Overlap B without WPRE (Bx), Overlap C (C) and Overlap D (D). Detection of ABCA4 protein by western blot 6 weeks post-injection was influenced by both the overlap region and the inclusion of a 5′UTR in the upstream transgene (FIG. 11). FIG. 11 shows a comparison of ABCA4 detection following sub-retinal injection in Abca4-eyes of four dual vector variants with and without a 5′UTR in the upstream transgene. Nucleotides of the ABCA4 cDNA sequence included in each transgene are shown. Detection of full length ABCA4 protein was normalized to GAPDH per sample and presented as levels above untreated negative control samples. In FIG. 11a, Abca4^{-/-} eyes injected with AAV2/8 Y733F variants were assessed 6 weeks post-injection and ABCA4 levels for the effect of a 5′UTR in the upstream transgene (two-way ANOVA, n=3 (Bx/5′D), 5 (B/C), 6 (5′Bx/D), 7 (5′B/5′C), 5′UTR influence p=0.03, overlap influence p=0.005, interaction ns). In FIG. 11b, more full length ABCA4 was detected from Abca4^{-/-} eyes that received a sub-retinal injection of 2E+10 total genome copies of the optimized dual vector variant 5′C compared to eyes that received 2E+9 total genome copies (unpaired non-parametric Mann Whitney test, n=9 & 17, *p=0.01).

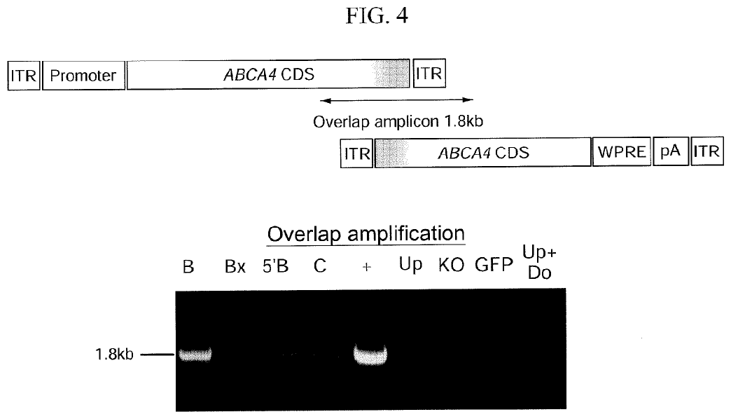

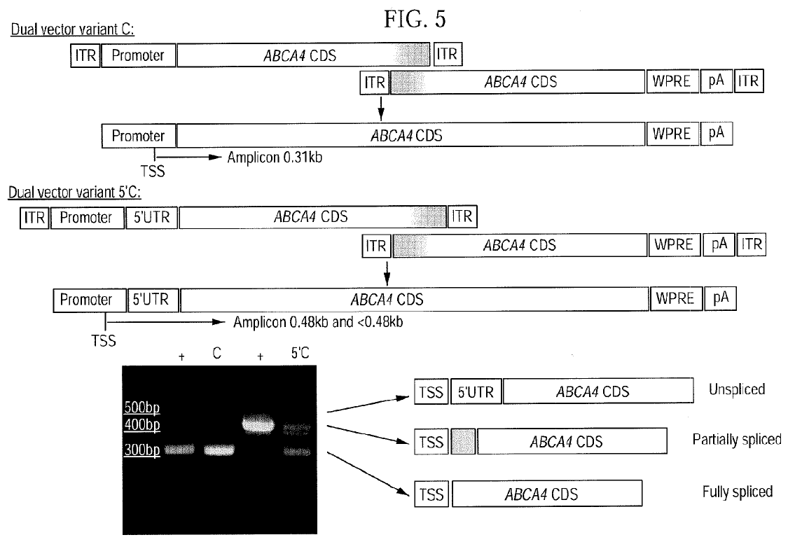

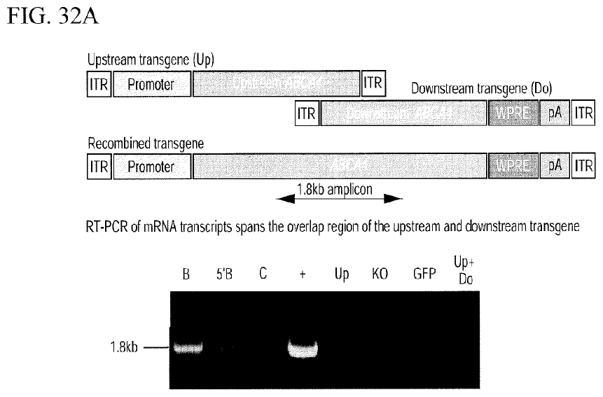

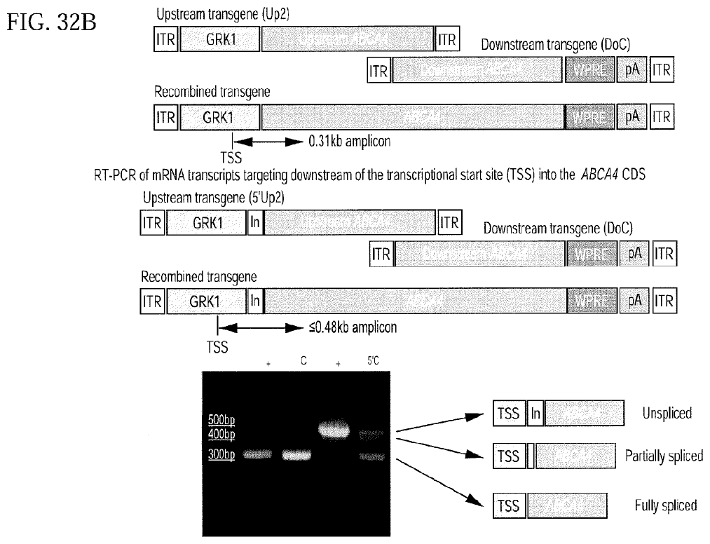

Neural retinae were harvested for mRNA extraction and subsequent RT-PCR and sequencing analysis spanning the overlap zones confirmed that ABCA4 transcripts generated from recombined transgenes did not carry mutations (FIG. 32A). Further RT-PCRs confirmed the intron included in optimized dual vector system (5′C) was successfully spliced from mRNA transcripts (FIG. 32B). RT-PCR analysis spanning the overlap zones confirmed that mRNA ABCA4 transcripts from recombined transgenes were present and of the correct sequence (FIG. 4). Further RT-PCRs were conducted to confirm splicing of the 5′UTR in eyes injected with the optimized dual vector system (FIG. 5). The 5′UTR sequence selected for use in the upstream transgene was predicted to be a spliceable element and to confirm this, pooled cDNA from four Abca4^{-/-} eyes injected with either dual vector variant C or variant 5′C were amplified using a forward primer binding downstream of the GRK1 transcription start site (TSS) and a reverse primer binding within the ABCA4 coding sequence. Exemplary primers to assess 5′ UTR splicing comprise FW 5′CCACTCCTAAGCGTCCTC and REV 5′CAGGGATTGTTCACATTGC. The cDNA from variant C injected eyes generated a single amplicon that sequencing confirmed to exactly match the reference sequence from the GRK TSS to the ABCA4 coding sequence. Amplifications of cDNA from eyes injected with the optimized dual vector variant 5′C generated three products. Sequencing determined these to be three splice variations: one form was unspliced with the 5′UTR intact between the TSS and the ABCA4 coding sequence; the second product represented a partially-spliced form; the final amplification product exhibited complete removal of the 5′UTR and matched the cDNA sequence from variant C injected eyes.

Following the Kozak consensus in the upstream vector is the ABCA4 CDS from nucleotide 1 to 3,701 (105 to 3,805 in NCBI reference file NM_000350). The final 208 nucleotides of the ABCA4 CDS form the first 208 nucleotides of CDS contained in the downstream vector and serve as the overlap zone. The coding sequence fragment contained in the upstream vector matches the reference sequence NM_000350 with the exception of a base change at nucleotide 1,536 (NM_000350 1,640) G>T. This is the third base of the codon and does not result in an amino acid sequence change. The ABCA4 CDS is truncated within exon 25 with the 3′ITR downstream of this.

### Downstream Vector

This vector contains the downstream segment of ABCA4 CDS, a Woodchuck hepatitis virus post-transcriptional response element (WPRE) and bovine growth hormone poly-adenylation signal (bGH polyA) with an AAV2 ITR at each end of the transgene (FIG. 1 and FIG. 2). The ABCA4 CDS begins downstream of the 5′ITR at position 3,494 (NM_000350 3,598) and continues to the stop codon at 6,822 (NM_000350 6,926). The first 208 nucleotides of the ABCA4 CDS are the same as the final 208 ABCA4 CDS nucleotides contained in the upstream vector and serve as the overlap zone between transgenes. The coding sequence fragment contained in the downstream vector matches the reference sequence NM_000350 with the exception of a base change at nucleotide 5,175 (NM_000350 5,279) G>A and 6,069 (NM_000350 6,173) T>C. These changes both occur in the third base of a codon and do not result in an amino acid sequence change.

The restriction site HindIII separates the ABCA4 CDS stop codon from the WPRE. This element is 593 nucleotides in length and matches the X antigen inactivated WPRE contained in the REP1 clinical trial vector. A restriction site for SphI then separates the WPRE from the bGH poly A signal, which is 269 nucleotides in length and matches the bGH poly A signal present in the REP1 clinical trial vector. The 3′ITR then lies downstream of the polyA signal.

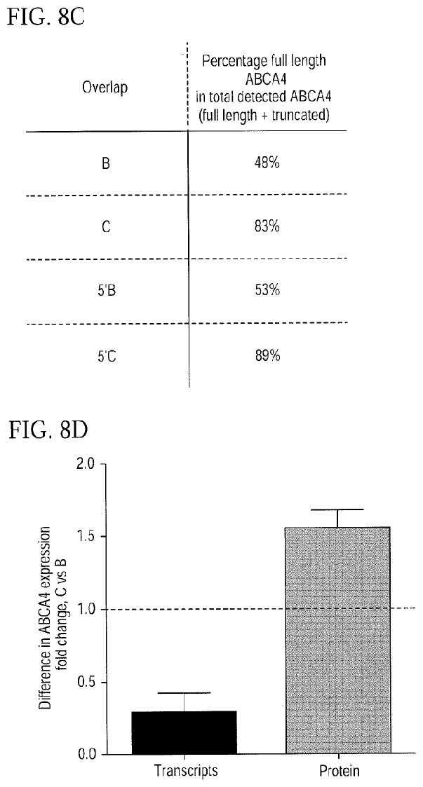

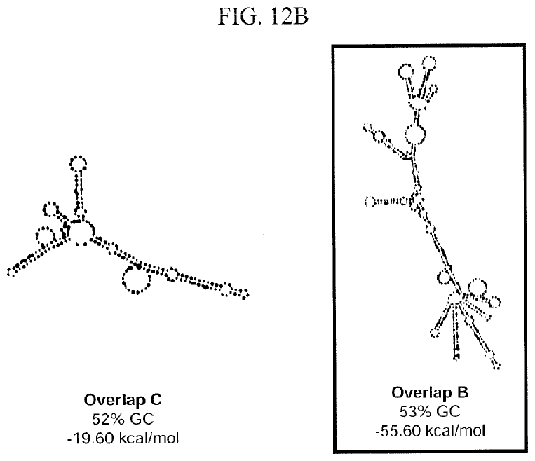

The AAV2 5′ITR is known to have promoter activity and with the WPRE and bGH poly A signal within the downstream transgene, stable transcripts will be generated from unrecombined downstream vectors. The wild-type ABCA4 CDS contained in the downstream transgene carries multiple in-frame AUG codons that cannot be substituted for other codons without altering the amino acid sequence. This creates the possibility of translation occurring from the stable transcripts, leading to the presence of truncated ABCA4 peptides that are detectable by western blot (FIG. 8a). The starting sequence of the chosen overlap zone was carefully selected to include an out-of-frame AUG codon in good context (regarding potential Kozak consensus) prior to an in-frame AUG codon in weaker context (FIG. 12a) in order to encourage the translational machinery to initiate from an out-of-frame site. There are in total four out-of-frame AUG codons in various contexts prior to the in-frame AUG. All of these would translate to a STOP codon within 10 amino acids. The existence of these out-of-frame AUG codons may prevent translation of truncated ABCA4 proteins from unrecombined downstream transgenes.

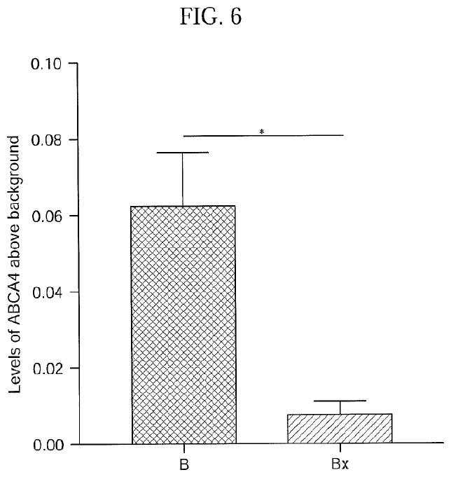

In some embodiments of the dual overlapping vectors, the presence or absence of a WPRE affected protein expression from the dual overlapping vectors. Protein expression from the vectors with overlap zone B is shown in FIG. 6. Full length ABCA4 protein was detected in from HEK293T cells transduced with the AAV2/8 Y733F dual vector variant B. Dual vector variant B with the WPRE generated more ABCA4 than those treated with dual vector variant B without the WPRE (Bx) (unpaired two-tailed parametric t test, n=3, *p=0.01, F(2,2)=17.06). Error bars represent SEM.

### Example 3—Assessment of Overlap Zones

Having identified an optimal vector and ABCA4 sequence to use for recombination, the effects of varying the overlap length of base pairing between plus and minus strands were assessed.

TABLE 2
Transgene Information (nucleotide numbering in Table 2 is relative to ABCA4 CDS, SEQ ID NO: 11).

| Upstream | Short | Transgene | ABCA4 | Downstream | Short | Transgene | ABCA4 | Overlap | | GC content | Dual vector/ |
|---|---|---|---|---|---|---|---|---|---|---|---|
| transgene | name | length | CDS | transgene | name | length | CDS | length | | of overlap | overlap name |
| GRK1.coABC | COu | 4.9 kb | 1-4,326 | coCA4.WPRE.pA | COd | 4.9 kb | 3,154-6,822 | 1.1 | kb | 55% | coA |
| GRK1.ABCAa | Up1 | 4.9 kb | 1-4,326 | aCA4.WPRE.pA | DoA | 4.9 kb | 3,154-6,822 | 1.1 | kb | 55% | A |
| GRK1.ABCb | Up2 | 4.3 kb | 1-3,701 | bCA4.WPRE.pA | DoB | 4.8 kb | 3,196-6,822 | 0.5 | kb | 54% | B |
| GRK1.ABCb | Up2 | 4.3 kb | 1-3,701 | cCA4.WPRE.pA | DoC | 4.6 kb | 3,494-6,822 | 0.2 | kb | 52% | C |
| GRK1.ABCb | Up2 | 4.3 kb | 1-3,701 | dCA4.WPRE.pA | DoD | 4.5 kb | 3,603-6,822 | 0.1 | kb | 48% | D |
| GRK1.ABCb | Up2 | 4.3 kb | 1-3,701 | eCA4.WPRE.pA | DoE | 4.4 kb | 3,653-6,822 | 0.05 | kb | 47% | E |
| GRK1.ABCb | Up2 | 4.3 kb | 1-3,701 | fCA4.WPRE.pA | DoF | 4.4 kb | 3,678-6,822 | 0.02 | kb | 38% | F |
| GRK1.ABCb | Up2 | 4.3 kb | 1-3,701 | xCA4.WPRE.pA | DoX | 4.3 kb | 3,702-6,822 | 0 | kb | N/A | X |
| GRK1.5′ABCb | 5′Up2 | 4.5 kb | 1-3,701 | cCA4.WPRE.pA | DoC | 4.6 kb | 3,494-6,822 | 0.2 | kb | 52% | 5′C |

Table 2 contains transgene details for the dual vector combinations tested, numbered relative to the ABCA4 coding sequence (SEQ ID NO: 11). The final row contains the details for the optimized overlapping dual vector system. ABCA4=ATP-binding cassette transporter protein family member 4; CDS=coding sequence; GRK1=human rhodopsin kinase promoter; pA=polyA signal; WPRE=Woodchuck hepatitis virus post-transcriptional regulatory element.

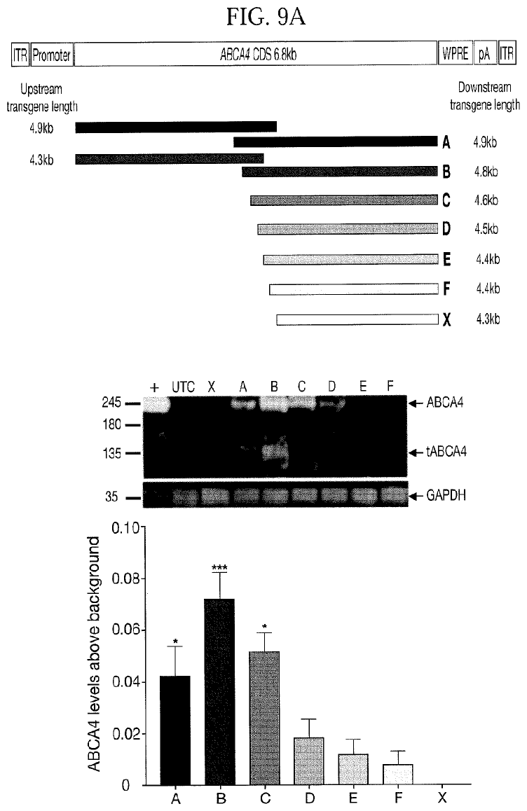

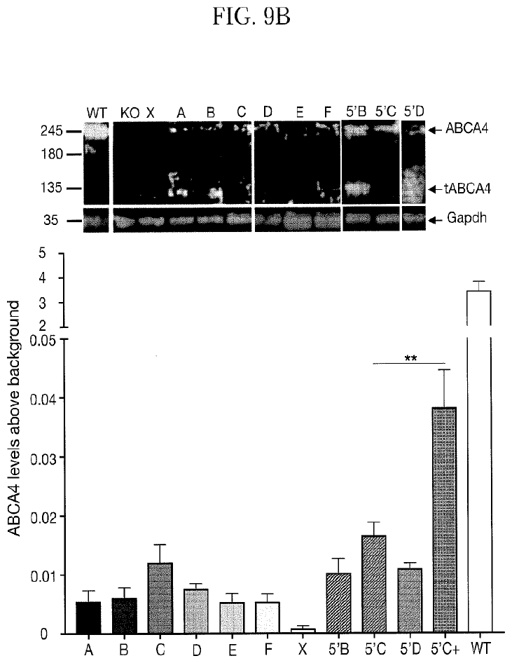

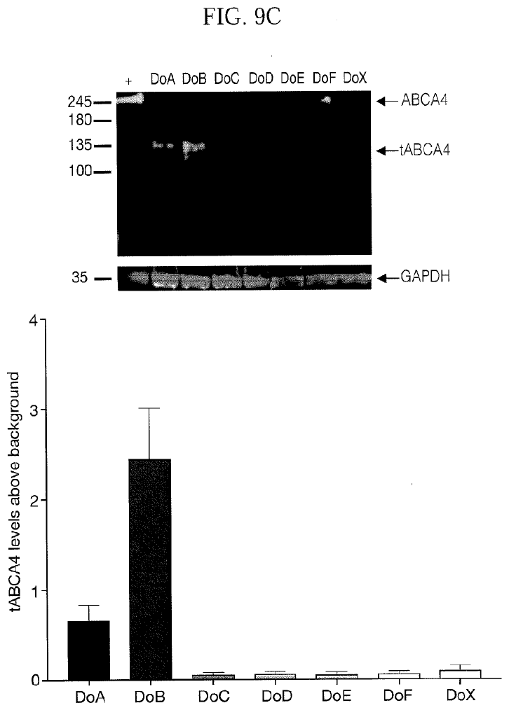

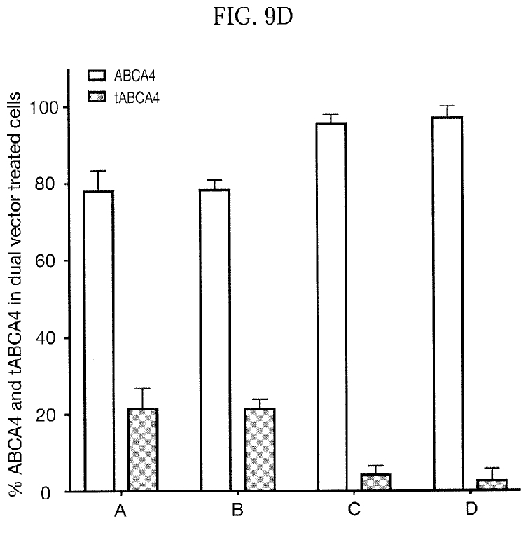

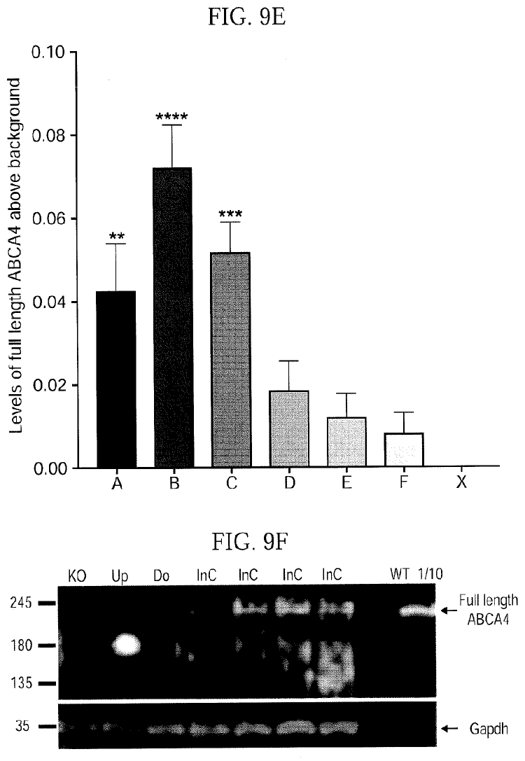

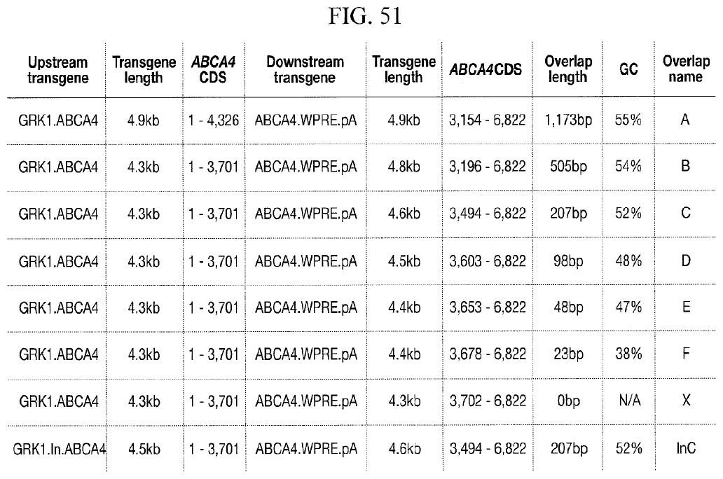

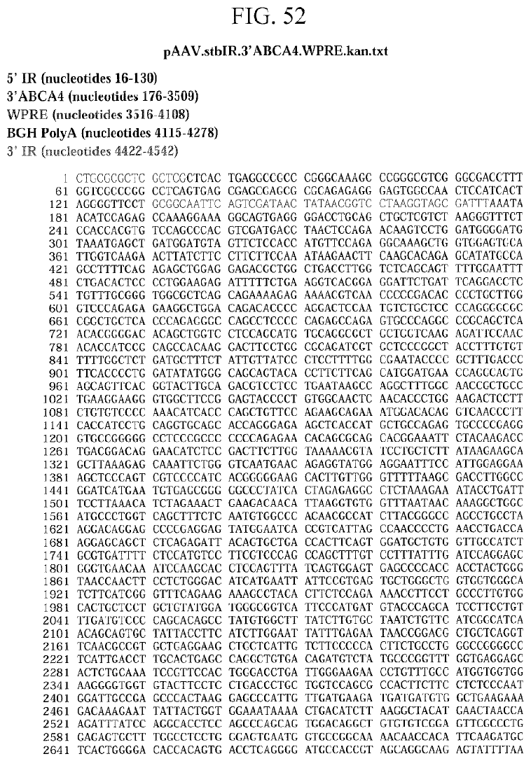

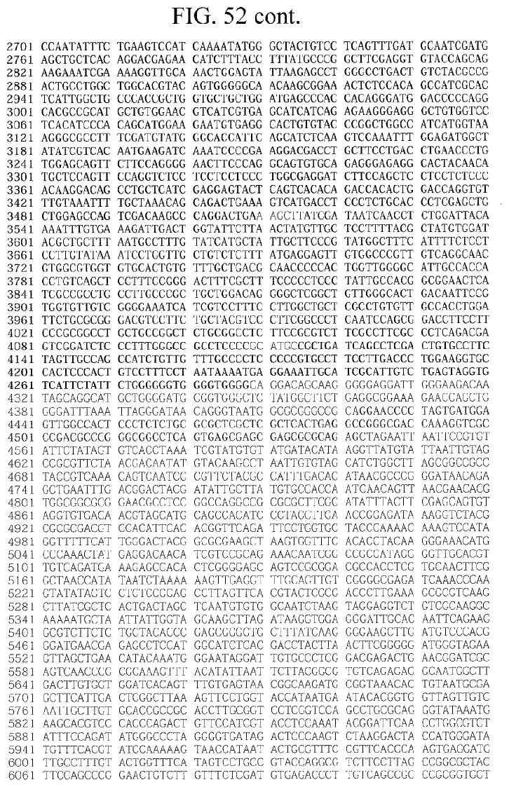

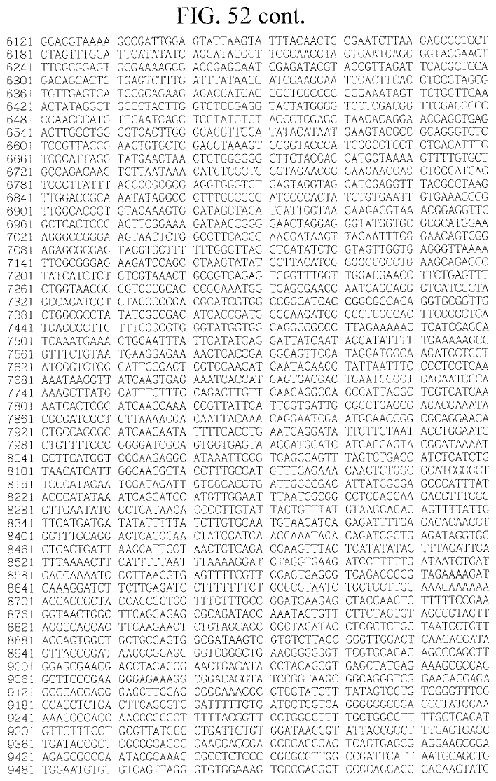

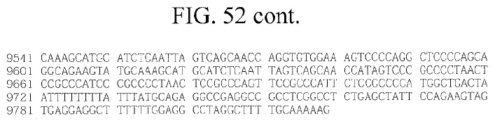

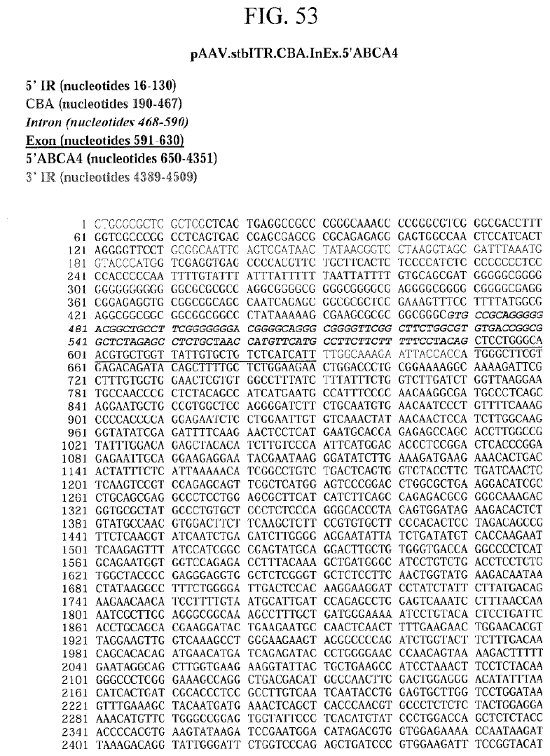

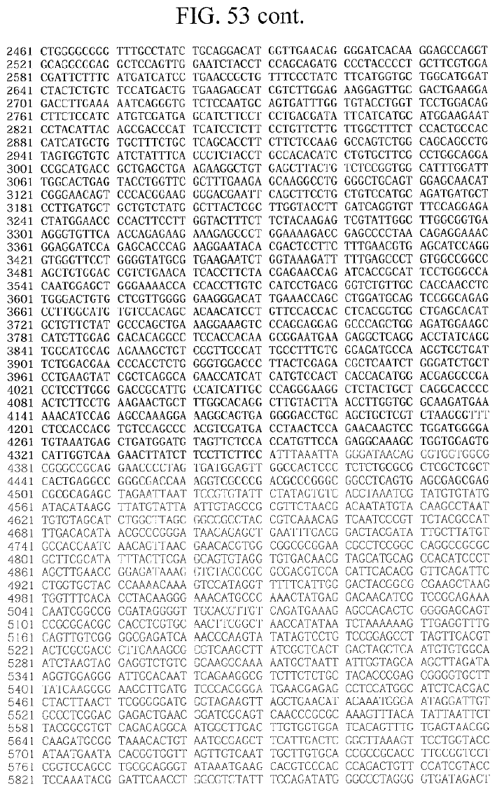

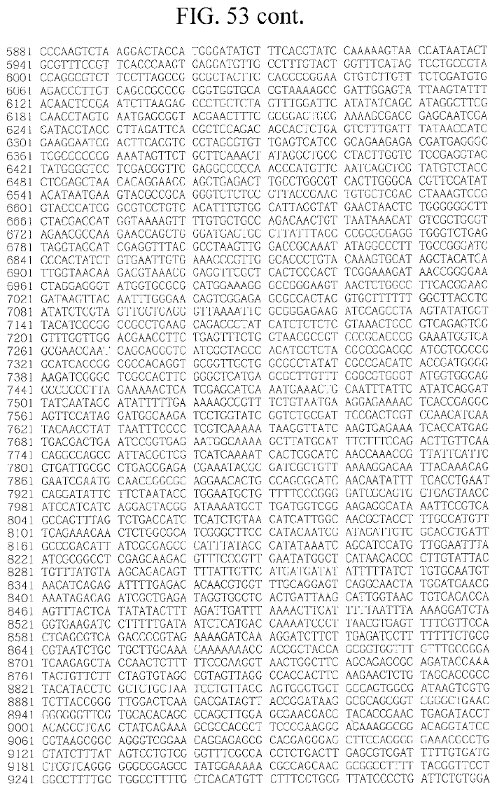

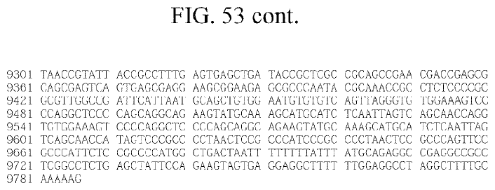

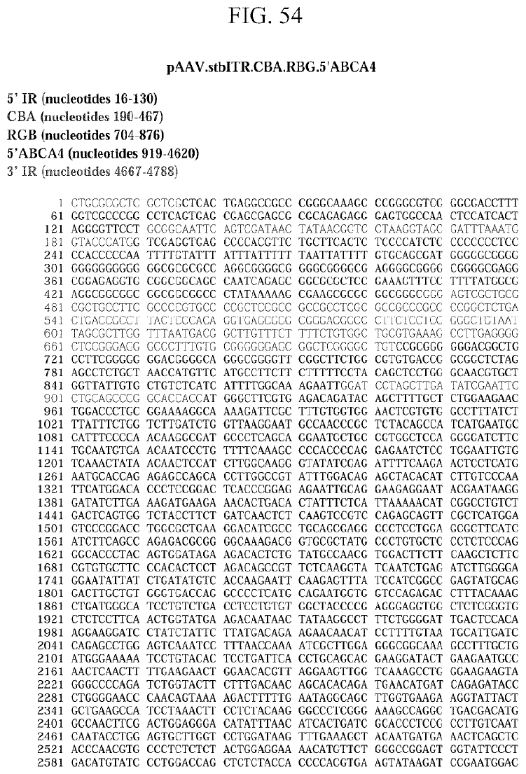

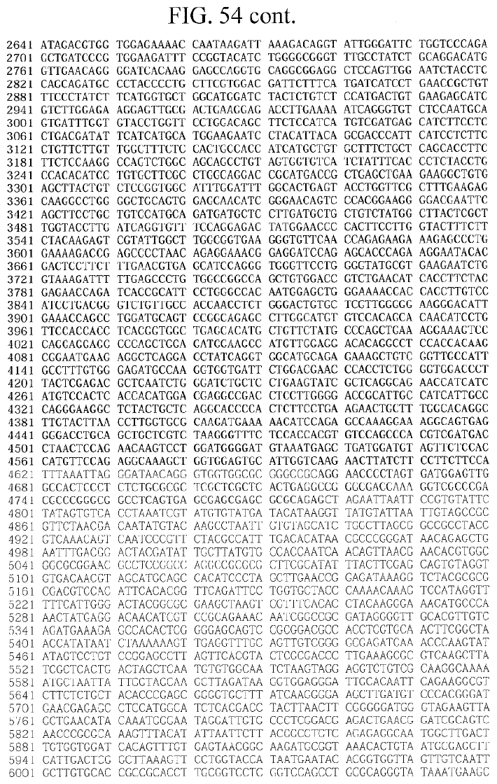

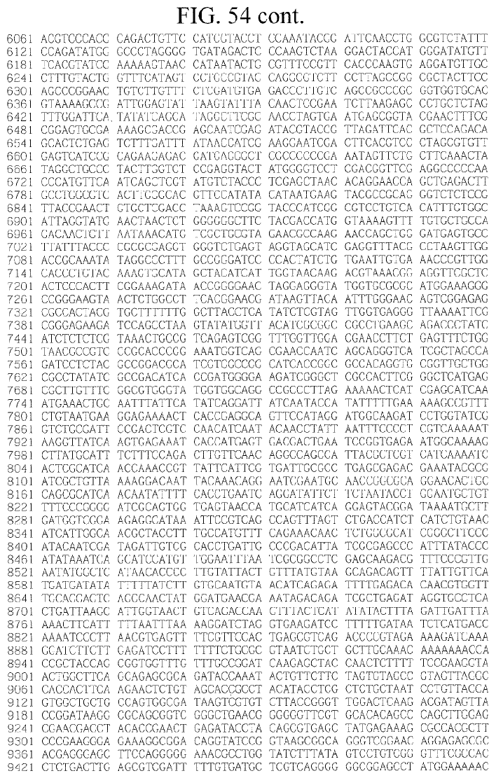

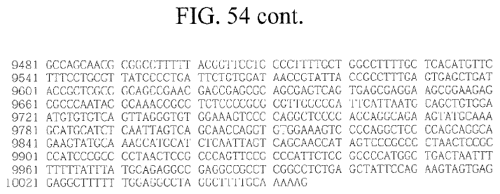

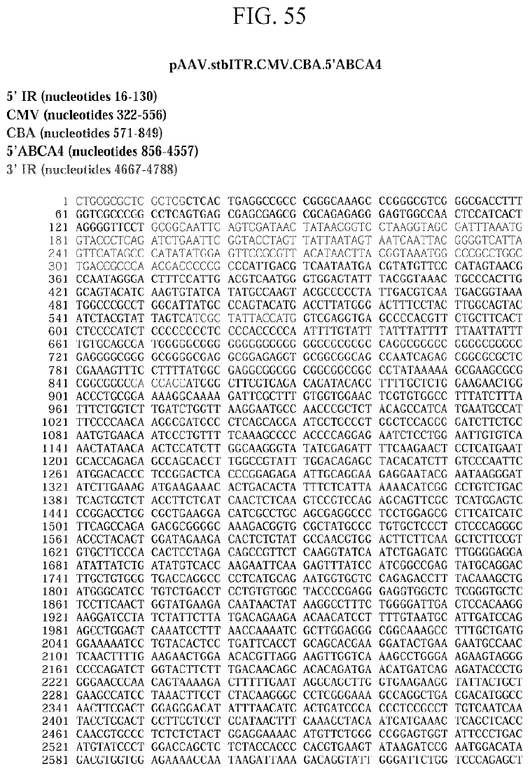

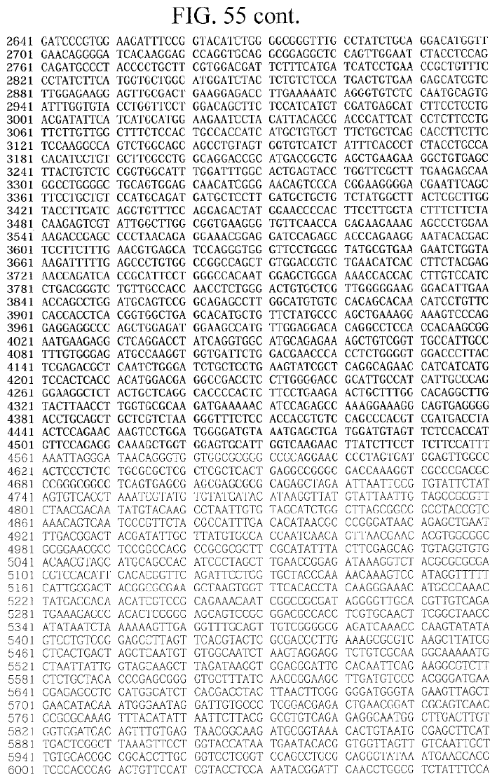

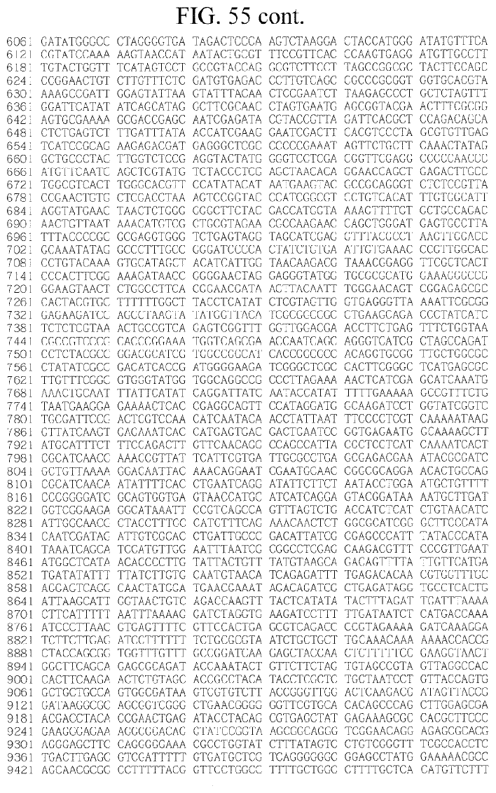

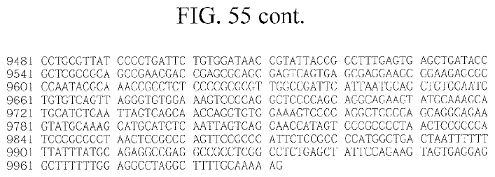

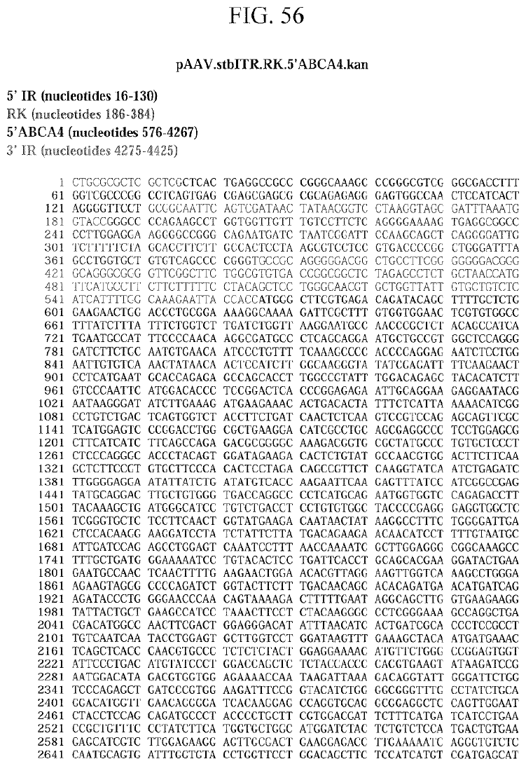

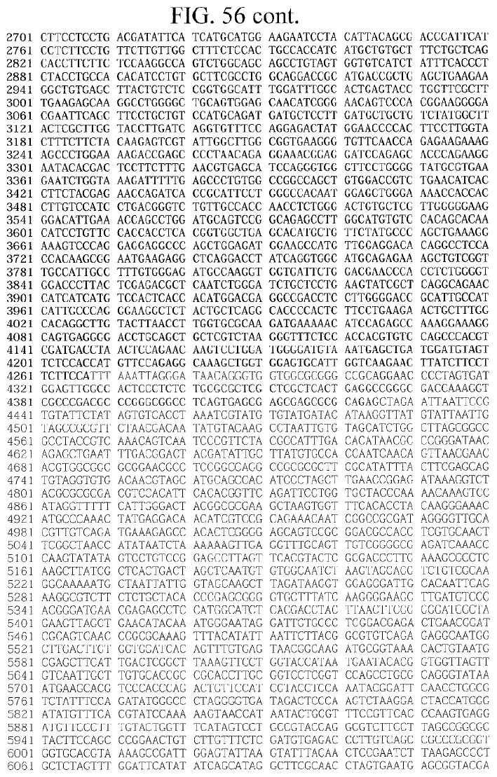

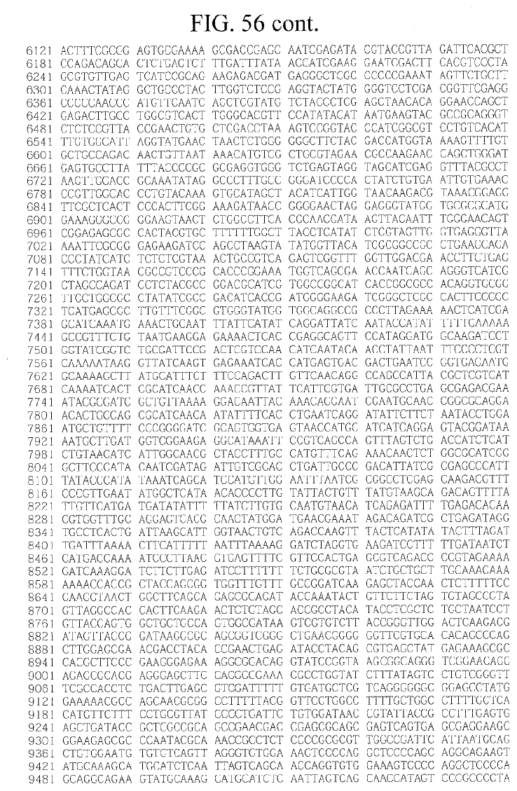

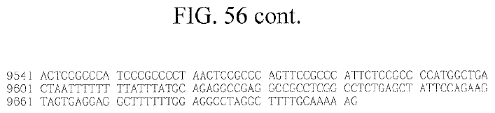

Transgene length affects AAV packaging efficiency and therefore titre. A maximum transgene size of 4.9 kb was therefore targeted (Table 2). Six overlap variants were prepared (A-F) with an additional variant designed with no overlapping region between transgenes (X) in attempt to identify an optimal overlap zone within the ABCA4 coding sequence (FIGS. 9 and 30B, Table 2).The additional variant (X) designed with no overlapping region between transgenes acted as a negative control (Table 2 and FIG. 51). Details of the overlap variants are provided in Table 2 and FIG. 51. The six overlap variants were prepared (see FIG. 51. A-F), with overlaps ranging from the maximum possible length within the AAV transgenes (1,173 bp) to a minimum consistent with maintaining specificity (23 bp). Overlap variants B-X shared the same upstream transgene (Up2) and differed only in the ABCA4 coding sequence contained in their downstream transgene. To obtain the maximal overlap zone for variant A, an extended upstream transgene was required (Up1). Having reached the maximum coding capacity of the downstream vector, the upstream transgene was extended further in the 3′ direction to obtain the maximal 1,173 bp overlap zone for variant A.

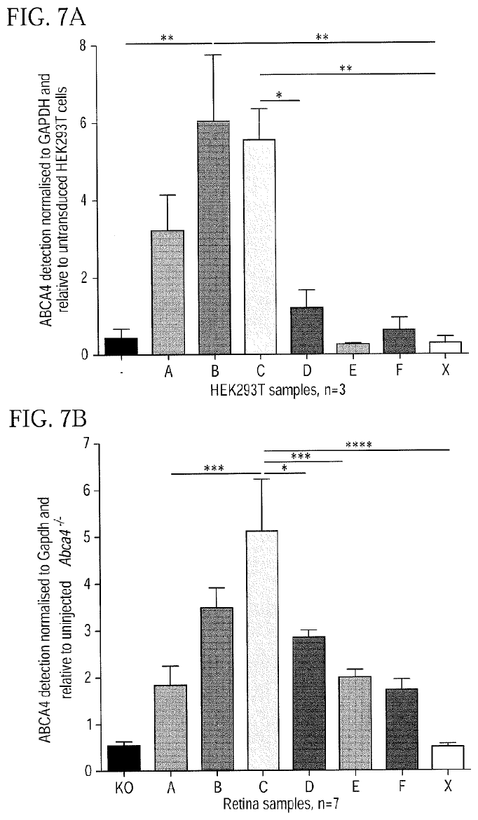

The optimal overlap zone was determined following in vitro and in vivo assessments of six overlap variants (FIG. 7a & 7b, respectively). These are referred to as A, B, C, D, E and F and represent the following overlap zones (X represents no overlap): A. 1,173 nucleotides (3259-4430); B. 506 nucleotides (3300-3805); C. 208 nucleotides (3598-3805); D. 99 nucleotides (3707-3805); E. 49 nucleotides (3757-3805) and; F. 24 nucleotides (3782-3805). (Overlap zones here are numbered relative to SEQ ID NO: 1). Downstream transgenes for overlap zones B to X are all paired with the same upstream transgene. Overlap variants B and C performed better than all other variants and to a similar extent but dual vector version C was selected for various reasons. The first is due to its limited production of truncated ABCA4 from unrecombined downstream transgenes (FIG. 8a). The unrecombined downstream transgenes from C, D, E, F and X variants generate reduced levels of truncated ABCA4 protein than the A or B versions. In a dual vector context, overlap C generates the lowest proportion of truncated ABCA4 compared to full length ABCA4 (FIGS. 8b and 8c). This suggests the overlap C transgene design is not only limiting unwanted expression from unrecombined transgenes but is also recombining with greater efficiency than the overlap B. Further evidence of this arises by comparing transcript fold change and protein fold change differences between overlap C and B injected ABCA4^{-/-} retinae. Primers targeting the upstream portion of ABCA4 CDS (therefore detecting transcripts from unrecombined upstream transgenes in addition to full length ABCA4 transcripts from recombined transgenes) detected very high levels of transcripts present in both overlap B and C dual vector injected retinae. However, overlap C generated less than half the transcript levels of overlap B yet produced 1.5 times the level of ABCA4 protein (FIG. 8d). Given that both share the same upstream vector and differ only in their downstream transgene sequence, this suggests the overlap zone selected for overlap C recombines with greater efficiency than overlap B.

The overlap zone selected has a GC content of 52% and free energy prediction of −19.60 kcal/mol, which is nearly three times less that of overlap zone B at −55.60 kcal/mol (53% GC content), FIG. 12b. This reduction in free energy suggests a secondary structure formed by unrecombined overlap C will be easier to resolve than for overlap B, which we predict leaves it more available for interaction with the overlap zone on the opposing transgene.

HEK293T cells were transduced with each dual vector overlap variant (with expression driven from a CMV enhancer, CBA promoter element) at an MOI of 10,000. Cells were harvested 5 days post-transduction and ABCA4 expression was measured by western blot analysis with ABCA4 detection levels normalized to GAPDH and presented as values above background levels of the untransduced samples. The overlap region was observed to have a significant influence on the levels of ABCA4 generated (p<0.0001, FIG. 9A). The AAV2/8 Y733F dual vector transductions of HEK293T cells identified an influence of the overlap region on the levels of ABCA4 generated (one-way ANOVA, n=3, p=0.0001, F(6,14)=10.89). Variant B generated more ABCA4 than variants A, D, E, F and X while variant C generated more ABCA4 than variants D, E, F and X (one-way ANOVA, Tukey's multiple comparisons test, n=3, X ***p=0.009/D, E, F **p≤0.009/A *p≤0.04). Dual vector variant B generated more ABCA4 than variants A, D, E, F and X while dual vector variant C generated more ABCA4 than variants D, E, F and X (FIG. 9a).

HEK293T cells were transduced with each dual vector overlapping variant and ABCA4 expression was measured by western blot analysis. The overlap region was observed to have a significant influence on the levels of ABCA4 generated (p<0.0001, FIG. 9). Overlap variants A, B and C provided 4.2 (p=0.004), 7.2 (p<0.0001) and 5.2 (p=0.0004) times more full length ABCA4 than overlap X treated cells, respectively (FIG. 9E). These dual vector variants were injected into the sub-retinal space of Abca4−/− mice and the neural retinae removed for ABCA4 detection. Efficacy in vivo was confirmed with full length ABCA4 production evident by western blot with all dual vector variants A-F.

The dual vector variants were then injected into the sub-retinal space of Abca4^{-/-} mice and the neural retinae removed 6 weeks post-injection for ABCA4 detection. Data were compiled from multiple injection groups with a total of 3-16 eyes per dual vector. Comparisons of ABCA4 protein levels in these retina samples indicated that as with the in vitro study, the overlap region influenced the levels of ABCA4 generated (p=0.001, FIG. 9b). Abca4^{-/-} mice received sub-retinal injections of AAV2/8 Y733F dual vector variants with ABCA4 expression assessed 6 weeks post-injection. The overlap region influenced the levels of ABCA4 detected (one-way ANOVA, n=5(A/B/C), 6(D/E/X) or 3(F), p=0.001, F(6,36)=4.453) and variants C and D gave more ABCA4 than control variant X (one-way ANOVA, Tukey's multiple comparisons test ***p=0.0003 and *p=0.04, respectively). Dual vector variants C and D generated more ABCA4 than variant X in vivo but all variants A-F led to detectable levels of ABCA4, unlike from in vitro samples in which A-D dual vector overlap variants generated detectable ABCA4 (FIG. 9). Including an intron between the promoter and start codon also had a significant influence on the levels of ABCA4, as did the dose of vector (p=0.04 and p=0.006, respectively, FIG. 9B).

The effect of adding an intron immediately after the promoter, which may augment gene expression, was explored. Including an intron (In) between the promoter and start codon had a significant influence on levels of full length ABCA4 achieved, with a minimum 1.5-fold increase in detection observed following treatment with overlapping vector variants (p=0.004), in the presence of a WPRE element. Subsequent injections with the optimized dual vector variant InC revealed consistent detection of full length ABCA4 in Abca4−/− injected eyes (FIG. 9F).

Neural retinae were harvested for mRNA extraction and subsequent RT-PCR and sequencing analysis spanning the overlap zones confirmed that ABCA4 transcripts generated from recombined transgenes did not carry mutations. Further RT-PCRs confirmed that the intron included in the optimized dual vector system (InC) had been successfully spliced from mRNA transcripts. Hence the dual vector system is optimized in terms of capsid, overlap zone and transgene regulatory elements.

While the data confirm previous findings of improvements in transduction when using AAV8 Y733F compared to wild-type capsids to deliver transgenes to photoreceptor cells of the mouse retina, one aspect of the overlapping dual vector strategy is the event of recombination between two transgenes. The event of recombination between two transgenes is effected by changing the overlap region length. Six different overlap regions ranging from 1.1 kb to 0.02 kb were compared (FIG. 9). All overlap variants led to ABCA4 expression with the best performing variants being B and C, representing 0.5 kb and 0.2 kb overlap lengths, respectively. The six different overlap regions ranged from 1,173-23 bp, and were compared with the best performing being 207-505 bp. A recent report compared 1.0 kb, 0.6 kb and 0.3 kb overlap regions of a lacZ gene in an overlapping dual vector system and identified the largest overlap region as being the most efficient. However, in another report a 0.07 kb F1 phage-derived sequence has proven to be efficient for achieving recombination between hybrid dual vectors in photoreceptor cells. However, with no specific investigation into overlap length presented in the data set, the reason for the largest 1.1 kb lacZ overlap providing the best reconstitution efficiency is not known. There are currently no clearly defined characteristics of what makes a region efficient at recombination. One possibility is that GC content could contribute. The GC content of the best overlap variants were comparable at 54% in variant B and 52% in variant C, yet the steric hindrance between the two regions may differ considerably. Whilst a longer region of overlap may seem logical to increase the opportunity for intermolecular interactions, by being longer it may also be less available for such interactions due to secondary structure formation whereas shorter overlaps might be problematic in the strength of their binding to the opposing transgene molecule. A shorter overlap requires a shorter run of nucleotides to be available for complementary binding, which may be less impeded by secondary structure formation. Where shorter overlaps might be problematic would then be in the strength of their binding to the opposing transgene molecule. This provides reasoning as to why the optimal overlap sequences identified in this study were determined to be in the middle of the range of overlap regions tested. This study highlights the importance of assessing multiple overlapping regions to determine the optimal sequence for a given dual vector system.

### Example 4—Experimental Protocols

FIGS. 3, 7b, 8b: Abca4^{-/-} mice received a 2 μl subretinal injection of a dual vector mix (1:1), delivering 1E+9 genome copies of each vector per eye. Enucleation of the eye was performed 6 weeks post-injection with the neural retina dissected from the eye cup and lysed in RIPA buffer. The tissue was homogenized and the supernatant extracted following centrifugation. Supernatants were mixed with denaturing loading buffer and run on a 7.5% TGX gel under denaturing conditions. Proteins were transferred to a PVDF membrane and ABCA4 detected with rabbit polyclonal anti-ABCA4 (Abcam) and Gapdh detected with mouse monoclonal anti-GAPDH (Origene). Bands were visualized and analyzed using the LICOR imaging system. ABCA4 levels were normalized to Gapdh for each sample and then represented relative to uninjected Abca4^{-/-} eyes.

FIG. 7a: All in vitro experiments were performed with HEK293T cells, which were passaged using standard protocols and transfected at 60-70% confluence with equal molarities of plasmid using the TransIT-LT1 transfection reagent (Mirus Bio, US). Cells were incubated for 48 hours at 37° C. with 5% C02 after which the media was removed and the cell layer gently rinsed with cold PBS and aspirated. Cells were loosened and lifted in a fresh volume of cold PBS then spun at 1,000×g for 10 minutes at 4° C. before removing the PBS and re-suspending in a fresh volume of cold PBS and spinning again. The PBS was removed and the cell pellets frozen at −80° C. until required.

Transductions were performed by plating HEK293T cells then lifting one well of cells 24 hours after plating (being at 80-90% confluence) to count the cells in one well. HEK293T cells were used to seed 6 well culture plates at 2E5 cells per well. After 24 hours, one well of cells was lifted and counted. This count was used to determine the appropriate amount of vector to provide to each well to give a multiplicity of infection (MOI) of 20,000 per vector. Each AAV was applied at an MOI of 20,000 based on this count. Each AAV was applied at an MOI of 20,000 based on this count. The AAV vector was added at the desired MOI to a half well volume of culture media without FBS and with 200 nM doxorubicin (Sigma-Aldrich, UK). Wells were aspirated and the volume containing AAV and doxorubicin was added to the cells, which were incubated at 37° C., 5% C02 for one hour. The remaining volume of culture media was then added to each well containing 20% FBS and cells were incubated at 37° C., 5% C02 with a media change conducted 2 and 4 days post-transduction. 48 hours post-transduction the media was removed and fresh media containing 10% FBS applied. Cells were incubated for a further 48 hours after which another media change occurred. 24 hours later, cells were harvested and washed three times in cold PBS using a gentle centrifugation cycle. The final PBS wash was removed and the cell pellets frozen. Cell pellets were thawed on ice then lysed in RIPA buffer. Cells were harvested as described above one week post-transduction. Lysates were treated as per the retina samples described above for western blot analysis.

FIG. 8a: HEK293T cells were used to seed 6 well culture plates at 1E6 cells per well. After 24 hours, a transfection mix containing 1 g of plasmid complexed to transfection reagent LT1 (GeneFlow) was applied to the cells. Test plasmids carried the downstream transgenes used in the creation of AAV vectors. 48 hours post-transfection, cells were washed, harvested and assessed by western blot as described above.

FIG. 8d: ABCA4 protein levels were obtained from western blot analyses as described in FIG. 3 and the fold change compared between overlap variant C and B dual vector treatments. For transcript level comparisons, tissue samples were collected in RNAlater (Ambion) and the mRNA extracted using Dynabeads-oligodT mRNA DIRECT (Life Technologies). cDNA synthesis was performed with 500 ng mRNA using an oligodT primer and SuperScript III (Life Technologies). Samples were cleaned using PCR Purification Spin Columns (QIAGEN) and eluted in 50 μl DEPC-treated water. The cDNA was assessed by qPCR targeting an upstream portion of the ABCA4 CDS. Levels of ABCA4 were normalized to Actin levels and expressed relative to uninjected Abca4^{-/-} samples. The fold change in ABCA4 transcript levels between overlap variant C and B dual vector treatments were then compared.

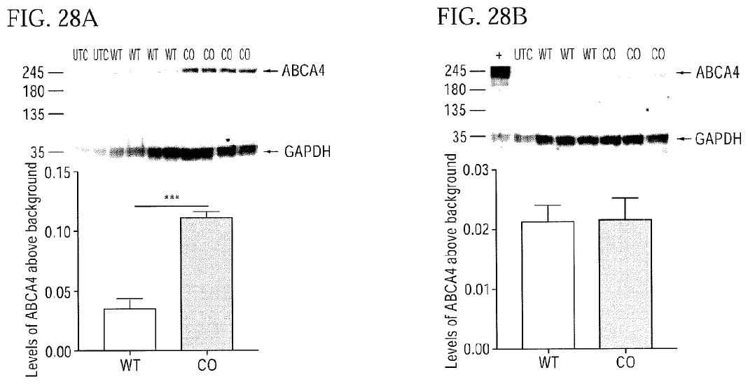

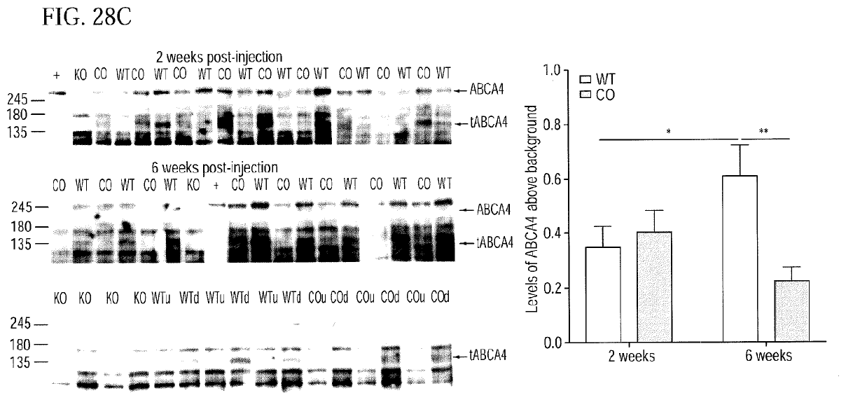

In vivo experiments: All animal breeding and experimental procedures were performed under approval of local and national ethical and legal authorities and were conducted in compliance with the ARVO statement for the Use of Animals in Ophthalmic and Vision Research. Pigmented Abca4^{-/-} mice (129S4/SvJae-Abca^{4tm1Ght}) were provided by Gabriel Travis (David Geffen School of Medicine, University of California, Los Angeles, Calif.) and bred in the Biomedical Sciences division, University of Oxford. Pigmented WT control mice (129S2/SvHsd) were purchased from ENVIGO (Hillcrest, UK). Animals were kept in a 12 hour light (<100 lux)/dark cycle with food and water available ad libitum. Sub-retinal injections were performed by delivering 2 μl of reagent under direct visual guidance using an operating microscope (Leica Microsystems, Germany). Early experiments used a scleral tunnel approach through the posterior pole to the superior retina with a Hamilton syringe and 34-gauge needle (ESS labs, UK), this injection system and method was used for eyes assessed in FIG. 28. All subsequent sub-retinal injections involved performing an anterior chamber paracentesis with a 33G needle prior to the sub-retinal injection using a WPI syringe and a bevelled 35G-needle system (World Precision Instruments, UK). Animals were anaesthetized by intraperitoneal injection containing ketamine (80 mg/kg) and xylazine (10 mg/kg) and pupils fully dilated with tropicamide eye drops (Mydriaticum 1%, Bausch & Lomb, UK) and phenylephrine eye drops (phenylephrine hydrochloride 2.5%, Bausch & Lomb, UK). Proxymetacaine eye drops (proxymetacaine hydrochloride 0.5%, Bausch & Lomb, UK) were also applied prior to sub-retinal injection. Post-injection, chloramphenicol eye drops were applied (chloramphenicol 0.5%, Bausch & Lomb, UK) and anaesthesia was reversed with atipamezole (2 mg/kg) and carbomer gel applied (Viscotears, Novartis, UK) to prevent cataract formation.

Transcript analysis: Samples were either HEK293T frozen cell pellets or neural retina stored in RNAlater (ThermoFisher Scientific, UK). Neural retina samples were achieved by dissection of eye cups following enucleation and were placed in RNA immediately following dissection. Samples were thawed on ice and mRNA extracted using mRNA DIRECT Dynabeads-oligodT (Life Technologies, UK) with 500 ng of mRNA then used in a SuperScript III cDNA synthesis reaction with oligodT primer as per the manufacturers guidelines. The cDNA was cleaned in QIAGEN spin columns and eluted in 50 μl DEPC-treated water. Transcripts were assessed by qPCR using 2 μl of each cDNA preparation (qPCR primers listed above). ABCA4 levels were normalized to ACTINActin levels. For RT-PCR, 2 μl of cDNA was used to identify upstream only transcript length (FW 5′GATTACAAAGATGACG (SEQ ID NO: 71), REV 5′GCAATTCAGTCGATAACTA (SEQ ID NO: 72)), overlap (FW 5′ACCTTGATCAGGTGTTTCCA (SEQ ID NO: 73), REV 5′ACAGAAGGAGTCTTCCA (SEQ ID NO: 74)) and 5′UTR assessments (FW 5′CCACTCCTAAGCGTCCTC, REV 5′CAGGGATTGTTCACATTGC (SEQ ID NO: 75)). Amplicons for sequence analysis were PCR-purified or cloned and purified before Sanger sequencing.

Western blot assessment: Samples were either HEK293T frozen cell pellets or frozen neural retina tissue. Neural retina samples were achieved by dissection of eye cups following enucleation and were frozen in lysis buffer (RIPA buffer (MerckMillipore, UK), plus proteasome inhibitor (Roche, UK)) on dry ice immediately following dissection. Samples were thawed on ice and lysed using a hand-held homogeniser and rotated at 4° C. for one hour prior to spinning at 17,000×g for 30 mins, 4° C. The supernatant was removed with 20 μl added to 5 μl protein loading buffer (GeneFlow, UK). This was left at room temperature for 15 minutes prior to loading on a 7.5% TGX gel (BioRad, UK). Proteins were transferred to a PVDF membrane using a TransBlotTurbo with subsequent ABCA4/Abca4 (ab72955, Abcam, UK) and GAPDH/Gapdh detection (TA802519, Origene, US) conducted using a SNAPiD system (MerckMillipore, UK). Blots in FIG. 28 used HRP-conjugated secondary antibodies (Abcam, UK) and were developed with Luminata Forte HRP substrate (MerckMillipore, UK). Other blots were detected using IRDye fluorescent secondary antibodies (LI-COR Biosciences, UK). Membrane signals were recorded with the Odyssey imaging system (LI-COR Biosciences, UK) and band densities were assessed using Image Studio Lite software with ABCA4/Abca4 levels normalized to GAPDH/Gapdh levels and values presented relative to background readings of negative control samples.

Data sets was assessed for normal distribution (Shapiro-Wilk test) and variance (Brown-Forsythe test). Ff data for comparison exhibited unequal variance (skewed and unpaired) then non-parametric tests were performed (Mann-Whitney U-test or Kruskal Wallis). If variances were equal (the data were normally distributed and paired) then parametric tests were used (Student's t-test or ANOVA). Multiple comparisons were conducted with correction using either Tukey's or Sidak's comparisons test (if ANOVA) or Dunn's comparisons (if Kruskal Wallis). Brief descriptions of the drawings indicate the test used to analyses each specific data set with n, p and F values provided where appropriate. Data are shown as mean and SEM.

### Example 5—AAV-Mediated Delivery of ABCA4 to the Photoreceptors of Abca4^{-/-} Mice Using an Overlapping Dual Vector Strategy

The data presented in this Example demonstrate the expression of ABCA4 protein specifically localized in the photoreceptor outer segments of the Abca4^{-/-} mouse model following sub retinal injection with an overlapping dual vector system of the disclosure.

Transgene design and production: Overlapping ABCA4 transgenes were packaged into AAV8 Y733F capsids. The upstream transgene contained the human rhodopsin kinase (GRK1) promoter and an upstream portion of the ABCA4 coding sequence (CDS) between AAV2 inverted terminal repeats (ITRs). The downstream transgene contained a downstream portion of the ABCA4 CDS, Woodchuck hepatitis virus post-transcriptional regulatory element (WPRE) and a polyA signal (pA). Both the upstream and downstream transgenes carried a region of ABCA4 CDS overlap.

Injections: Abca4^{-/-} mice received a 2 μl sub retinal injection at 4-5 weeks of age containing a 1:1 mix of the upstream and downstream vectors (1×10¹³ gc/ml). Eyes were harvested at 6 weeks post-injection for immunohistochemical (IHC) assessments.

Initial sub-retinal injections delivered 2E+9 total genome copies of the various dual vector combinations. Abca4^{-/-} eyes injected with 2E+9 or 2E+10 total genome copies were compared in relation to ABCA4 protein levels achieved. More ABCA4 was detected in eyes that received the higher dose (FIG. 11b), indicating that increasing delivery of transgenes enabled more successful recombination events. All subsequent in vivo studies were conducted with the 2E+10 dose.

Immunohistochemical staining: Whole eye cups with the lens removed were fixed in 4% paraformaldehyde (PFA) for 20 minutes at room temperature. Eye cups were transferred to a 10% sucrose solution for one hour, followed by a 20% solution for one hour, and then incubated in 30% sucrose overnight at 4° C. Eye cups were placed in O.C.T compound (VWR, UK), incubated for 30 mins then frozen on dry ice and stored at −80° C. Eyes were sectioned using a cryostat and dried overnight on slides at room temperature under dark conditions before use. Tissue slices were dried overnight at room temperature then rinsed in phosphate buffered saline (PBS) for 5 minutes, three times. Samples were permeabilized with 0.2% Triton-X-100 for 20 minutes then washed three times in PBS before incubating with 10% donkey serum (DS), 10% bovine serum albumin (BSA) for one hour. Antibodies were diluted 1/200 in 1% DS, 0.1% BSA, applied to sections and left for two hours at room temperature or overnight at 4° C. Abca4/ABCA4 detection was achieved with goat anti-ABCA4 (AntibodiesOnline), hyperpolarization activated cyclic nucleotide gated potassium channel 1 (Hcn1) detection with mouse anti-Hcn1 (Abcam) and rhodopsin detection with mouse anti-1D4 (Abcam). Sections were rinsed three times with 0.05% Tween-20 then secondary antibodies applied (diluted 1/400) for two hour under dark conditions at room temperature. Sections were rinsed twice with 0.05% Tween-20 then incubated with Hoescht stain (1/1,000) for half an hour. Sections were rinsed in PBS then leg to air dry. ProLong Diamond anti-fade mounting medium was applied to each section and slides were left overnight before imaging. Primary antibodies used were: ABCA4/Abca4 detection (ABIN343052, AntibodiesOnline, Germany), Hcn1 detection (mouse monoclonal ab84816, Abcam, UK), Rho detection (ab5417, Abcam, UK). Anti-ABCA4 specificity was confirmed by western blot analysis using pCAG.ABCA4.pA transfected HEK293T lysate, wild-type mouse retinal lysate and commercially available human ABCA4 peptide (Abcam, ab114660). Secondary antibodies used were all donkey Alexa Fluor: anti-goat 488 (ab1050129, Abcam, UK) and anti-mouse 568 (ab175472, Abcam, UK).

### ABCA4 Expression Localized to Photoreceptor Cell Outer Segments.

FIG. 17 shows Abca4/ABCA4 (green) and Hcn1 (red) staining in wild-type (WT) and Abca4^{-/-} eyes. WT SVEV 129, uninjected and injected Abca4^{-/-} eyes were stained for the photoreceptor inner segment marker Hcn1 and Abca4/ABCA4. WT and dual vector treated Abca4^{-/-} eyes revealed specific localization of Abca4/ABCA4 in the photoreceptor cell outer segments.

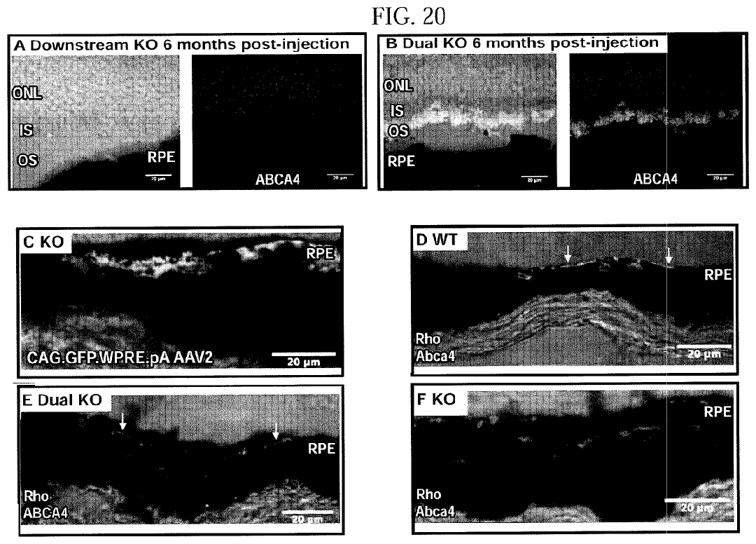

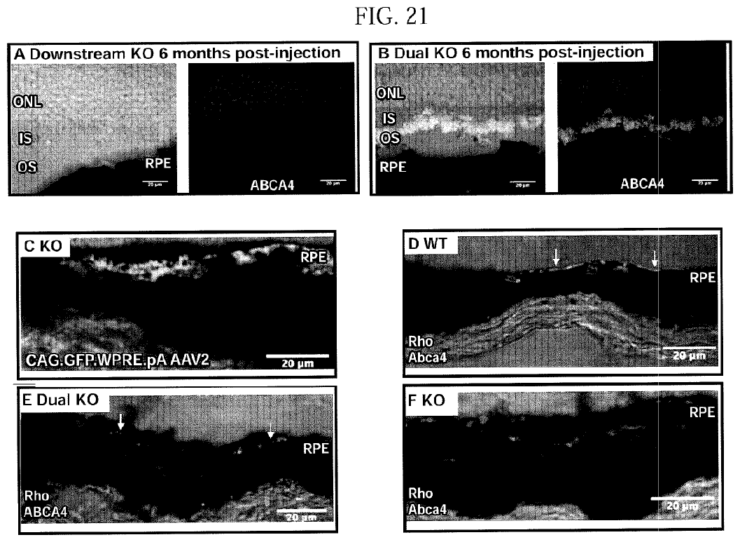

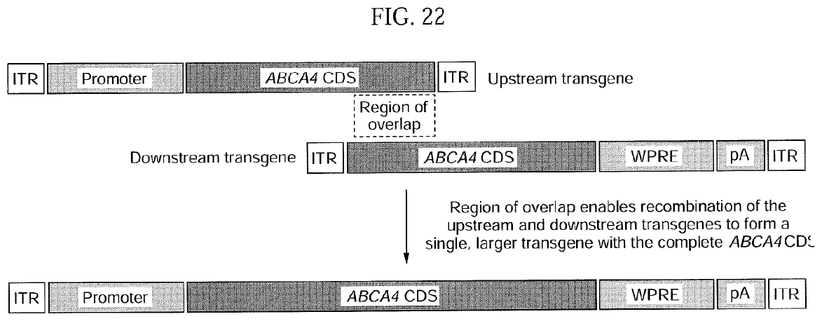

Since ABCA4 is a large and complex folded protein that undergoes post-translational modification and is trafficked to the cell membrane of the specialized compartment of the photoreceptor outer segment, immuno-histochemical localization provides additional indirect information on protein structure beyond the western blot. Immunohistochemical staining was performed to confirm correct localization of ABCA4 in the photoreceptor cell outer segments of the retina after dual vector transduction by sub-retinal injection. Anti-Hcn1 was used to highlight the limit of the photoreceptor inner segments. Images were taken on a confocal microscope and focusing on the outer segments masked the RPE layer, which required imaging on a different focal plane to observe any staining therefore RPE images are presented separately (FIG. 21). For FIG. 20, eyes were harvested 6 weeks post-injection and prepared for immunohistochemical staining. Anti-Hcn1 was used to highlight the limit of the photoreceptor inner segments as correct ABCA4 localization would be expected predominantly at the outer segment structures. SVEV 129 wild-type (WT) eyes were used as positive controls and revealed abundant Abca4 presence in the photoreceptor cell outer segments (FIG. 20a). Uninjected Abca4^{-/-} eyes and those that received a sub-retinal injection of either upstream or downstream vector only exhibited no detectable ABCA4 staining (FIG. 20b, c & d, respectively). The absence of staining in downstream vector injected eyes aligned with the reduction of truncated ABCA4 (tABCA4) observed by western blot analysis with the optimized downstream vector C variant. A feature of truncated ABCA4 (tABCA4) production from unrecombined downstream transgenes is that it would generate a non-specific expression pattern given the absence of a cell-specific promoter. No truncated ABCA4 staining in the eye cups of downstream vector only injected mice was observed up to 6 months post-injection (FIG. 21A). For Abca4^{-/-} eyes injected with the optimized dual vector system, ABCA4 staining was evident in the outer segments of the photoreceptor cells (FIG. 20F) with expression detected up to 6 months post-injection.

ABCA4 Co-Localization with Rhodopsin.

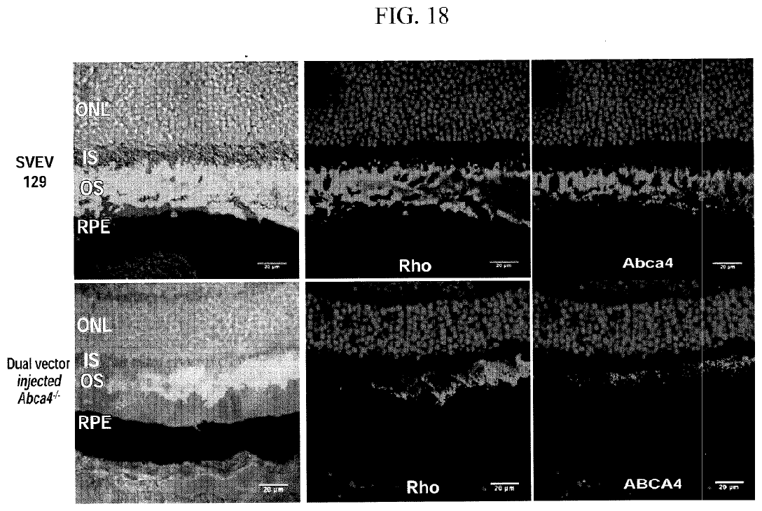

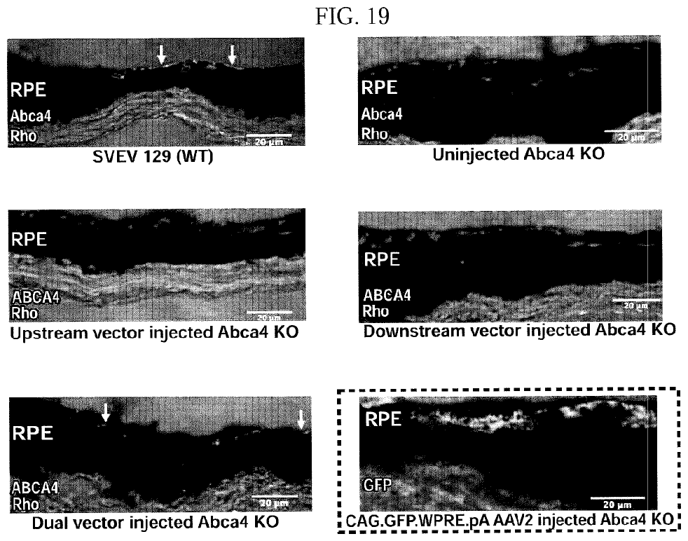

FIG. 18 shows Abca4/ABCA4 (green) and rhodopsin (red) staining in photoreceptor cell outer segments in wild-type (WT) and Abca4^{-/-} eyes. WT and dual vector treated Abca4^{-/-} eyes revealed co-localization of rhodopsin and Abca4/ABCA4 in the photoreceptor cell outer segments.

FIG. 19 shows Abca4/ABCA4 (green) and rhodopsin (red) apical RPE staining in wild-type (WT) and Abca4^{-/-} eyes. WT and dual vector treated Abca4^{-/-} eyes revealed co-localization of rhodopsin and Abca4/ABCA4 in the apical regions of RPE cells, hypothesized to originate from shed outer segment discs. Abca4^{-/-} eyes not treated with the dual vector showed only rhodopsin staining in the apical region of RPE cells. Boxed image shows the expression pattern achieved from transduced RPE cells (GFP staining in Green), revealing a diffuse staining pattern in contrast to the Abca4/ABCA4/rho staining.

For Abca4^{-/-} eyes injected with the optimized dual vector system (5′C), ABCA4 staining was evident in the outer segments of the photoreceptor cells (FIG. 20e & f) and was shown to co-localize with rhodopsin (FIG. 20h) and be detectable up to 6 months post-injection (FIG. 20j). In some dual vector injected eyes, ABCA4 staining in the apical region of the RPE was observed but the staining was not diffuse throughout the RPE cells (FIG. 20f). If this ABCA4 staining were due to expression occurring from within the RPE cells, we would anticipate ABCA4 presence throughout the cell and not isolated in the apical region (FIG. 21C). For example, transduction of the RPE cells with a CAG.GFP.WPRE.pA AAV2 reporter vector led to diffuse GFP staining throughout the cells (FIG. 21a). It was therefore hypothesized that the apical staining of ABCA4 was originating from sheared or shed photoreceptor outer segment discs in contact with the RPE cells. To confirm this, rhodopsin staining was conducted and revealed an identical staining pattern in WT eyes that co-localized with Abca4 (FIG. 21D). This co-localization was also observed in some dual vector injected Abca4^{-/-} eyes (FIG. 21E) and a similar pattern of apical rhodopsin staining was observed in uninjected Abca4^{-/-} eyes (FIG. 21F).

An optimized overlapping dual vector system can be used to generate ABCA4 expression in photoreceptor cells where it is trafficked to the desired outer segment structures at levels detectable by IHC.

### Example 6—Bisretinoid/A2E Assessments in Dual Vector Treated Abca4^{-/-} Mice

Accumulation of bisretinoids are hallmarks of Stargardt disease and believed to play a primary role, or be the major driver for in retinal degeneration in humans. A reduction in these molecules provides a functional assay in which to assess the efficacy of the dual vector AAV system in Abca4^{-/-} mice. The specific localization of dual vector delivered ABCA4 to the outer segment discs of the Abca4^{-/-} photoreceptor cells implies correct folding of the full length ABCA4 protein, particularly given that the two transmembrane domains (TMD) were encoded across the two vectors, TMD1 in the upstream vector and TMD2 in the downstream. This study used pigmented Abca4^{-/-}, a mouse model without significant photoreceptor cell loss, at least up to one year of age. The pigmented mouse model used in this study does not suffer from retinal degeneration nor does it have a detectable ERG phenotype. However, a consistent feature of these mice is the extensive accumulation of quantifiable bisretinoids over time, thus recapitulating a pathological hallmark of the human disease.

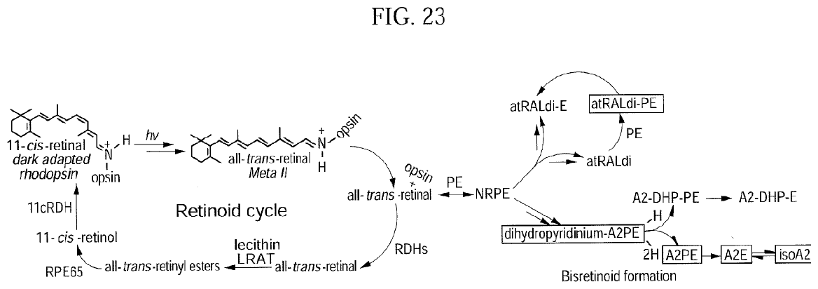

The Abca4^{-/-} mouse model exhibits an increase with age in levels of bisretinoids and A2E compared to wild type mice. In contrast to humans, however, the increase in bisretinoids does not reach a level that would be required to cause any significant retinal degeneration. This suggests that other compensatory mechanisms may exist in the Abca4 deficient mouse eye. In a wild type retina, Abca4 facilitates the movement of retinal out of the photoreceptor cell outer segment disc membranes for recycling. When there is an absence of functional Abca4, as in the Abca4^{-/-} mouse model, the retinal is maintained in the outer segment disc membranes where it undergoes biochemical changes into various bisretinoid forms (FIG. 23). Photoreceptor cells constantly generate new outer segment discs and in doing so there is movement of the older more distal discs towards the RPE cells, which subsequently degrade them by phagocytosis. In the Abca4 deficient mouse the phagocytosed discs contain elevated levels of bisretinoids. Within the RPE cells these are further converted into A2E isoforms, the accumulation of which leads to lipofuscin. Hence although the bisretinoid accumulation in the Abca4 deficient mouse is insufficient to cause a retinal degeneration, the resulting elevated levels above baseline may nevertheless be quantified and thus provide a biomarker of Abca4 function.

Bisretinoid and A2E compounds can be accurately measured by high-performance liquid chromatography (HPLC). A measure of therapeutic efficacy in mice treated with ABCA4 gene therapy would therefore be to achieve a reduction in the levels of bisretinoids and A2E compared to untreated eyes. There are however two considerations that need to be addressed. In the first instance, for clinical application we need to use a human ABCA4 coding sequence and a human photoreceptor promoter and this is unlikely to be as efficacious in the mouse. Furthermore HPLC measurements are taken from the whole eye and not just the region exposed to the vector by the subretinal injection. Hence the overall reduction in bisretinoids in the Abca4 deficient mouse is unlikely to reach wild type levels. The second consideration is the subretinal injection, which may lead to damage of the outer segment discs. Since these structures are rich in bisretinoids, the effects of ABCA4 gene therapy need to be compared with a similar sham injection. Ideally the contralateral eye of the same mouse should be used for this to control for eye size and lifetime light exposure, which may also influence bisretinoid accumulation.

For this reason we compared the bisretinoid/A2E levels in a cohort of Abca4−/− mice that received a sham injection in one eye and a similar treatment injection in the contralateral eye. Each sham eye received the upstream vector at the same total AAV dose as that which was received in the paired dual vector treatment eye. Both eyes of each mouse therefore received a 2 μl subretinal injection, forming a bleb containing 2×10¹⁰ genome particles of AAV vector.

A total of 13 Abca4 knockout mice were injected at 4-5 weeks of age and eyes were harvested 3 months post-injection. To investigate whether the ABCA4 present in the photoreceptor outer segments of treated Abca4^{-/-} mice was functioning and providing therapeutic effect, eyes were assessed for bisretinoid/A2E levels by HPLC analysis. Mice were dark adapted for 16 hours prior to tissue collection, which was conducted in the dark under dim red light.

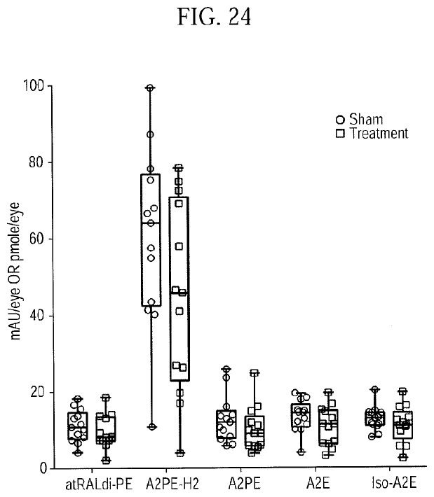

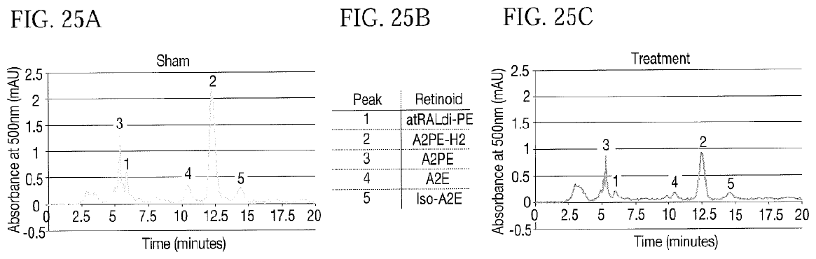

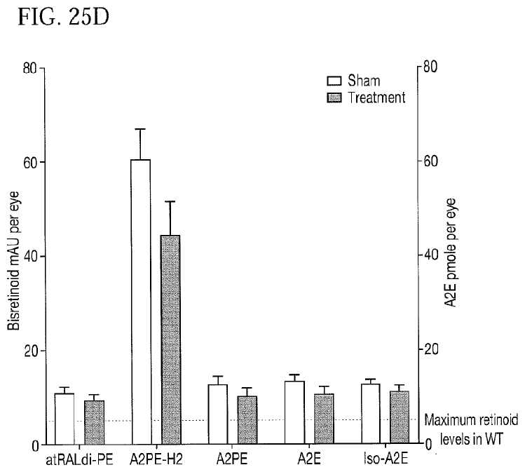

In a completely blinded study, whole eyes of treated Abca4^{-/-} mice were harvested then anonymized and shipped frozen to the Jules Stein Eye Institute for processing of the levels of all-trans-retinal dimer-phosphatidylethanolamine (atRALdi-PE), N-retinylidene-N-retinylphosphatidylethanolamine (A2PE), di-hydro-A2PE (A2PE-H2), conjugated N-retinylidene-N-retinylphosphatidylethanolamine (A2E) and a double bond isomer of A2E (iso-A2E) in Abca4^{-/-} mice. Anonymized eyes of 13 Abca4^{-/-} mice received the upstream vector-only in one eye (sham) and the dual vector in the contralateral eye (treatment), with each eye receiving the same total AAV dose. Eyes were harvested in dark conditions, processed, and analyzed by high performance liquid chromatography at another center (JSEI) to determine levels of all-trans-retinal dimer-phosphatidylethanolamine (atRALdi-PE), N-retinylidene-N-retinylphosphatidylethanolamine (A2PE), di-hydro-A2PE (A2PE-H2), conjugated N-retinylidene-N-retinylphosphatidylethanolamine (A2E) and its major cis-isomer (iso-A2E). Each whole eye was taken and processed without dissection. These assessments were performed with the identity of each eye masked. Enucleation was conducted under dim red light and eyes were immediately frozen and stored at −80° C. protected from light. Eyes were then shipped frozen at −70° C. by World Courier Services. The travel time was less than 48 hours and temperature logs confirmed the specimens remained frozen throughout. Bisretinoid extraction and assessment by HPLC was performed on the eyes as previously described. Following HPLC assessments of all 26 eyes, the identities were subsequently unmasked and bisretinoid/A2E levels for each treated eye were compared to their paired sham injected eye. Two-way ANOVA determined the treatment to have an effect on the levels of bisretinoid/A2E with a reduction in dual vector treated eyes observed compared to paired sham injected eyes (p=0.0171), FIG. 24.

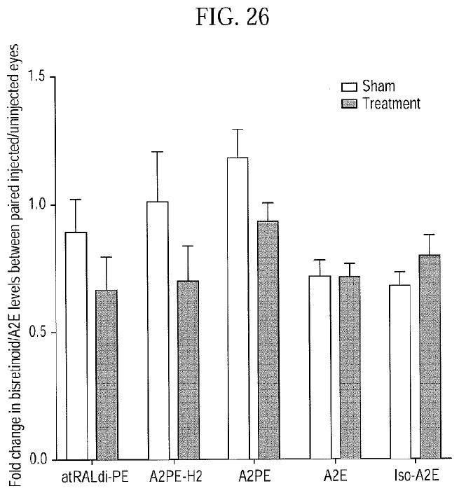

An initial study consisted of two groups of 11 mice, the first group had an uninjected eye and a dual vector treated eye whilst the second group had an uninjected eye plus a sham injected eye. The sham injection contained the upstream vector only of the same total dose as that which was received in the dual vector injected group (2E+10 total genome copies). ). Levels of each bisretinoid marker in the uninjected eye were compared to levels in the paired injected eye of each mouse and presented as the fold change between eyes with “1” therefore representing the bisretinoid value in the paired uninjected eye (FIG. 26). A difference in the fold change of bisretinoid levels in the treatment group compared to the sham group was identified (p=0.05, FIG. 26). Variability in baseline bisretinoid levels were noted between uninjected control eyes so a second confirmatory study was conducted.

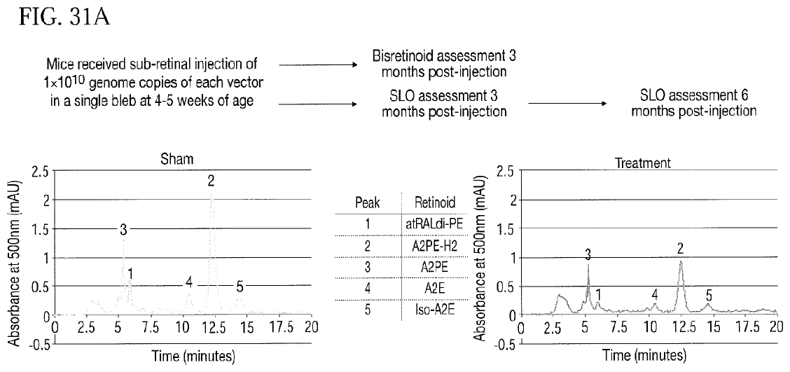

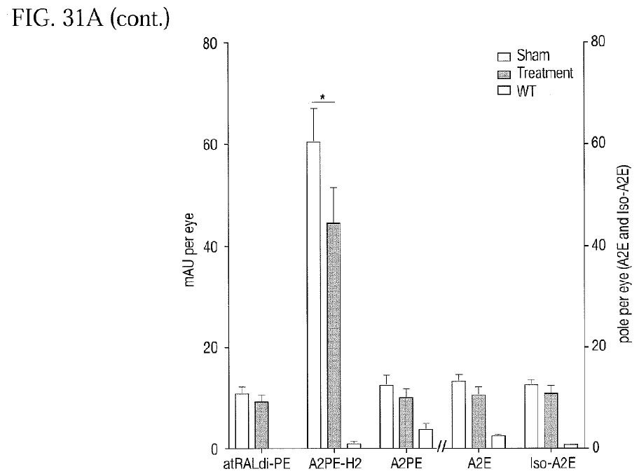

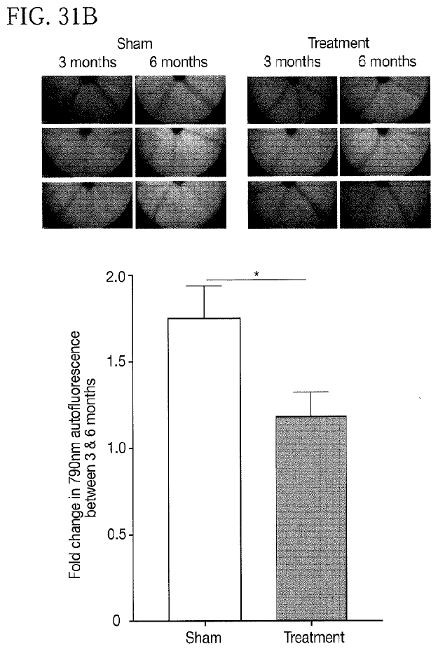

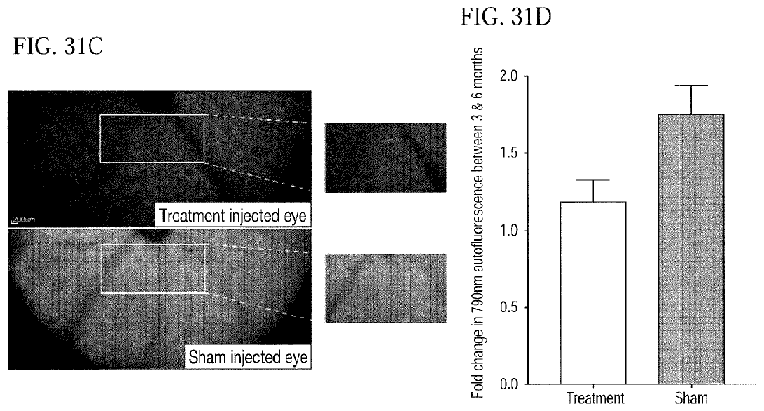

Variability in baseline bisretinoid/A2E levels were noted between uninjected control eyes. To reduce the influence of this natural variability, which may have related to different eye cup sizes in animals of different ages, a second confirmatory study was performed which used the fellow eye in each animal as an internal control. In the second study, Abca4^{-/-} mice received the upstream vector in one eye (sham) and the dual vector in the contralateral eye (treatment), with each eye receiving the same total AAV dose. 13 mice received the upstream vector in one eye (sham) and the dual vector in the contralateral eye (treatment), both eyes received the same total vector dose of 2E+10 genome copies. The same bisretinoids were assessed with the data presented as a comparison of the levels of each biomarker detected in paired eyes. The treatment was observed to have an influence on the bisretinoid/A2E levels in Abca4^{-/-} mouse eyes compared to sham injected eyes (p=0.03, FIG. 31A). Combining the data of sham injected and treated eyes from the two experiments conducted and using a three-way ANOVA test to compare across the experiments, the influence of the treatment was identified as having p=0.0002. This is the first presentation of a dual vector treatment for Stargardt disease affecting the biochemistry of the Abca4^{-/-} mouse model, providing an indication that functional ABCA4 was delivered to the photoreceptor cells and inducing a positive therapeutic effect. Example 7—Upstream and downstream transgene related expression

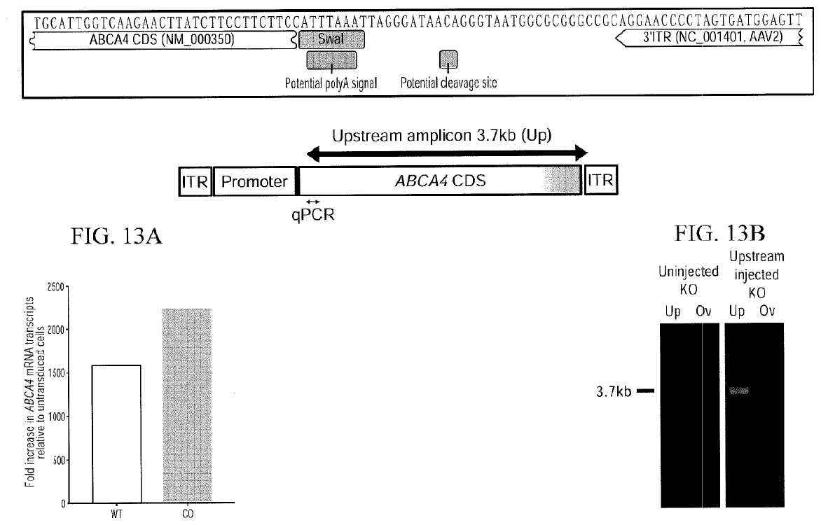

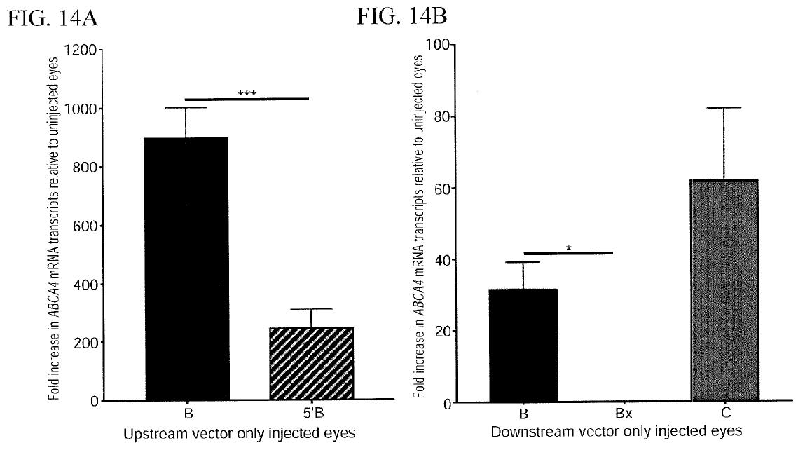

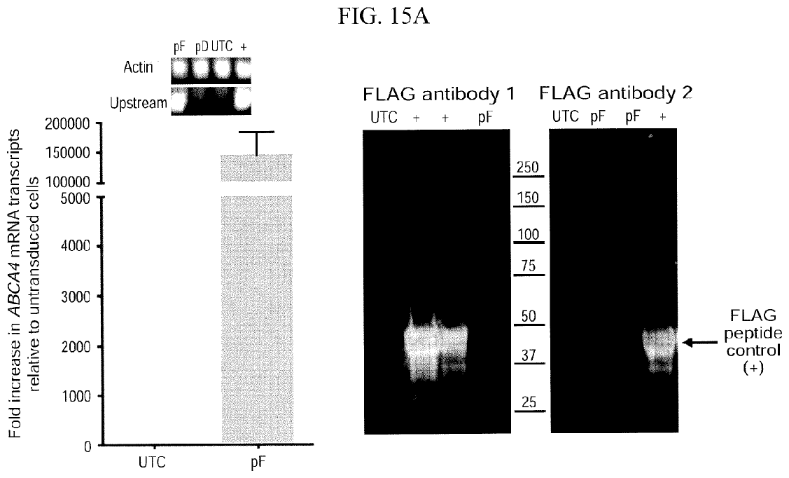

Using antibodies directed against the C-terminus of the ABCA4 protein, western blot assessment identified additional truncated ABCA4 (tABCA4) protein −135 kDa following downstream vector only injection in Abca4^{-/-} mice (FIG. 28c). QPCR assessments of single vector injected retinae confirmed stable mRNA transcripts were generated from upstream and downstream vectors (FIG. 14). Given that mRNA was directly extracted from all samples using the polyA tails of transcripts, these data implied existence of an element that was enabling polyA tail addition to transcripts from unrecombined upstream transgenes. Analysis of the nucleotide sequences contained in these upstream transgenes revealed two potential cryptic polyA signals of ATTAAA beginning at nucleotides 502 and 1,750 of the ABCA4 coding sequence (SEQ ID NO: 11), respectively. If either of these sites were responsible for stable transcript formation then short mRNA transcripts would be anticipated only from WT upstream vectors as the CO version was modified to remove these cryptic elements. In vitro testing of WT and CO ABCA4 vectors indicated that transcripts were present from upstream vector only treated HEK293T cells samples treated with either variant (FIG. 13) and in vivo assessment of transcript length showed the transcripts to extend beyond these internal cryptic polyA sites (FIG. 13). These data indicate that another cryptic element of the transgene structure consistent to all upstream transgene variants could be enabling the addition of a polyA tail to transcripts. The likely feature was determined to be a SwaI restriction site (ATTTAAAT, SEQ ID NO: 76) outside of the ABCA4 coding sequence and used in the cloning process. While AATAAA (SEQ ID NO: 77) and ATTAAA (SEQ ID NO: 78) are the most common polyA signals, TTTAAA is also a potential polyA hexamer. This sequence was consistent in all upstream transgenes and given the additional presence of a potential CA cleavage point 10 nucleotides downstream of the TTTAAA sequence (FIG. 13), this was considered plausible as a polyA site in all upstream transgenes.

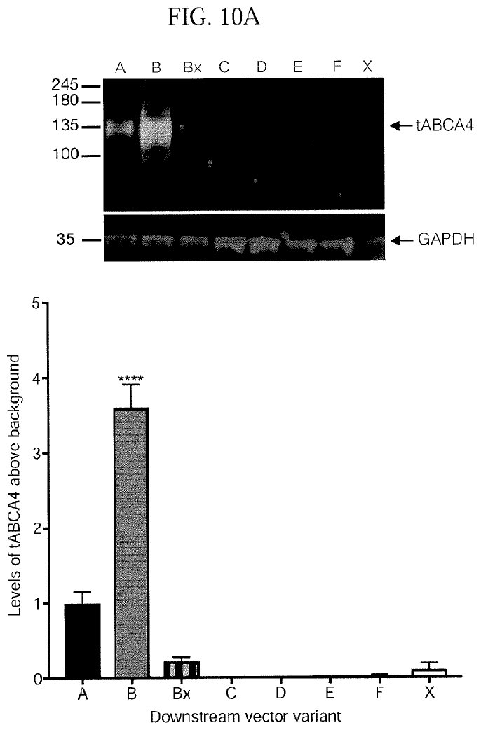

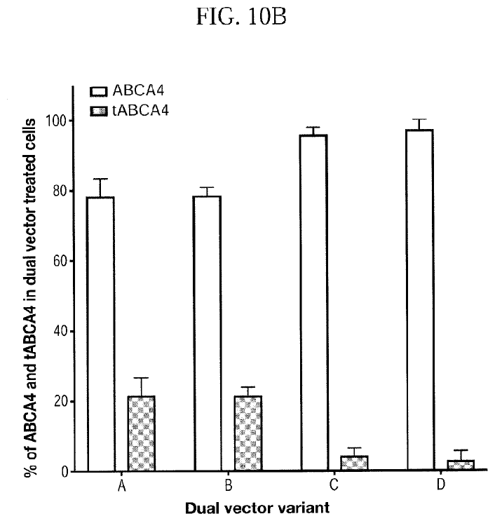

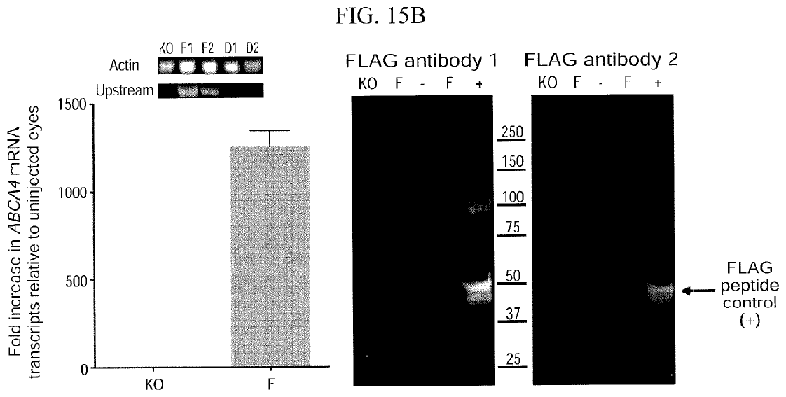

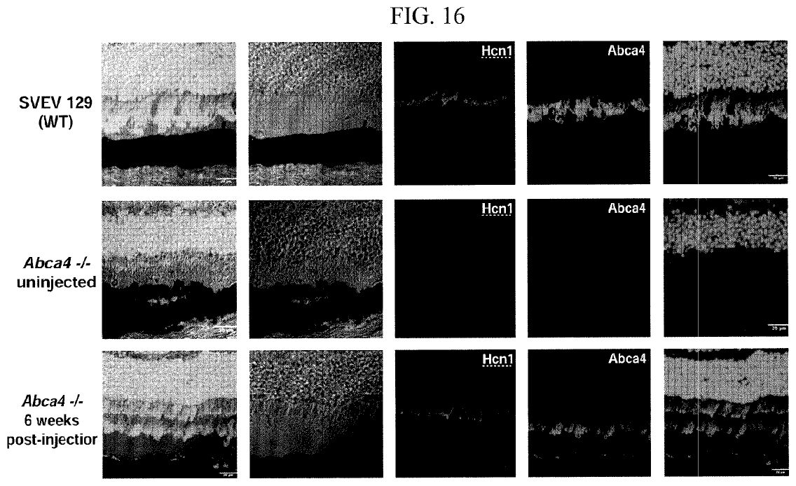

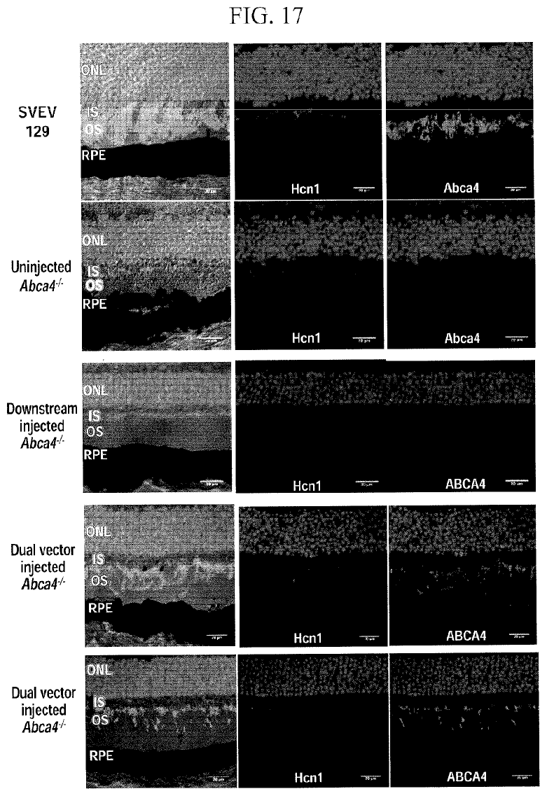

One aspect in the optimization of the dual vector system was limiting unwanted expression from unrecombined transgenes. Despite the detection of ABCA4 mRNA transcripts from upstream vector only injected eyes, ABCA4 protein forms were not detected (FIG. 15). Absence of truncated ABCA4 protein forms was confirmed with constructs containing an N-terminus FLAG tag. However, western blot assessments using polyclonal antibodies directed to the C-terminus identified truncated ABCA4 (tABCA4) protein ˜135 kDa following dual and downstream vector only injection in Abca4^{-/-}mice (FIG. 28C). Truncated ABCA4 transcripts were also identified following transduction with the downstream vector. Expression from unrecombined downstream transgenes was anticipated to result from the native promoter activity of the AAV2 ITR, which can then read out into the polyA signal. The ABCA4 coding sequence contained in downstream vector transgenes A and B carried in-frame ATG codons at a distance from the 5′ITR from which translation of the resulting mRNA transcript to the ABCA4 stop codon could be anticipated, and both A and B unrecombined downstream constructs generated tABCA4 (an AUG within 100-200 nucleotides of the 5′ITR D sequence, FIG. 9). The ABCA4 coding sequence contained in the downstream transgene significantly influenced the levels of truncated ABCA4 detected with only A and B unrecombined downstream constructs generating consistently detectable truncated ABCA4 (p=0.0003). The original vector designs were variants A and B, which both generated tABCA4 (FIG. 10). Downstream constructs C to X were subsequently designed such that the ABCA4 coding sequence immediately downstream of the 5′ITR contained out-of-frame ATG codons prior to in-frame ATG codons. This required no alterations to the native ABCA4 coding sequence and significantly influenced the levels of tABCA4 produced (p=0.003, FIG. 9C). Through detailed assessment of the ABCA4 coding sequence we identified out-of-frame and in-frame ATG codons in good context, i.e. resembling a Kozak consensus, contained in the ABCA4 coding sequence. Fragments of ABCA4 coding sequence to be packaged on downstream vectors were selected based on these assessments and ensured that an out-of-frame ATG codon in good context was present within 100-200 nucleotides of the 5′ITR prior to any in-frame ATG in good context. Designing the new downstream transgenes using these criteria influenced the levels of tABCA4 observed as the original unrecombined downstream A and B transgenes generated greater levels of tABCA4 protein than all other variants (FIG. 10a). In a dual vector context, variants A-D generated detectable ABCA4 forms (FIG. 9a) and of the total detected ABCA4 population, cells treated with variants A and B achieved a tABCA4 proportion of 21±5% and 25±0.8%, respectively, whereas the truncated ABVA4 (tABCA4) population from variant C and D treated samples were 4±2% and 3±3%, respectively (FIG. 9D).

AAV doses in vitro were consistent for all variants and Abca4^{-/-} eyes received 1×10⁹ genome copies of each vector per eye except dual vector variant A, which due to its larger transgene size packaged less efficiently and therefore the final dose was 8×10⁸ genome copies per eye. Dual vector variant 5′C was identified as the optimal dual vector system and when used at a higher dose of 1×10¹⁰ genome copies per eye, a significant improvement in levels of ABCA4 was achieved (p=0.006, FIG. 9B). These data indicated that the changes implemented in the downstream transgene variants C-X limited the generation of tABCA4 forms from unrecombined transgenes. In contrast to the presence of in-frame ATG codons in downstream constructs A and B, variants C to X contained out-of-frame ATG codons with a Kozak consensus prior to any in-frame ATG codons downstream of the 5′ITR.

In one case, to reduce expression of truncated ABCA4 from the downstream vector overlap C (207 bp) was selected. Overlap C, although slightly less efficient than B (505 bp), gave a purer ABCA4 protein which could have safety benefits in the clinical scenario. Therefore, overlap C was combined with the intron-containing upstream vector and both were packaged into AAV8 Y733F capsids. This dual vector combination was used for subsequent testing in vivo in the Abca4−/− mouse at 10¹⁰ genome copies per eye.

Based on evidence that a WPRE increases AAV transcript expression levels, we initially included this element in our downstream transgene design. However, with the observations of mRNA transcripts and truncated protein being generated from unrecombined downstream transgenes, we contemplated removing the WPRE to potentially limit this unwanted expression. Levels of truncated ABCA4 protein were reduced in vitro when the WPRE from variant B was removed (variant Bx) (FIG. 10a). Furthermore, Abca4^{-/-} eyes injected with downstream vector only revealed a reduction in truncated ABCA4 mRNA transcript levels between downstream B and Bx treated eyes (FIG. 14b). Removing the WPRE did therefore reduce the expression levels of truncated ABCA4. However, the design of downstream transgenes also enabled a reduction in tABCA4 production.

Assessing the safety of a dual vector system includes the identification of unwanted byproducts from unrecombined transgenes. Assessments using either upstream or downstream vectors (not in combination) revealed that each vector in an unrecombined state can generate truncated ABCA4 mRNA transcript forms. The upstream transgene nucleotide sequence contained a SwaI restriction site used for cloning purposes that could be acting as a cryptic polyA signal: TTTAAA, which has been identified as a polyA signal in 1-2% of human genes. The absence of any protein detection following treatment with the upstream vector could be attributed to the lack of an in-frame stop codon in the resulting mRNA transcript, which would lead to degradation of any generated peptide. Truncated ABCA4 (tABCA4) protein was detected from original downstream vector designs (FIG. 9). One approach to reduce unwanted protein production from dual vectors is by inclusion of additional genetic sequences in the transgene design. Another approach employed sequence selection of coding regions that carried out-of-frame ATG codons with a Kozak consensus sequence prior to any in-frame ATG codons within 100 bases of the 5′ITR, reducing truncated ABCA4 (tABCA4) to negligible levels (FIG. 9). The ABCA4 coding sequence to be included in the downstream vector was analyzed and specifically selected coding sequence that carried out-of-frame ATG codons prior to any in-frame ATG codons within 100 bases of the 5′ITR was selected. This sequence design was based on the evidence that ribosomes favor initiating translation from the first AUG codon they encounter in good context. If the ribosomes attempted to initiate translation from unrecombined downstream transgenes at an out-of-frame AUG, we could predict that only short peptides would be formed before an out-of-frame stop codon was reached, and such short peptides would then be degraded by the cell due to their size. This strategy was successful as five new downstream transgene variants designed in this way revealed a reduction in tABCA4 production to almost negligible levels despite providing a high dose of single vector transgenes. In contrast, the original downstream transgenes (A and B) both generated tABCA4 and both carried in-frame ATG codons prior to any out-of-frame ATG codons in good context. This transgene design feature could be implemented in other dual vector strategies. If a given coding sequence carries no out-of-frame codons prior to any in-frame codons then selected codon-optimization could be a worthwhile option. It may also be that full sequence codon-optimization will be adopted in the future as this has been shown to increase translational rates and clinical trials have begun that use codon-optimized gene sequences including the recently initiated Phase I/II clinical trial for X-linked retinitis pigmentosa (NCT03116113).

Having improved recombination efficiency and determined an optimal overlap region using the WT ABCA4 sequence, further ways to increase expression from recombined transgenes were demonstrated. There is evidence indicating that including a WPRE in the transgene structure could increase ABCA4 expression from recombined transgenes. However, an added complication in including this genetic element was that in early transgene designs, unwanted protein expression of truncated (tABCA4) was observed from the unrecombined downstream vector, which did contain a WPRE. Removing the WPRE reduced the levels of tABCA4 but given that the chance of the dual vector approach achieving therapeutic levels of ABCA4 could rely on generating the most amount of protein from a given recombined transgene, keeping the WPRE was also effective. Subsequent changes to the downstream transgene design were able to reduce truncated ABCA4 to negligible levels whilst maintaining the WPRE in the construct.

Including a spliceable 5′UTR element improved levels of full length ABCA4 protein achieved following dual vector treatment. It has previously been shown that introns near the promoter can augment pre-mRNA synthesis and interact synergistically with the polyadenylation machinery to enhance 3′ end transcript processing. Studies have shown that mRNA transcripts which undergo splicing exhibit higher translational yields than equivalent intronless transcripts and placing the intron near the promoter enhances gene expression more than when used inside the coding sequence. This data supports and reinforces these findings, and encourage and support the standard use of introns in vector transgenes.

Other dual vector approaches and nanoparticle delivery have led to successful ABCA4 expression in adult Abca4^{-/-} mice and provided evidence of positive effects attributed to the ABCA4 expression. This study shows for the first time convincing expression of ABCA4 in the photoreceptor outer segments of adult Abca4^{-/-} mouse retinae following injection with an optimized overlapping dual vector system. This ABCA4 exhibited functional activity by reducing the levels of bisretinoids that accumulate in the disease model, an effect that was confirmed in two independent in vivo studies in the mouse model. In patients with Stargardt disease, the bisretinoid accumulation leads to death of the RPE cells and subsequently the degeneration and death of the photoreceptor cells, which results in blindness. Given the progressive degenerative nature of this disorder, providing therapeutic intervention at any age could be anticipated to be beneficial by preserving the surviving cells of the retina. By optimizing an overlapping dual vector system to increase the levels of therapeutic protein delivered to the target cells and, importantly, reducing the expression of unwanted products that often occur in dual vector strategies, AAV gene therapy clinical trial prospects for Stargardt disease are now looking increasingly achievable.

### Example 8—Codon Optimization

Initial comparisons of ABCA4 protein levels were compared from wild-type and codon-optimized ABCA4 coding sequences: In some cases, protein production could be enhanced through the use of a codon optimized (CO) ABCA4 coding sequence. Plasmids were generated carrying an expression cassette identical but for the inclusion of WT or CO coding sequence. Samples were harvested 48 hours post-transfection and lysates assessed by western blot analysis with ABCA4 detection standardized to GAPDH sample levels and data presented as values above background (of transfected samples). Constructs identical but for the inclusion of wild-type (WT) or codon-optimized (CO) ABCA4 coding sequence were compared and revealed a significant 3.1-fold increase in ABCA4 protein generated from CO coding sequence compared to WT coding sequence (FIG. 28a). The plasmids were identical except for the WT or CO ABCA4 coding sequence. A difference in generated ABCA4 protein levels determined (two-tailed unpaired t-test, n=4, ***p=0.0002, F(3,3)=2.973).

To investigate whether an increase in ABCA4 protein generation by codon optimization could also be achieved in a dual vector scenario, AAV2/2 in vitro transductions using overlapping dual vectors (identical but for the inclusion of WT or CO coding sequence) were performed. The protein analysis from these samples revealed no difference in ABCA4 levels (FIG. 28b, two-tailed unpaired t-test, n=3, F(2,2)=18.74).

To determine if such positive effects of the CO coding sequence could be achieved in vivo, Abca4-mice received a sub-retinal injection of AAV2/8 overlapping dual vectors carrying transgenes identical but for the coding sequence being WT or CO with expression driven by the GRK1 promoter (transgene details Table 2). The overlap zone for both dual vector systems started at position 3,154 of the ABCA4 cDNA and finished at nucleotide 4,326, the GC content of the overlap region for both WT and CO sequences was 55% (Table 2). ABCA4 detection from isolated retinae was assessed at 2 weeks and 6 weeks post-injection with an influence of the coding sequence observed (p=0.04, FIG. 28c). No difference was seen at 2 weeks post-injection in levels of ABCA4 protein detected from WT or CO overlapping dual vector injected eyes. At 6 weeks post-injection, more ABCA4 protein was detected from WT injected samples than CO injected samples and ABCA4 detection from WT injected retinae at this time point was consistently greater than at 2 weeks post-injection (p=0.005, FIG. 2c). These data showed that changing base pairs within an otherwise identical length of dual vector overlap led to changes in protein expression. This observation indicated that the efficiency of base pairing may be a contributing factor in dual vector recombination (rather than simply codon bias). With no enhancement observed in ABCA4 production with the use of CO ABCA4 coding sequence in the overlapping dual vector system, we opted to use the WT coding sequence in subsequent optimizations.

Side-by-side comparisons of intact full length WT and CO ABCA4 coding sequence indicated that the codon-optimization of ABCA4 did enable higher translational rates. However, given that the coding sequence was used as the region of overlap in the dual vector system, the changes made to the coding sequence in the CO variant may have influenced the success of recombination. If the CO transgenes were recombining as efficiently as the WT version, it would have been anticipated that from an equivalent number of transgenes, the CO variant would produce more protein. Yet when tested in vivo, dual vectors containing the WT coding sequence generated more ABCA4 than the equivalent CO dual vector system. This may indicate that WT dual vector injected eyes contained more successfully recombined transgenes than CO dual vector injected eyes. Alternatively, the transgenes could have recombined to a similar extent yet the CO ABCA4 coding sequence was translated less efficiently in mouse photoreceptor cells. The codon-optimization was weighted towards human expression therefore the translation rate in the mouse may have been negatively influenced. However, the WT sequence used was human-derived and would also have a different codon-bias preference than would be ideal for use in mouse cells therefore both sequences could be considered to be disadvantaged when translated in mouse cells. Another consideration is that only one overlap region for the WT and CO dual vector systems was compared, and the importance of the overlap region in the success of the overlapping dual vector system has since been shown. Finally, the codon changes in this scenario are not just limited to mRNA translation, but also to DNA repair, because the second strand synthesis is contributes to the success of the dual vector strategy and this may be favored by certain nucleotides being exposed.

Whilst ABCA4 detection was higher when using a codon-optimized construct in plasmid form, for dual vector AAV recombination it was found that the wild-type sequence was more efficacious. In some embodiments, changing codons also affects DNA base pairing and hence has a direct influence on dual vector recombination in this scenario.

With no enhancement observed in ABCA4 production with the use of CO ABCA4 coding sequence in the overlapping dual vector system, we opted to use the WT coding sequence in subsequent optimizations. It was noted that from retinae injected with only the downstream vector, either WT or CO coding sequence, that a truncated ABCA4 (tABCA4) protein ˜135 kDa was detectable (FIG. 28c). This protein band was also apparent in some dual vector injected eyes but was not easily identifiable in all samples.

### Example 9—Reduction in Lipofuscin and Melanin-Related Autofluorescence

Directly measuring bisretinoid levels in Abca4^{-/-} mice enabled quantifiable assessment of therapeutic efficacy in vivo. In addition to directly measuring bisretinoid/A2E levels of treated Abca4^{-/-} mice three months post-injection, scanning laser ophthalmoscopy (SLO) assessment of autofluorescence was also performed at 3 and 6 months post-injection using the 790 nm wavelength shown to be associated with melanin accumulation. Scanning laser ophthalmoscopy (SLO) assessment of autofluorescence was also performed using the 790 nm wavelength as an in vivo measure for melanolipofuscin accumulation. SLO assessment of autofluorescence is a potential human clinical trial endpoint. The mouse model exhibits an increase in lipofuscin and melanin-related autofluorescence over time, compared to WT control mice. The 488 nm wavelength autofluorescence measurements were shown to reflect an accumulation of lipofuscin whilst the 790 nm wavelength autofluorescence was associated with melanin accumulation.

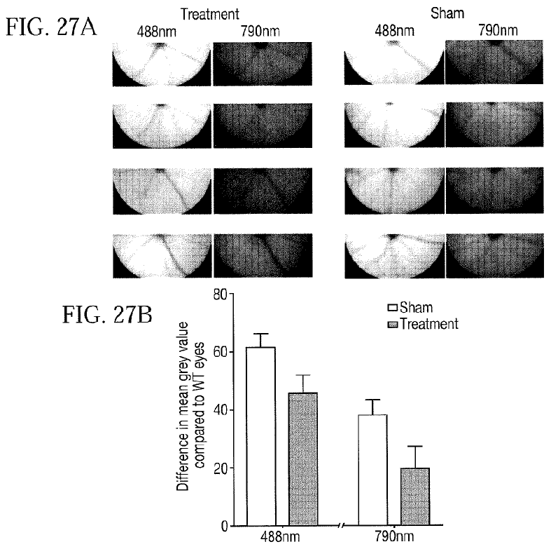

In this cohort, mice were injected in one eye with a sham injection (PBS) to control for the effects of retinal detachment, while the contralateral eye received the optimized overlapping dual vector system (2E+10 total genome copies), with the aim of observing a treatment-related decrease in lipofuscin and melanin-related autofluorescence. A standardized SLO protocol based on previous work was used (52) and when extracting the mean grey value of each image, a standardized area of measurement was taken only from the inferior retina to avoid disrupted autofluorescence caused by surgical damage or surgically induced changes around the site of injection which was in the superior hemi-retina. Twelve mice each received the sham injection in one eye and the treatment in the contralateral eye. The eyes that received treatment showed a reduction in mean grey values at both 488 nm and 790 nm wavelengths compared to the paired sham injected eyes (488 nm sham 221.4±4.7 and treatment 205.9±6.3; 790 nm sham 119.9±5.2 and treatment 101.4±7.1). Between 3 and 6 months post-injection, eyes exhibited an increase in 790 nm autofluorescence but the increase was greater in the sham injected eyes compared to the paired dual vector injected eyes (p=0.04, FIG. 31B). In these eyes, the increase in levels of 790 nm autofluorescence was significantly attenuated in the ABCA4 dual vector injected eyes compared with the paired sham injected eyes. These data reflected an influence of the treatment on the levels of lipofuscin and melanin-related autofluorescence in Abca4^{-/-} mice (FIG. 27).

Mouse fundus autofluorescence (AF) imaging using a confocal scanning laser ophthalmology (cSLO; SpectralisHRA, Heidelberg Engineering, Heidelberg, Germany) was performed using a standardized protocol. Fluorescence was excited using a 488 nm argon laser or a 790 nm diode laser. Animals were anaesthetized and pupils fully dilated as described. A custom-made contact lens was placed on the cornea with hypromellose eye drops (Hypromellose eye drops 1%, Alcon, UK) as a viscous coupling fluid. The NIR reflectance image (820 nm diode laser) was used to align the fundus camera relative to the pupil and to focus on the confocal plane of highest reflectivity in the outer retina. Images were recorded using the “automatic real time” (ART) mode, set to average 24 consecutive images in real time to reduce signal-to-noise ratio. The mean grey value of 488 nm and 790 nm AF images were extracted by measuring a standardized ring shaped area between 250 and 500 pixel radii from the optic disc center using ImageJ software. Each image was then cut to remove the superior retina and the standardized ring applied only to the inferior retina.

### Example 10—Capsid Variants

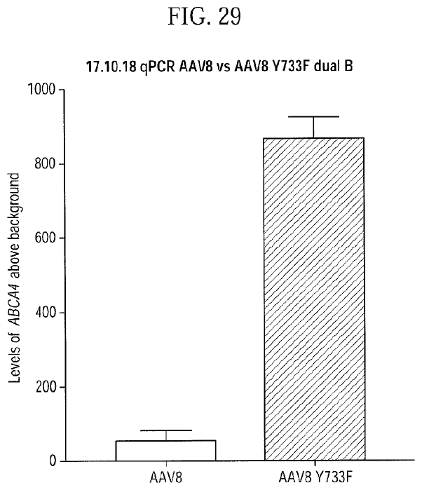

Initial in vivo experiments used the AAV2/8 serotype. For example, the WT vs CO comparisons employed AAV8 vectors. However, successful homologous recombination of overlapping plus and minus strands released from two separate AAV vectors might be optimized if the vectors remained within the cell for longer. In some embodiments, changes to the AAV capsid protein amino acids by substituting tyrosine (Y) for phenylalanine (F) have been shown slow down proteosomal degradation but without affecting tropism. In some embodiments, changes to the AAV capsid protein amino acids by substituting tyrosine (Y) for phenylalanine (F) have been shown to improve transduction and in the Abca4^{-/-} retina. The success of the overlapping dual vector approach in Abca4^{-/-} retinae using identical transgenes packaged in either AAV2/8 or AAV2/8 Y733F capsids was compared. FIG. 29 compares the overlapping dual vector approach in Abca4^{-/-} retinae using identical transgenes packaged in either AAV2/8 or AAV2/8 Y733F capsids. ABCA4 transcript levels were normalized to Actin levels per sample and are presented as fold increase relative to uninjected eyes. Fewer ABCA4 transcripts were detected in Abca4^{-/-} retinae injected with the AAV2/8 dual vectors (p=0.002, FIG. 29). 31.3±7.8 times more ABCA4 transcripts were detected in Abca4−/− retinae injected with the AAV8 Y733F dual vectors.

This study has addressed the need for improved dual vector strategies for the treatment of disorders caused by mutations in large genes by demonstrating step-by-step investigations to improve the success of a dual vector overlapping AAV treatment strategy for Stargardt disease. Previously, questions have been raised regarding whether these strategies could lead to production of enough target protein to provide therapeutic effect. Developing a treatment for Stargardt disease is a good example for assessing the possibility of achieving therapeutic effect because the target protein, ABCA4, is required in abundance in the photoreceptor cells of the retina. The optimizations achieved in this work include those universally applicable to AAV gene therapies but specific enhancements presented could also be recommended for implementation in other dual vector strategies.

This data confirmed previous findings of improvements in transduction when using AAV8 Y733F compared to AAV8 capsids to deliver ABCA4 coding sequence in dual vector transgenes. Enhancing transgene delivery and survival in the photoreceptor cells by capsid and dose selection is influential to the dual vector treatment success to increase the opportunity for intermolecular interactions between transgenes. This was further highlighted by comparison of eyes injected with dual vector at different doses in which a higher dose led to an increase in the detection of full length ABCA4 protein. An improvement in dual vector success by increasing dose has been previously shown with a hybrid dual vector approach and this data shows a similar result using an optimized overlapping dual vector system.

### Example 11—Assessment of AAV Dual Vector Safety in the Abca4 KO Mouse Model

The Abca4 KO mouse model presents no electroretinogram (ERG) phenotype or histological degeneration except for age-related changes, therefore signs of toxicity can be measured by changes to retinal function and loss of retinal structure.

In a blinded study, Abca4 KO mice received a subretinal injection in the superior retina of the right eye at 4-5 weeks of age (n=8-11 per group). Injected materials tested were: AAV diluent (PBS PF68 0.001%); GRK1.GFP.pA high dose (2E+10 genome copies); upstream vector low dose (2E+9 genome copies); upstream vector high dose (2E+10 genome copies); downstream vector (1E+10 genome copies); dual vector low dose (2E+9 total genome copies); dual vector high dose (2E+10 total genome copies). All vectors were AAV8 Y733F. Standardised optical coherence tomography (OCT) and ERG assessments were performed at 3 and 6 months post-injection.

Mice in all cohorts revealed varying degrees of post-injection damage. Performing a subretinal injection had a significant influence on superior total retinal thickness compared to uninjected paired eyes at both 3 and 6 months post-injection (3 months two-way ANOVA: eye p<0.001, cohort p=0.7012, interaction p=0.6203; 6 months two-way ANOVA: eye p<0.001, cohort p=0.6858, interaction p=0.6230). The reduction in total retinal thickness was not influenced by the injection material with no additional loss observed in vector injected mice compared to those that received AAV diluent only. All cohorts exhibited a significant reduction in ERG amplitude at 1cd.s/m2 between injected and uninjected eyes (3 months two-way ANOVA: eye p<0.0001, cohort p=0.0173, interaction p=0.5954; 6 months two-way ANOVA: eye p<0.0001, cohort p=0.0102, interaction p=0.4437). The change in the magnitude of response between paired eyes was not significantly different between the cohorts at either time point (two-way ANOVA: time point p=0.8507, cohort p=0.4014, interaction p=0.0491).

Performing a subretinal injection in Abca4 KO mice led to loss of total retinal thickness and a drop in ERG amplitude. The magnitude of such changes were no different in dual vector injected mice than those that received AAV diluent only.

All publications mentioned in the above specification are herein incorporated by reference. Various modifications and variations of the described products, systems, uses, processes and methods of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure, which are obvious to those skilled in biochemistry and biotechnology or related fields, are intended to be within the scope of the following claims.

1. An adeno-associated viral (AAV) vector system for expressing a human ABCA4 protein in a target cell, the AAV vector system comprising a first AAV vector comprising a first nucleic acid sequence and a second AAV vector comprising a second nucleic acid sequence;
wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS;
wherein the first nucleic acid sequence comprises a sequence of contiguous nucleotides corresponding to nucleotides 105 to 3597 of SEQ ID NO: 1;
wherein the second nucleic acid sequence comprises a sequence of contiguous nucleotides corresponding to nucleotides 3806 to 6926 of SEQ ID NO: 1;
wherein the first nucleic acid sequence and the second nucleic acid sequence each comprise a region of sequence overlap with the other; and
wherein the region of sequence overlap comprises at least about 20 contiguous nucleotides of a nucleic acid sequence corresponding to nucleotides 3598 to 3805 of SEQ ID NO: 1.

2. The AAV vector system of claim 1, wherein the region of sequence overlap is between 20 and 550 nucleotides in length.

3. The AAV vector system of claim 1, wherein the region of sequence overlap is between 50 and 250 nucleotides in length.

4. The AAV vector system of claim 1, wherein the region of sequence overlap is between 175 and 225 nucleotides in length.

5. The AAV vector system of claim 1, wherein the region of sequence overlap is between 195 and 215 nucleotides in length.

6. The AAV vector system of any one of claims 1-5, wherein the region of sequence overlap comprises at least about 50 contiguous nucleotides of a nucleic acid sequence corresponding to nucleotides 3598 to 3805 of SEQ ID NO: 1.

7. The AAV vector system of any one of claims 1-5, wherein the region of sequence overlap comprises at least about 75 contiguous nucleotides.

8. The AAV vector system of any one of claims 1-5, wherein the region of sequence overlap comprises at least about 100 contiguous nucleotides.

9. The AAV vector system of any one of claims 1-5, wherein the region of sequence overlap comprises at least about 150 contiguous nucleotides.

10. The AAV vector system of any one of claims 1-5, wherein the region of sequence overlap comprises at least about 200 contiguous nucleotides.

11. The AAV vector system of any one of claims 1-5, wherein the region of sequence overlap comprises 208 contiguous nucleotides.

12. The AAV vector system of any one of the preceding claims,
wherein the first nucleic acid sequence comprises a sequence of contiguous nucleotides corresponding to nucleotides 105 to 3805 of SEQ ID NO: 1; and
wherein the second nucleic acid sequence comprises a sequence of contiguous nucleotides corresponding to nucleotides 3598 to 6926 of SEQ ID NO: 1.

13. The AAV vector system of any one of the preceding claims, wherein the first nucleic acid sequence comprises a GRK1 promoter operably linked to the 5′ end portion of an ABCA4 coding sequence (CDS).

14. The AAV vector system of any one of the preceding claims, wherein the first nucleic acid sequence comprises a CBA promoter operably linked to the 5′ end portion of an ABCA4 coding sequence (CDS).

15. The AAV vector system of claim 14, wherein the first nucleic acid sequence further comprises a CMV enhancer.

16. The AAV vector system of claim 14 or 15, wherein the first nucleic acid sequence further comprises an intron and exon.

17. The AAV vector system of any one of claims 14-16, wherein the first nucleic acid sequence comprises a CAG promoter.

18. The AAV vector system of any one of the preceding claims, wherein the first nucleic acid sequence comprises an untranslated region (UTR) located upstream of the 5′ end portion of an ABCA4 coding sequence (CDS).

19. The AAV vector system of any one of the preceding claims, wherein the second nucleic acid sequence comprises a post-transcriptional response element (PRE).

20. The AAV vector system of any one of the preceding claims, wherein the second nucleic acid sequence comprises a Woodchuck hepatitis virus post-transcriptional response element (WPRE).

21. The AAV vector system of any one of the preceding claims, wherein the second nucleic acid sequence comprises a bovine Growth Hormone (bGH) poly-adenylation sequence.

22. The AAV vector system of any one of the preceding claims, wherein the first AAV vector comprises the nucleic acid sequence of SEQ ID NO: 9; and wherein the second AAV vector comprises the nucleic acid sequence of SEQ ID NO: 10.

23. A method for expressing a human ABCA4 protein in a target cell, the method comprising the steps of:
transducing the target cell with the first AAV vector and the second AAV vector of any one of claims 1-22, such that a functional ABCA4 protein is expressed in the target cell.

24. An AAV vector comprising a nucleic acid sequence comprising a 5′ end portion of an ABCA4 CDS, wherein the 5′ end portion of an ABCA4 CDS consists of a sequence of contiguous nucleotides corresponding to nucleotides 105 to 3805 of SEQ ID NO: 1.

25. The AAV vector of claim 11, wherein the AAV vector comprises the nucleic acid sequence of SEQ ID NO: 9.

26. An AAV vector comprising a nucleic acid sequence comprising a 3′ end portion of an ABCA4 CDS, wherein the 3′ end portion of an ABCA4 CDS consists of a sequence of contiguous nucleotides corresponding to nucleotides 3598 to 6926 of SEQ ID NO: 1.

27. The AAV vector of claim 13, wherein the AAV vector comprises the nucleic acid sequence of SEQ ID NO: 10.

28. The AAV vector of any one of claims 1-27, the first nucleic acid sequence or the second nucleic acid sequence further comprises a sequence encoding a 5′ inverted terminal repeat (ITR) and a sequence encoding a 3′ ITR.

29. The AAV vector of claim 28, wherein the sequence encoding a 5′ ITR comprises a wild type sequence isolated or derived of a serotype 2 AAV (AAV2).

30. The AAV vector of claim 28 or 29, wherein the sequence encoding the 5′ ITR comprises the sequence of SEQ ID NO: 27 or a deletion variant thereof.

31. The AAV vector of any one of claims 28-30, wherein the sequence encoding a 3′ ITR comprises a wild type sequence isolated or derived of an AAV2.

32. The AAV vector of claim 31, wherein the sequence encoding the 3′ ITR comprises the sequence of SEQ ID NO: 30 or a deletion variant thereof.

33. The AAV vector of claim 30 or 32, wherein the deletion variant comprises or consists of 10, 20, 30, 40, 50, 70, 80, 90, 100, 110, 120, 130, 140, 144 nucleotides or any number in between of nucleotides.

34. The AAV vector of any one of claims 30, 32, or 33, wherein the deletion variant comprises one or more deletions.

35. The AAV vector of claim 34, wherein the deletion variant comprises at least two deletions.

36. The AAV vector of claim 35, wherein the at least two deletions are not contiguous.

37. A nucleic acid comprising the first nucleic acid sequence of any one of claims 1 to 36.

38. A nucleic acid comprising the second nucleic acid sequence of any one of claims 1 to 36.

39. A nucleic acid comprising or consisting of the nucleic acid sequence of SEQ ID NO: 9.

40. A nucleic acid comprising or consisting of the nucleic acid sequence of SEQ ID NO: 10.

41. A kit comprising the first AAV vector of any one of claims 1 to 36 and the second AAV vector of any of claims 1 to 26.

42. A pharmaceutical composition comprising the AAV vector system of any of claims 1 to 36 and a pharmaceutically acceptable excipient.

43. An AAV vector system according to any one of claims 1-42 for use in gene therapy.

44. A pharmaceutical composition according to claim 43 for use in gene therapy.

45. An AAV vector system according to any one of claims 1-36 for use in preventing or treating a disease characterized by degradation of retinal cells.

46. An AAV vector system according to any one of claims 1-36 for use in preventing or treating Stargardt disease.

47. A pharmaceutical composition according to claim 42 for use in preventing or treating a disease characterized by degradation of retinal cells.

48. A pharmaceutical composition according to claim 42 for use in preventing or treating Stargardt disease

49. A method for preventing or treating a disease characterized by degradation of retinal cells, comprising administering to a subject in need thereof an effective amount of an AAV vector system according to any of claims 1-36.

50. A method for preventing or treating a disease characterized by degradation of retinal cells, comprising administering to a subject in need thereof an effective amount of a pharmaceutical composition according to claim 42.

51. The method of claim 49 or 50, wherein the disease is Stargardt disease.


SEQUENCE LISTING (75 sequences)

SEQ ID NO: 1 - DNA, 7326 bp, Homo sapiens
  Human ABCA4 (1)..(7326)
aggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaaa

SEQ ID NO: 2 - DNA, 7326 bp, Homo sapiens
  Human ABCA4 Mutation (1)..(7326): nucleotide 1640 G>T, nucleotide 5279 G>A, nucleotide 6173 T>C
aggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaaa

SEQ ID NO: 3 - DNA, 4464 bp, Artificial Sequence
  Recombinant synthesis
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggcaattcagtcgataactataacggtcctaaggtagcgatttaaatggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtaatggcgcgggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 4 - DNA, 4581 bp, Artificial Sequence
  Recombinant synthesis
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggcaattcagtcgataactataacggtcctaaggtagcgatttaaataacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatgccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggatttaaattagggataacagggtaatggcgcgggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 5 - DNA, 199 bp, Artificial Sequence
  Recombinant synthesis
  GRK1 promoter (1)..(199)
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggg

SEQ ID NO: 6 - DNA, 186 bp, Artificial Sequence
  Recombinant synthesis
  UTR (1)..(186)
gtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgg

SEQ ID NO: 7 - DNA, 593 bp, Artificial Sequence
  Recombinant synthesis WPRE element
  WPRE (1)..(593)
atcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcccc

SEQ ID NO: 8 - DNA, 269 bp, Artificial Sequence
  Recombinant synthesis
  BGH polyA (1)..(269)
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 9 - DNA, 4087 bp, Artificial Sequence
  Recombinant synthesis
ggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttcc

SEQ ID NO: 10 - DNA, 4203 bp, Artificial Sequence
  Recombinant synthesis
acatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatgccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 11 - DNA, 6822 bp, Homo sapiens
  ABCA4 cDNA (1)..(6822): Corresponds to nucleotides 105-6926 of SEQ ID NO: 1
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactga

SEQ ID NO: 12 - protein, 737 aa, adeno-associated virus 8
  AAV8 Capsid Protein (1)..(737): Corresponds to nucleotides 105-6926 of SEQ ID NO: 1
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRN

SEQ ID NO: 13 - DNA, 123 bp, Artificial Sequence
  Recombinant synthesis
  Intron (1)..(123)
gtgccgcagggggacggctgccttcgggggggacggggcagggcggggttcggcttctggcgtgtgaccggcggctctagagcctctgctaaccatgttcatgccttcttctttttcctacag

SEQ ID NO: 14 - DNA, 53 bp, Artificial Sequence
  Recombinant synthesis
  exon (1)..(53)
ctcctgggcaacgtgctggttattgtgctgtctcatcattttggcaaagaatt

SEQ ID NO: 15 - DNA, 235 bp, Artificial Sequence
  Recombinant synthesis
  CMV enhancer (1)..(235)
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtca

SEQ ID NO: 16 - DNA, 372 bp, Artificial Sequence
  Recombinant synthesis
  CBA promoter (1)..(372)
gtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacag

SEQ ID NO: 17 - DNA, 7496 bp, Artificial Sequence
  Recombinant synthesis
ctgcgcgctcgctcgctcactgaggccgcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattgccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagagctcctcgaggcggcccgctcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagggtgaccgatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtagggtgacctaagccagatgctacacaattaggcttgtacatattgtcgttagaacgcggctacaattaatacataaccttatgtatcatacacatacgatttaggtgacactatagaatacacggaattaattctag

SEQ ID NO: 18 - DNA, 7321 bp, Artificial Sequence
  Recombinant synthesis
ctgcgcgctcgctcgctcactgaggccgcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattgccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagagctcctcgaggcggcccgctcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagggtgaccgatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtagggtgacctaagccagatgctacacaattaggcttgtacatattgtcgttagaacgcggctacaattaatacataaccttatgtatcatacacatacgatttaggtgacactatagaatacacggaattaattctag

SEQ ID NO: 19 - DNA, 7483 bp, Artificial Sequence
  Recombinant synthesis
ctgcgcgctcgctcgctcactgaggccgcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagagctcctcgaggcggcccgctcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagggtgaccgatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtagggtgacctaagccagatgctacacaattaggcttgtacatattgtcgttagaacgcggctacaattaatacataaccttatgtatcatacacatacgatttaggtgacactatagaatacacggaattaattctag

SEQ ID NO: 20 - DNA, 7728 bp, Artificial Sequence
  Recombinant synthesis
ctgcgcgctcgctcgctcactgaggccgcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattgccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagagctcctcgaggcggcccgctcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagggtgaccgatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtagggtgacctaagccagatgctacacaattaggcttgtacatattgtcgttagaacgcggctacaattaatacataaccttatgtatcatacacatacgatttaggtgacactatagaatacacggaattaattctag

SEQ ID NO: 21 - DNA, 10070 bp, Artificial Sequence
  Recombinant synthesis
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggcaattcagtcgataactataacggtcctaaggtagcgatttaaatggtaccccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 22 - DNA, 9895 bp, Artificial Sequence
  Recombinant synthesis
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggcaattcagtcgataactataacggtcctaaggtagcgatttaaatggtaccccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 23 - DNA, 10057 bp, Artificial Sequence
  Recombinant synthesis
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggcaattcagtcgataactataacggtcctaaggtagcgatttaaatggtacccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 24 - DNA, 279 bp, Artificial Sequence
  Recombinant synthesis
  promoter (1)..(279)
gtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 25 - DNA, 263 bp, Artificial Sequence
  Recombinant synthesis
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccag

SEQ ID NO: 26 - DNA, 4464 bp, Artificial Sequence; identical to SEQ ID NO: 3
  Recombinant synthesis

SEQ ID NO: 27 - DNA, 145 bp, Artificial Sequence
  Recombinant synthesis
  ITR 55′ (1)..(145): 5′ ITR of SEQ ID NO: 26
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 28 - DNA, 199 bp, Artificial Sequence; identical to SEQ ID NO: 5
  Recombinant synthesis
  promoter (1)..(199): GRK1 promoter of SEQ ID NO: 26

SEQ ID NO: 29 - DNA, 3703 bp, Artificial Sequence
  Recombinant synthesis
catgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttcca

SEQ ID NO: 30 - DNA, 145 bp, Artificial Sequence
  Recombinant synthesis
  3′ ITR (1)..(145)
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 31 - DNA, 3334 bp, Artificial Sequence
  Recombinant synthesis
  3′ ABCA4 gene (1)..(3334)
aaataacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactga

SEQ ID NO: 32 - DNA, 593 bp, Artificial Sequence; identical to SEQ ID NO: 7
  Recombinant synthesis
  WPRE (1)..(593)

SEQ ID NO: 33 - DNA, 269 bp, Artificial Sequence; identical to SEQ ID NO: 8
  Recombinant synthesis
  BGH polyA (1)..(269)

SEQ ID NO: 34 - DNA, 119 bp, Artificial Sequence
  Recombinant synthesis
  5′ ITR (1)..(119)
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 35 - DNA, 130 bp, Artificial Sequence
  Recombinant synthesis
  5′ ITR (1)..(130)
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 36 - DNA, 130 bp, Artificial Sequence
  Recombinant synthesis
  5′ ITR (1)..(130)
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 37 - DNA, 121 bp, Artificial Sequence
  Recombinant synthesis
  3′ ITR (1)..(121)
aggaacccctagtgatggagttggccactccctctctgcgcgctcgctcgctcactgaggccgggcgaccaaaggtcgcccgacgcccgggcggcctcagtgagcgagcgagcgcgcagag

SEQ ID NO: 38 - skipped

SEQ ID NO: 39 - DNA, 188 bp, Artificial Sequence
  Recombinant synthesis
  RBG (1)..(188)
agccccgggtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacca

SEQ ID NO: 40 - DNA, 9819 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaataacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatgccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggatttaaattagggataacagggtaatggcgcgggccgcaggaacccctagtgatggagttggccactccctctctgcgcgctcgctcgctcactgaggccgggcgaccaaaggtcgcccgacgcccgggcggcctcagtgagcgagcgagcgcgcagagctagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 41 - DNA, 120 bp, Artificial Sequence
  Recombinant synthesis
gctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 42 - DNA, 3334 bp, Artificial Sequence; identical to SEQ ID NO: 31
  Recombinant synthesis

SEQ ID NO: 43 - DNA, 593 bp, Artificial Sequence; identical to SEQ ID NO: 7
  Recombinant synthesis

SEQ ID NO: 44 - DNA, 174 bp, Artificial Sequence
  Recombinant synthesis
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggg

SEQ ID NO: 45 - DNA, 121 bp, Artificial Sequence; identical to SEQ ID NO: 37
  Recombinant synthesis

SEQ ID NO: 46 - DNA, 9786 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatggtacccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgggcgaccaaaggtcgcccgacgcccgggcggcctcagtgagcgagcgagcgcgcagagctagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 47 - DNA, 115 bp, Artificial Sequence
  Recombinant synthesis
  5′ ITR (1)..(115)
ctcactgaggccgcccgggcaaagcccgggcgtcgggcgacctttggtcgcccggcctcagtgagcgagcgagcgcgcagagagggagtggccaactccatcactaggggttcct

SEQ ID NO: 48 - DNA, 278 bp, Artificial Sequence
  Recombinant synthesis
  CBA (1)..(278)
gtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggc

SEQ ID NO: 49 - DNA, 123 bp, Artificial Sequence; identical to SEQ ID NO: 13
  Recombinant synthesis
  Intron (1)..(123)

SEQ ID NO: 50 - DNA, 40 bp, Artificial Sequence
  Recombinant synthesis
  exon (1)..(40)
ctcctgggcaacgtgctggttattgtgctgtctcatcatt

SEQ ID NO: 51 - DNA, 3702 bp, Artificial Sequence
  Recombinant synthesis
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttcca

SEQ ID NO: 52 - DNA, 121 bp, Artificial Sequence; identical to SEQ ID NO: 37
  Recombinant synthesis
  3′ ITR (1)..(121)

SEQ ID NO: 53 - DNA, 10055 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatggtacccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcctagcttgatatcgaattcctgcagcccggcaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgggcgaccaaaggtcgcccgacgcccgggcggcctcagtgagcgagcgagcgcgcagagctagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 54 - DNA, 115 bp, Artificial Sequence; identical to SEQ ID NO: 47
  Recombinant synthesis

SEQ ID NO: 55 - DNA, 278 bp, Artificial Sequence; identical to SEQ ID NO: 48
  Recombinant synthesis

SEQ ID NO: 56 - DNA, 173 bp, Artificial Sequence
  Recombinant synthesis
ccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 57 - DNA, 3702 bp, Artificial Sequence; identical to SEQ ID NO: 51
  Recombinant synthesis

SEQ ID NO: 58 - DNA, 121 bp, Artificial Sequence; identical to SEQ ID NO: 37
  Recombinant synthesis

SEQ ID NO: 59 - DNA, 9992 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatggtaccctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgggcgaccaaaggtcgcccgacgcccgggcggcctcagtgagcgagcgagcgcgcagagctagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 60 - DNA, 115 bp, Artificial Sequence; identical to SEQ ID NO: 47
  Recombinant synthesis

SEQ ID NO: 61 - DNA, 235 bp, Artificial Sequence; identical to SEQ ID NO: 15
  Recombinant synthesis
  CMV Enhancer (1)..(235)

SEQ ID NO: 62 - DNA, 278 bp, Artificial Sequence; identical to SEQ ID NO: 48
  Recombinant synthesis
  CBA promoter (1)..(278)

SEQ ID NO: 63 - DNA, 3702 bp, Artificial Sequence; identical to SEQ ID NO: 51
  Recombinant synthesis

SEQ ID NO: 64 - DNA, 121 bp, Artificial Sequence; identical to SEQ ID NO: 37
  Recombinant synthesis

SEQ ID NO: 65 - DNA, 9702 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgggcgaccaaaggtcgcccgacgcccgggcggcctcagtgagcgagcgagcgcgcagagctagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 66 - DNA, 115 bp, Artificial Sequence; identical to SEQ ID NO: 47
  Recombinant synthesis

SEQ ID NO: 67 - DNA, 199 bp, Artificial Sequence; identical to SEQ ID NO: 5
  Recombinant synthesis

SEQ ID NO: 68 - DNA, 3702 bp, Artificial Sequence; identical to SEQ ID NO: 51
  Recombinant synthesis

SEQ ID NO: 69 - DNA, 121 bp, Artificial Sequence; identical to SEQ ID NO: 37
  Recombinant synthesis

SEQ ID NO: 70 - protein, 2273 aa, Homo sapiens [sequence omitted]

SEQ ID NO: 71 - DNA, 16 bp, Artificial Sequence
  primer
gattacaaagatgacg

SEQ ID NO: 72 - DNA, 19 bp, Artificial Sequence
  primer
gcaattcagtcgataacta

SEQ ID NO: 73 - DNA, 20 bp, Artificial Sequence
  primer
accttgatcaggtgtttcca

SEQ ID NO: 74 - DNA, 17 bp, Artificial Sequence
  primer
acagaaggagtcttcca

SEQ ID NO: 75 - DNA, 37 bp, Artificial Sequence
  primer
  FW primer (1)..(18)
  RV primer (19)..(37)
ccactcctaagcgtcctccagggattgttcacattgc